(19) United States
(12) Patent Application Publication
(10) Pub. No.: US 2026/0021178 A1
(43) Pub. Date: Jan. 22, 2026

(54) BCMA-TARGETING AGENT, AND COMBINATION THERAPY WITH A GAMMA SECRETASE INHIBITOR

(71) Applicant: Novartis AG, Basel (CH)

(72) Inventors: Michael Daley, West Springfield, MA (US); Haihui Lu, Winchester, MA (US)

(21) Appl. No.: 19/348,293

(22) Filed: Oct. 2, 2025

Related U.S. Application Data
(63) Continuation of application No. 16/608,515.
(60) Provisional application No. 62/491,587, filed on Apr. 28, 2017.
(60) Provisional application No. 62/593,643, filed on Dec. 1, 2017.

(51) Int. Cl. A61K 39/395; A61K 39/00; A61P 35/00; C07K 16/28

(52) U.S. Cl. CPC A61K 39/39558; A61P 35/00; C07K 16/2809; C07K 16/2878; A61K 2039/505; C07K 2317/31; C07K 2317/55; C07K 2317/622

(57) ABSTRACT

The invention provides compositions and methods for treating diseases associated with expression of BCMA. The invention also relates to a method of administering a BCMA-targeting agent which is an anti-BCMA antibody molecule or a recombinant non-antibody protein that binds to BCMA, and a gamma secretase inhibitor.

### RELATED APPLICATIONS

This application is a continuation of U.S. application Ser. No. 16/608,515, filed Oct. 25, 2019, which is a U.S. National Stage Application under 35 U.S.C. § 371 of International Application No. PCT/US2018/029955, filed Apr. 27, 2018, which claims priority to U.S. Ser. No. 62/491,587 filed Apr. 28, 2017, and U.S. Ser. No. 62/593,643 filed Dec. 1, 2017, the contents of each of which are incorporated herein by reference in their entirety.

### SEQUENCE LISTING

The instant application contains a Sequence Listing which has been submitted electronically in XML format compliant with WIPO Standard ST.26 and is hereby incorporated by reference in its entirety. Said XML copy, created on Oct. 1, 2025, is named N2067-712620_SL.xml and is 1,270,803 bytes in size.

### FIELD OF THE INVENTION

The present invention relates generally to the use of an agent targeting B-cell maturation antigen protein (BCMA), optionally in combination with a gamma secretase inhibitor, to treat a disease associated with the expression of BCMA.

### BACKGROUND OF THE INVENTION

Gamma secretase is a multi-subunit protease complex that cleaves single-pass transmembrane proteins at residues within the transmembrane domain. The gamma secretase complex comprises four subunits: presenilin, nicastrin, gamma-secretase subunit APH-1, and gamma-secretase subunit PEN-2. Another protein, CD147, has been reported as a non-essential regulator of the gamma secretase complex. Exemplary gamma secretase substrates include amyloid precursor protein, Notch, ErbB4, E-cadherin, N-cadherin, and CD44 (Haapasalo et al., J Alzheimers Dis. 2011; 25(1):3-28). Recently, B-cell maturation antigen (BCMA) was identified as another substrate of gamma secretase (Laurent et al., Nat Commun. 2015 Jun. 11; 6:7333).

BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma.

Given the ongoing need for improved strategies for targeting diseases such as cancer, new compositions and methods for improving therapeutic agents that target BCMA, e.g., an anti-BCMA antibody molecule or a recombinant non-antibody protein that binds to BCMA, are highly desirable.

### SUMMARY OF THE INVENTION

The disclosure features, at least in part, a method of treating a disease or disorder associated with expression of B-cell maturation antigen (BCMA, also known as TNFRSF17, BCM, or CD269). In certain embodiments, the disorder is a cancer, e.g., a hematological cancer. In some embodiments, the method comprises administering to a subject a BCMA-targeting agent in combination with a gamma secretase inhibitor (GSI). In some embodiments, the BCMA-targeting agent is an anti-BCMA antibody molecule or a recombinant non-antibody protein that binds to BCMA. In some embodiments, the combination maintains or has better clinical effectiveness as compared to either therapy alone. The disclosure additionally features a BCMA-targeting agent, e.g., as a monotherapy or in a combination therapy.

In some embodiments, the disclosure provides a method of treating a subject having a disease associated with expression of B-cell maturation antigen (BCMA) comprising administering to the subject an effective amount of:
(i) a BCMA-targeting agent which is an anti-BCMA antibody molecule or a recombinant non-antibody protein that binds to BCMA, and
(ii) a gamma secretase inhibitor (GSI).

In some embodiments, the BCMA-targeting agent is not a chimeric antigen receptor (CAR) therapy.

In some embodiments, the disclosure provides a method of treating a subject having a disease associated with expression of B-cell maturation antigen (BCMA) comprising administering to the subject an effective amount of:
(i) a BCMA-targeting agent which is an anti-BCMA antibody molecule or a recombinant non-antibody protein that binds to BCMA, and
(ii) a gamma secretase inhibitor (GSI), wherein:
(a) the anti-BCMA antibody molecule is a multispecific antibody molecule,
(b) the GSI does not reduce gamma secretase-mediated cleavage of Notch, or reduces gamma secretase-mediated cleavage of Notch less efficiently, e.g., at least 2-fold, 5-fold, 10-fold, 50-fold, or 100-fold less efficiently, than the GSI reduces gamma secretase-mediated cleavage of BCMA,
(c) the GSI reduces gamma secretase-mediated cleavage of BCMA more efficiently, e.g., at least 2-fold, 5-fold, 10-fold, 50-fold, or 100-fold more efficiently, than the GSI reduces gamma secretase-mediated cleavage of another substrate of gamma secretase, e.g., Cadherins, ErbB, or CD44,
(d) the GSI is an antibody molecule that reduces the expression and/or function of gamma secretase,
(e) the GSI is (1) a gene editing system targeted to one or more sites within a gene encoding a subunit of gamma secretase (e.g., presenilin, nicastrin, APH-1, or PEN-2) or a regulatory element thereof; (2) a nucleic acid encoding one or more components of the gene editing system; or (3) a combination thereof,
(f) the GSI is an agent that mediates RNA interference, or
(g) the method comprises a first treatment regimen and a second treatment regimen, wherein the first treatment regimen is performed prior to the second treatment regimen, wherein:
(1) the first treatment regimen comprises administering a first dose of the BCMA-targeting agent, and
(2) the second treatment regimen comprises administering a dose of GSI followed by a second dose of the BCMA-targeting agent,
optionally wherein after the administration of the dose of GSI and prior to the administration of the second dose of the BCMA-targeting agent, the subject shows an increase in cell surface BCMA expression levels and/or a decrease in soluble BCMA levels.

In one aspect, provided herein is a method of treating a subject having a disease associated with expression of B-cell maturation antigen (BCMA) comprising administering to the subject an effective amount of:
(i) a BCMA-targeting agent comprising an anti-BCMA antibody molecule or a BCMA ligand, and
(ii) a gamma secretase inhibitor (GSI), wherein:
the anti-BCMA antibody molecule is a multispecific (e.g., bispecific) antibody molecule that binds to BCMA and a second antigen, wherein the second antigen is:
   (1) an antigen on an immune cell, e.g., a T cell or a NK cell,
   (2) an antigen chosen from CD3 (e.g., CD3 epsilon, CD3 delta, or CD3 gamma), CD16 (e.g., CD16A), CD64, NKG2D, or CD47,
   (3) an antigen on a tumor cell, e.g., an antigen on a multiple myeloma cell, or
   (4) an antigen chosen from Fc receptor-like protein (FCRL), transmembrane activator and CAML interactor (TACI), or MHC class I polypeptide-related sequence A (MICA), or
the BCMA ligand comprises B-cell activating factor (BAFF), a proliferation-inducing ligand (APRIL), or variant thereof.

In one aspect, provided herein is a method of treating a subject having a disease associated with expression of B-cell maturation antigen (BCMA) comprising administering to the subject an effective amount of
(i) a BCMA-targeting agent comprising an anti-BCMA antibody molecule or a BCMA ligand, and
(ii) a gamma secretase inhibitor (GSI), wherein:
the GSI is an antibody molecule that reduces the expression and/or function of gamma secretase, optionally wherein the GSI is an antibody molecule that specifically binds to a subunit of gamma secretase (e.g., presenilin, nicastrin, APH-1, or PEN-2);
the GSI is (1) a gene editing system targeted to one or more sites within a gene encoding a subunit of gamma secretase (e.g., presenilin, nicastrin, APH-1, or PEN-2) or a regulatory element thereof; (2) a nucleic acid encoding one or more components of the gene editing system; or (3) a combination thereof; or
the GSI is an agent that mediates RNA interference, e.g., an siRNA or shRNA specific for a gene encoding a subunit of gamma secretase (e.g., presenilin, nicastrin, APH-1, or PEN-2), or a nucleic acid encoding the siRNA or shRNA.

In one aspect, provided herein is a method of treating a subject having a disease associated with expression of B-cell maturation antigen (BCMA) comprising administering to the subject an effective amount of
(i) a multispecific (e.g., bispecific) antibody molecule that binds to BCMA and CD3, and
(ii) a gamma secretase inhibitor (GSI). In some embodiments, CD3 is chosen from CD3 epsilon, CD3 delta, or CD3 gamma, optionally wherein. In some embodiments, CD3 is CD3 epsilon.

In certain embodiments, the GSI is an agent that reduces the expression and/or function of BCMA.

In one embodiment, the gamma secretase inhibitor (GSI) has one or more (e.g., 1, 2, 3, 4, 5, 6, 7, 8, 9, 10, 11, 12, or all) of the following properties:
(i) the GSI reduces gamma secretase-mediated cleavage of BCMA;
(ii) the GSI, when incubated with BCMA-expressing cells, increases cell surface expression of BCMA, e.g., by at least 2, 4, 6, 8, 10, 15, or 20-fold, e.g., as measured by a method described herein, e.g., a flow cytometry assay, e.g., as measured using methods described in Example 1 with respect to FIG. 2;
(iii) the GSI, when incubated with BCMA-expressing cells, changes conformation and/or posttranslational modification of the extracellular domain of cell surface-expressed BCMA;
(iv) the GSI, when incubated with BCMA-expressing cells, decreases the level of soluble BCMA in the cell supernatant, e.g., by at least 80, 85, 90, 95, 99, or 99.5%, e.g., as measured by a method described herein, e.g., an ELISA assay, e.g., as measured using methods described in Example 1 with respect to Table 12;
(v) the GSI, when administered in vivo, increases cell surface expression of BCMA, e.g., as measured by a method described herein, e.g., a flow cytometry assay;
(vi) the GSI, when administered in vivo, changes conformation and/or posttranslational modification of the extracellular domain of cell surface-expressed BCMA;
(vii) the GSI, when administered in vivo, decreases the level of soluble BCMA in the serum and/or bone marrow, e.g., as measured by a method described herein, e.g., an ELISA assay;
(viii) the GSI is capable of increasing the activity of the BCMA-targeting agent, e.g., an antibody molecule that binds to BCMA, e.g., a BCMA×CD3 bispecific antibody molecule, e.g., by decreasing EC50 of cell killing by at least 70, 75, 80, 85, 90, 95, 99, or 99.5%, e.g., as measured by a method described herein, e.g., a redirected T-cell cytotoxicity (RTCC) killing assay, e.g., as measured using methods described in Example 1 with respect to FIGS. 3-6, 9A or Table 16;
(ix) the GSI is capable of increasing the activity of the BCMA-targeting agent, e.g., an antibody molecule that binds to BCMA, e.g., an anti-BCMA antibody drug conjugate, e.g., by decreasing IC50 of cell killing by at least 80, 85, 90, 95, 99, or 99.5%, e.g., as measured by a method described herein, e.g., an antibody drug conjugate (ADC) killing assay, e.g., as measured using methods described in Example 1 with respect to FIG. 10A or 10B, or Table 16;
(x) the GSI does not reduce gamma secretase-mediated cleavage of Notch, or reduces gamma secretase-mediated cleavage of Notch less efficiently, e.g., at least 2-fold, 5-fold, 10-fold, 50-fold, or 100-fold less efficiently, than the GSI reduces gamma secretase-mediated cleavage of BCMA;
(xi) the GSI reduces gamma secretase-mediated cleavage of BCMA more efficiently, e.g., at least 2-fold, 5-fold, 10-fold, 50-fold, or 100-fold more efficiently, than the GSI reduces gamma secretase-mediated cleavage of another substrate of gamma secretase, e.g., Cadherins, ErbB, or CD44;
(xii) the GSI specifically binds to Presenilin-1, e.g., the GSI binds to Presenilin-1 with higher affinity, e.g., at least 2-fold, 5-fold, 10-fold, 50-fold, or 100-fold higher affinity, than the GSI binds to another subunit of gamma secretase, e.g., nicastrin, anterior pharynx-defective 1, or presenilin enhancer 2; or
(xiii) the GSI exhibits low gastrointestinal toxicity.

In some embodiments, the GSI is chosen from a small molecule, an antibody molecule, an agent that mediates gene editing, or an agent that mediates RNA interference.

In one embodiment, the GSI is a small molecule that reduces the expression and/or function of gamma secretase, e.g., a small-molecule GSI disclosed herein. In one embodiment, the GSI is chosen from LY-450139, PF-5212362, BMS-708163, MK-0752, ELN-318463, BMS-299897, LY-411575, DAPT, BMS-906024, PF-3084014, R04929097, or LY3039478. In one embodiment, the GSI is chosen from PF-5212362, ELN-318463, BMS-906024, or LY3039478. Exemplary GSIs are disclosed in Takebe et al., Pharmacol Ther. 2014 February; 141(2):140-9; and Ran et al., EMBO Mol Med. 2017 July; 9(7):950-966, both of which are incorporated herein by reference in their entirety.

In some embodiments, MK-0752 is administered in combination with docetaxel. In some embodiments, MK-0752 is administered in combination with gemcitabine. In some embodiments, BMS-906024 is administered in combination with chemotherapy.

In one embodiment, the GSI is:

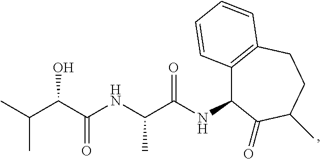 [SMILES: CC1CCc2ccccc2[C@H](NC(=O)[C@H](C)NC(=O)[C@@H](O)C(C)C)C1=O]

or a pharmaceutically acceptable salt thereof.

In one embodiment, the GSI is:

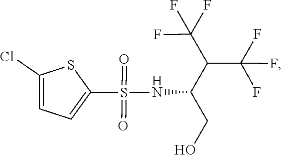 [SMILES: O=S(=O)(N[C@@H](CO)C(C(F)(F)F)C(F)(F)F)c1ccc(Cl)s1]

or a pharmaceutically acceptable salt thereof.

In one embodiment, the GSI is

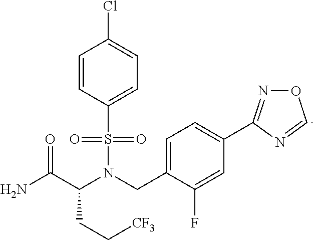 [SMILES: NC(=O)[C@@H](CCC(F)(F)F)N(Cc1ccc(-c2ncon2)cc1F)S(=O)(=O)c1ccc(Cl)cc1]

or a pharmaceutically acceptable salt thereof.

In one embodiment, the GSI is:

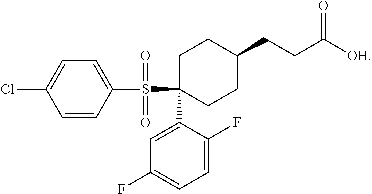 [SMILES: O=C(O)CC[C@H]1CC[C@@](c2cc(F)ccc2F)(S(=O)(=O)c2ccc(Cl)cc2)CC1]

or a pharmaceutically acceptable salt thereof.

In one embodiment, the GSI is:

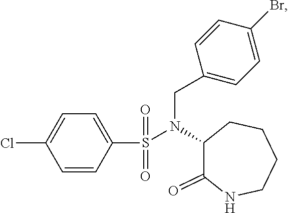 [SMILES: O=C1NCCCC[C@H]1N(Cc1ccc(Br)cc1)S(=O)(=O)c1ccc(Cl)cc1]

or a pharmaceutically acceptable salt thereof.

In one embodiment, the GSI is:

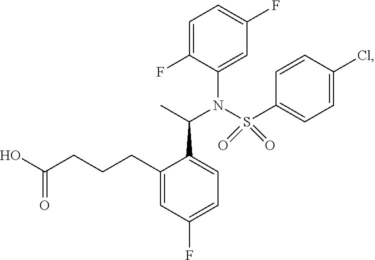 [SMILES: C[C@H](c1ccc(F)cc1CCCC(=O)O)N(c1cc(F)ccc1F)S(=O)(=O)c1ccc(Cl)cc1]

or a pharmaceutically acceptable salt thereof.

In one embodiment, the GSI is:

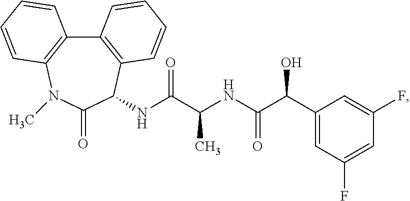 [SMILES: C[C@H](NC(=O)[C@@H](O)c1cc(F)cc(F)c1)C(=O)N[C@@H]1C(=O)N(C)c2ccccc2-c2ccccc21]

or a pharmaceutically acceptable salt thereof.

In one embodiment, the GSI is:

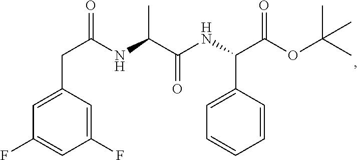 [SMILES: C[C@H](NC(=O)Cc1cc(F)cc(F)c1)C(=O)N[C@H](C(=O)OC(C)(C)C)c1ccccc1]

or a pharmaceutically acceptable salt thereof.

In one embodiment, the GSI is:

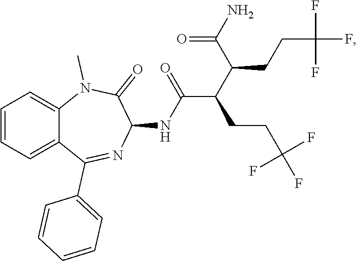 [SMILES: CN1C(=O)[C@@H](NC(=O)[C@H](CCC(F)(F)F)[C@H](CCC(F)(F)F)C(N)=O)N=C(c2ccccc2)c2ccccc21]

or a pharmaceutically acceptable salt thereof.

In one embodiment, the GSI is:

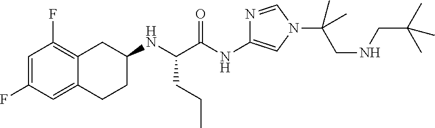 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof.

In one embodiment, the GSI is:

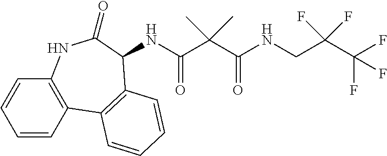 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof.

In one embodiment, the GSI is:

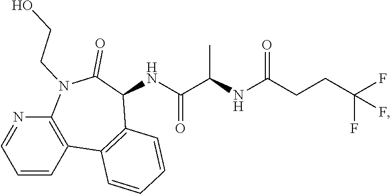 [SMILES: C[C@@H](NC(=O)CCC(F)(F)F)C(=O)N[C@@H]1C(=O)N(CCO)c2ncccc2-c2ccccc21]

or a pharmaceutically acceptable salt thereof.

In one embodiment, the GSI is an antibody molecule that reduces the expression and/or function of gamma secretase, e.g., an antibody-molecule GSI disclosed herein. In one embodiment, the GSI is an antibody molecule that specifically binds to a subunit of gamma secretase (e.g., presenilin, nicastrin, APH-1, or PEN-2).

In one embodiment, the GSI is an agent that mediates gene editing, e.g., a gene editing system disclosed herein. In one embodiment, the GSI is (1) a gene editing system targeted to one or more sites within a gene encoding a subunit of gamma secretase (e.g., presenilin, nicastrin, APH-1, or PEN-2) or a regulatory element thereof; (2) a nucleic acid encoding one or more components of the gene editing system; or (3) a combination thereof. In one embodiment, the gene editing system is chosen from a CRISPR/Cas9 system, a zinc finger nuclease system, a TALEN system, or a meganuclease system.

In one embodiment, the GSI is an agent that mediates RNA interference, e.g., an siRNA or shRNA disclosed herein. In one embodiment, the GSI is an siRNA or shRNA specific for a gene encoding a subunit of gamma secretase (e.g., presenilin, nicastrin, APH-1, or PEN-2), or a nucleic acid encoding the siRNA or shRNA. In one embodiment, the siRNA or shRNA comprises a sequence complementary to a sequence of an mRNA of the gene encoding a subunit of gamma secretase (e.g., presenilin, nicastrin, APH-1, or PEN-2).

In certain embodiments, the BCMA-targeting agent comprises an anti-BCMA antibody molecule. In one embodiment, the anti-BCMA antibody molecule comprises:
a heavy chain variable region (VH) comprising a heavy chain complementarity determining region 1 (VHCDR1), a VHCDR2, and a VHCDR3 of any anti-BCMA heavy chain binding domain amino acid sequence listed in Tables 2, 9, 3, 5, 7, and 1 (or a sequence at least about 85%, 90%, 95%, 99% or more identical thereto, and/or having one, two, three or more substitutions, insertions or deletions, e.g., conserved substitutions), and/or
a light chain variable region (VL) comprising a light chain complementarity determining region 1 (VLCDR1), a VLCDR2, and a VLCDR3 of any anti-BCMA light chain binding domain amino acid sequence listed in Tables 2, 9, 4, 6, 8, and 1 (or a sequence at least about 85%, 90%, 95%, 99% or more identical thereto, and/or having one, two, three or more substitutions, insertions or deletions, e.g., conserved substitutions).

In one embodiment, the anti-BCMA antibody molecule comprises:
a VH comprising a VH of any anti-BCMA heavy chain binding domain amino acid sequence listed in Tables 2, 9, and 1 (or a sequence at least about 85%, 90%, 95%, 99% or more identical thereto, and/or having one, two, three or more substitutions, insertions or deletions, e.g., conserved substitutions), and/or
a VL comprising a VL of any anti-BCMA light chain binding domain amino acid sequence listed in Tables 2, 9, and 1 (or a sequence at least about 85%, 90%, 95%, 99% or more identical thereto, and/or having one, two, three or more substitutions, insertions or deletions, e.g., conserved substitutions).

In one embodiment, the anti-BCMA antibody molecule comprises:
an anti-BCMA heavy chain amino acid sequence listed in Table 1 (or a sequence at least about 85%, 90%, 95%, 99% or more identical thereto, and/or having one, two, three or more substitutions, insertions or deletions, e.g., conserved substitutions), and/or
an anti-BCMA light chain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the anti-BCMA antibody molecule, when bound to BCMA-expressing cells, is capable of inducing antibody-dependent cellular cytotoxicity (ADCC) or complement-dependent cytotoxicity (CDC). In one embodiment, the anti-BCMA antibody molecule comprises an Fc region comprising at least one mutation, e.g., substitution, deletion, or addition, e.g., conserved substitution, that increases the ability of the anti-BCMA antibody molecule to induce ADCC or CDC. In some embodiments, the anti-BCMA antibody molecule comprises an Fc region comprising one or more mutations disclosed herein. In one embodiment, the anti-BCMA antibody molecule comprises an afucosylated Fc region.

In one embodiment, the anti-BCMA antibody molecule is linked, e.g., via a linker, to a drug moiety. In one embodiment, the drug moiety exerts a cytotoxic or cytostatic activity. In one embodiment, the drug moiety is chosen from a maytansinoid, a kinesin-like protein KIF 11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor. In one embodiment, the linker is chosen from a cleavable linker, a non-cleavable linker, a hydrophilic linker, a procharged linker, or a dicarboxylic acid based linker.

In one embodiment, the anti-BCMA antibody molecule is a multispecific antibody molecule. In one embodiment, the multispecific antibody molecule binds to two different epitopes on BCMA. In one embodiment, the multispecific antibody molecule binds to BCMA and a second antigen. In one embodiment, the second antigen is not BCMA. In one embodiment, the second antigen is an antigen on an immune cell, e.g., a T cell or a NK cell. In one embodiment, the second antigen is an antigen on a tumor cell, e.g., an antigen on a multiple myeloma cell. In one embodiment, the second antigen is an antigen chosen from CD3 (e.g., CD3 epsilon, CD3 delta, or CD3 gamma), CD16 (e.g., CD16A), CD64, NKG2D, or CD47. In one embodiment, the second antigen is an antigen chosen from Fc receptor-like protein (FCRL), transmembrane activator and CAML interactor (TACI), or MHC class I polypeptide-related sequence A (MICA). In one embodiment, the multispecific antibody molecule comprises two binding moieties, wherein the first binding moiety binds to BCMA and the second binding moiety binds to CD47. In one embodiment, the second binding moiety that binds to CD47 comprises Signal regulatory protein α (SIRPα) or a fragment thereof.

In one embodiment, the multispecific antibody molecule binds to BCMA, a second antigen, and a third antigen. In one embodiment, the second antigen is not BCMA, the third antigen is not BCMA, and the second antigen is different from the third antigen. In one embodiment, the multispecific antibody molecule binds to BCMA, a second antigen, and a third antigen, wherein:
(i) the second antigen is:
   (a) an antigen on an immune cell, e.g., a T cell or a NK cell, or
   (b) an antigen chosen from CD3 (e.g., CD3 epsilon, CD3 delta, or CD3 gamma), CD16 (e.g., CD16A), CD64, NKG2D, or CD47; and
(ii) the third antigen is:
   (c) an antigen on a tumor cell, e.g., an antigen on a multiple myeloma cell, or
   (d) an antigen chosen from Fc receptor-like protein (FCRL), transmembrane activator and CAML interactor (TACI), or MHC class I polypeptide-related sequence A (MICA).

In one embodiment, the multispecific antibody molecule binds to BCMA, a second antigen, and a third antigen, wherein:
(i) the second antigen is CD3 (e.g., CD3 epsilon, CD3 delta, or CD3 gamma), and
(ii) the third antigen is an antigen on a multiple myeloma cell, optionally an antigen chosen from Fc receptor-like protein (FCRL), transmembrane activator and CAML interactor (TACI), or MHC class I polypeptide-related sequence A (MICA).

In one embodiment, the multispecific antibody molecule comprises a first binding moiety and a second binding moiety. In one embodiment, the first or second binding moiety comprises (i) a first chain comprising a VH, a CH1, and optionally an Fc domain, linked, e.g., via a linker, and (ii) a second chain comprising a VL and a CL, linked, e.g., via a linker. In one embodiment, the first or second binding moiety comprises a single chain Fv region (scFv), optionally wherein the scFv is linked, e.g., via a linker, to an Fc domain. In one embodiment, the first or second binding moiety comprises (i) a first chain comprising a VH, a CH1, an scFv, and optionally an Fc domain, linked, e.g., via a linker, and (ii) a second chain comprising a VL and a CL, linked, e.g., via a linker. In one embodiment, the first or second binding moiety comprises (i) a first chain comprising a first VH, a first CH1, a second VH, a second CH1, and optionally an Fc domain, linked, e.g., via a linker, (ii) a second chain comprising a first VL and a first CL, and (iii) a third chain comprising a second VL and a second CL.

In one embodiment, the multispecific antibody molecule comprises:
a first binding moiety comprising (i) a first chain comprising a VH, a CH1, and a first Fc domain, linked, e.g., via a linker, and (ii) a second chain comprising a VL and a CL, linked, e.g., via a linker; and
a second binding moiety comprising an scFv linked, e.g., via a linker, to a second Fc domain.

In one embodiment, the multispecific antibody molecule comprises:
a first binding moiety comprising a first scFv linked, e.g., via a linker, to a first Fc domain; and
a second binding moiety comprising a second scFv linked, e.g., via a linker, to a second Fc domain.

In one embodiment, the first binding moiety specifically binds to BCMA. In one embodiment, the first binding moiety comprises:
a heavy chain variable region (VH) comprising a heavy chain complementarity determining region 1 (VHCDR1), a VHCDR2, and a VHCDR3 of any anti-BCMA heavy chain binding domain amino acid sequence listed in Tables 2, 9, 3, 5, 7, and 1 (or a sequence at least about 85%, 90%, 95%, 99% or more identical thereto, and/or having one, two, three or more substitutions, insertions or deletions, e.g., conserved substitutions), and/or
a light chain variable region (VL) comprising a light chain complementarity determining region 1 (VLCDR1), a VLCDR2, and a VLCDR3 of any anti-BCMA light chain binding domain amino acid sequence listed in Tables 2, 9, 4, 6, 8, and 1 (or a sequence at least about 85%, 90%, 95%, 99% or more identical thereto, and/or having one, two, three or more substitutions, insertions or deletions, e.g., conserved substitutions).

In one embodiment, the first binding moiety comprises:
a VH comprising a VH of any anti-BCMA heavy chain binding domain amino acid sequence listed in Tables 2, 9, and 1 (or a sequence at least about 85%, 90%, 95%, 99% or more identical thereto, and/or having one, two, three or more substitutions, insertions or deletions, e.g., conserved substitutions), and/or
a VL comprising a VL of any anti-BCMA light chain binding domain amino acid sequence listed in Tables 2, 9, and 1 (or a sequence at least about 85%, 90%, 95%, 99% or more identical thereto, and/or having one, two, three or more substitutions, insertions or deletions, e.g., conserved substitutions).

In one embodiment, the first binding moiety comprises:
an anti-BCMA heavy chain amino acid sequence listed in Table 1 (or a sequence at least about 85%, 90%, 95%, 99% or more identical thereto, and/or having one, two, three or more substitutions, insertions or deletions, e.g., conserved substitutions), and/or
an anti-BCMA light chain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the second binding moiety specifically binds to an antigen on an immune cell, e.g., a T cell or a NK cell. In one embodiment, the second binding moiety specifically binds to an antigen on a tumor cell, e.g., an antigen on a multiple myeloma cell. In one embodiment, the second binding moiety specifically binds to an antigen chosen from CD3 (e.g., CD3 epsilon, CD3 delta, or CD3 gamma), CD16 (e.g., CD16A), CD64, NKG2D, or CD47. In one embodiment, the second binding moiety specifically binds to an antigen chosen from Fc receptor-like protein (FCRL), transmembrane activator and CAML interactor (TACI), or MHC class I polypeptide-related sequence A (MICA).

In one embodiment, the second binding moiety specifically binds to CD3. In one embodiment, the second binding moiety comprises:
a heavy chain variable region (VH) comprising a heavy chain complementarity determining region 1 (VHCDR1), a VHCDR2, and a VHCDR3 of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions), and/or
a light chain variable region (VL) comprising a light chain complementarity determining region 1 (VLCDR1), a VLCDR2, and a VLCDR3 of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the second binding moiety comprises:
a VH comprising a VH of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions), and/or
a VL comprising a VL of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the second binding moiety comprises:
an anti-CD3 heavy chain amino acid sequence listed in Table 1 (or a sequence at least about 85%, 90%, 95%, 99% or more identical thereto, and/or having one, two, three or more substitutions, insertions or deletions, e.g., conserved substitutions), and/or an anti-CD3 light chain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the multispecific antibody molecule comprises:
a first binding moiety comprising (i) a first chain comprising a VH, a CH1, an scFv, and a first Fc domain, linked, e.g., via one or more linkers, and (ii) a second chain comprising a VL and a CL, linked, e.g., via a linker; and
a second binding moiety comprising (iii) a third chain comprising a VH, a CH1, and a second Fc domain, linked, e.g., via a linker, and (iv) a fourth chain comprising a VL and a CL, linked, e.g., via a linker.

In one embodiment, the VH and VL of the first binding moiety specifically bind to BCMA, and the VH and VL of the second binding moiety specifically bind to BCMA.

In one embodiment, the VH of the first binding moiety and/or the VH of the second binding moiety comprises a heavy chain complementarity determining region 1 (VHCDR1), a VHCDR2, and a VHCDR3 of any anti-BCMA heavy chain binding domain amino acid sequence listed in Tables 2, 9, 3, 5, 7, and 1 (or a sequence at least about 85%, 90%, 95%, 99% or more identical thereto, and/or having one, two, three or more substitutions, insertions or deletions, e.g., conserved substitutions), and/or the VL of the first binding moiety and/or the VL of the second binding moiety comprises a light chain complementarity determining region 1 (VLCDR1), a VLCDR2, and a VLCDR3 of any anti-BCMA light chain binding domain amino acid sequence listed in Tables 2, 9, 4, 6, 8, and 1 (or a sequence at least about 85%, 90%, 95%, 99% or more identical thereto, and/or having one, two, three or more substitutions, insertions or deletions, e.g., conserved substitutions).

In one embodiment, the VH of the first binding moiety and/or the VH of the second binding moiety comprises a VH of any anti-BCMA heavy chain binding domain amino acid sequence listed in Tables 2, 9, and 1 (or a sequence at least about 85%, 90%, 95%, 99% or more identical thereto, and/or having one, two, three or more substitutions, insertions or deletions, e.g., conserved substitutions), and/or the VL of the first binding moiety and/or the VL of the second binding moiety comprises a VL of any anti-BCMA light chain binding domain amino acid sequence listed in Tables 2, 9, and 1 (or a sequence at least about 85%, 90%, 95%, 99% or more identical thereto, and/or having one, two, three or more substitutions, insertions or deletions, e.g., conserved substitutions).

In one embodiment, the scFv of the first binding moiety specifically binds to an antigen on an immune cell, e.g., a T cell or a NK cell. In one embodiment, the scFv of the first binding moiety specifically binds to an antigen on a tumor cell, e.g., an antigen on a multiple myeloma cell. In one embodiment, the scFv of the first binding moiety specifically binds to an antigen chosen from CD3, CD16 (e.g., CD16A), CD64, NKG2D, or CD47. In one embodiment, the scFv of the first binding moiety specifically binds to an antigen chosen from Fc receptor-like protein (FCRL), transmembrane activator and CAML interactor (TACI), or MHC class I polypeptide-related sequence A (MICA).

In one embodiment, the scFv of the first binding moiety specifically binds to CD3. In one embodiment, the scFv of the first binding moiety comprises:
a heavy chain variable region (VH) comprising a heavy chain complementarity determining region 1 (VHCDR1), a VHCDR2, and a VHCDR3 of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions), and/or
a light chain variable region (VL) comprising a light chain complementarity determining region 1 (VLCDR1), a VLCDR2, and a VLCDR3 of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the scFv of the first binding moiety comprises:
a VH comprising a VH of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions), and/or
a VL comprising a VL of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the multispecific antibody molecule comprises:
a first binding moiety comprising (i) a first chain comprising a first VH, a first CH1, a second VH, a second CH1, and a first Fc domain, linked, e.g., via one or more linkers, (ii) a second chain comprising a first VL and a first CL, linked, e.g., via a linker, and (iii) a third chain comprising a second VL and a second CL, linked, e.g., via a linker; and
a second binding moiety comprising (iv) a fourth chain comprising a VH, a CH1, and a second Fc domain, linked, e.g., via a linker, and (v) a fifth chain comprising a VL and a CL, linked, e.g., via a linker.

In one embodiment, the first VH and the first VL of the first binding moiety specifically bind to BCMA, and the VH and VL of the second binding moiety specifically bind to BCMA.

In one embodiment, the first VH of the first binding moiety and/or the VH of the second binding moiety comprises a heavy chain complementarity determining region 1 (VHCDR1), a VHCDR2, and a VHCDR3 of any anti-BCMA heavy chain binding domain amino acid sequence listed in Tables 2, 3, 5, 7, and 1 (or a sequence at least about 85%, 90%, 95%, 99% or more identical thereto, and/or having one, two, three or more substitutions, insertions or deletions, e.g., conserved substitutions), and/or the first VL of the first binding moiety and/or the VL of the second binding moiety comprises a light chain complementarity determining region 1 (VLCDR1), a VLCDR2, and a VLCDR3 of any anti-BCMA light chain binding domain amino acid sequence listed in Tables 2, 4, 6, 8, and 1 (or a sequence at least about 85%, 90%, 95%, 99% or more identical thereto, and/or having one, two, three or more substitutions, insertions or deletions, e.g., conserved substitutions).

In one embodiment, the first VH of the first binding moiety and/or the VH of the second binding moiety comprises a VH of any anti-BCMA heavy chain binding domain amino acid sequence listed in Tables 2 and 1 (or a sequence at least about 85%, 90%, 95%, 99% or more identical thereto, and/or having one, two, three or more substitutions, insertions or deletions, e.g., conserved substitutions), and/or the first VL of the first binding moiety and/or the VL of the second binding moiety comprises a VL of any anti-BCMA light chain binding domain amino acid sequence listed in Tables 2 and 1 (or a sequence at least about 85%, 90%, 95%, 99% or more identical thereto, and/or having one, two, three or more substitutions, insertions or deletions, e.g., conserved substitutions).

In one embodiment, the second VH and the second VL of the first binding moiety specifically bind to an antigen on an immune cell, e.g., a T cell or a NK cell. In one embodiment, the second VH and the second VL of the first binding moiety specifically bind to an antigen on a tumor cell, e.g., an antigen on a multiple myeloma cell. In one embodiment, the second VH and the second VL of the first binding moiety specifically bind to an antigen chosen from CD3, CD16 (e.g., CD16A), CD64, NKG2D, or CD47. In one embodiment, the second VH and the second VL of the first binding moiety specifically bind to an antigen chosen from Fc receptor-like protein (FCRL), transmembrane activator and CAML interactor (TACI), or MHC class I polypeptide-related sequence A (MICA).

In one embodiment, the second VH and the second VL of the first binding moiety specifically bind to CD3 (e.g., CD3 epsilon, CD3 delta, or CD3 gamma).

In one embodiment, the second VH of the first binding moiety comprises a heavy chain complementarity determining region 1 (VHCDR1), a VHCDR2, and a VHCDR3 of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions), and/or the second VL of the first binding moiety comprises a light chain complementarity determining region 1 (VLCDR1), a VLCDR2, and a VLCDR3 of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the second VH of the first binding moiety comprises a VH of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions), and/or the second VL of the first binding moiety comprises a VL of any anti-CD3 binding domain amino acid sequence listed in Table 1 (or a sequence at least about 85%, 90%, 95%, 99% or more identical thereto, and/or having one, two, three or more substitutions, insertions or deletions, e.g., conserved substitutions).

In one embodiment, the first and second Fc domains are different. In one embodiment, wherein the first and second Fc domains each comprises one or more mutations that favor heterodimer formation, e.g., formation of a heterodimer between the first and second Fc domains, over homodimer formation, e.g., formation of a homodimer between two of the first Fc domains or a homodimer between two of the second Fc domains.

In one embodiment, the first and second Fc domains comprise one or more amino acid mutations that reduce the interaction of the first and second Fc domains with an Fcγ receptor, e.g., reduce the interaction by at least 2-fold, 5-fold, 10-fold, 50-fold, 100-fold, or 1000-fold. In one embodiment, the first and second Fc domains comprise one or more amino acid mutations that reduce the ability of the multispecific antibody molecule to induce antibody-dependent cell-mediated cytotoxicity (ADCC) or complement-dependent cytotoxicity (CDC).

In one embodiment, the multispecific antibody molecule is linked, e.g., via a linker, to a drug moiety. In one embodiment, the drug moiety exerts a cytotoxic or cytostatic activity. In one embodiment, the drug moiety is chosen from a maytansinoid, a kinesin-like protein KIF 11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor. In one embodiment, the linker is chosen from a cleavable linker, a non-cleavable linker, a hydrophilic linker, a procharged linker, or a dicarboxylic acid based linker.

In one embodiment, the BCMA-targeting agent is a recombinant non-antibody protein that binds to BCMA. In one embodiment, the recombinant non-antibody protein that binds to BCMA comprises a BCMA ligand, e.g., B-cell activating factor (BAFF), a proliferation-inducing ligand (APRIL), or fragment thereof.

In one embodiment, the BCMA-targeting agent comprises a BCMA ligand. In one embodiment, the BCMA ligand comprises B-cell activating factor (BAFF), a proliferation-inducing ligand (APRIL), or variant thereof.

In one embodiment, the recombinant non-antibody protein (e.g., the BCMA ligand) is linked, e.g., via a linker, to a drug moiety. In one embodiment, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor. In one embodiment, the linker is chosen from a cleavable linker, a non-cleavable linker, a hydrophilic linker, a procharged linker, or a dicarboxylic acid based linker.

In one embodiment, the recombinant non-antibody protein (e.g., the BCMA ligand) is linked, e.g., via a linker, to a binding moiety that binds:
(1) an antigen on an immune cell, e.g., a T cell or a NK cell,
(2) an antigen chosen from CD3 (e.g., CD3 epsilon, CD3 delta, or CD3 gamma), CD16 (e.g., CD16A), CD64, NKG2D, or CD47,
(3) an antigen on a tumor cell, e.g., an antigen on a multiple myeloma cell, or
(4) an antigen chosen from Fc receptor-like protein (FCRL), transmembrane activator and CAML interactor (TACI), or MHC class I polypeptide-related sequence A (MICA).

In one embodiment, the disease associated with expression of BCMA is:
(i) a cancer or malignancy, or a precancerous condition chosen from one or more of a myelodysplasia, a myelodysplastic syndrome or a preleukemia, or
(ii) a non-cancer related indication associated with expression of BCMA.

In one embodiment, the disease is chosen from acute leukemia, B-cell acute lymphoid leukemia (BALL), T-cell acute lymphoid leukemia (TALL), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or large cell-follicular lymphoma, a malignant lymphoproliferative condition, mucosa associated lymphoid tissue (MALT) lymphoma, mantle cell lymphoma, marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome)), prostate cancer (e.g., castrate-resistant or therapy-resistant prostate cancer, or metastatic prostate cancer), pancreatic cancer, or lung cancer.

In one embodiment, the disease is a hematologic cancer. In one embodiment, the disease is multiple myeloma, e.g., CD19-negative multiple myeloma.

In one embodiment, the BCMA-targeting agent and the GSI are administered simultaneously or sequentially. In one embodiment, the BCMA-targeting agent is administered prior to the administration of the GSI. In one embodiment, the GSI is administered prior to the administration of the BCMA-targeting agent. In one embodiment, the BCMA-targeting agent and the GSI are administered simultaneously.

In one embodiment, the GSI is administered prior to the administration of the BCMA-targeting agent (e.g., GSI is administered 1, 2, 3, 4, or 5 days prior to the administration of the BCMA-targeting agent), optionally wherein after the administration of the GSI and prior to the administration of the BCMA-targeting agent, the subject shows an increase in cell surface BCMA expression levels and/or a decrease in soluble BCMA levels.

In one embodiment, the method comprises a first treatment regimen and a second treatment regimen, wherein the first treatment regimen is performed prior to the second treatment regimen, wherein:
(i) the first treatment regimen comprises administering a first dose of the BCMA-targeting agent, and
(ii) the second treatment regimen comprises administering a dose of GSI followed by a second dose of the BCMA-targeting agent,
optionally wherein after the administration of the dose of GSI and prior to the administration of the second dose of the BCMA-targeting agent, the subject shows an increase in cell surface BCMA expression levels and/or a decrease in soluble BCMA levels.

In one embodiment, after the administration of GSI, the cell surface BCMA expression level in the subject is increased by at least 1-fold, 2-fold, 3-fold, 4-fold, 5-fold, 10-fold, 20-fold, 50-fold, or 100-fold.

In one embodiment, after the administration of GSI, the soluble BCMA level in the subject is decreased by at least 5%, 10%, 20%, 30%, 40%, 50%, 60%, 70%, 80%, 90%, 95%, or 99%.

In one embodiment, the BCMA-targeting agent and the GSI are administered in combination with a third therapeutic agent or procedure, optimally wherein the third therapeutic agent or procedure is chosen from one or more of chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule (e.g., an inhibitor of a checkpoint inhibitor), or a vaccine.

In one embodiment, the third therapeutic agent or procedure is chosen from:
(i) Dexamethasone;
(ii) a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224;
(iii) a PD-L1 inhibitor, optionally wherein the PD-L1 inhibitor is selected from the group consisting of FAZ053, Atezolizumab, Avelumab, Durvalumab, and BMS-936559;
(iv) a CTLA-4 inhibitor, optionally wherein the CTLA-4 inhibitor is Ipilimumab or Tremelimumab;
(v) a TIM-3 inhibitor, optionally wherein the TIM-3 inhibitor is MGB453 or TSR-022;
(vi) a LAG-3 inhibitor, optionally wherein the LAG-3 inhibitor is selected from the group consisting of LAG525, BMS-986016, and TSR-033;
(vii) an mTOR inhibitor, optionally wherein the mTOR inhibitor is RAD001 or rapamycin; or
(viii) an agent chosen from HetIL-15, an anti-TGFβ antibody, an anti-CD47 antibody, an IDO inhibitor, a STING agonist, a TLR agonist, an immunomodulatory drug (IMiD) (e.g., Thalidomide, Lenalidomide, or Pomalidomide), a proteasome inhibitor (e.g., Bortezomib), or an ADCC-competent antibody (e.g., Daratumumab or Elotuzumab).

In one aspect, provided herein a composition comprising a B-cell maturation antigen (BCMA)-targeting agent and a gamma secretase inhibitor (GSI), wherein the BCMA-targeting agent is an anti-BCMA antibody molecule or a recombinant non-antibody protein that binds to BCMA. In one embodiment, the BCMA-targeting agent is not a chimeric antigen receptor (CAR) therapy.

In one embodiment, provided herein is a composition comprising a B-cell maturation antigen (BCMA)-targeting agent and a gamma secretase inhibitor (GSI), wherein the BCMA-targeting agent is an anti-BCMA antibody molecule or a recombinant non-antibody protein that binds to BCMA, wherein:
(a) the anti-BCMA antibody molecule is a multispecific antibody molecule,
(b) the GSI does not reduce gamma secretase-mediated cleavage of Notch, or reduces gamma secretase-mediated cleavage of Notch less efficiently, e.g., at least 2-fold, 5-fold, 10-fold, 50-fold, or 100-fold less efficiently, than the GSI reduces gamma secretase-mediated cleavage of BCMA,
(c) the GSI reduces gamma secretase-mediated cleavage of BCMA more efficiently, e.g., at least 2-fold, 5-fold, 10-fold, 50-fold, or 100-fold more efficiently, than the GSI reduces gamma secretase-mediated cleavage of another substrate of gamma secretase, e.g., Cadherins, ErbB, or CD44,
(d) the GSI is an antibody molecule that reduces the expression and/or function of gamma secretase,
(e) the GSI is (1) a gene editing system targeted to one or more sites within a gene encoding a subunit of gamma secretase (e.g., presenilin, nicastrin, APH-1, or PEN-2) or a regulatory element thereof; (2) a nucleic acid encoding one or more components of the gene editing system; or (3) a combination thereof, or
(f) the GSI is an agent that mediates RNA interference.

In one embodiment, provided herein is a composition comprising a B-cell maturation antigen (BCMA)-targeting agent and a gamma secretase inhibitor (GSI), wherein the BCMA-targeting agent comprises an anti-BCMA antibody molecule or a BCMA ligand, wherein:
the anti-BCMA antibody molecule is a multispecific (e.g., bispecific) antibody molecule that binds to BCMA and a second antigen, wherein the second antigen is:
   (1) an antigen on an immune cell, e.g., a T cell or a NK cell,
   (2) an antigen chosen from CD3 (e.g., CD3 epsilon, CD3 delta, or CD3 gamma), CD16 (e.g., CD16A), CD64, NKG2D, or CD47,
   (3) an antigen on a tumor cell, e.g., an antigen on a multiple myeloma cell, or
   (4) an antigen chosen from Fc receptor-like protein (FCRL), transmembrane activator and CAML interactor (TACI), or MHC class I polypeptide-related sequence A (MICA), or
the BCMA ligand comprises B-cell activating factor (BAFF), a proliferation-inducing ligand (APRIL), or variant thereof.

In one embodiment, the BCMA-targeting agent is an anti-BCMA antibody molecule. In one embodiment, the anti-BCMA antibody molecule is a monospecific antibody molecule. In one embodiment, the anti-BCMA antibody molecule is a multispecific antibody molecule, e.g., a BCMA×CD3 multispecific antibody molecule. In one embodiment, the BCMA-targeting agent is a recombinant non-antibody protein that binds to BCMA, e.g., a recombinant non-antibody protein comprising a BCMA ligand, e.g., B-cell activating factor (BAFF), a proliferation-inducing ligand (APRIL), or fragment thereof. In one embodiment, the recombinant non-antibody protein is linked, e.g., via a linker, to a drug moiety.

In one embodiment, the BCMA-targeting agent and the GSI are present in a single dose form, or as two or more dose forms.

In one embodiment, provided herein is a composition comprising a B-cell maturation antigen (BCMA)-targeting agent and a gamma secretase inhibitor (GSI) for use as a medicament, wherein the BCMA-targeting agent is an anti-BCMA antibody molecule or a recombinant non-antibody protein that binds to BCMA.

In one embodiment, provided herein is a composition comprising a B-cell maturation antigen (BCMA)-targeting agent and a gamma secretase inhibitor (GSI) for use in the treatment of a disease associated with expression of BCMA, wherein the BCMA-targeting agent is an anti-BCMA antibody molecule or a recombinant non-antibody protein that binds to BCMA.

In one aspect, provided herein is a kit comprising a B-cell maturation antigen (BCMA)-targeting agent and a gamma secretase inhibitor (GSI), wherein the BCMA-targeting agent is an anti-BCMA antibody molecule or a recombinant non-antibody protein that binds to BCMA.

In one embodiment, provided herein is a kit comprising a B-cell maturation antigen (BCMA)-targeting agent and a gamma secretase inhibitor (GSI), wherein the BCMA-targeting agent is an anti-BCMA antibody molecule or a recombinant non-antibody protein that binds to BCMA, wherein:
(a) the anti-BCMA antibody molecule is a multispecific antibody molecule,
(b) the GSI does not reduce gamma secretase-mediated cleavage of Notch, or reduces gamma secretase-mediated cleavage of Notch less efficiently, e.g., at least 2-fold, 5-fold, 10-fold, 50-fold, or 100-fold less efficiently, than the GSI reduces gamma secretase-mediated cleavage of BCMA,
(c) the GSI reduces gamma secretase-mediated cleavage of BCMA more efficiently, e.g., at least 2-fold, 5-fold, 10-fold, 50-fold, or 100-fold more efficiently, than the GSI reduces gamma secretase-mediated cleavage of another substrate of gamma secretase, e.g., Cadherins, ErbB, or CD44,
(d) the GSI is an antibody molecule that reduces the expression and/or function of gamma secretase,
(e) the GSI is (1) a gene editing system targeted to one or more sites within a gene encoding a subunit of gamma secretase (e.g., presenilin, nicastrin, APH-1, or PEN-2) or a regulatory element thereof; (2) a nucleic acid encoding one or more components of the gene editing system; or (3) a combination thereof, or
(f) the GSI is an agent that mediates RNA interference.

In one embodiment, provided herein is a kit comprising a B-cell maturation antigen (BCMA)-targeting agent and a gamma secretase inhibitor (GSI), wherein the BCMA-targeting agent comprises an anti-BCMA antibody molecule or a BCMA ligand, wherein:
the anti-BCMA antibody molecule is a multispecific (e.g., bispecific) antibody molecule that binds to BCMA and a second antigen, wherein the second antigen is:
   (1) an antigen on an immune cell, e.g., a T cell or a NK cell,
   (2) an antigen chosen from CD3 (e.g., CD3 epsilon, CD3 delta, or CD3 gamma), CD16 (e.g., CD16A), CD64, NKG2D, or CD47,
   (3) an antigen on a tumor cell, e.g., an antigen on a multiple myeloma cell, or
   (4) an antigen chosen from Fc receptor-like protein (FCRL), transmembrane activator and CAML interactor (TACI), or MHC class I polypeptide-related sequence A (MICA), or
the BCMA ligand comprises B-cell activating factor (BAFF), a proliferation-inducing ligand (APRIL), or variant thereof.

The materials, methods, and examples are illustrative only and not intended to be limiting.

Headings, sub-headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIGS. 1A-ID are schematics showing exemplary BCMA×CD3 multispecific antibodies. In FIG. 1A, the first arm of the multispecific antibody comprises an anti-BCMA Fab and a first Fc domain. The second arm of the multispecific antibody comprises an anti-CD3 scFv and a second Fc domain. In FIG. 1B, the multispecific antibody comprises an anti-BCMA scFv linked to a first Fc domain, and an anti-CD3 scFv linked to a second Fc domain. In FIG. 1C, the first arm of the multispecific antibody comprises a first anti-BCMA Fab, an anti-CD3 scFv, and a first Fc domain. The second arm of the multispecific antibody comprises a second anti-BCMA Fab and a second Fc domain. The first and second anti-BCMA Fabs can be the same or different. In FIG. 1D, the first arm of the multispecific antibody comprises a first anti-BCMA Fab, an anti-CD3 Fab, and a first Fc domain. The second arm of the multispecific antibody comprises a second anti-BCMA Fab and a second Fc domain. The first and second anti-BCMA Fabs can be the same or different.

FIG. 2 is a set of histogram plots showing the staining of BCMA-expressing human cancer cell lines, NCI-H929, MM1S, and U266B1, using a PE conjugated anti-BCMA antibody. The cell lines were untreated, or treated with 1.0 μM DAPT or 0.1 μM LY-411,575 before subjected to a flow cytometry analysis.

FIG. 3 is a series of graphs showing killing of BCMA-expressing cell lines by T cells mediated by BCMA×CD3 bispecific antibodies ER26, BU76, BQ76, and EE 11, as measured in a redirected T-cell cytotoxicity (RTCC) killing assay. The assay was conducted in the presence or absence of the gamma secretase inhibitor (GSI) DAPT or LY-411,575. % RTCC target cell killing is plotted against a titration of antibody concentrations tested. A gH×CD3 bispecific antibody was used as a control in the assay.

FIG. 4 is a series of bar graphs showing EC50 values as measured in the RTCC killing assay shown in FIG. 3.

FIG. 5 is similar to FIG. 3, showing killing of a BCMA-expressing cell line by T cells mediated by a BCMA×CD3 bispecific antibody EM90, in the absence or presence of the GSI LY-411,575, as measured in a redirected T-cell cytotoxicity (RTCC) killing assay. % RTCC target cell killing is plotted against a titration of antibody concentrations tested. A gH×CD3 bispecific antibody was used as a control in the assay.

FIG. 6 is a bar graph showing EC50 values as measured in the RTCC killing assay shown in FIG. 5.

FIG. 7 is a panel of graphs showing the viability of BCMA-expressing cells (NCI-H929^{[High]}, MM1S^{[Med]}, U266B1^{[low]}) following 72-hour treatment with various gamma secretase inhibitors.

FIG. 8 is a panel of graphs comparing soluble/shed BCMA (sBCMA) levels to membrane-bound BCMA (mBCMA) levels in MM1S cell cultures treated with various gamma secretase inhibitors for 72 hours.

FIG. 9A is a graph showing killing of a BCMA-expressing cell line KMS11 by T cells mediated by a BCMA×CD3 bispecific antibody FP31, in the absence or presence of GSI Semangacestat, Nirogacestat, or Begacestat, as measured in a redirected T-cell cytotoxicity (RTCC) killing assay. RTCC target cell killing is plotted against a titration of antibody concentrations tested. A RSV×CD3 bispecific antibody was used as a negative antibody control (FIGS. 9B and 9D). A BCMA negative cell line NALM6 was used as a negative cell line control (FIGS. 9C and 9D).

FIGS. 10A and 10B are a pair of graphs showing killing of BCMA-expressing cell lines MM1S and U266B1 by anti-BCMA J6M0 antibody drug conjugate, in the absence or presence of a GSI LY-411,575. % Cell Death is plotted against a titration of antibody concentrations tested. An anti-CD19 antibody drug conjugate was used as a negative antibody control. A BCMA negative, CD19 positive cell line NALM6 was used as a negative cell line control (FIG. 0C).

### DETAILED DESCRIPTION

Membrane-bound BCMA is cleaved by gamma secretase. Without wishing to be bound by theory, BCMA shedding may create challenges for therapeutic agents that target BCMA. Some of the challenges include the following. First, BCMA shedding may decrease surface BCMA expression on tumor cells, reducing target binding sites for BCMA-targeting therapeutic agents. Second, BCMA shedding may generate a soluble BCMA sink that binds to BCMA-targeting therapeutic agents. Third, soluble BCMA molecules may also sequester circulating BCMA ligands, e.g., B-cell activating factor (BAFF) and a proliferation-inducing ligand (APRIL), and prevent them from stimulating BCMA expressed on the surface of B cells and plasma cells, thereby leading to deficient humoral immune responses in patients.

Accordingly, the present invention provides, at least in part, a method of treating a subject having a disease associated with BCMA expression, comprising administering to the subject an effective amount of a BCMA-targeting agent and a gamma secretase inhibitor (GSI). In one embodiment, the BCMA-targeting agent is an anti-BCMA antibody molecule or a recombinant non-antibody protein that binds to BCMA.

In one embodiment, the BCMA-targeting agent is an anti-BCMA antibody molecule that, when bound to BCMA-expressing cells, e.g., BCMA-expressing tumor cells, can induce antibody-dependent cellular cytotoxicity (ADCC) or complement-dependent cytotoxicity (CDC) of BCMA-expressing cells, e.g., BCMA-expressing tumor cells. In one embodiment, the anti-BCMA antibody molecule is linked, e.g., via a linker, to a drug moiety, e.g., a drug moiety that exerts a cytotoxic or cytostatic activity. In one embodiment, the anti-BCMA antibody molecule is a multispecific antibody molecule, e.g., a multispecific antibody molecule comprising a first binding moiety that specifically binds to BCMA and a second binding moiety that specifically binds to an antigen on an immune effector cell, e.g., a BCMA×CD3, BCMA×CD16 (e.g., CD16A), BCMA×CD64, or BCMA×NKG2D multispecific antibody molecule. In one embodiment, the BCMA-targeting agent comprises a BCMA ligand, e.g., B-cell activating factor (BAFF), a proliferation-inducing ligand (APRIL), or fragment thereof, optionally linked, e.g., via a linker, to a drug moiety, e.g., a drug moiety that exerts a cytotoxic or cytostatic activity. In one embodiment, the disease associated with expression of BCMA is a hematologic cancer, e.g., multiple myeloma. In one embodiment, the BCMA-targeting agent and the GSI are administered simultaneously or sequentially. The present invention also provides a composition or kit comprising a BCMA-targeting agent and a GSI.

As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells and plasma cells. Its ligands include B-cell activating factor (BAFF) and a proliferation-inducing ligand (APRIL). The protein BCMA is encoded by the gene TNFRSF17. Exemplary BCMA sequences are available at the Uniprot database under accession number Q02223.

As used herein, the term “BAFF” refers to B-cell activating factor, also known as Tumor necrosis factor ligand superfamily member 13B, B lymphocyte stimulator (BLyS), dendritic cell-derived TNF-like molecule, TNF- and APOL-related leukocyte expressed ligand 1 (TALL-1), and CD257. The protein BAFF is encoded by the gene TNFSF13B. Exemplary BAFF sequences are available at the Uniprot database under accession number Q9Y275.

As used herein, the term “APRIL” refers to a proliferation-inducing ligand, also known as Tumor necrosis factor ligand superfamily member 13, TNF- and APOL-related leukocyte expressed ligand 2 (TALL-2), TNF-related death ligand 1 (TRDL-1), and CD256. The protein APRIL is encoded by the gene TNFSF13. Exemplary APRIL sequences are available at the Uniprot database under accession number 075888.

As used herein, the term “gamma secretase” refers to any protein or protein complex that exhibits gamma secretase activities including binding to a substrate having a gamma secretase cleavage sequence, and catalyzing the cleavage of the gamma secretase cleavage sequence, at a gamma secretase cleavage site, to produce substrate cleavage products. In one embodiment, gamma secretase is a protein complex comprising one or more of the following subunits: presenilin, nicastrin, gamma-secretase subunit APH-1, and gamma-secretase subunit PEN-2.

As used herein, the term “gamma secretase inhibitor” or “GSI” refers to any molecule capable of inhibiting or reducing expression and/or function of gamma secretase. In certain embodiment, the GSI reduces expression and/or function of a subunit of gamma secretase (e.g., presenilin, nicastrin, APH-1, or PEN-2). Any form of a “gamma secretase inhibitor” such as a salt, a co-crystal, a crystalline form, a pro-drug, etc., is included within this term.

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a some embodiments, the percent identity between two amino acid sequences is determined using the Needleman and Wunsch ((1970) J Mol. Biol. 48:444-453) algorithm which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See ncbi.nlm.nih.gov.

It is understood that the molecules of the invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function” or “immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effective amount” or “therapeutically effective amount” are used interchangeably herein, and refer to an amount of a compound, formulation, material, or composition, as described herein effective to achieve a particular biological result.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The phrase “disease associated with expression of BCMA” includes, but is not limited to, a disease associated with a cell which expresses BCMA (e.g., wild-type or mutant BCMA) or condition associated with a cell which expresses BCMA (e.g., wild-type or mutant BCMA) including, e.g., proliferative diseases such as a cancer or malignancy or a precancerous condition such as a myelodysplasia, a myelodysplastic syndrome or a preleukemia; or a noncancer related indication associated with a cell which expresses BCMA (e.g., wild-type or mutant BCMA). For the avoidance of doubt, a disease associated with expression of BCMA may include a condition associated with a cell which does not presently express BCMA, e.g., because BCMA expression has been downregulated, e.g., due to treatment with a molecule targeting BCMA, e.g., a BCMA inhibitor described herein, but which at one time expressed BCMA.

In one aspect, a cancer associated with expression of BCMA (e.g., wild-type or mutant BCMA) is a hematological cancer. In one aspect, the hematological cancer is a leukemia or a lymphoma. In one aspect, a cancer associated with expression of BCMA (e.g., wild-type or mutant BCMA) is a malignancy of differentiated plasma B cells. In one aspect, a cancer associated with expression of BCMA (e.g., wild-type or mutant BCMA) includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of BMCA (e.g., wild-type or mutant BCMA) comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the cancer is multiple myeloma, Hodgkin's lymphoma, non-Hodgkin's lymphoma, or glioblastoma. In embodiments, a disease associated with expression of BCMA includes a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). Further diseases associated with expression of BCMA (e.g., wild-type or mutant BCMA) expression include, but not limited to, e.g., atypical and/or non-classical cancers, malignancies, precancerous conditions or proliferative diseases associated with expression of BCMA (e.g., wild-type or mutant BCMA), e.g., a cancer described herein, e.g., a prostate cancer (e.g., castrate-resistant or therapy-resistant prostate cancer, or metastatic prostate cancer), pancreatic cancer, or lung cancer.

Non-cancer related conditions that are associated with BCMA (e.g., wild-type or mutant BCMA) include viral infections; e.g., HIV, fungal infections, e.g., C. neoformans; autoimmune disease; e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologics (e.g., Factor VIII) where humoral immunity is important. In embodiments, a non-cancer related indication associated with expression of BCMA includes but is not limited to, e.g., autoimmune disease, (e.g., lupus), inflammatory disorders (allergy and asthma) and transplantation.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

Certain chemical definitions are provided below.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). Alkyl may be optionally substituted. Suitable substituents on an alkyl include, without limitation, halo, alkoxy, haloalkoxy (e.g., perfluoroalkoxy such as OCF₃), hydroxy, carboxy, carboxylate, cyano, nitro, amino, alkyl amino, SO₃H, sulfate, phosphate, oxo, thioxo (e.g., C═S), imino (alkyl, aryl, aralkyl), S(O)ₙalkyl (where n is 0-2), S(O)naryl (where n is 0-2), S(O)ₙheteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). As used herein, the term “lower alkyl” denotes a saturated straight- or branched-chain alkyl group containing from 1 to 7 carbon atoms, for example, methyl, ethyl, propyl, isopropyl, n-butyl, i-butyl, 2-butyl, t-butyl and the like. Preferred lower alkyl groups are groups with 1-4 carbon atoms. As used herein, the term “lower alkinyl” denotes a unsaturated straight- or branched-carbon chain containing from 2 to 7 carbon atoms and containing at least one triple bond.

The term “alkoxy” refers to an —O-alkyl radical. The term “lower alkoxy” denotes a group wherein the alkyl residues is as defined above, and which is attached via an oxygen atom.

The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

Unless otherwise indicated, as used herein, the term “alkenoxy”, means “alkenyl-O—”, wherein “alkenyl” is as defined above. Unless otherwise indicated, as used herein, the term “alkynoxy”, means “alkynyl-O—”, wherein “alkynyl” is as defined above.

The terms “alkylamino” and “dialkylamino” refer to —NH(alkyl) and —NH(alkyl)₂ radicals respectively. The term “aralkylamino” refers to a —NH(aralkyl) radical. The term alkylaminoalkyl refers to a (alkyl)NH-alkyl- radical; the term dialkylaminoalkyl refers to a (alkyl)₂N-alkyl- radical. The term “alkoxy” refers to an —O-alkyl radical. The term “mercapto” refers to an SH radical. The term “thioalkoxy” refers to an —S-alkyl radical. The term thioaryloxy refers to an —S-aryl radical. “Hydroxy” refers to the radical —OH.

The term “aralkyl”, as used herein, refers to an alkyl group substituted with an aryl group (e.g., an aromatic or heteroaromatic group).

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbomyl.

“Bicycloalkyl” and “tricycloalkyl” groups are non-aromatic saturated carbocyclic groups consisting of two or three rings respectively, wherein said rings share at least one carbon atom. Unless otherwise indicated, for purposes of the present invention, bicycloalkyl groups include spiro groups and fused ring groups. Examples of bicycloalkyl groups include, but are not limited to, bicyclo-[3.1.0]-hexyl, bicyclo-2.2.1]-hept-1-yl, norbomyl, spiro[4.5]decyl, spiro[4.4]nonyl, spiro[4.3]octyl, and spiro[4.2]heptyl. An example of a tricycloalkyl group is adamantanyl. Other cycloalkyl, bicycloalkyl, and tricycloalkyl groups are known in the art, and such groups are encompassed by the definitions “cycloalkyl”, “bicycloalkyl” and “tricycloalkyl” herein.

“Cycloalkenyl”, “bicycloalkenyl”, and “tricycloalkenyl” refer to non-aromatic carbocyclic cycloalkyl, bicycloalkyl, and tricycloalkyl moieties as defined above, except comprising one or more carbon-carbon double bonds connecting carbon ring members (an “endocyclic” double bond) and/or one or more carbon-carbon double bonds connecting a carbon ring member and an adjacent non-ring carbon (an “exocyclic” double bond). Examples of cycloalkenyl groups include, but are not limited to, cyclopentenyl, cyclobutenyl, and cyclohexenyl, and a non-limiting example of a bicycloalkenyl group is norbornenyl. Other cycloalkenyl, bicycloalkenyl, and tricycloalkenyl groups are known in the art, and such groups are included within the definitions “cycloalkenyl”, “bicycloalkenyl” and “tricycloalkenyl” herein. Cycloalkyl, cycloalkenyl, bicycloalkyl, and bicycloalkenyl groups also include groups that are substituted with one or more oxo moieties. Examples of such groups with oxo moieties are oxocyclopentyl, oxocyclobutyl, oxocyclopentenyl, and norcamphoryl.

The term “heteroaryl” refers to a fully aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms selected independently from N, O, or S if monocyclic, bicyclic, or tricyclic, respectively). Any ring atom can be substituted (e.g., by one or more substituents). The point of attachment of a heteroaryl is on the ring containing said heteroatom(s).

The term “heterocyclyl” or “heterocyloalkyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The point of attachment of a heterocyclyl is on the ring containing said heteroatom(s). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, and pyrrolidinyl.

Bicyclic and tricyclic ring systems containing one or more heteroatoms and both aromatic and non-aromatic rings are considered to be heterocyclyl groups according to the present definition.

The term “saturated or partially saturated heterocyclyl” refers to a non-aromatic cyclic structure that includes at least one heteroatom. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heterocyclylalkyl”, as used herein, refers to an alkyl group substituted with a heterocycle group. The term “cycloalkylalkyl”, as used herein, refers to an alkyl group substituted with a cycloalkyl group.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The terms “hetaralkyl” and “heteroaralkyl”, as used herein, refers to an alkyl group substituted with a heteroaryl group.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

The term “substituents” refers to a group “substituted” on an alkyl, alkoxy, cycloalkyl, cycloalkylalkyl, alkenyl, alkynyl, heterocyclyl, heterocyclylalkyl, heterocycloalkenyl, cycloalkenyl, aryl, aralkyl, heteroaryl or heteroaralkyl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O), aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like (see, e.g., Berge, et al., J. Pharm. Sci. 66(1): 1-79 (January '77).

Various aspects of the compositions and methods herein are described in further detail below. Additional definitions are set out throughout the specification.

### Gamma Secretase Inhibitor (GSI)

The present invention provides compositions comprising, e.g., a gamma secretase inhibitor (GSI), and methods for enhancing the function of a BCMA-targeting agent (e.g., an anti-BCMA antibody molecule or a recombinant non-antibody protein that binds to BCMA), by using such compositions and/or other means as described herein. Any inhibitor of gamma secretase (e.g., a subunit of gamma secretase, e.g., presenilin, nicastrin, APH-1, or PEN-2), and any modulator of a gene encoding gamma secretase (e.g., a subunit of gamma secretase, e.g., presenilin, nicastrin, APH-1, or PEN-2), known in the art, can be used according to the present invention. Examples of GSIs are described below.

### Small Molecules Targeting Gamma Secretase

The compositions, methods and uses described herein comprise a gamma secretase inhibitor (GSI). In some embodiments, the GSI is a small molecule that reduces the expression and/or function of gamma secretase.

In some embodiments, the compound is a compound of formula (I) or a pharmaceutically acceptable salt thereof;

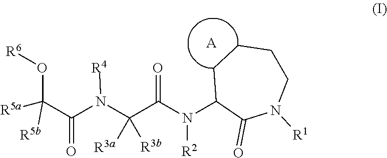

wherein ring A is aryl or heteroaryl; each of R¹, R², and R⁴ is independently hydrogen, C₁-C₆ alkyl, cycloalkyl, heterocyclyl, cycloalkylalkyl, heterocyclylalkyl, aryl, heteroaryl, aralkyl, or heteroaralkyl, wherein each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{A}, —SR^{A}, —C(O)OR^{A}, —C(O)N(R^{A})(R^{B}), —N(R^{A})(R^{B}), or —C(NR^{C})N(R^{A})(R^{B}); each R^{3a}, R^{3b}, R^{5a}, and R^{5b} is independently hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, —OR^{A}, —SR^{A}, —C(O)OR^{A}, —C(O)N(R^{A})(R^{B}), —N(R^{A})(R^{B}), or —C(NR^{C})N(R^{A})(R^{B}); R⁶ is hydrogen, C₁-C₆ alkyl, cycloalkyl, heterocyclyl, cycloalkylalkyl, heterocyclylalkyl, aryl, heteroaryl, aralkyl, or heteroaralkyl, wherein each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and each R^{A}, R^{B}, and R^{C} is independently hydrogen, C₁-C₆ alkyl, cycloalkyl, heterocyclyl, cycloalkylalkyl, heterocyclylalkyl, aryl, heteroaryl, aralkyl, or heteroaralkyl, wherein each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy.

In some embodiments, ring A is aryl (e.g., phenyl). In some embodiments, R¹ is —CH₃. In some embodiments, each of R² and R⁴ is independently hydrogen. In some embodiments, R^{3a} is —CH₃ and R^{3b} is hydrogen. In some embodiments, R^{5a} is hydrogen and R^{5b} is —CH(CH₃)₂. In some embodiments, R6 is hydrogen.

In some embodiments, the compound of formula (I) is a compound described in U.S. Pat. No. 7,468,365, which is herein incorporated by reference in its entirety. In one embodiment, the compound of formula (I) is LY-450139, i.e., semagacestat, (S)-2-hydroxy-3-methyl-N-((S)-1-(((S)-3-methyl-2-oxo-2,3,4,5-tetrahydro-1H-benzo[d]azepin-1-yl)amino)-1-oxopropan-2-yl)butanamide, or a pharmaceutically acceptable salt thereof. In one embodiment, the compound is

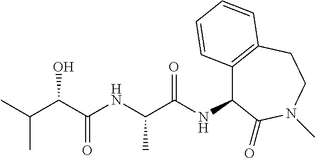 [SMILES: CC(C)[C@H](O)C(=O)N[C@@H](C)C(=O)N[C@@H]1C(=O)N(C)CCc2ccccc21]

or a pharmaceutically acceptable salt thereof.

In some embodiments, the compound is a compound of formula (II) or a pharmaceutically acceptable salt thereof;

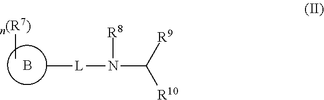

wherein ring B is aryl or heteroaryl; L is a bond, C₁-C₆ alkylene, —S(O)₂—, —C(O)—, —N(R^{E})(O)C, or —OC(O)—; each R⁷ is independently halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is independently substituted with 0-6 occurrences of halogen, —OR^{D}, —SR^{D}, —C(O)OR^{D}, —C(O)N(R^{D})(R^{E}), —N(R^{D})(R^{E}), or —C(NR^{F})N(R^{D})(R^{E}); R is hydrogen, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{D}, —SR^{D}, —C(O)OR^{D}, —C(O)N(R^{D})(R^{E}), —N(R^{D})(R^{E}), or —C(NR^{F})N(R^{D})(R^{E}); each of R9 and R¹⁰ is independently hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, aryl, heteroaryl, aralkyl, or heteroaralkyl is substituted with 0-6 independent occurrences of halogen, —OR^{D}, —SR^{D}, —C(O)OR^{D}, —C(O)N(R^{D})(R^{E}), —N(R^{D})(R^{E}), or —C(NR^{I})N(R^{G})(R^{H}); each R^{D}, R^{E}, and R^{F} is independently hydrogen, C₁-C₆ alkyl, cycloalkyl, heterocyclyl, cycloalkylalkyl, heterocyclylalkyl, aryl, heteroaryl, aralkyl, or heteroaralkyl, wherein each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and n is 0, 1, 2, 3, 4, or 5.

In some embodiments, ring B is heteroaryl (e.g., thiofuranyl). In some embodiments, L is —S(O)₂. In some embodiments, R⁷ is chloro and n is 1. In some embodiments, R⁸ is —CH₂OH. In some embodiments, each of R⁹ and R¹⁰ is independently —CF₃.

In some embodiments, the compound of formula (II) is a compound described in U.S. Pat. No. 7,687,666, which is herein incorporated by reference in its entirety. In one embodiment, the compound of formula (II) is PF-5212362, i.e., begacestat, GSI-953, or (R)-5-chloro-N-(4,4,4-trifluoro-1-hydroxy-3-(trifluoromethyl)butan-2-yl)thiophene-2-sulfonamide, or a pharmaceutically acceptable salt thereof. In one embodiment, the compound is

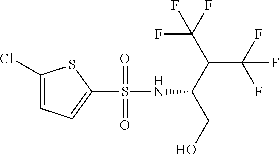 [SMILES: O=S(=O)(N[C@@H](CO)C(C(F)(F)F)C(F)(F)F)c1ccc(Cl)s1]

or a pharmaceutically acceptable salt thereof.

In some embodiments, the compound is a compound is a compound of formula (III) or a pharmaceutically acceptable salt thereof;

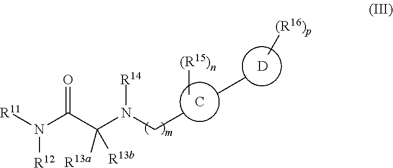

wherein each of rings C and D is independently aryl or heteroaryl; each of R¹¹, R¹², and R¹⁴ is independently hydrogen, C₁-C₆ alkyl, C₁-C₆ alkoxy, —S(O)R^{G}—, S(O)₂R^{G}—, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each of R^{13a} and R^{13b} is hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each R¹⁵ and R¹⁶ is independently halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, wherein each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each R^{G}, R^{H}, and R^{I} is independently hydrogen, C₁-C₆ alkyl, cycloalkyl, heterocyclyl, cycloalkylalkyl, heterocyclylalkyl, aryl, heteroaryl, aralkyl, or heteroaralkyl, wherein each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and each of m, n, and p is independently 0, 1, 2, 3, 4, or 5.

In some embodiments, ring C is aryl (e.g., phenyl). In some embodiments, ring D is heteroaryl (e.g., 1,2,4-oxadiazole). In some embodiments, R¹⁵ is fluoro and n is 1. In some embodiments, p is 0. In some embodiments, m is 1. In some embodiments, R¹⁴ is —S(O)₂R^{G} and R^{G} is chlorophenyl. In some embodiments, R^{13a} is —CH₂CH₂CF₃ and R^{13b} is hydrogen. In some embodiments, each R¹¹ and R¹² is independently hydrogen.

In some embodiments, the compound of formula (III) is a compound described in U.S. Pat. No. 8,084,477, which is herein incorporated by reference in its entirety. In one embodiment, the compound of formula (III) is BMS-708163, i.e., avagacestat, or (R)-2-((4-chloro-N-(2-fluoro-4-(1,2,4-oxadiazol-3-yl)benzyl)phenyl)sulfonamido)-5,5,5-trifluoropentanamide, or a pharmaceutically acceptable salt thereof. In one embodiment, the compound is

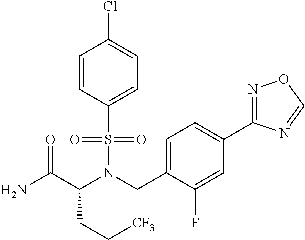 [SMILES: NC(=O)[C@@H](CCC(F)(F)F)N(Cc1ccc(-c2ncon2)cc1F)S(=O)(=O)c1ccc(Cl)cc1]

or a pharmaceutically acceptable salt thereof.

In some embodiments, the compound is a compound of formula (IV);

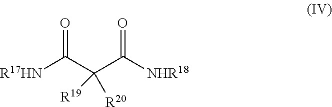

wherein R¹⁷ is selected from

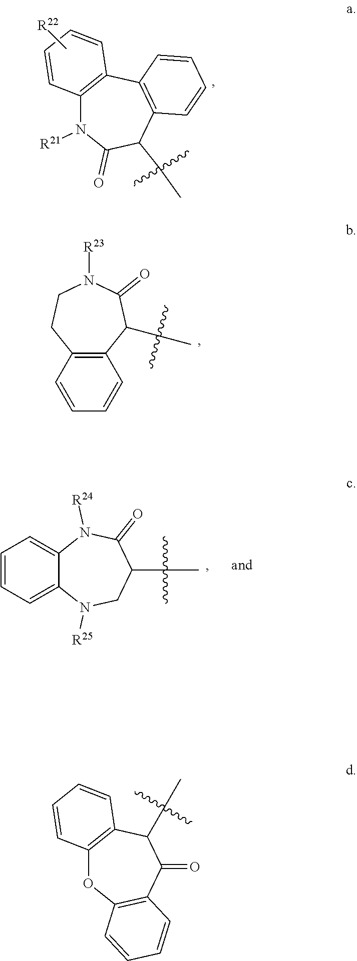

R¹⁸ is lower alkyl, lower alkinyl, —(CH₂)ₙ—O-lower alkyl, —(CH₂)ₙ—S-lower alkyl, —(CH₂)ₙ—CN, —(CR′R″)ₙ CF₃, —(CR′R″)ₙ—CHF₂, —(CR′R″)ₙ—CH₂F, —(CH₂)ₙ, —C(O)O-lower alkyl, —(CH₂)ₙ-halogen, or is —(CH2)ₙ-cycloalkyl optionally substituted by one or more substituents selected from the group consisting of phenyl, halogen and CF₃; R′,R″ are each independently hydrogen, lower alkyl, lower alkoxy, halogen or hydroxy; R¹⁹, R²⁰ are each independently hydrogen, lower alkyl, lower alkoxy, phenyl or halogen; R²¹ is hydrogen, lower alkyl, —(CH2)ₙ—CF₃ or —(CH₂)ₙ-cycloalkyl; R²² is hydrogen or halogen; R²³ is hydrogen or lower alkyl; R²⁴ is hydrogen, lower alkyl, lower alkinyl, —(CH2)ₙ—CF₃, —(CH₂)ₙ-cycloalkyl or —(CH2)ₙ-phenyl optionally substituted by halogen; R²⁵ is hydrogen, lower alkyl, —C(O)H, —C(O)-lower alkyl, —C(O)—CF₃, —C(O)—CH₂F, —C(O)—CHF₂, —C(O)-cycloalkyl, —C(O)—(CH₂)ₙ—O-lower alkyl, —C(O)O—(CH₂)ₙ-cycloalkyl, —C(O)-phenyl optionally substituted by one or more substituents selected from the group consisting of halogen and —C(O)O-lower alkyl, or is —S(O)₂-lower alkyl, —S(O)₂—CF₃, —(CH2)ₙ-cycloalkyl or is —(CH₂)ₙ-phenyl optionally substituted by halogen; n is 0, 1, 2, 3 or 4.

In some embodiments, R¹⁷ is 5,7-dihydro-6H-dibenzo[b,d]azepin-6-onyl. In some embodiments, each R¹⁹ and R²⁰ is independently —CH₃. In some embodiments, R¹⁸ is CH₂CF₂CF₃.

In some embodiments, the compound of formula (IV) is described in a compound described in U.S. Pat. No. 7,160,875, which is herein incorporated by reference in its entirety. In one embodiment, the compound is R04929097, i.e., (S)-2,2-dimethyl-N1-(6-oxo-6,7-dihydro-5H-dibenzo[b,d]azepin-7-yl)-N3-(2,2,3,3,3-pentafluoropropyl)malonamide, or a pharmaceutically acceptable salt thereof. In one embodiment, the compound is

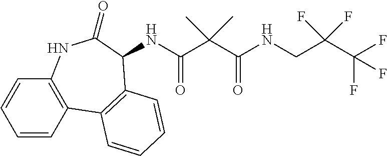 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof.

In some embodiments, the compound is a compound of formula (V) or a pharmaceutically acceptable salt thereof;

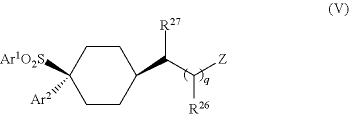

wherein q is 0 or 1; Z represents halogen, —CN, —NO₂, —N₃, —CF₃, —OR^{2a}, —N(R^{2a})₂, —CO₂R^{2a}, —OCOR^{2a}, —COR^{2a}, —CON(R^{2a})₂, —OCON(R^{2a})₂, —CONR^{2a}(OR^{2a}), —CON(R^{2a})N(R^{2a})₂, —CONHC(═NOH)R^{2a}, heterocyclyl, phenyl or heteroaryl, said heterocyclyl, phenyl or heteroaryl bearing 0-3 substituents selected from halogen, —CN, —NO₂, —CF₃, —OR^{2a}, —N(R^{2a})₂, —CO₂R^{2a}, —COR^{2a}, —CON(R^{2a})₂ and C₁₋₄ alkyl; R²⁷ represents H, C₁₋₄ alkyl, or OH; R²⁶ represents H or C₁₋₄ alkyl; with the proviso that when m is 1, R²⁶ and R²⁷ do not both represent C₁₋₄ alkyl; Ar¹ represents C₆₋₁₀ aryl or heteroaryl, either of which bears 0-3 substituents independently selected from halogen, —CN, —NO₂, —CF₃, —OH, —OCF₃, C₁₋₄ alkoxy or C₁₋₄ alkyl which optionally bears a substituent selected from halogen, CN, NO₂, CF₃, OH and C₁₋₄ alkoxy; Ar² represents C₆₋₁₀ aryl or heteroaryl, either of which bears 0-3 substituents independently selected from halogen, —CN, —NO₂, —CF₃, —OH, —OCF₃, C₁₋₄ alkoxy or C₁₋₄ alkyl which optionally bears a substituent selected from halogen, —CN, —NO₂, —CF₃, —OH and C₁₋₄ alkoxy; R^{2a} represents H, C₁₋₆ alkyl, C₃₋₆ cycloalkyl, C₃₋₆cycloalkyl, C₁₋₆ alkyl, C₂₋₆ alkenyl, any of which optionally bears a substituent selected from halogen, —CN, —NO₂, —CF₃, —OR^{2b}, —CO₂R^{2b}, —N(R^{2b})₂, —CON(R^{2b})₂, Ar and COAr; or R^{2a} represents Ar; or two R^{2a} groups together with a nitrogen atom to which they are mutually attached may complete an N-heterocyclyl group bearing 0-4 substituents independently selected from ═O, ═S, halogen, C₁₋₄ alkyl, —CN, —NO₂, —CF₃, —OH, C₁₋₄ alkoxy, C₁₋₄ alkoxycarbonyl, CO₂H, amino, C₁₋₄ alkylamino, di(C₁₋₄ alkyl)amino, carbamoyl, Ar and COAr; R^{2b} represents H, C₁₋₆ alkyl, C₃₋₆ cycloalkyl, C₃₋₆ cycloalkylC₁₋₆ alkyl, C₂₋₆ alkenyl, any of which optionally bears a substituent selected from halogen, —CN, —NO₂, —CF₃, —OH, C₁₋₄ alkoxy, C₁₋₄ alkoxycarbonyl, —CO₂H, amino, C₁₋₄ alkylamino, di(C₁₋₄ alkyl)amino, carbamoyl, Ar and COAr; or R^{2b} represents Ar; or two R^{2b} groups together with a nitrogen atom to which they are mutually attached may complete an N-heterocyclyl group bearing 0-4 substituents independently selected from ═O, ═S, halogen, C₁₋₄ alkyl, —CN, —NO₂, CF₃, —OH, C₁₋₄ alkoxy, C₁₋₄ alkoxycarbonyl, —CO₂H, amino, C₁₋₄ alkylamino, di(C₁₋₄ alkyl)amino, carbamoyl, Ar and COAr; Ar represents phenyl or heteroaryl bearing 0-3 substituents selected from halogen, C₁₋₄ alkyl, —CN, —NO₂, —CF₃, —OH, C₁₋₄ alkoxy, C₁₋₄ alkoxycarbonyl, amino, C₁₋₄ alkylamino, di(C₁₋₄ alkyl)amino, carbamoyl, C₁₋₄ alkylcarbamoyl and di(C₁₋₄ alkyl)carbamoyl.

In some embodiments, q is 1. In some embodiments, Z is CO₂H. In some embodiments, each of R²⁷ and R²⁶ is independently hydrogen. In some embodiments, Ar¹ is chlorophenyl. In some embodiments, Ar² is difluorophenyl.

In some embodiments, the compound of formula (V) is described in U.S. Pat. No. 6,984,663, which is herein incorporated by reference in its entirety. In one embodiment, the compound of formula (V) is MK-0752, i.e., 3-((1S,4R)-4-((4-chlorophenyl)sulfonyl)-4-(2,5-difluorophenyl)cyclohexyl)propanoic acid, or a pharmaceutically acceptable salt thereof. In one embodiment, the compound is

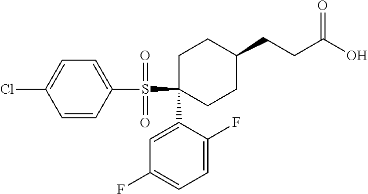 [SMILES: O=C(O)CC[C@H]1CC[C@@](c2cc(F)ccc2F)(S(=O)(=O)c2ccc(Cl)cc2)CC1]

or a pharmaceutically acceptable salt thereof.

In some embodiments, the compound is a compound of formula (VI):

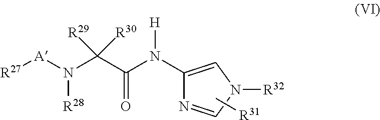

or a pharmaceutically acceptable salt thereof, wherein A′ is absent or selected from

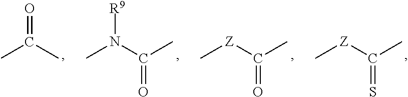

and —S(O)₂—; Z is selected from —CH₂, —CH(OH), —CH(C₁-C₆ alkyl), —CH(C₁-C₆ alkoxy), —CH(NR³³R³⁴), —CH(CH₂(OH)), —CH(CH(C₁-C₄ alkyl)(OH)) and —CH(C(C₁-C₄ alkyl)(C₁-C₄ alkyl)(OH)), for example —CH(C(CH₃)(CH₃)(OH)) or —CH(C(CH₃)(CH₂CH₃)(OH)); R²⁷ is selected from C₁-C₂₀ alkyl, C₂-C₂₀ alkenyl, C₂-C₂₀ alkynyl, C₁-C₂₀ alkoxy, C₂-C₂₀ alkenoxy, C₁-C₂₀ hydroxyalkyl, C₃-C₈ cycloalkyl, benzo(C₃-C₈ cycloalkyl), benzo(C₃-C₈ heterocycloalkyl), C₄-C₈ cycloalkenyl, (C₅-C₁)bi- or tricycloalkyl, benzo(C₅-C₁₁)bi- or tricycloalkyl, C₇-C₁₁tricycloalkenyl, (3-8 membered) heterocycloalkyl, C₆-C₁₄ aryl and (5-14 membered) heteroaryl, wherein each hydrogen atom of said alkyl, alkenyl, alkynyl, alkoxy and alkenoxy is optionally independently replaced with halo, and wherein said cycloalkyl, benzo(C₃-C₈ cycloalkyl), cycloalkenyl, (3-8 membered) heterocycloalkyl, C₆-C₁₄ aryl and (5-14 membered) heteroaryl is optionally independently substituted with from one to four substituents independently selected from C₁-C₁₀ alkyl optionally substituted with from one to three halo atoms, C₁-C₁₀ alkoxy optionally substituted with from one to three halo atoms, C₁-C₁₀ hydroxyalkyl, halo, preferably fluorine, —OH, —CN, —NR³³R³⁴, —C(═O)NR³³R³⁴, —C(═O)R³⁵, C₃-C₈ cycloalkyl and (3-8 membered) heterocycloalkyl; R²⁸ is selected from hydrogen, C₁-C₆ alkyl, C₂-C₆ alkenyl, C₃-C₈ cycloalkyl and C₅-C₈ cycloalkenyl, wherein R²⁸ is optionally independently substituted with from one to three substituents independently selected from C₁-C₄ alkyl optionally substituted with from one to three halo atoms, C₁-C₄ alkoxy optionally substituted with from one to three halo atoms, halo and —OH; or R²⁷ and R²⁸ together with the A′ group when present and the nitrogen atom to which R²⁸ is attached, or R²⁷ and R²⁸ together with the nitrogen atom to which R²⁷ and R²⁸ are attached when A′ is absent, may optionally form a four to eight membered ring; R²⁹ is selected from hydrogen, C₁-C₆ alkyl, C₂-C₆ alkenyl, C₂-C₆ alkynyl, C₃-C₆ cycloalkyl, C₅-C₆ cycloalkenyl and (3-8 membered) heterocycloalkyl, wherein said alkyl, alkenyl, alkynyl, cycloalkyl, cycloalkenyl and heterocycloalkyl are each optionally independently substituted with from one to three substituents independently selected from C₁-C₄alkoxy, halo, —OH—S(C₁-C₄)alkyl and (3-8 membered) heterocycloalkyl; R³⁰ is hydrogen, C₁-C₆ alkyl or halo; or R²⁹ and R³⁰ may together with the carbon atom to which they are attached optionally form a moiety selected from cyclopropyl, cyclobutyl, cyclopentyl, cyclohexyl, morpholino, piperidino, pyrrolidino, tetrahydrofuranyl and perhydro-2H-pyran, wherein said moiety formed by R²⁹ and R³⁰ is optionally substituted with from one to three substituents independently selected from C₁-C₆ alkyl optionally substituted with from one to three halo atoms, C₁-C₆ alkoxy optionally substituted with from one to three halo atoms, halo, —OH, —CN and allyl; R³¹ is selected from hydrogen, C₁-C₆ alkyl, C₂-C₆ alkylene, C₁-C₆ alkoxy, halo, —CN, C₃-C₁₂ cycloalkyl, C₄-C₁₂ cycloalkenyl and C₆-C₁₀ aryl, (5-10 membered) heteroaryl, wherein said alkyl, alkylene and alkoxy of R³¹ are each optionally independently substituted with from one to three substituents independently selected from halo and —CN, and wherein said cycloalkyl, cycloalkenyl and aryl and heteroaryl of R³¹ are each optionally independently substituted with from one to three substituents independently selected from C₁-C₄ alkyl optionally substituted with from one to three halo atoms, C₁-C₄ alkoxy optionally substituted with from one to three halo atoms, halo and —CN; R³² is selected from hydrogen, C₁-C₂₀ alkyl, C₁-C₂₀ alkoxy, C₁-C₂₀ hydroxyalkyl, C₃-C₁₂ cycloalkyl, C₄-C₁₂ cycloalkenyl, (C₅-C₂₀) bi- or tricycloalkyl, (C₇-C₂₀)bi- or tricycloalkenyl, (3-12 membered) heterocycloalkyl, (7-20 membered) hetero bi- or heterotricycloalkyl, C₆-C₁₄ aryl and (5-15 membered) heteroaryl, wherein R³² is optionally independently substituted with from one to four substituents independently selected from C₁-C₂₀ alkyl optionally substituted with from one to three halo atoms, C₁-C₂₀ alkoxy, —OH, —CN, —NO₂, —NR³³R³⁴, —C(═O)NR³³R³⁴, —C(═O)R³⁵, —C(═O)OR³⁵, —S(O)ₙNR³³R³⁴, —S(O)nR³⁵, C₃-C₁₂ cycloalkyl, (4-12 membered) heterocycloalkyl optionally substituted with from one to three OH or halo groups, (4-12 membered) heterocycloalkoxy, C₆-C₁₄ aryl, (5-15 membered) heteroaryl, C₆-C₁₂ aryloxy and (5-12 membered) heteroaryloxy; or R³³ and R³⁴ may together with the carbon and nitrogen atoms to which they are respectively attached optionally form a (5-8 membered) heterocycloalkyl ring, a (5-8 membered) heterocycloalkenyl ring or a (6-10 membered) heteroaryl ring, wherein said heterocycloalkyl, heterocycloalkenyl and heteroaryl rings are each optionally independently substituted with from one to three substituents independently selected from halo, C₁-C₆ alkyl, optionally substituted with from one to three halo atoms, C₁-C₆ alkoxy optionally substituted with from one to three halo atoms, C₁-C₆ hydroxyalkyl, —OH, —(CH₂)_{zero-10}NR³³R³⁴, (CH₂)_{zero-10}C(═O)NR³³R³⁴, —S(O)₂NR³³R³⁴ and C₃-C₁₂ cycloalkyl; R³³ and R³⁴ are each independently selected from hydrogen, C₁-C₁₀ alkyl wherein each hydrogen atom of said C₁-C₁₀ alkyl is optionally independently replaced with a halo atom, preferably a fluorine atom, C₂-C₁₀ alkenyl, C₂-C₁₀ alkynyl, C₁-C₆ alkoxy wherein each hydrogen atom of said C₁-C₆ alkoxy is optionally independently replaced with a halo atom, C₂-C₆ alkenoxy, C₂-C₆ alkynoxy, —C(═O)R11, —S(O)R11, C₃-C₈ cycloalkyl, C₄-C₈ cycloalkenyl, (C₅-C₁₁)bi-ortricycloalkyl, (C₇-C₁₁)bi- or tricycloalkenyl, (3-8 membered) heterocycloalkyl, C₆-C₁₄ aryl and (5-14 membered) heteroaryl, wherein said alkyl and alkoxy are each optionally independently substituted with from one to three substituents independently selected from halo and —OH, and wherein said cycloalkyl, cycloalkenyl, bi- or tricycloalkyl, bi- or tricycloalkenyl, heterocycloalkyl, aryl and heteroaryl are each optionally independently substituted with from one to three substituents independently selected from halo, —OH, C₁-C₆ alkyl optionally independently substituted with from one to six halo atoms, C₂-C₆ alkenyl, C₂-C₆ alkynyl, C₁-C₆ alkoxy, C₂-C₆ alkenoxy, C₂-C₆ alkynoxy and C₁-C₆ hydroxyalkyl; or NR³³R³⁴ may form a (4-7 membered) heterocycloalkyl, wherein said heterocycloalkyl optionally comprises from one to two further heteroatoms independently selected from N, O, and S, and wherein said heterocycloalkyl optionally contains from one to three double bonds, and wherein said heterocycloalkyl is optionally independently substituted with from one to three substituents independently selected from C₁-C₆ alkyl optionally substituted with from one to six halo atoms, C₂-C₆ alkenyl, C₂-C₆ alkynyl, C₁-C₆ alkoxy, C₂-C₆ alkenoxy, C₂-C₆ alkynoxy, C₁-C₆ hydroxyalkyl, C₂-C₆ hydroxyalkenyl, C₂-C₆ hydroxyalkynyl, halo, —OH, —CN, —NO₂, —C(═O)R³⁵, —C(═O)OR³⁵, —S(O)nR³⁵ and —S(O)ₙNR³³R³⁴; R³⁵ is selected from H, C₁-C₈ alkyl, C₃-C₈ cycloalkyl, C₄-C₈ cycloalkenyl, (C₅-C₁₁)bi- or tricycloalkyl, —(C₇-C₁₁)bi- or tricycloalkenyl, (3-8 membered) heterocycloalkyl, C₆-C₁₀ aryl and (5-14 membered) heteroaryl, wherein said alkyl of R³⁵ is optionally independently substituted with from one to three substituents independently selected from —OH, —CN and C₃-C₈ cycloalkyl, and wherein each hydrogen atom of said alkyl is optionally independently replaced with a halo atom, preferably a fluorine atom, and wherein said cycloalkyl, cycloalkenyl, heterocycloalkyl, aryl and hetereoaryl of R³⁵ are each optionally independently substituted with from one to three substituents independently selected from halo, C₁-C₈ alkyl optionally substituted with from one to three halo atoms, —OH, —CN and C₃-C₈cycloalkyl; n is in each instance an integer independently selected from zero, 1, 2 and 3; and the pharmaceutically acceptable salts of such compounds.

In some embodiments, the compound of formula (VI) is described in U.S. Pat. No. 7,795,447, which is herein incorporated by reference in its entirety. In one embodiment, the compound of formula (VI) is PF-3084014, i.e., nirogacestat or (S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentanamide, or a pharmaceutically acceptable salt thereof.

In some embodiments, the compound is

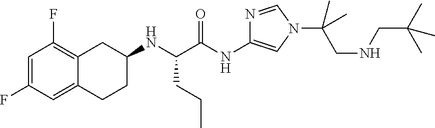 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof.

In some embodiments, the compound is a compound of formula (VII):

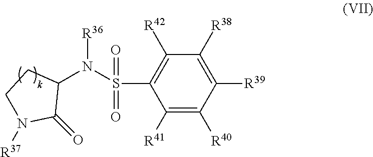

or a pharmaceutically acceptable salt thereof wherein k is 1, 2, or 3; R³⁶ is aryl C₁-C₈ alkyl, aryl C₂-C₆ alkenyl, or arylalkynyl, wherein the aryl group is substituted with 0-5 occurrences of C₁-C₆ alkyl, C₁-C₆ alkoxy, halogen, haloalkyl, haloalkoxy, heteroaryl, heteroaryl(C₁-C₆)alkoxy, arylalkoxy, aryloxy, C₁-C₆ alkoxycarbonyl, —OCH₂CH₂O—, —OCH₂O—, —C(O)NR⁴³R⁴⁴, —NHR′, —NR′R″, —N(R¹⁶)C(O)R¹⁷, heterocycloalkyl, phenyl, aryl C₁-C₆ alkanoyl, phenylalkoxy, phenyloxy, CN, —SO₂-aryl, —S(O)ₙR²⁵, —(C₁-C₄ alkyl)-S(O)ₓR²⁵, —(C₁-C₄ alkyl)-SO₂-aryl, OH, C₁-C₆ thioalkoxy, C₂-C₆ alkenyl, —OSO₂-aryl, or CO₂H, wherein each heteroaryl is independently substituted with 0-3 occurrences of C₁-C₆ alkyl, heteroaryl substituted with 0-2 occurrences of halogen, alkyl, alkoxy, haloalkyl, haloalkoxy, alkoxyalkyl or CN, C₁-C₆ alkoxy, C₁-C₄ alkoxy C₁-C₄ alkyl, C₃-C₆ cycloalkyl, halogen, or phenyl substituted with 0-5 occurrences of halogen, OH, C₁-C₆ alkyl, C₁-C₄ alkoxy, CF₃, OCF₃, CN, or C₁-C₆ thioalkoxy, wherein each heterocycloalkyl and aryl are independently substituted with 0-2 occurrences of halogen, alkyl, alkoxy, haloalkyl, haloalkoxy, alkoxyalkyl or CN, C₁-C₆ alkyl, C₁-C₆ alkoxy, C₁-C₄ alkoxy C₁-C₄ alkyl, C₃-C₆ cycloalkyl, halogen, or phenyl substituted with 0-5 occurrences of halogen, OH, C₁-C₆ alkyl, C₁-C₄ alkoxy, CF₃, OCF₃, CN, or C₁-C₆ thioalkoxy; R¹⁶ is hydrogen or C₁-C₆ alkyl; R¹⁷ is C₁-C₆ alkyl, aryl, heteroaryl, C₁-C₆ alkoxy, OH, aryloxy, heteroaryloxy, aryl(C₁-C₆)alkoxy, —NR¹⁸R¹⁹, cycloalkyl, or arylalkyl, wherein the cyclic portions of each are independently substituted with 0-5 occurrences of alkyl, alkoxy, halo, haloalkyl, haloalkoxy, CN, NH₂, NH(alkyl), N(alkyl) (alkyl), CO₂H, or C₁-C₆ alkoxycarbonyl; R¹⁸ and R¹⁹ are independently hydrogen, C₁-C₆ alkyl, aryl, heteroaryl, heterocycloalkyl or aryl(C₁-C₆)alkyl, wherein the cyclic portions of each are substituted with 0-3 occurrences of alkyl, alkoxy, halogen, hydroxyl, CF₃, or OCF₃; each R′ is independently hydrogen, C₁-C₆ alkyl, aryl, aryl(C₁-C₄)alkyl, C₁-C₆ alkanoyl, C₃-C₈ cycloalkyl, aryl(C₁-C₆)alkanoyl, heterocycloalkyl, heteroaryl(C₁-C₄)alkyl, —SO₂-alkyl, —SO₂-aryl, —SO₂-heteroaryl, heterocycloalkyl(C₁-C₆)alkanoyl, or heteroaryl(C₁-C₆)alkanoyl, wherein the alkyl portion of the alkyl and alkanoyl groups are optionally substituted with halogen or C₁-C₆ alkoxy and the aryl and heteroaryl groups are optionally substituted with alkyl, alkoxy, halogen, haloalkyl, haloalkoxy; each R″ is independently hydrogen or C₁-C₆ alkyl, wherein the alkyl group is optionally substituted with halogen;
   R³⁶ is C₃-C₇ cycloalkyl(C₁-C₆ alkyl) wherein the cyclic portion is substituted with 0-5 occurrences of halogen, C₁-C₆ alkyl, OH, alkoxycarbonyl, or C₁-C₆ alkoxy; or R³⁶ is C₁-C₁₄ alkyl, C₂-C₁₆ alkenyl, or C₂-C₈ alkynyl, each of which is substituted 0-5 occurrences of OH, halogen, C₁-C₆ alkoxy, aryl, arylalkoxy, aryloxy, heteroaryl, heterocycloalkyl, aryl(C₁-C₆)alkyl, —CO₂(C₁-C₆ alkyl), —NR′R″, C₁-C₆ thioalkoxy, —NHS(O)ₓR²⁵, —N(C₁-C₆ alkyl)-S(O)ₙR²⁵, —S(O)ₓR²⁵, —C(O)NR⁴³R⁴⁴, —N(R¹⁶)C(O)NR¹⁶R¹⁷, or —N(R₁₆)C(O)R¹⁷; wherein the above aryl groups are substituted with 0-3 occurrences of OH, C₁-C₆ alkoxy, C₁-C₆ alkyl, or halogen; R⁴³ and R⁴⁴ are independently hydrogen, C₁-C₆ alkyl, aryl, arylalkyl, heteroaryl, heteroarylalkyl, heterocycloalkyl, heterocycloalkylalkyl, arylalkanoyl, alkenyl, cycloalkyl, alkynyl, cycloalkenyl, pyridyl, imidazolyl, thiazolyl, oxazolyl, or indolyl, wherein each alkyl is substituted with 0-3 occurrences of NH₂, NH(C₁-C₆ alkyl), N(C₁-C₆ alkyl) (C₁-C₆ alkyl), OH, C₁-C₆ thioalkoxy, heterocycloalkyl, aryl, heteroaryl, CN, halogen, or alkoxy optionally substituted with OH or phenyl, wherein the aryl, heteroaryl and heterocycloalkyl groups are substituted with 0-3 occurrences of C₁-C₄ alkyl, C₁-C₄ alkoxy, CF₃, OCF₃, OH, halogen, thioalkoxy, phenyl or heteroaryl; or R⁴³, R⁴⁴, and the nitrogen to which they are attached form a heterocycloalkyl ring containing from 3 to 7 ring members, wherein the cyclic portions of R⁴³ and R⁴⁴ or the heterocyclic ring formed from R⁴³, R⁴⁴, and the nitrogen to which they are attached are substituted with 0-3 occurrences of alkyl, alkoxy, halo, OH, thioalkoxy, NH₂, NH(C₁-C₆alkyl), N(C₁-C₆alkyl) (C₁-C₆alkyl), CF₃, OCF₃, phenyl optionally substituted with a halogen, —(C₁-C₄ alkyl)-N(H or C₁-C₄ alkyl)-phenyl, C₁-C₄ hydroxyalkyl, arylalkoxy, arylalkyl, arylalkanoyl, C(O)NH₂, C(O)NH(C₁-C₆alkyl), C(O)N(C₁-C₆alkyl) (C₁-C₆alkyl), heterocycloalkylalkyl, C₁-C₆ alkoxycarbonyl, C₂-C₆ alkanoyl, heteroaryl, or —SO₂(C₁-C₆alkyl); x is 0, 1, or 2; R²⁵ is C₁-C₆alkyl, OH, NR²⁶R²⁷; R²⁶ and R²⁷ are independently hydrogen, C₁-C₆alkyl, phenyl(C₁-C₄ alkyl), aryl, or heteroaryl; or R²⁶, R²⁷ and the nitrogen to which they are attached form a heterocycloalkyl ring;
   R³⁶ is heteroaryl(C₁-C₆)alkyl wherein the cyclic portion is substituted 0-5 occurrences of halogen, C₁-C₆alkyl, C₁-C₆alkoxy, C₁-C₄haloalkyl, C₁-C₄haloalkoxy, aryl, arylalkyl, aryloxy, heteroaryl, —SO₂-aryl, —S(O)ₓR²⁵, (C₁-C₄alkyl)-S(O)ₓR²⁵, CN, C₁-C₆ thioalkoxy, C₁-C₆alkoxycarbonyl, —NR′R″, —C(O)NR′R″, heterocycloalkyl, wherein the above aryl groups are substituted with 0-4 occurrences of halogen, C₁-C₆alkyl, C₁-C₆ alkoxy, C₁-C₄haloalkyl, C₁-C₄haloalkoxy, or CN; wherein the above heteroaryl and heterocycloalkyl groups are substituted with 0-3 occurrences of halogen, CF₃, (C₁-C₄)alkyl, C₁-C₆ thioalkoxy, OH, C₁-C₄ hydroxyalkyl, or C₁-C₄alkoxy; or
   R³⁶ is heterocycloalkyl(C₁-C₆ alkyl) wherein the cyclic portion is substituted with 0-3 occurrences of halogen, C₁-C₆alkyl, C₁-C₆ alkoxy, C₁-C₄haloalkyl, C₁-C₄haloalkoxy, aryl, arylalkyl, aryloxy, heteroaryl, —SO₂-aryl, —S(O)ₓR²⁵, (C₁-C₄alkyl)-S(O)ₓR²⁵, CN, C₁-C₆ thioalkoxy, C₁-C₆ alkoxycarbonyl, —NR′R″, —C(O)NR′R″, heterocycloalkyl;
   R³⁷ is hydrogen, C₁-C₆alkyl, or phenyl(C₁-C₄)alkyl; R³⁸ is hydrogen, halogen, C₁-C₆alkyl, C₁-C₆ alkoxy, C₁-C₆haloalkyl, CN; R³⁹ is hydrogen, halogen, C₁-C₆alkyl optionally substituted with —CO₂—(C₁-C₆alkyl), C₁-C₆alkoxy, C₁-C₆haloalkyl, C₁-C₆haloalkoxy, CN, aryloxy, isocyanato, —SO₂(C₁-C₆alkyl), —NHR′, —NR′R″, C₁-C₆ alkanoyl, heteroaryl, aryl; or
R³⁸ and R³⁹ and the carbons to which they are attached form a heterocycloalkyl ring which is substituted with 0-3 occurrences of C₁-C₄ alkyl, C₁-C₄ alkoxy, halogen, or C₁-C₄ alkanoyl wherein the alkanoyl group is substituted with 0-3 halogen atoms; R⁴⁰ is hydrogen, —SO₂NR′R″, halogen; or R³⁹ and R⁴⁰ and the carbons to which they are attached form a benzo ring; or R³⁹ and R⁴⁰ and the carbons to which they are attached form a 1-oxa-2,3-diazacyclopentyl ring;
R⁴⁰ and R⁴¹ are independently hydrogen or F; or R⁴⁰, R⁴¹, and the carbons to which they are attached for a 1,2,5-oxadiazolyl ring; or R⁴⁰, R⁴¹, and the carbons to which they are attached form a naphthyl ring.

In some embodiments, R³⁶ is 4-bromobenzyl. In some embodiments, R³⁷ is hydrogen. In some embodiments, k is 2. In some embodiments, each of R³⁸, R⁴⁰, R⁴¹, and R⁴² is independently hydrogen. In some embodiments, R³⁹ is chloro.

In some embodiments, the compound of formula (VI) is described in U.S. Pat. No. 7,939,657, which is herein incorporated by reference in its entirety. In one embodiment, the compound of formula (VI) is ELN-318463, i.e., HY-50882 or (R)-N-(4-bromobenzyl)-4-chloro-N-(2-oxoazepan-3-yl)benzenesulfonamide, or a pharmaceutically acceptable salt thereof. In some embodiments, the compound is

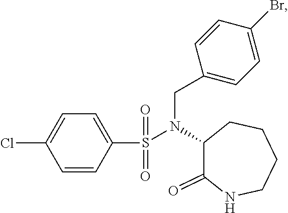 [SMILES: O=C1NCCCC[C@H]1N(Cc1ccc(Br)cc1)S(=O)(=O)c1ccc(Cl)cc1]

or a pharmaceutically acceptable salt thereof.

In some embodiments, the compound is a compound of formula (VII):

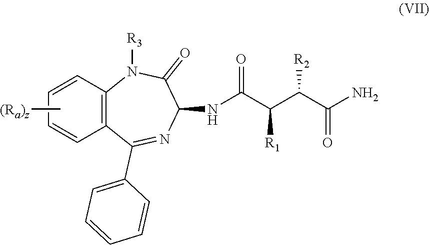

or a pharmaceutically acceptable salt thereof, wherein R₁ is —CH₂CF₃ or —CH₂CH₂CF₃; R₂ is —CH₂CF₃, —CH₂CH₂CF₃, or —CH₂CH₂CH₂CF₃; R₃ is hydrogen or —CH₃; each Rₐ is independently F, Cl, —CN, —OCH₃, and/or —NHCH₂CH₂OCH₃; and z is 0, 1, or 2.

In some embodiments, R₁ is —CH₂CH₂CF₃CH₂CH₂CF₃. In some embodiments, R₂—CH₂CH₂CF₃. In some embodiments, R₃ is —CH₃. In some embodiments, z is 0.

In some embodiments, the compound of formula (VII) is described in U.S. Pat. No. 8,629,136, which is herein incorporated by reference in its entirety. In one embodiment, the compound of formula (VII) is BMS-906024, i.e., (2R,3S)-N-[(3S)-1-methyl-2-oxo-5-phenyl-2,3-dihydro-1H-1,4-benzodiazepin-3-yl]-2,3-bis(3,3,3-trifluoropropyl)succinamide, or a pharmaceutically acceptable salt thereof. In one embodiment, the compound is

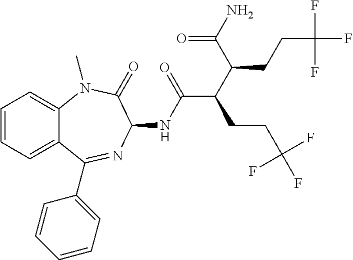 [SMILES: CN1C(=O)[C@@H](NC(=O)[C@H](CCC(F)(F)F)[C@H](CCC(F)(F)F)C(N)=O)N=C(c2ccccc2)c2ccccc21]

or a pharmaceutically acceptable salt thereof.

In some embodiments, the compound is described in U.S. Pat. No. 8,629,136, which is herein incorporated by reference in its entirety. In one embodiment, the compound is LY3039478, i.e., crenigacestat or 4,4,4-trifluoro-N-((R)-1-(((S)-5-(2-hydroxyethyl)-6-oxo-6,7-dihydro-5H-benzo[d]pyrido[2,3-b]azepin-7-yl)amino)-1-oxopropan-2-yl)butanamide, or a pharmaceutically acceptable salt thereof. In some embodiments, the compound is:

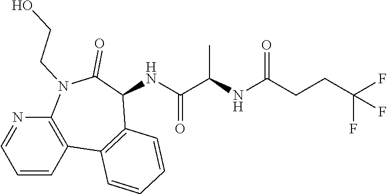 [SMILES: C[C@@H](NC(=O)CCC(F)(F)F)C(=O)N[C@@H]1C(=O)N(CCO)c2ncccc2-c2ccccc21]

or a pharmaceutically acceptable salt thereof.

In some embodiments, the compound is BMS-299897, i.e., 2-[(1R)-1-[[(4-chlorophenyl)sulfonyl](2,5-difluorophenyl)amino]ethyl-5-fluorobenzenebutanoic acid or a pharmaceutically acceptable salt thereof. In some embodiments, the compound is

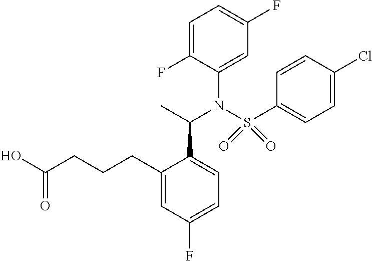 [SMILES: C[C@H](c1ccc(F)cc1CCCC(=O)O)N(c1cc(F)ccc1F)S(=O)(=O)c1ccc(Cl)cc1]

or a pharmaceutically acceptable salt thereof.

In some embodiments, the compound is LY-411575, i.e., LSN-411575, (S)-2-((S)-2-(3,5-difluorophenyl)-2-hydroxyacetamido)-N-((S)-5-methyl-6-oxo-6,7-dihydro-5H-dibenzo[b,d]azepin-7-yl)propanamide, or a pharmaceutically acceptable salt thereof. In some embodiments, the compound is

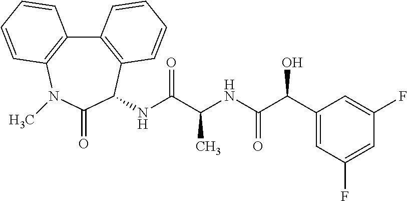 [SMILES: C[C@H](NC(=O)[C@@H](O)c1cc(F)cc(F)c1)C(=O)N[C@@H]1C(=O)N(C)c2ccccc2-c2ccccc21]

or a pharmaceutically acceptable salt thereof.

In some embodiments, the compound is DAPT, i.e., N-[(3,5-difluorophenyl)acetyl]-L-alanyl-2-phenyl]glycine-1,1-dimethylethyl ester or a pharmaceutically acceptable salt thereof. In some embodiments, the compound is

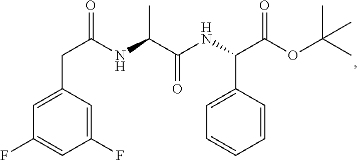 [SMILES: C[C@H](NC(=O)Cc1cc(F)cc(F)c1)C(=O)N[C@H](C(=O)OC(C)(C)C)c1ccccc1]

or a pharmaceutically acceptable salt thereof.

In some embodiments, the compound is a compound of the following formulae:

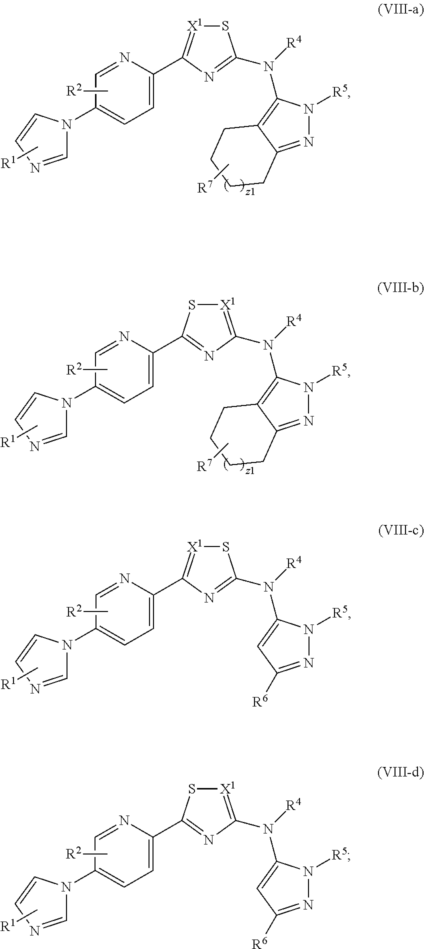

wherein, z1 is 0, 1 or 2; R¹ is C(R³) or N; R¹ is hydrogen, halogen, —N₃, —CF₃, —CCl₃, —CBr₃, —CI₃, —CN, —CHO, —OR^{1A}, —NR^{1A}R^{1B}, —COOR^{A}, —C(O)NR^{A}R^{1B}, —NO₂, —SR^{A}, —S(O)ₙ₁OR^{1A}, —S(O)ₙ₁NR^{1A}R^{1B}, —NHNR^{1A}R^{1B}, —ONR^{1A}R^{1B}, —NHC(O)NHNR^{1A}R^{1B} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, halogen, —N₃, —CF₃, —CCl₃, —CBr₃, —CI₃, —CN, —CHO, —OR^{2A}, —NR^{2A}R^{2B}, —COOR^{2A}, —C(O)NR^{2A}R^{2B}, —NO₂, —SR^{2A}, —S(O)ₙ₂R^{2A}, —S(O)ₙ₂OR_{2A}, —S(O)ₙ₂NR^{2A}R^{2B}, —NHNR^{2A}R^{2B}, —ONR^{2A}R^{2B}, —NHC(O)NHNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is hydrogen, halogen, —N₃, —CF₃, —CCl₃, —CBr₃, —CI₃, —CN, —CHO, —OR^{3A}, —N^{2A}R^{3B}, —COOR^{3A}, —C(O)NR^{3A}R^{3B}, —NO₂, —SR^{3A}, —S(O)ₙ₃R^{3A}, —S(O)ₙ₃OR^{3A}, —S(O)ₙ₃ONR^{3A}R^{3B}, —NHNR^{3A}R^{3B}, —ONR^{3A}R^{3B}, —NHC(O)NHNR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is hydrogen, halogen, —N₃, —CF₃, —CCl₃, —CBr₃, —CI₃, —CN, —CHO, —OR⁴, —NR^{4A}R^{4B}, —COOR^{4A}, —C(O)NR^{4A}R^{4B}, —NO₂, —SR^{4A}, —S(O)ₙ₄R^{4A}, —S(O)ₙ₄R^{4A}, —S(O)ₙ₄NR^{4A}R^{4B}, —NHNR^{4A}R^{4B}, —ONR^{4A}R^{4B}, —NHC(O)NHNR^{4A}R^{4B} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R is hydrogen, halogen, —N₃, —CF₃, —CCl₃, —CBr₃, —CI₃, —CN, —CHO, —OR^{5A}, —NR^{5A}R^{5B}, —COOR^{5A}, —C(O)NR^{5A}R^{5B}, —NO₂, —SR^{5A}, —S(O)ₙ₅R^{5A}, —S(O)ₙ₅OR^{5A}, —S(O)ₙ₅NR^{5A}R^{5B}, —NHNR^{5A}R^{5B}, —ONR^{5A}R^{5B}, —NHC(O)NHNR^{5A}R^{5B} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R⁴ and R⁵ are optionally joined together to form a substituted or unsubstituted heterocycloalkyl, or substituted or unsubstituted heteroaryl; R⁶ is —CF₃, substituted or unsubstituted cyclopropyl, or substituted or unsubstituted cyclobutyl; R⁷ is independently hydrogen, halogen, —N₃, —CF₃, —CCl₃, —CBr₃, —CI₃, —CN, —CHO, —OR^{7A}, —NR^{A}R^{7B}, —COOR^{7A}, —C(O)NR^{7A}R^{7B}, —NO₂, —SR^{7A}, —S(O)ₙ₇R^{7A}, —S(O)ₙ₇OR^{7A}, —S(O)ₙ₇NR^{7A}R^{7B}, —NHNR^{7A}R^{7B}, —ONR^{7A}R^{7B}, —NHC(O)NHNR^{7A}R^{7B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A}, R^{1B}, R^{2A}, R^{2B}, R^{3A}, R^{3B}, R^{4A}, R^{4B}, R^{5A}, R^{5B}, R^{7A} and R^{7B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and n1, n2, n3, n4, n5 and n7 are independently 1 or 2.

In some embodiments, the compound of formulae (VIII-a), (VIII-b), (VIII-c), or (VIII-d) is described in International Patent Publication No. WO 2014/165263 (e.g., in embodiments P1-P12), which is herein incorporated by reference in its entirety. In some embodiments, the compound of formulae (VIII-a), (VIII-b), (VIII-c), or (VIII-d) is selected from:

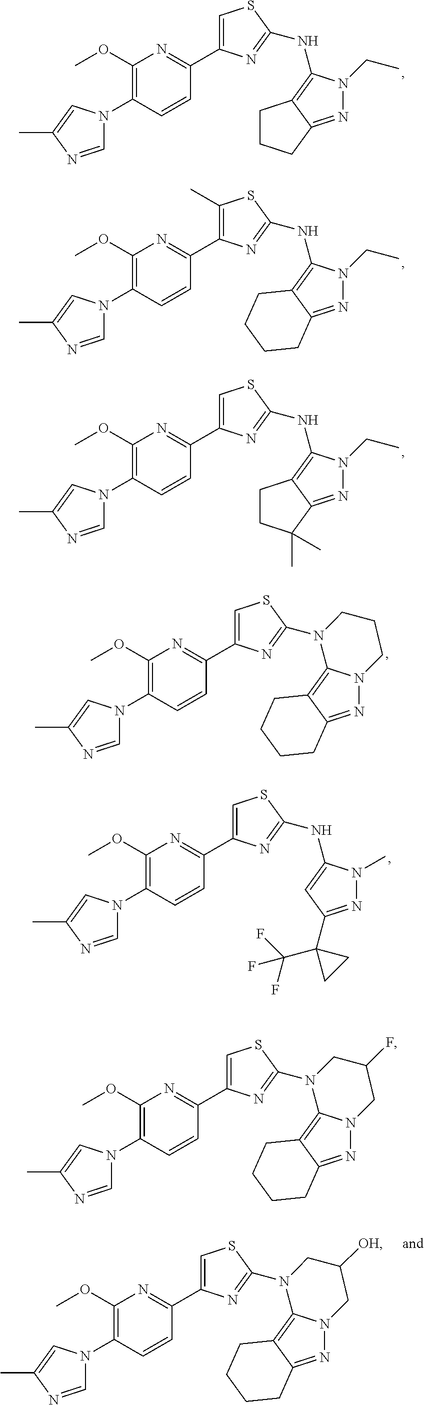

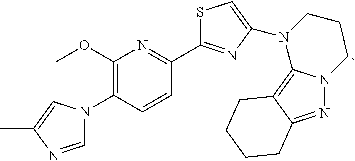 [SMILES: COc1nc(-c2nc(N3CCCn4nc5c(c43)CCCC5)cs2)ccc1-n1cnc(C)c1]

or a pharmaceutically acceptable salt thereof.

In some embodiments, the compound is a compound of formula (IX):

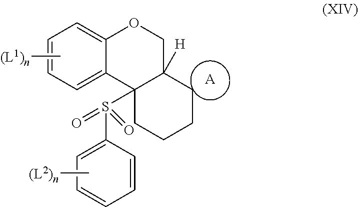

or a pharmaceutically acceptable salt thereof, wherein A is a 4 to 7 membered spirocyclic ring comprising at least one heteroatom selected from the group consisting of N, O, S, S(O)₂, P(O)R′, and N—S(O)₂—R′, wherein the spirocyclic ring is optionally substituted with 1 to 3 substituents selected from the group consisting of C1-3alkyl and ═O; R¹ is C1-6alkyl optionally substituted with halo; each L¹ is independently selected from the group consisting of 1) C1-3alkyl optionally substituted with halo, and 2) halo; each L² is independently selected from the group consisting of 1) C1-3alkyl optionally substituted with halo, and 2) halo; and
n is 0 to 3.

In some embodiments, the compound of formulae (IX) is described in U.S. Patent Publication No. US-2015-307533 (e.g., in the Table on pages 13-16), which is herein incorporated by reference in its entirety. In some embodiments, the compound of formula (IX) is selected from:

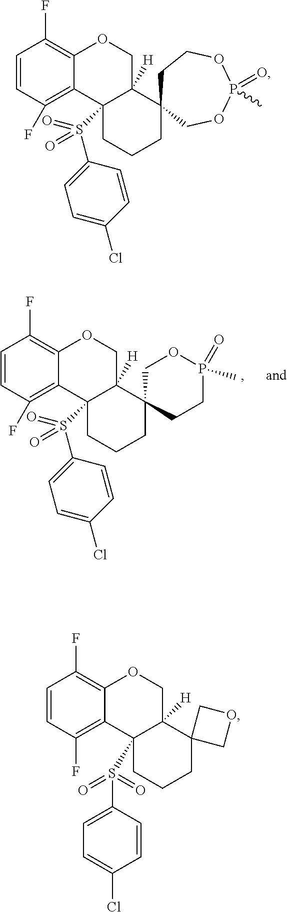

or a pharmaceutically acceptable salt thereof.

In some embodiments, the compound is a compound of formula (X):

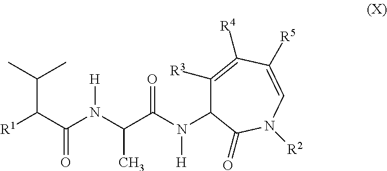

or a pharmaceutically acceptable salt thereof, wherein R¹ is hydroxy or fluoro; R² is C₁-C₄ alkyl; R³ is hydrogen or phenyl; R⁴ is hydrogen, phenyl, or C₁-C₄ alkyl; R⁵ is hydrogen or phenyl; provided that one of R³, R⁴, and R⁵ is other than hydrogen and the other two are hydrogen.

In some embodiments, the compound of formula (X) is described in U.S. Pat. No. 8,188,069, which is herein incorporated by reference in its entirety. In one embodiment, the compound is

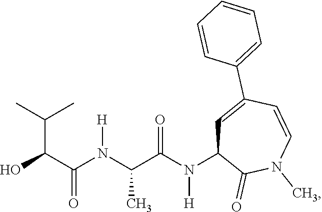 [SMILES: CC(C)[C@H](O)C(=O)N[C@@H](C)C(=O)N[C@H]1C=C(c2ccccc2)C=CN(C)C1=O]

or a pharmaceutically acceptable salt thereof.

In some embodiments, the compound is a compound of formula (XI):

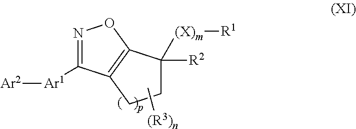

or a pharmaceutically acceptable salt thereof, wherein: R¹ is 1) hydrogen, 2) (C1-C6)alkyl optionally substituted with 1 to 5 halogens or phenyl, wherein the phenyl is optionally substituted with 1 to 3 halogens, 3) phenyl optionally substituted with 1 to 3 (C1-C6)alkyls or 1 to 5 halogens, or 4) (C4-C6)cycloalkyl optionally substituted with 1 to 3 (C1-C6)alkyls or 1 to 5 halogens; R² is 1) hydrogen, 2) (C1-C6)alkyl optionally substituted with 1 to 5 halogens or phenyl, wherein the phenyl is optionally substituted with 1 to 3 halogens, or 3) phenyl optionally substituted with 1 to 3 halogens; R³ is (C1-C6)alkyl, —OH or halogen;
X is —NR⁴—, —O—, —S—, or —SO₂—; R⁴ is hydrogen or (C1-C3)alkyl;
p is 1 to 3; m is 0 or 1; n is 0 to 3; and Ar²—Ar¹ is selected from the group consisting of

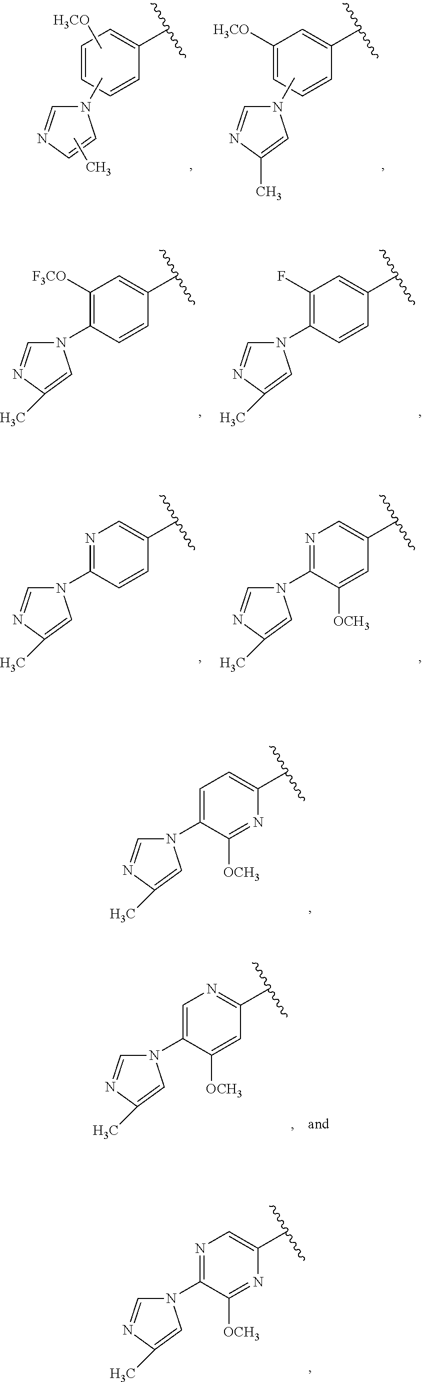

or a pharmaceutically acceptable salt thereof.

In some embodiments, the compound of formula (XI) is described in U.S. Pat. No. 9,096,582 (e.g., in the Table on pages 13-17), which is herein incorporated by reference in its entirety. In some embodiments, the compound of formula (XI) is selected from:

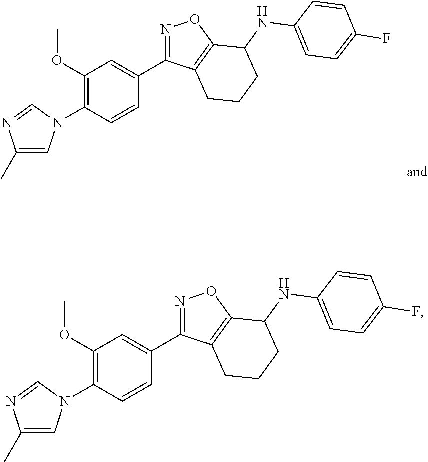 [SMILES: COc1cc(-c2noc3c2CCCC3Nc2ccc(F)cc2)ccc1-n1cnc(C)c1 ; COc1cc(-c2noc3c2CCCC3Nc2ccc(F)cc2)ccc1-n1cnc(C)c1]

or a pharmaceutically acceptable salt thereof.

In some embodiments, the compound is a compound of formula (XII):

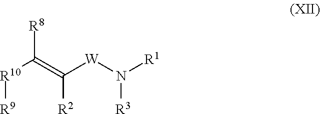

or a pharmaceutically acceptable salt thereof, wherein or the pharmaceutically acceptable salts thereof, wherein: R¹, R², R³, R⁸, R⁹, R¹⁰, and W are independently selected; W is selected from the group consisting of; —S(O)—, and —S(O)₂—; R¹ is selected from the group consisting of H, alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfused-heterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups; R² and R³ are each independently selected from the group consisting of H, alkyl-, alkenyl-, alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl-, and heterocyclyalkyl-; wherein each of said alkyl-, alkenyl- and alkynyl-, aryl-, arylalkyl-, alkylaryl-, cycloalkyl-, cycloalkenyl, cycloalkylalkyl-, cycloalkenyl-, heteroaryl-, heteroarylalkyl-, heterocyclyl-, heterocyclenyl- and heterocyclyalkyl-R¹ groups is optionally substituted with 1-5 independently selected R²¹ groups; or R² and R³ taken together, along with the atoms to which they are bound, form a ring selected from the group consisting of: (a) a 5 to 6 membered heterocycloalkyl ring, said heterocycloalkyl ring optionally comprising, in addition to W and in addition to the N adjacent to W, at least one other heteroatom independently selected from the group consisting of: —O—, —S(O)—, —S(O)₂, and —C(O)—, and (b) a 5 to 6 membered heterocycloalkenyl ring, said heterocycloalkenyl ring optionally comprising, in addition to W and in addition to the N adjacent to W, at least one other heteroatom independently selected from the group consisting of: —O—, —S(O)—, —S(O)₂, and —C(O)—; wherein said ring is optionally substituted with 1-5 independently selected R²¹ groups; or R² and R taken together along with the atoms to which they are bound, and R¹ and R³ are taken together along with the atoms to which they are bound, form the fused ring moiety:

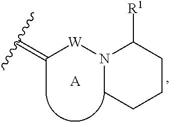

Wherein Ring A is a ring selected from the group consisting of: (a) a 5 to 6 membered heterocycloalkyl ring, said heterocycloalkyl ring optionally comprising, in addition to W and in addition to the N adjacent to W, at least one other heteroatom independently selected from the group consisting of: —O—, —NR¹⁴—, —S(O)—, —S(O)₂, and —C(O)—, and (b) a to 6 membered heterocycloalkenyl ring, said heterocycloalkenyl ring optionally comprising, in addition to W and in addition to the N adjacent to W, at least one other heteroatom independently selected from the group consisting of: —O—, —NR¹⁴—, —S(O)—, —S(O)₂, and —C(O)—, and wherein said fused ring moiety is optionally substituted with 1-5 independently selected R²¹ groups; or R¹ and R taken together with the atoms to which they are bound form a fused benzoheterocycloalkyl ring, and wherein said fused ring is optionally substituted with 1-5 independently selected R²¹ groups, R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl-; wherein each of said R⁸ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl, heterocyclenyl- and heterocyclyalkyl- is optionally substituted with 1-3 independently selected R²¹ groups; R⁹ is selected from the group consisting of: alkyl-, alkenyl-, alkynyl-, aryl-, arylalkyl-, alkylaryl-, cycloalkyl-, cycloalkenyl, cycloalkylalkyl, heteroaryl-, heteroarylalkyl-, heterocyclyl-, heterocyclenyl-, and heterocyclyalkyl-, wherein each of said R⁹ alkyl-, alkenyl- and alkynyl-, aryl-, arylalkyl-, alkylaryl-, cycloalkyl-, cycloalkenyl, cycloalkyl alkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;
R¹⁰ is selected from the group consisting of: a bond, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl-, heterocyclyalkenyl-,

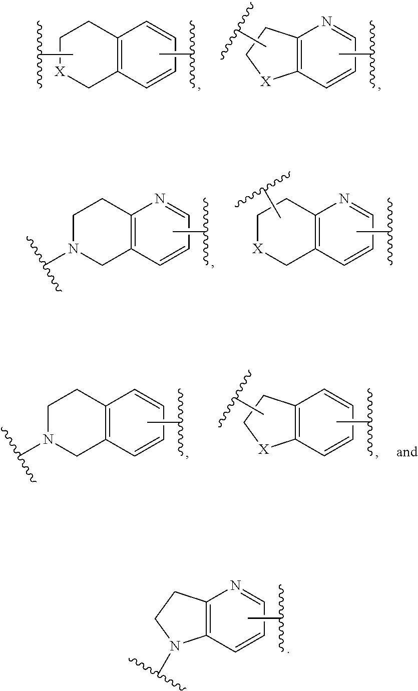

wherein X is selected from the group consisting of: O, —N(R¹⁴)— or —S—; and wherein each of said R¹⁰ moieties is optionally substituted with 1-3 independently selected R²¹ groups; R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —ON, —C(O)R″, —C(O)OR″, —C(O)N(R″)(R¹⁶), —S(O)N(R″)(R¹⁶), —S(O)₂N(R¹⁵)(R¹⁶), —C(═NOR¹⁵)R¹⁶, and —P(O)(OR¹⁵)(OR¹⁶); R′, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl; each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or two R¹⁸ moieties on adjacent carbons can be linked together to form a

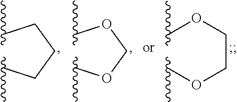

R¹⁹ is selected from the group consisting of: alkyl, cycloalkyl, aryl, arylalkyl and heteroarylalkyl; R²⁰ is selected from the group consisting of: alkyl, cycloalkyl, aryl, halo substituted aryl, arylalkyl, heteroaryl and heteroarylalkyl; each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —ON, —OR¹⁵, —C(O)R¹⁵, —C(O)OR¹⁵, —C(O)N(R¹⁵)(R¹⁶), —SR¹⁵, —S(O)N(R¹⁶)(R¹⁶), —CH(R¹⁵)(R¹⁶), —S(O)₂N(R¹⁶)(R¹⁶), —C(═NOR¹⁶)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —OH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups; and each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷); —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In some embodiments, the compound of formula (XII) is described in U.S. Patent Publication No. US-2011-0257163 (e.g., in paragraphs [0506] to [0553]), which is herein incorporated by reference in its entirety. In some embodiments, the compound of formula (XII) is a pharmaceutically acceptable ester. In some embodiments, the compound of formula (XII) is selected from:

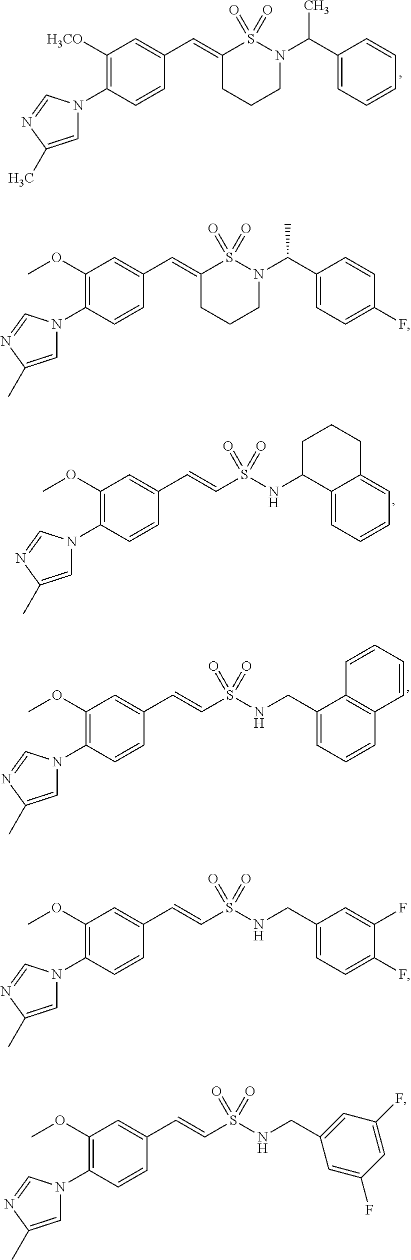 [SMILES: COc1cc(/C=C2\CCCN(C(C)c3ccccc3)S2(=O)=O)ccc1-n1cnc(C)c1 ; COc1cc(/C=C2\CCCN([C@H](C)c3ccc(F)cc3)S2(=O)=O)ccc1-n1cnc(C)c1 ; COc1cc(/C=C/S(=O)(=O)NC2CCCc3ccccc32)ccc1-n1cnc(C)c1 ; COc1cc(/C=C/S(=O)(=O)NCc2cccc3ccccc23)ccc1-n1cnc(C)c1 ; COc1cc(/C=C/S(=O)(=O)NCc2ccc(F)c(F)c2)ccc1-n1cnc(C)c1 ; COc1cc(/C=C/S(=O)(=O)NCc2cc(F)cc(F)c2)ccc1-n1cnc(C)c1]

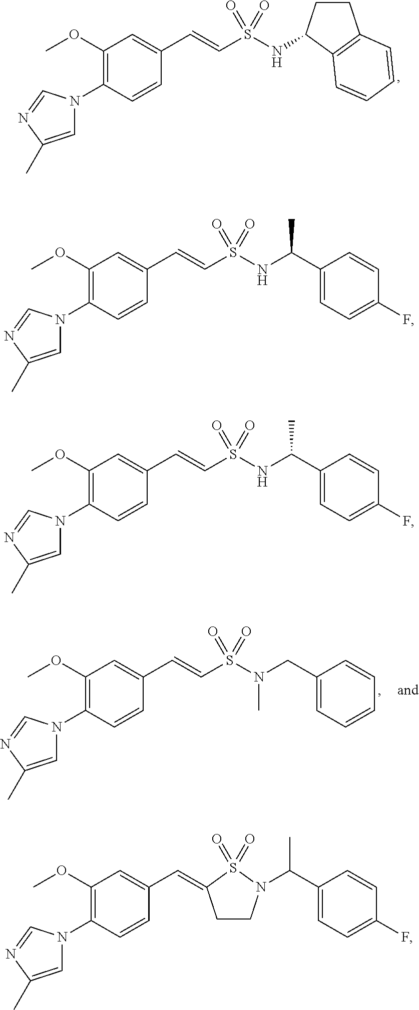 [SMILES: COc1cc(/C=C/S(=O)(=O)N[C@@H]2CCc3ccccc32)ccc1-n1cnc(C)c1 ; COc1cc(/C=C/S(=O)(=O)N[C@@H](C)c2ccc(F)cc2)ccc1-n1cnc(C)c1 ; COc1cc(/C=C/S(=O)(=O)N[C@H](C)c2ccc(F)cc2)ccc1-n1cnc(C)c1 ; COc1cc(/C=C/S(=O)(=O)N(C)Cc2ccccc2)ccc1-n1cnc(C)c1 ; COc1cc(/C=C2\CCN(C(C)c3ccc(F)cc3)S2(=O)=O)ccc1-n1cnc(C)c1]

or a pharmaceutically acceptable salt thereof.

In some embodiments, the compound is a compound of formula (XIII):

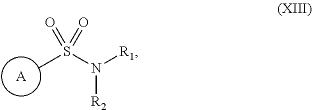

or a pharmaceutically acceptable salt thereof, wherein the A-ring is aryl, cycloalkyl, heteroaryl or heterocycloalkyl, where each ring is optionally substituted at a substitutable position with halogen, C₁-C₆ alkyl, C₂-C₆ alkenyl, C₂-C₆ alkynyl, C₁-C₆ alkoxy, haloalkyl, haloalkoxy, hydroxyl, hydroxyalkyl, CN, phenoxy, —S(O)₀₋₂—(C₁-C₆alkyl), —NR₁₀R₁₁, C₁-C₆alkanoyl, C₀-C₃alkylCO₂R′, heteroaryl, heterocycloalkyl, aryl, aralkyl, or —SO₂NR₁₀R₁₁; R₁ and R₂ combine to form a [3.3.1] or a [3.2.1] ring system, where 0 or 1 of the carbons in the ring system is optionally replaced with an —O—, —S(O)ₓ—, or —NR₁₅— group; and where the [3.3.1] or [3.2.1] ring system is optionally substituted with 1, 2, 3, or 4 groups that are independently oxo, halogen, C₁-C₆ alkyl, —O(C₁-C₂ alkyl)O—, —S(C₁-C₂ alkyl)S—, C₂-C₆ alkenyl, C₁-C₆ haloalkyl, C₂-C₆ alkynyl, hydroxy, hydroxyalkyl, C₁-C₆ alkoxy, haloalkoxy, —C(O)OR₁₃, —(C₁-C₄alkyl)-C(O)OR₁₆, —CONR₁₀R₁₁, —OC(O)NR₁₀R₁₁, —NR′C(O)OR″, —NR′S(O)₂R″, —OS(O)₂R′, —NR′COR″, CN, ═N—NR₁₂, or ═N—O—R₁₃; where x is 0, 1, or 2; R₁₀ and R₁₁ at each occurrence are independently hydrogen or C₁-C₆ alkyl, where the alkyl is optionally substituted with an aryl, where the aryl is optionally substituted with 1 to 5 groups that are independently halogen, hydroxyl, alkyl, alkoxy, haloalkyl, haloalkoxy, CN or NO₂; or
R₁₀ and R₁₁ together may form a 3-8 membered ring optionally including an additional heteroatom such as N, O or S; R12 is hydrogen, C₁-C₆ alkyl or —SO₂-aryl, where the aryl is optionally substituted with 1 to 5 groups that are independently halogen, hydroxyl, alkyl, alkoxy, haloalkyl, haloalkoxy, CN or NO₂;
R₁₃ is hydrogen or C₁-C₆ alkyl optionally substituted with aryl, hydroxyl, or halogen, where the aryl is optionally substituted with 1 to 5 groups that are independently halogen, hydroxyl, alkyl, alkoxy, haloalkyl, haloalkoxy, CN or NO₂;
R₁₅ is hydrogen, aryl, heteroaryl, —SO₂R′, —C(O)R′, —C(O)OR′, or C₁-C₆ alkyl optionally substituted with aryl, hydroxyl, or halogen, where the aryl groups are optionally substituted with 1 to 5 groups that are independently halogen, hydroxyl, alkyl, alkoxy, haloalkyl, haloalkoxy, CN or NO₂; and R′ and R″ are independently hydrogen, C₁-C₆ alkyl, haloalkyl, C₂-C₆ alkenyl or phenyl optionally substituted with 1 to 5 groups that are independently halogen, C₁-C₆ alkyl, —C(O)OR′, C₁-C₆ alkoxy, haloalkyl, haloalkoxy, hydroxyl, CN, phenoxy, —SO₂—(C₁-C₆ alkyl), —NR₁₀R₁₁, C₁-C₆ alkanoyl, pyridyl, phenyl, NO₂, or —SO₂NR₁₀R₁₁.

In some embodiments, the compound of formula (XIII) is described in U.S. Patent Publication No. US-2011-178199 (e.g., in paragraphs [0798] to [0799] and Tables 1-4), which is herein incorporated by reference in its entirety. In some embodiments, the compound of formula (XIII) comprises a bridged n-bicyclic sulfonamide or a pharmaceutically acceptable salt thereof. In some embodiments, the compound of formula (XIII) is selected from:

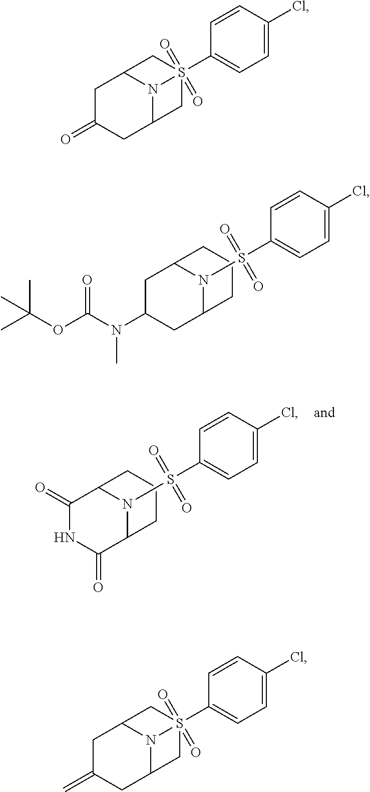

or a pharmaceutically acceptable salt thereof.

In some embodiments, the compound is a compound of formula (XIV):

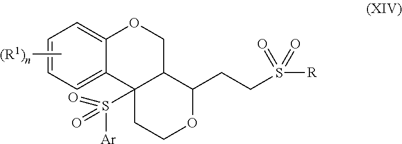

or a pharmaceutically acceptable salt thereof, wherein R is selected from the group consisting of: (1) -pyridinyl, (2) -pyrazolinyl, (3) -1,2,4-oxadiazolyl, (4) —(C1-C2)alkyl-pyridinyl, (5) —(C1-C2)alkyl-pyrazolinyl, and (6) —(C1-C2)alkyl-1,2,4-oxadiazolyl, wherein the pyridinyl, pyrazolinyl, and -1,2,4-oxadiazolyl, is unsubstituted or substituted with one L¹ group; R¹ is independently selected from the group consisting halogen, (C1-C6)alkyl, —CN, —CF₃, —O—(C1-C6)alkyl, —O-(halo(C1-C6)alkyl), —C(O)—O—(C1-C6)-OH-substituted (C1-C4)alkyl, halo(C1-C6)alkyl, —(C1-C4)alkoxy-OH, —(C1-C4)alkoxy(C1-C4)alkoxy and —S(O)₂(C1-C6)alkyl; n is 0, 1, 2, or 3; Ar is selected from the group consisting of phenyl optionally substituted with 1 or 2 L² groups, and pyridyl optionally substituted with 1 or 2 L² groups;
L¹ is independently selected from the group consisting of —OCH₃, —NH₂, =0, and (C1-C5)alkyl; and
L² is independently selected from the group consisting of halogen, (C1-C6)alkyl, —CN, —CF₃, —O—(C1-C6)alkyl, —O-(halo(C1-C6)alkyl), —C(O)—(C1-C6)alkyl, —OH-substituted(C1-C6)alkyl, halo(C1-C6)alkyl, —OH-substituted (C1-C4)alkoxy, —(C1-C4)alkoxy(C1-C4)alkoxy and —S(O)₂(C1-C6)alkyl.

In some embodiments, the compound of formula (XIV) is described in U.S. Pat. No. 9,226,927 (e.g., compound 4, 8a, 8b, 11, 14, 25a, 25b, 25c, 25d, 25e, 25f, 25g, 25h, 27a, or 27b), which is herein incorporated by reference in its entirety. In some embodiments, the compound of formula (XIV) comprises a bridged n-bicyclic sulfonamide or a pharmaceutically acceptable salt thereof. In some embodiments, the compound of formula (XIV) is selected from:

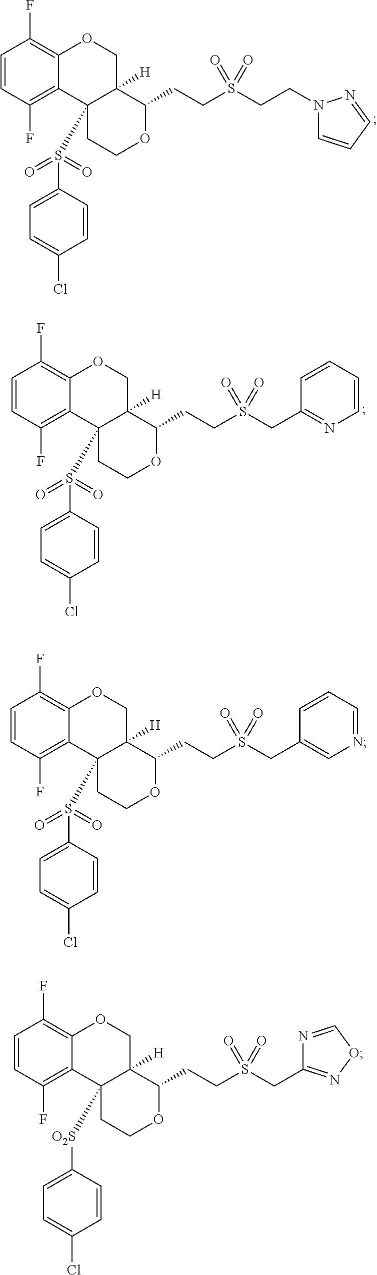

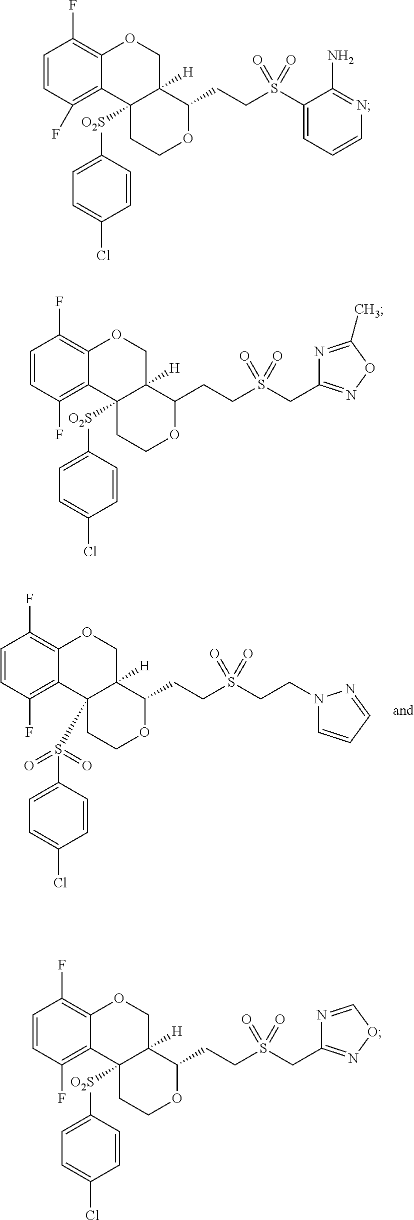 [SMILES: Nc1ncccc1S(=O)(=O)CC[C@@H]1OCC[C@@]2(S(=O)(=O)c3ccc(Cl)cc3)c3c(F)ccc(F)c3OC[C@@H]12 ; Cc1nc(CS(=O)(=O)CCC2OCC[C@@]3(S(=O)(=O)c4ccc(Cl)cc4)c4c(F)ccc(F)c4OC[C@@H]23)no1 ; O=S(=O)(CC[C@@H]1OCC[C@@]2(S(=O)(=O)c3ccc(Cl)cc3)c3c(F)ccc(F)c3OC[C@@H]12)CCn1cccn1 ; O=S(=O)(CC[C@@H]1OCC[C@@]2(S(=O)(=O)c3ccc(Cl)cc3)c3c(F)ccc(F)c3OC[C@@H]12)Cc1ncon1]

or a pharmaceutically acceptable salt thereof.

### Antibody Molecules Targeting Gamma Secretase

The compositions, methods and uses described herein comprise a gamma secretase inhibitor (GSI). In some embodiments, the GSI is an antibody molecule that reduces the expression and/or function of gamma secretase. In some embodiments, the GSI is an antibody molecule targeting a subunit of gamma secretase. In some embodiments, the GSI is chosen from an anti-presenilin antibody molecule, an anti-nicastrin antibody molecule, an anti-APH-1 antibody molecule, or an anti-PEN-2 antibody molecule.

Exemplary antibody molecules that target a subunit of gamma secretase (e.g., e.g., presenilin, nicastrin, APH-1, or PEN-2) are described in U.S. Pat. Nos. 8,394,376, 8,637,274, and 5,942,400, incorporated by reference herein in their entirety.

### Gene Editing Systems Targeting Gamma Secretase

According to the present invention, gene editing systems can be used as inhibitors of gamma secretase (e.g., a subunit of gamma secretase, e.g., presenilin, nicastrin, APH-1, or PEN-2). Also contemplated by the present invention are the uses of a nucleic acid encoding one or more components of a gene editing system targeting gamma secretase (e.g., a subunit of gamma secretase, e.g., presenilin, nicastrin, APH-1, or PEN-2).

### CRISPR/Cas9 Gene Editing Systems

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system has been modified for use in gene editing (silencing, enhancing or changing specific genes) in eukaryotes such as mice or primates. Wiedenheft et al. (2012) Nature 482: 331-8. This is accomplished by, for example, introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence. In an exemplary CRISPR/Cas system targeting gamma secretase (e.g., a subunit of gamma secretase, e.g., presenilin, nicastrin, APH-1, or PEN-2), the spacers are derived from the gene sequence of gamma secretase (e.g., a subunit of gamma secretase, e.g., presenilin, nicastrin, APH-1, or PEN-2), or a sequence of its regulatory elements.

RNA from the CRISPR locus is constitutively expressed and processed into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stem et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

The CRISPR/Cas system can thus be used to modify, e.g., delete one or more nucleic acids, e.g., a gene encoding gamma secretase (e.g., a subunit of gamma secretase, e.g., presenilin, nicastrin, APH-1, or PEN-2), or a regulatory element of a gene encoding gamma secretase (e.g., a subunit of gamma secretase, e.g., presenilin, nicastrin, APH-1, or PEN-2), or introduce a premature stop which thus decreases expression of a functional gamma secretase (e.g., a subunit of gamma secretase, e.g., presenilin, nicastrin, APH-1, or PEN-2). The CRISPR/Cas system can alternatively be used like RNA interference, turning off a gene encoding gamma secretase (e.g., a subunit of gamma secretase, e.g., presenilin, nicastrin, APH-1, or PEN-2) in a reversible fashion. In a mammalian cell, for example, the RNA can guide the Cas protein to a promoter of a gene encoding gamma secretase (e.g., a subunit of gamma secretase, e.g., presenilin, nicastrin, APH-1, or PEN-2), sterically blocking RNA polymerases.

CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each includes a hybridization domain which allows the molecules to associate, e.g., through hybridization.

Artificial CRISPR/Cas systems can be generated which inhibit a gene encoding gamma secretase (e.g., a subunit of gamma secretase, e.g., presenilin, nicastrin, APH-1, or PEN-2), using technology known in the art, e.g., that are described in U.S. Publication No. 20140068797, WO2015/048577, and Cong (2013) Science 339: 819-823. Other artificial CRISPR/Cas systems that are known in the art may also be generated which inhibit a gene encoding gamma secretase (e.g., a subunit of gamma secretase, e.g., presenilin, nicastrin, APH-1, or PEN-2), e.g., that described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated which inhibit a gene encoding gamma secretase (e.g., a subunit of gamma secretase, e.g., presenilin, nicastrin, APH-1, or PEN-2), by, for example, engineering a CRISPR/Cas system to include a gRNA molecule comprising a targeting sequence that hybridizes to a sequence of a target gene, e.g., a gene encoding gamma secretase (e.g., a subunit of gamma secretase, e.g., presenilin, nicastrin, APH-1, or PEN-2). In embodiments, the gRNA comprises a targeting sequence which is fully complementarity to 15-25 nucleotides, e.g., 20 nucleotides, of a target gene, e.g., a gene encoding gamma secretase (e.g., a subunit of gamma secretase, e.g., presenilin, nicastrin, APH-1, or PEN-2). In embodiments, the 15-25 nucleotides, e.g., 20 nucleotides, of a target gene, e.g., a gene encoding gamma secretase (e.g., a subunit of gamma secretase, e.g., presenilin, nicastrin, APH-1, or PEN-2), are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C).

In an embodiment, the CRISPR/Cas system of the present invention comprises Cas9, e.g., S. pyogenes Cas9, and a gRNA comprising a targeting sequence which hybridizes to a sequence of a gene encoding gamma secretase (e.g., a subunit of gamma secretase, e.g., presenilin, nicastrin, APH-1, or PEN-2). In an embodiment, the CRISPR/Cas system comprises nucleic acid encoding a gRNA specific for a gene encoding gamma secretase (e.g., a subunit of gamma secretase, e.g., presenilin, nicastrin, APH-1, or PEN-2), and a nucleic acid encoding a Cas protein, e.g., Cas9, e.g., S. pyogenes Cas9. In an embodiment, the CRISPR/Cas system comprises a gRNA specific for a gene encoding gamma secretase (e.g., a subunit of gamma secretase, e.g., presenilin, nicastrin, APH-1, or PEN-2), and a nucleic acid encoding a Cas protein, e.g., Cas9, e.g., S. pyogenes Cas9.

### TALEN Gene Editing Systems

TALENs are produced artificially by fusing a TAL effector DNA binding domain to a DNA cleavage domain. Transcription activator-like effects (TALEs) can be engineered to bind any desired DNA sequence, including a portion of the HLA or TCR gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A TALEN specific for a gene encoding gamma secretase (e.g., a subunit of gamma secretase, e.g., presenilin, nicastrin, APH-1, or PEN-2), can be used inside a cell to produce a double-stranded break (DSB). A mutation can be introduced at the break site if the repair mechanisms improperly repair the break via non-homologous end joining. For example, improper repair may introduce a frame shift mutation.

TALENs specific to sequences in a gene encoding gamma secretase (e.g., a subunit of gamma secretase, e.g., presenilin, nicastrin, APH-1, or PEN-2), can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorporated by reference in their entirety.

### Zinc Finger Nucleases

“ZFN” or “Zinc Finger Nuclease” refers to a zinc finger nuclease, an artificial nuclease which can be used to modify, e.g., delete one or more nucleic acids of, a desired nucleic acid sequence, e.g., a gene encoding gamma secretase (e.g., a subunit of gamma secretase, e.g., presenilin, nicastrin, APH-1, or PEN-2).

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression of a gene encoding gamma secretase (e.g., a subunit of gamma secretase, e.g., presenilin, nicastrin, APH-1, or PEN-2), in a cell. ZFNs can also be used with homologous recombination to mutate a gene encoding gamma secretase (e.g., a subunit of gamma secretase, e.g., presenilin, nicastrin, APH-1, or PEN-2).

ZFNs specific to sequences in a gene encoding gamma secretase (e.g., a subunit of gamma secretase, e.g., presenilin, nicastrin, APH-1, or PEN-2), can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. In embodiments, The ZFN gene editing system may also comprise nucleic acid encoding one or more components of the ZFN gene editing system, e.g., a ZFN gene editing system targeted to a gene encoding gamma secretase (e.g., a subunit of gamma secretase, e.g., presenilin, nicastrin, APH-1, or PEN-2).

Double-Stranded RNA, e.g., siRNA or shRNA, Targeting Gamma Secretase

According to the present invention, double stranded RNA (“dsRNA”), e.g., siRNA or shRNA can be used to inhibit a gene encoding gamma secretase (e.g., a subunit of gamma secretase, e.g., presenilin, nicastrin, APH-1, or PEN-2). Also contemplated by the present invention are the uses of a nucleic acid encoding said dsRNA inhibitors of a gene encoding gamma secretase (e.g., a subunit of gamma secretase, e.g., presenilin, nicastrin, APH-1, or PEN-2).

In an embodiment, the GSI is a nucleic acid, e.g., a dsRNA, e.g., a siRNA or shRNA specific for a nucleic acid encoding gamma secretase (e.g., a subunit of gamma secretase, e.g., presenilin, nicastrin, APH-1, or PEN-2).

An aspect of the invention provides a composition comprising a dsRNA, e.g., a siRNA or shRNA, comprising at least 15 contiguous nucleotides, e.g., 15, 16, 17, 18, 19, 20, 21, 22, 23, 24 or 25 contiguous nucleotides, e.g., 21 contiguous nucleotides. It is understood that some of the target sequences and/or shRNA molecules are presented as DNA, but the dsRNA agents targeting these sequences or comprising these sequences can be RNA, or any nucleotide, modified nucleotide or substitute disclosed herein and/or known in the art, provided that the molecule can still mediate RNA interference.

In embodiments, the GSI is a nucleic acid, e.g., DNA, encoding a dsRNA inhibitor, e.g., shRNA or siRNA, of any of the above embodiments. In embodiments, the nucleic acid, e.g., DNA, is disposed on a vector, e.g., any conventional expression system, e.g., as described herein, e.g., a lentiviral vector.

### Antibody Molecules Targeting BCMA

In one embodiment, the BCMA-targeting agent is an anti-BCMA antibody molecule. In one embodiment, the antibody molecule binds to mammalian, e.g., human, BCMA. In one embodiment, the anti-BCMA antibody molecule, when bound to BCMA-expressing cells, e.g., BCMA-expressing tumor cells, can induce antibody-dependent cellular cytotoxicity (ADCC) or complement-dependent cytotoxicity (CDC) of BCMA-expressing cells, e.g., BCMA-expressing tumor cells. In one embodiment, the anti-BCMA antibody molecule is linked, e.g., via a linker, to a drug moiety, e.g., a drug moiety that exerts a cytotoxic or cytostatic activity. In one embodiment, the anti-BCMA antibody molecule is a multispecific antibody molecule, e.g., a multispecific antibody molecule comprising a first binding moiety that specifically binds to BCMA and a second binding moiety that specifically binds to an antigen on an immune effector cell.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3)(see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

In an embodiment, an antibody molecule is a monospecific antibody molecule and binds a single epitope, e.g., a monospecific antibody molecule having a plurality of immunoglobulin variable domain sequences, each of which binds the same epitope.

In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibodies disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region” and “CDR” as used herein refer to the sequences of amino acids within antibody variable regions which confer antigen specificity and binding affinity. In some embodiments, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme). As used herein, the CDRs defined according to the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Under all definitions, each VH and VL typically includes three CDRs and four FRs, arranged from amino-terminus to carboxy-terminus in the following order: FR1, CDR1, FR2, CDR2, FR3, CDR3, FR4.

Generally, unless specifically indicated, the anti-BCMA antibodies can include any combination of one or more Kabat CDRs, Chothia CDRs, combination of Kabat and Chothia CDRs, and/or IMGT CDRs.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to a BCMA polypeptide, or an epitope thereof. With respect to proteins (or protein mimetics), the antigen-binding site typically includes one or more loops (of at least, e.g., four amino acids or amino acid mimics) that form an interface that binds to a BCMA polypeptide. Typically, the antigen-binding site of an antibody molecule includes at least one or two CDRs and/or hypervariable loops, or more typically at least three, four, five or six CDRs and/or hypervariable loops.

As used herein, the term “Eu numbering” refers to the Eu numbering convention for the constant regions of an antibody, as described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991.

The terms “compete” or “cross-compete” are used interchangeably herein to refer to the ability of an antibody molecule to interfere with binding of an anti-BCMA antibody molecule, e.g., an anti-BCMA antibody molecule provided herein, to a target, e.g., human BCMA. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In some embodiments, a competition binding assay is a quantitative competition assay. In some embodiments, a first anti-BCMA antibody molecule is said to compete for binding to the target with a second anti-BCMA antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

As used herein, the term “epitope” refers to the moieties of an antigen (e.g., human BCMA) that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

In an embodiment, an epitopic determinant is a moiety on the antigen, e.g., such as amino acid side chain or sugar side chain, or part thereof, which, when the antigen and antibody molecule are co-crystallized, is within a predetermined distance, e.g., within 5 Angstroms, of a moiety on the antibody molecule, referred to herein as a “crystallographic epitopic determinant.” The crystallographic epitopic determinants of an epitope are collectively referred to as the “crystallographic epitope.”

A first antibody molecule binds the same epitope as a second antibody molecule (e.g., a reference antibody molecule, e.g., an antibody molecule disclosed herein) if the first antibody specifically interacts with the same epitopic determinants on the antigen as does the second or reference antibody, e.g., when interaction is measured in the same way for both the antibody and the second or reference antibody. Epitopes that overlap share at least one epitopic determinant. A first antibody molecule binds an overlapping epitope with a second antibody molecule (e.g., a reference antibody molecule, e.g., an antibody disclosed herein) when both antibody molecules specifically interact with a common epitopic determinant. A first and a second antibody molecule (e.g., a reference antibody molecule, e.g., an antibody molecule disclosed herein) bind substantially overlapping epitopes if at least half of the epitopic determinants of the second or reference antibody are found as epitopic determinants in the epitope of the first antibody. A first and a second antibody molecule (e.g., a reference antibody molecule, e.g., an antibody molecule disclosed herein) bind substantially the same epitope if the first antibody molecule binds at least half of the core epitopic determinants of the epitope of the second or reference antibody, wherein the core epitopic determinants are defined by, e.g., crystallography or hydrogen-deuterium exchange.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by yeast display, phage display, or by combinatorial methods.

In one embodiment, the antibody is a fully human antibody (e.g., an antibody produced by yeast display, an antibody produced by phage display, or an antibody made in a mouse which has been genetically engineered to produce an antibody from a human immunoglobulin sequence), or a non-human antibody, e.g., a rodent (mouse or rat), goat, primate (e.g., monkey), or camel antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Antibodies can be produced by any suitable recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to BCMA. In some embodiments, the donor is a rodent antibody, e.g., a rat or mouse antibody, and the recipient is a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has effector function and can fix complement. In other embodiments the antibody does not recruit effector cells or fix complement. In certain embodiments, the antibody has reduced or no ability to bind an Fc receptor. For example, it may be an isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (Eu numbering) in human IgG4, are also contemplated. Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the invention may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., immunomodulatory, immunostimularoty, cytotoxic, or cytostatic) agent or moiety. Radioactive isotopes can be used in diagnostic or therapeutic applications. Radioactive isotopes that can be coupled to the anti-BCMA antibodies include, but are not limited to α-, β-, or γ-emitters, or β- and γ-emitters. Such radioactive isotopes include, but are not limited to iodine (¹³¹I or ¹²⁵), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (^{99m}Tc), phosphorus (³²P), carbon (¹⁴C), and tritium (³H), or one or more of the therapeutic isotopes listed above.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs orhomologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In other embodiments, the anti-BCMA antibody molecule (e.g., a monospecific, bispecific, or multispecific antibody molecule) is covalently linked, e.g., fused, to another partner e.g., a protein e.g., one, two or more cytokines, e.g., as a fusion molecule for example a fusion protein.

A “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

This invention provides an isolated nucleic acid molecule encoding the above antibody molecule, vectors and host cells thereof. The nucleic acid molecule includes but is not limited to RNA, genomic DNA and cDNA.

### Exemplary Multispecific Antibody Molecules Targeting BCMA and Methods of Making the Same

In certain embodiments, the antibody or antibody-like molecule is a multispecific (e.g., a bispecific or a trispecific) antibody or antibody-like molecule. Protocols for generating bispecific or heterodimeric antibody or antibody-like molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody or antibody-like molecule determinants generated by recombining half antibodies (heavy-light chain pairs or Fabs) from different antibodies or antibody-like molecules through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; VL and VH domains, scFvs, or Fabs wherein one of the antigens is bound monovalently and one of the antigens is bound bivalently, optionally comprising heterodimeric Fc regions, as described in, e.g., WO2011/028952; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFv or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties. Accordingly, in some embodiments, the anti-BCMA multispecific molecules of the present invention comprises an anti-BCMA binding domain in any one of the multispecific or bispecific formats known in the art and described above. Additional formats contemplated herein are described in more detail below.

Within each antibody or antibody fragment (e.g., scFv) of a multispecific antibody or antibody-like molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody or antibody-like molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody or antibody-like molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄Ser)n linker, wherein n is 1, 2, 3, 4, 5, or 6, preferably 4 (SEQ ID NO: 1156). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, the anti-BCMA bispecific molecule of the present invention comprises VLs, VHs, (e.g., VLs and/or VHs from any of the anti-BCMA binding domains described herein) and optionally one or more linkers in an arrangement as described herein, together with, for example, VLs, VHs targeting a second epitope or antigen (e.g., a second epitope or antigen described herein, e.g., CD3 or CD16).

In certain embodiments, the multispecific antibody molecule, e.g. a BCMA×CD3, BCMA×CD16 (e.g., CD16A), BCMA×CD64, BCMA×NKG2D, or BCMA×CD47 multispecific antibody molecule, used in the compositions, methods, or uses disclosed herein is a BCMA×CD3 multispecific antibody molecule disclosed in WO 2014/110601, WO 2014/145806, WO 2016/086189, WO 2016/182751, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, incorporated herein by reference in their entireties.

In certain embodiments, the present invention provides a multispecific antibody molecule, e.g. a BCMA×CD3, BCMA×CD16 (e.g., CD16A), BCMA×CD64, BCMA×NKG2D, or BCMA×CD47 multispecific antibody molecule.

In one embodiment, the multispecific antibody molecule, e.g., a BCMA×CD3, BCMA×CD16 (e.g., CD16A), BCMA×CD64, BCMA×NKG2D, or BCMA×CD47 multispecific antibody molecule, is in a Dualbody format, e.g., a Dualbody format disclosed in WO2008/119353 and WO2011/131746, incorporated herein by reference in their entireties.

In one embodiment, the multispecific antibody molecule, e.g., a BCMA×CD3, BCMA×CD16 (e.g., CD16A), BCMA×NKG2D, or BCMA×CD47 multispecific antibody molecule, is in a format disclosed in WO 2014/110601, WO 2014/145806, WO 2016/086189, and WO 2016/182751, incorporated herein by reference in their entireties.

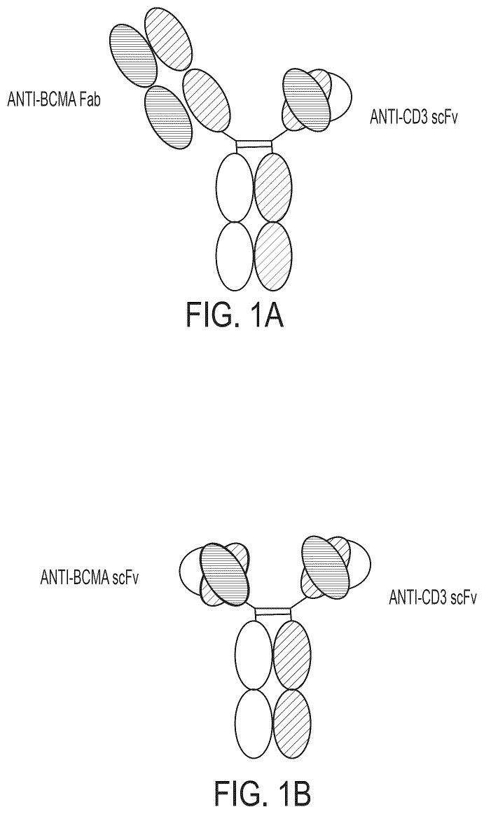

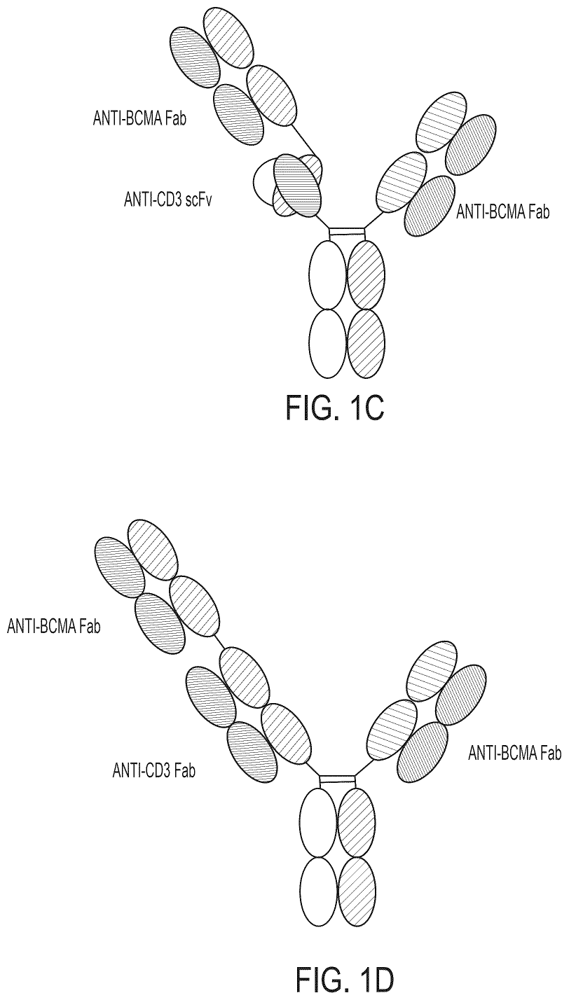

In one embodiment, the multispecific antibody molecule, e.g., the BCMA×CD3 multispecific antibody molecule, is in a format depicted in FIGS. 1A-1C of WO 2016/182751. In one embodiment, the multispecific antibody molecule, e.g., the BCMA×CD3 multispecific antibody molecule, is in a bottle-opener format (also known as a triple F format) depicted in FIG. 1A of WO 2016/182751. In one embodiment, the multispecific antibody molecule, e.g., the BCMA×CD3 multispecific antibody molecule, is in a central-scFv format depicted in FIG. 1B of WO 2016/182751. In one embodiment, the multispecific antibody molecule, e.g., the BCMA×CD3 multispecific antibody molecule, is in a dual-scFv format depicted in FIG. 1B of WO 2016/182751.

In one embodiment, the multispecific antibody molecule, e.g., the BCMA×CD3 multispecific antibody molecule, is in a format depicted in FIG. 1A. In one embodiment, the first half of the BCMA×CD3 multispecific antibody molecule comprises an anti-BCMA Fab, the CH1 domain of which is linked to a first Fc domain; and the second half of the multispecific antibody molecule comprises an anti-CD3 scFv linked to a second Fc domain. In one embodiment, the multispecific antibody molecule, e.g., the BCMA×CD3 multispecific antibody molecule, is in a format depicted in FIG. 1B. In one embodiment, the first half of the BCMA×CD3 multispecific antibody molecule comprises an anti-BCMA scFv linked to a first Fc domain; and the second half of the multispecific antibody molecule comprises an anti-CD3 scFv linked to a second Fc domain. In one embodiment, the multispecific antibody molecule, e.g., the BCMA×CD3 multispecific antibody molecule, is in a format depicted in FIG. 1C. In one embodiment, the first half of the BCMA×CD3 multispecific antibody molecule comprises a first anti-BCMA Fab, the CH1 domain of which is linked to an anti-CD3 scFv, which is further linked to a first Fc domain; and the second half of the multispecific antibody molecule comprises a second anti-BCMA Fab, the CH1 domain of which is linked to a second Fc domain. In one embodiment, the multispecific antibody molecule, e.g., the BCMA×CD3 multispecific antibody molecule, is in a format depicted in FIG. 1D. In one embodiment, the first half of the BCMA×CD3 multispecific antibody molecule comprises a first anti-BCMA Fab, an anti-CD3 scFv, and a first Fc domain; and the second half of the multispecific antibody molecule comprises a second anti-BCMA Fab and a second Fc domain.

In one embodiment, the two halves of the multispecific antibody molecule are brought together by the use of amino acid variants in the constant regions (e.g. the Fc domain and/or the hinge region) that promote the formation of heterodimeric antibodies. A variety of approaches available in the art can be used in for enhancing dimerization of the two heavy chain domains of bispecific or multispecific antibody or antibody-like molecules, as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1, incorporated by reference herein in their entirety.

The present invention provides methods of enhancing dimerization (hetero-dimerization) of two interacting heterologous polypeptides and/or reducing dimerization (homo-dimerization) of two identical polypeptides. Typically, each of the two interacting polypeptides comprises a CH3 domain of an antibody. The CH3 domains are derived from the constant region of an antibody of any isotype, class or subclass, and preferably of IgG (IgG1, IgG2, IgG3 and IgG4) class.

Typically, the polypeptides comprise other antibody fragments in addition to CH3 domains, such as, CH1 domains, CH2 domains, hinge domain, VH domain(s), VL domain(s), CDR(s), and/or antigen-binding fragments described herein. These antibody fragments are derived from various types of antibodies described herein, for example, polyclonal antibody, monoclonal antibodies, chimeric antibodies, humanized antibodies, human antibodies, bispecific or multispecific antibodies, camelised antibodies, anti-idiotypic (anti-Id) antibodies and antibody conjugates. In some embodiments, the two hetero-polypeptides are two heavy chains forming a bispecific or multispecific molecules. Heterodimerzation of the two different heavy chains at CH3 domains give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific molecule Formats described above of the present invention. In an embodiment, the two hetero-polypeptide chains comprising CH3 domains comprise modifications that favor heterodimeric association of the polypeptides, relative to unmodified chains. Various examples of modification strategies are provided below.

### Knob-in-Hole (KIH)

Multispecific molecules, e.g., multispecific antibody or antibody-like molecules, of the present invention may comprise one or more, e.g., a plurality, of mutations to one or more of the constant domains, e.g., to the CH3 domains. In one example, the multispecific molecule of the present invention comprises two polypeptides that each comprise a heavy chain constant domain of an antibody, e.g., a CH2 or CH3 domain. In an example, the two heavy chain constant domains, e.g., the CH2 or CH3 domains of the multispecific molecule comprise one or more mutations that allow for a heterodimeric association between the two chains. In one aspect, the one or more mutations are disposed on the CH2 domain of the two heavy chains of the multispecific, e.g., bispecific, antibody or antibody-like molecule. In one aspect, the one or more mutations are disposed on the CH3 domains of at least two polypeptides of the multispecific molecule. In one aspect, the one or more mutations to a first polypeptide of the multispecific molecule comprising a heavy chain constant domain creates a “knob” and the one or more mutations to a second polypeptide of the multispecific molecule comprising a heavy chain constant domain creates a “hole,” such that heterodimerization of the polypeptide of the multispecific molecule comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” As the term is used herein, a “knob” refers to at least one amino acid side chain which projects from the interface of a first polypeptide of the multispecific molecule comprising a heavy chain constant domain and is therefore positionable in a compensatory “hole” in the interface with a second polypeptide of the multispecific molecule comprising a heavy chain constant domain so as to stabilize the heteromultimer, and thereby favor heteromultimer formation over homomultimer formation, for example. The knob may exist in the original interface or may be introduced synthetically (e.g. by altering nucleic acid encoding the interface). The preferred import residues for the formation of a knob are generally naturally occurring amino acid residues and are preferably selected from arginine (R), phenylalanine (F), tyrosine (Y) and tryptophan (W). Most preferred are tryptophan and tyrosine. In the preferred embodiment, the original residue for the formation of the protuberance has a small side chain volume, such as alanine, asparagine, aspartic acid, glycine, serine, threonine or valine.

A “hole” refers to at least one amino acid side chain which is recessed from the interface of a second polypeptide of the multispecific molecule comprising a heavy chain constant domain and therefore accommodates a corresponding knob on the adjacent interfacing surface of a first polypeptide of the multispecific molecule comprising a heavy chain constant domain. The hole may exist in the original interface or may be introduced synthetically (e.g. by altering nucleic acid encoding the interface). The preferred import residues for the formation of a hole are usually naturally occurring amino acid residues and are preferably selected from alanine (A), serine (S), threonine (T) and valine (V). Most preferred are serine, alanine or threonine. In the preferred embodiment, the original residue for the formation of the hole has a large side chain volume, such as tyrosine, arginine, phenylalanine or tryptophan.

In a preferred embodiment, a first CH3 domain is mutated at residue 366, 405 or 407 according to the EU numbering scheme of Kabat et al. (pp. 688-696 in Sequences of proteins of immunological interest, 5th ed., Vol. 1 (1991; NIH, Bethesda, Md.)) to create either a “knob” or a hole” (as described above), and the second CH3 domain that heterodimerizes with the first CH3 domain is mutated at: residue 407 if residue 366 is mutated in the first CH3 domain, residue 394 if residue 405 is mutated in the first CH3 domain, or residue 366 if residue 407 is mutated in the first CH3 domain, according to the EU numbering scheme of Kabat et al. (pp. 688-696 in Sequences of proteins of immunological interest, 5th ed., Vol. 1 (1991; NIH, Bethesda, Md.)), to create a “hole” or “knob” complementary to the “knob” or “hole” of the first CH3 domain.

In another preferred embodiment, a first CH3 domain is mutated at residue 366 according to the EU numbering scheme of Kabat et al. (pp. 688-696 in Sequences of proteins of immunological interest, 5th ed., Vol. 1 (1991; NIH, Bethesda, Md.)) to create either a “knob” or a hole” (as described above), and the second CH3 domain that heterodimerizes with the first CH3 domain is mutated at residues 366, 368 and/or 407, according to the EU numbering scheme of Kabat et al. (pp. 688-696 in Sequences of proteins of immunological interest, 5th ed., Vol. 1 (1991; NIH, Bethesda, Md.)), to create a “hole” or “knob” complementary to the “knob” or “hole” of the first CH3 domain. In one embodiment, the mutation to the first CH3 domain introduces a tyrosine (Y) residue at position 366. In an embodiment, the mutation to the first CH3 is T366Y. In one embodiment, the mutation to the first CH3 domain introduces a tryptophan (W) residue at position 366. In an embodiment, the mutation to the first CH3 is T366W. In embodiments, the mutation to the second CH3 domain that heterodimerizes with the first CH3 domain mutated at position 366 (e.g., has a tyrosine (Y) or tryptophan (W) introduced at position 366, e.g., comprises the mutation T366Y or T366W), comprises a mutation at position 366, a mutation at position 368 and a mutation at position 407, according to the EU numbering scheme of Kabat et al. (pp. 688-696 in Sequences of proteins of immunological interest, 5th ed., Vol. 1 (1991; NIH, Bethesda, Md.)) In embodiments, the mutation at position 366 introduces a serine (S) residue, the mutation at position 368 introduces an alanine (A), and the mutation at position 407 introduces a valine (V). In embodiments, the mutations comprise T366S, L368A and Y407V. In one embodiment the first CH3 domain of the multispecific molecule comprises the mutation T366Y, and the second CH3 domain that heterodimerizes with the first CH3 domain comprises the mutations T366S, L368A and Y407V, or vice versa. In one embodiment the first CH3 domain of the multispecific molecule comprises the mutation T366W, and the second CH3 domain that heterodimerizes with the first CH3 domain comprises the mutations T366S, L368A and Y407V, or vice versa.

Additional knob in hole mutation pairs suitable for use in any of the multispecific molecules of the present invention are further described in, for example, WO1996/027011, and Merchant et al., Nat. Biotechnol., 16:677-681 (1998), the contents of which are hereby incorporated by reference in their entirety.

In any of the embodiments described herein, the CH3 domains may be additionally mutated to introduce a pair of cysteine residues. Without being bound by theory, it is believed that the introduction of a pair of cysteine residues capable of forming a disulfide bond provide stability to the heterodimerized multispecific molecule. In embodiments, the first CH3 domain comprises a cysteine at position 354, according to the EU numbering scheme of Kabat et al. (pp. 688-696 in Sequences of proteins of immunological interest, 5th ed., Vol. 1 (1991; NIH, Bethesda, Md.)), and the second CH3 domain that heterodimerizes with the first CH3 domain comprises a cysteine at position 349, according to the EU numbering scheme of Kabat et al. (pp. 688-696 in Sequences of proteins of immunological interest, 5th ed., Vol. 1 (1991; NIH, Bethesda, Md.)) In embodiments, the first CH3 domain of the multispecific molecule comprises a cysteine at position 354 (e.g., comprises the mutation S354C) and a tyrosine (Y) at position 366 (e.g., comprises the mutation T366Y), and the second CH3 domain that heterodimerizes with the first CH3 domain comprises a cysteine at position 349 (e.g., comprises the mutation Y349C), a serine at position 366 (e.g., comprises the mutation T366S), an alanine at position 368 (e.g., comprises the mutation L368A), and a valine at position 407 (e.g., comprises the mutation Y407V). In embodiments, the first CH3 domain of the multispecific molecule comprises a cysteine at position 354 (e.g., comprises the mutation S354C) and a tryptophan (W) at position 366 (e.g., comprises the mutation T366W), and the second CH3 domain that heterodimerizes with the first CH3 domain comprises a cysteine at position 349 (e.g., comprises the mutation Y349C), a serine at position 366 (e.g., comprises the mutation T366S), an alanine at position 368 (e.g., comprises the mutation L368A), and a valine at position 407 (e.g., comprises the mutation Y407V).

### IgG Heterodimerization

In one aspect, heterodimerization of the polypeptide chains (e.g., of the half antibodies) of the multispecific molecule is increased by introducing one or more mutations in a CH3 domain which is derived from the IgG1 antibody class. In an embodiment, the mutations comprise a K409R mutation to one CH3 domain paired with F405L mutation in the second CH3 domain, according to the EU numbering scheme of Kabat et al. (pp. 688-696 in Sequences of proteins of immunological interest, 5th ed., Vol. 1 (1991; NIH, Bethesda, Md.)). Additional mutations may also, or alternatively, be at positions 366, 368, 370, 399, 405, 407, and 409 according to the EU numbering scheme of Kabat et al. (pp. 688-696 in Sequences of proteins of immunological interest, 5th ed., Vol. 1 (1991; NIH, Bethesda, Md.)). Preferably, heterodimerization of polypeptides comprising such mutations is achieved under reducing conditions, e.g., 10-100 mM 2-MEA (e.g., 25, 50, or 100 mM 2-MEA) for 1-10, e.g., 1.5-5, e.g., 5, hours at 25-37C, e.g., 25C or 37C.

The amino acid replacements described herein are introduced into the CH3 domains using techniques which are well known in the art. Normally the DNA encoding the heavy chain(s) is genetically engineered using the techniques described in Mutagenesis: a Practical Approach. Oligonucleotide-mediated mutagenesis is a preferred method for preparing substitution variants of the DNA encoding the two hybrid heavy chains. This technique is well known in the art as described by Adelman et al., (1983) DNA, 2:183.

The IgG heterodimerization strategy is described in, for example, WO2008/119353, WO2011/131746, and WO2013/060867, the contents of which are hereby incorporated by reference in their entirety.

In any of the embodiments described herein, the CH3 domains may be additionally mutated to introduce a pair of cysteine residues. Without being bound by theory, it is believed that the introduction of a pair of cysteine residues capable of forming a disulfide bond provide stability to the heterodimerized multispecific molecule. In embodiments, the first CH3 domain comprises a cysteine at position 354, according to the EU numbering scheme of Kabat et al. (pp. 688-696 in Sequences of proteins of immunological interest, 5th ed., Vol. 1 (1991; NIH, Bethesda, Md.)), and the second CH3 domain that heterodimerizes with the first CH3 domain comprises a cysteine at position 349, according to the EU numbering scheme of Kabat et al. (pp. 688-696 in Sequences of proteins of immunological interest, 5th ed., Vol. 1 (1991; NIH, Bethesda, Md.))

### Polar Bridge

In one aspect, heterodimerization of the polypeptide chains (e.g., of the half antibodies) of the multispecific molecule is increased by introducing mutations based on the “polar-bridging” rational, which is to make residues at the binding interface of the two polypeptide chains to interact with residues of similar (or complimentary) physical property in the heterodimer configuration, while with residues of different physical property in the homodimer configuration. In particular, these mutations are designed so that, in the heterodimer formation, polar residues interact with polar residues, while hydrophobic residues interact with hydrophobic residues. In contrast, in the homodimer formation, residues are mutated so that polar residues interact with hydrophobic residues. The favorable interactions in the heterodimer configuration and the unfavorable interactions in the homodimer configuration work together to make it more likely for CH3 domains to form heterodimers than to form homodimers.

In an exemplary embodiment, the above mutations are generated at one or more positions of residues 364, 368, 399, 405, 409, and 411 of CH3 domain, amino acid numbering according to the EU numbering scheme of Kabat et al. (pp. 688-696 in Sequences of proteins of immunological interest, 5th ed., Vol. 1 (1991; NIH, Bethesda, Md.)).

In one aspect, one or more mutations selected from a group consisting of: Ser364Leu, Thr366Val, Leu368Gln, Asp399Lys, Phe405Ser, Lys409Phe and Thr411Lys are introduced into one of the two CH3 domains. (Ser364Leu: original residue of serine at position 364 is replaced by leucine; Thr366Val: original residue of threonine at position 366 is replaced by valine; Leu368Gln: original residue of leucine at position 368 is replaced by glutamine; Asp399Lys: original residue aspartic acid at position 399 is replaced by lysine; Phe405Ser: original residue phenylalanine at position 405 is replaced by serine; Lys409Phe: original residue lysine at position 409 is replaced by phenylalanine; Thr411Lys: original residue of threonine at position 411 is replaced by lysine.).

In another aspect, the other CH3 can be introduced with one or more mutations selected from a group consisting of: Tyr407Phe, Lys409Gln and Thr411Asp (Tyr407Phe: original residue tyrosine at position 407 is replaced by phenyalanine; Lys409Glu: original residue lysine at position 409 is replaced by glutamic acid; Thr411Asp: original residue of threonine at position 411 is replaced by aspartic acid).

In a further aspect, one CH3 domain has one or more mutations selected from a group consisting of: Ser364Leu, Thr366Val, Leu368Gln, Asp399Lys, Phe405Ser, Lys409Phe and Thr411Lys, while the other CH3 domain has one or more mutations selected from a group consisting of: Tyr407Phe, Lys409Gln and Thr411Asp.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein are introduced into the CH3 domains using techniques which are well known in the art. Normally the DNA encoding the heavy chain(s) is genetically engineered using the techniques described in Mutagenesis: a Practical Approach. Oligonucleotide-mediated mutagenesis is a preferred method for preparing substitution variants of the DNA encoding the two hybrid heavy chains. This technique is well known in the art as described by Adelman et al., (1983) DNA, 2:183.

The polar bridge strategy is described in, for example, WO2006/106905, WO2009/089004 and K. Gunasekaran, et al. (2010) The Journal of Biological Chemistry, 285:19637-19646, the contents of which are hereby incorporated by reference in their entirety.

In any of the embodiments described herein, the CH3 domains may be additionally mutated to introduce a pair of cysteine residues. Without being bound by theory, it is believed that the introduction of a pair of cysteine residues capable of forming a disulfide bond provide stability to the heterodimerized multispecific molecule. In embodiments, the first CH3 domain comprises a cysteine at position 354, according to the EU numbering scheme of Kabat et al. (pp. 688-696 in Sequences of proteins of immunological interest, 5th ed., Vol. 1 (1991; NIH, Bethesda, Md.)), and the second CH3 domain that heterodimerizes with the first CH3 domain comprises a cysteine at position 349, according to the EU numbering scheme of Kabat et al. (pp. 688-696 in Sequences of proteins of immunological interest, 5th ed., Vol. 1 (1991; NIH, Bethesda, Md.))

Additional heavy chain constant region mutations can be used to promote the formation of heterodimeric antibodies.

Exemplary heterodimerization variants (e.g., skew variants and pi variants) for use in promoting heterodimerization are provided in WO 2016/182751 (e.g., FIGS. 29A-29E of WO 2016/182751). In one embodiment, the first half of the heterodimeric antibody (e.g., the heavy chain constant region of the first half of the heterodimeric antibody) and the second half of the heterodimeric antibody (e.g., the heavy chain constant region of the second half of the heterodimeric antibody) have a set of amino acid substitutions selected from the group consisting of: S364K/E357Q; L368D/370S; L368D/K370S; S364K; L368E/370S; S364K; T411T/E360E/Q362E; D401K; L368D/K370S; S364K E357L, and K370S; S364K/E357Q. The nomenclature “S364K/E357Q; L368D/370S” means that one heavy chain constant region has the amino acid substitutions S364K/E357Q and the other heavy chain constant region has the amino acid substitutions L368D/370S.

In one embodiment, the heavy chain constant region of the first half of the heterodimeric antibody and the heavy chain constant region of the second half of the heterodimeric antibody comprise an amino acid sequence independently selected from:

(SEQ ID NO: 1149)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVAVSHEDPEVKFNWYVDGVEVHNAKTKPREEQYNSTYRVVSVLTVL
HQDWLNGKEYKCKVSNKALAAPIEKTISKAKGQPREPQVCTLPPSREE
MTKNQVSLSCAVKGFYPSDIAVEWESNGQPENNYKTTPPVLDSDGSFF
LVSKLTVDKSRWQQGNVFSCSVMHEALHNHYTQKSLSLSPG,
and
(SEQ ID NO: 1150)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTC
VVVAVSHEDPEVKFNWYVDGVEVHNAKTKPREEQYNSTYRVVSVLTVL
HQDWLNGKEYKCKVSNKALAAPIEKTISKAKGQPREPQVYTLPPCREE
MTKNQVSLWCLVKGFYPSDIAVEWESNGQPENNYKTTPPVLDSDGSFF
LYSKLTVDKSRWQQGNVFSCSVMHEALHNHYTQKSLSLSPG

In some embodiments, the first half of the heterodimeric antibody (e.g., the heavy chain constant region of the first half of the heterodimeric antibody) comprises the amino acid sequence of SEQ ID NO: 1149, and the second half of the heterodimeric antibody (e.g., the heavy chain constant region of the second half of the heterodimeric antibody) comprises the amino acid sequence of SEQ ID NO: 1150, or vice versa.

In another embodiment, the first half of the heterodimeric antibody (e.g., the heavy chain constant region of the first half of the heterodimeric antibody) comprises the following sequence:

(SEQ ID NO: 1151)
GGGGSDKTHTCPPCPAPELLGGPSVFLFPPKPKDTLMISRTPEVTCVV
VAVSHEDPEVKFNWYVDGVEVHNAKTKPREEQYNSTYRVVSVLTVLHQ
DWLNGKEYKCKVSNKALAAPIEKTISKAKGQPREPQVCTLPPSRDELT
KNQVSLSCAVKGFYPSDIAVEWESNGQPENNYKTTPPVLDSDGSFFLV
SKLTVDKSRWQQGNVFSCSVMHEALHNHYTQKSLSLSPG;

and the second half of the heterodimeric antibody (e.g., the heavy chain constant region of the second half of the heterodimeric antibody) comprises the following sequence:

(SEQ ID NO: 1152)
GGGGSDKTHTCPPCPAPELLGGPSVFLFPPKPKDTLMISRTPEVTCVV
VAVSHEDPEVKFNWYVDGVEVHNAKTKPREEQYNSTYRVVSVLTVLHQ
DWLNGKEYKCKVSNKALAAPIEKTISKAKGQPREPQVYTLPPCRDELT
KNQVSLWCLVKGFYPSDIAVEWESNGQPENNYKTTPPVLDSDGSFFLY
SKLTVDKSRWQQGNVFSCSVMHEALHNHYTQKSLSLSPG.

Alternatively, the first half of the heterodimeric antibody (e.g., the heavy chain constant region of the first half of the heterodimeric antibody) comprises the amino acid sequence of SEQ ID NO: 1152, and the second half of the heterodimeric antibody (e.g., the heavy chain constant region of the second half of the heterodimeric antibody) comprises the amino acid sequence of SEQ ID NO: 1151.

In certain embodiments, the present invention provides a BCMA×CD3 multispecific antibody molecule. Exemplary anti-CD3 amino acid sequences are provided in WO2014110601, WO2014145806, WO 2016/086189, WO 2016/182751, WO 2016/020332, WO 2016/087531, WO 2016/079177, WO 2015/172800, WO 2017/008169, U.S. Pat. Nos. 9,340,621, 8,846,042, US 2013/0273055, US 2016/0176973, US 2015/0368351, US 2017/0051068, US 2016/0368988, and US 2015/0232557, incorporated herein by reference in their entireties.

In certain embodiments, the BCMA×CD3 multispecific antibody molecule comprises a CD3-binding moiety. In one embodiment, the CD3-binding moiety comprises at least one, two, three, four, five or six CDRs (or collectively all of the CDRs) from a heavy and light chain variable region comprising an amino acid sequence shown in Table 1 (e.g., from the heavy and light chain variable region sequences of SEQ ID NO: 1122, 1134, or 1136 disclosed in Table 1). In one embodiment, one or more of the CDRs (or collectively all of the CDRs) have one, two, three, four, five, six or more changes, e.g., amino acid substitutions (e.g., conservative amino acid substitutions) or deletions, relative to the heavy and light chain variable region sequences of SEQ ID NO: 1122, 1134, or 1136. In one embodiment, the CD3-binding moiety comprises the heavy and/or light chain variable region sequence of SEQ ID NO: 1122, 1134, or 1136, or a sequence at least about 85%, 90%, 95%, 99% or more identical thereto, and/or having one, two, three or more substitutions, insertions or deletions, e.g., conserved substitutions. In one embodiment, the CD3-binding moiety comprises the scFv portion of SEQ ID NO: 1122, 1134, or 1136, or a sequence at least about 85%, 90%, 95%, 99% or more identical thereto, and/or having one, two, three or more substitutions, insertions or deletions, e.g., conserved substitutions. In one embodiment, the CD3-binding moiety is an anti-CD3 scFv fused to an Fc domain. In one embodiment, the CD3-binding moiety comprises the amino acid sequence of SEQ ID NO: 1122, 1134, or 1136, or a sequence at least about 85%, 90%, 95%, 99% or more identical thereto, and/or having one, two, three or more substitutions, insertions or deletions, e.g., conserved substitutions.

### Antibody or Recombinant Non-Antibody Protein Linked to a Drug Moiety

In one aspect, the present invention discloses a BCMA-targeting agent (e.g., an anti-BCMA antibody molecule or a recombinant non-antibody protein that binds to BCMA) linked, e.g., via a linker, to a drug moiety. In one embodiment, the recombinant non-antibody protein that binds to BCMA comprises a BCMA ligand, e.g., B-cell activating factor (BAFF), a proliferation-inducing ligand (APRIL), or fragment thereof. In one embodiment, the drug moiety exerts a cytotoxic or cytostatic activity.

The term “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the antibody drug conjugate. Additionally, the antibody drug conjugate can be provided in the form of a fusion protein that may be expressed from a polynucleotide encoding the antibody drug conjugate.

The term “drug moiety” or “payload” as used herein refers to a moiety that is conjugated to a protein (e.g., a recombinant protein, or an antibody or antigen binding fragment), and can include any therapeutic or diagnostic agent, for example, an anti-cancer, anti-inflammatory, anti-infective (e.g., anti-fungal, antibacterial, anti-parasitic, anti-viral), or an anesthetic agent. In certain embodiments, the the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

Drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin, a protein such as tumor necrosis factor, α-interferon, β-interferon, nerve growth factor, platelet derived growth factor, tissue plasminogen activator, a cytokine, an apoptotic agent, an anti-angiogenic agent, or, a biological response modifier such as, for example, a lymphokine.

In one embodiment, the drug moiety is a cytotoxin. Examples of cytotoxin include but are not limited to, taxanes (see, e.g., International (PCT) Patent Application Nos. WO 01/38318 and PCT/US03/02675), DNA-alkylating agents (e.g., CC-1065 analogs), anthracyclines, amantins, tubulysin analogs, duocarmycin analogs, auristatin E, auristatin F, and cytotoxic agents comprising a reactive polyethylene glycol moiety (see, e.g., Sasse et al, J. Antibiot. (Tokyo), 53, 879-85 (2000), Suzawa et al, Bioorg. Med. Chem., 8, 2175-84 (2000), Ichimura et al, J. Antibiot. (Tokyo), 44, 1045-53 (1991), Francisco et al, Blood 2003 15; 102(4): 1458-65), U.S. Pat. Nos. 5,475,092, 6,340,701, 6,372,738, and 6,436,931, U.S. Patent Application Publication No. 2001/0036923 A1, Pending U.S. patent application Ser. Nos. 10/024,290 and 10/116,053, and International (PCT) Patent Application No. WO 01/49698), taxon, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, anti-metabolites (e.g., methotrexate, 6-mercaptopurine, 6-thioguanine, cytarabine, 5-fluorouracil decarbazine), ablating agents (e.g., mechlorethamine, thiotepa chlorambucil, meiphalan, carmustine (BSNU) and lomustine (CCNU), cyclophosphamide, busulfan, dibromomannitol, streptozotocin, mitomycin C, and cis-dichlorodiamine platinum (II) (DDP) cisplatin, anthracyclines (e.g. daunorubicin (formerly daunomycin) and doxorubicin), antibiotics (e.g., dactinomycin (formerly actinomycin), bleomycin, mithramycin, and anthramycin (AMC), and antimitotic agents (e.g., vincristine and vinblastine). See, e.g., Seattle Genetics US20090304721.

Other examples of cytotoxins that can be conjugated to the antibodies, antibody fragments, recombinant proteins, or functional equivalents of the present disclosure include duocarmycins, calicheamicins, and auristatins, and derivatives thereof.

Various types of cytotoxins, linkers and methods for conjugating therapeutic agents to antibodies are known in the art, see, e.g., Saito et al, (2003) Adv. Drug Deliv. Rev. 55: 199-215; Trail et al, (2003) Cancer Immunol. Immunother. 52:328-337; Payne, (2003) Cancer Cell 3:207-212; Allen, (2002) Nat. Rev. Cancer 2:750-763; Pastan and Kreitman, (2002) Curr. Opin. Investig. Drugs 3: 1089-1091; Senter and Springer, (2001) Adv. Drug Deliv. Rev. 53:247-264.

The antibodies, antibody fragments, recombinant proteins, or functional equivalents of the present disclosure can also be conjugated to a radioactive isotope to generate cytotoxic radiopharmaceuticals, referred to as radioimmunoconjugates. Examples of radioactive isotopes that can be conjugated to antibodies or proteins for use diagnostically or therapeutically include, but are not limited to, iodine-131, indium-111, yttrium-90, and lutetium-177. Methods for preparing radioimmunoconjugates are established in the art. Examples of radioimmunoconjugates are commercially available, including Zevalin (IDEC Pharmaceuticals) and Bexxar (Corixa Pharmaceuticals), and similar methods can be used to prepare radioimmunoconjugates using the antibodies or proteins disclosed herein. In certain aspects, the macrocyclic chelator is 1,4,7, 10-tetraazacyclododecane-N,N′,N″,N′″-tetraacetic acid (DOTA) which can be attached to an antibody or protein via a linker molecule. Such linker molecules are commonly known in the art and described in Denardo et al, (1998) Clin Cancer Res. 4(10):2483-90; Peterson et al, (1999) Bioconjug. Chem. 10(4):553-7; and Zimmerman et al., (1999) Nucl. Med. Biol. 26(8):943-50.

### DESCRIPTION

Provided herein are compositions of matter and methods of use for the treatment of a disease associated with expression of BCMA using a BCMA-targeting agent and a gamma secretase inhibitor (GSI), wherein the BCMA-targeting agent is an anti-BCMA antibody molecule or a recombinant non-antibody protein that binds to BCMA.

In one aspect, the present invention provides compositions and their use in medicaments or methods for treating, among other diseases, cancer or any malignancy or autoimmune diseases involving cells or tissues that express BCMA.

In one aspect, the compositions of the invention can be used to eradicate BCMA-expressing normal cells, thereby applicable for use as a conditioning therapy prior to cell transplantation. In one aspect, the BCMA-expressing normal cell is a BCMA-expressing normal stem cell and the cell transplantation is a stem cell transplantation.

### Exemplary BCMA-Targeting Sequences

In one aspect, disclosed herein is an anti-BCMA antibody molecule (e.g., a monospecific or multispecific antibody molecule). In one aspect, disclosed herein is an anti-BCMA antibody molecule linked, e.g., via a linker, to a drug moiety (an anti-BCMA antibody-drug conjugate).

Exemplary sequences that can be used in the anti-BCMA antibody molecule (e.g., a monospecific or multispecific anti-BCMA antibody molecule) or the anti-BCMA antibody-drug conjugate are disclosed below.

TABLE 1
Amino acid sequences of exemplary BCMA × CD3 or gH × CD3
bispecific antibodies

| | | |
|---|---|---|
| ER26 | | |
| SEQ ID NO: | J6M0 VH | QVQLVQSGAEVKKPGSSVKVSCKASGGTFSNYWMHWVRQAPGQ |
| 1118 | | GLEWMGATYRGHSDTYYNQKFKGRVTITADKSTSTAYMELSSLRS |
| | | EDTAVYYCARGAIYDGYDVLDNWGQGTLVTVSS |
| SEQ ID NO: | J6M0 VL | DIQMTQSPSSLSASVGDRVTITCSASQDISNYLNWYQQKPGKAPKL |
| 1119 | | LIYYTSNLHSGVPSRFSGSGSGTDFTLTISSLQPEDFATYYCQQYRK |
| | | LPWTFGQGTKLEIKR |
| SEQ ID NO: | Anti-BCMA | QVQLVQSGAEVKKPGSSVKVSCKASGGTFSNYWMHWVRQAPGQ |
| 1120 | heavy chain | GLEWMGATYRGHSDTYYNQKFKGRVTITADKSTSTAYMELSSLRS |
| | of ER26 | EDTAVYYCARGAIYNGYDVLDNWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVAVSH |
| | | EDPEVKFNWYVDGVEVHNAKTKPREEQYNSTYRVVSVLTVLHQD |
| | | WLNGKEYKCKVSNKALAAPIEKTISKAKGQPREPQVCTLPPSREEM |
| | | TKNQVSLSCAVKGFYPSDIAVEWESNGQPENNYKTTPPVLDSDGSF |
| | | FLVSKLTVDKSRWQQGNVFSCSVMHEALHNHYTQKSLSLSPGX, |
| | | wherein X is K or absent |
| SEQ ID NO: | Anti-BCMA | DIQMTQSPSSLSASVGDRVTITCSASQDISNYLNWYQQKPGKAPKL |
| 1121 | light chain | LIYYTSNLHSGVPSRFSGSGSGTDFTLTISSLQPEDFATYYCQQYRK |
| | of ER26 | LPWTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |
| SEQ ID NO: | Anti-CD3 | EVQLVESGGGLVQPGGSLKLSCAASGFTFNTYAMNWVRQASGKG |
| 1122 | scFv-Fc | LEWVGRIRSKYNNYATYYADSVKDRFTISRDDSKSTLYLQMNSLK |
| | of ER26 | TEDTAVYYCVRHGNFGNSYVSWFAYWGQGTLVTVSSGGGGSGG |
| | | GGSGGGGSGGGGSQAVVTQEPSLTVSPGGTVTLTCRSSTGAVTTSN |
| | | YANWVQQKPGQAPRGLIGGTNKRAPWTPARFSGSLLGDKAALTLS |
| | | GAQPEDEAEYFCALWYSNLWVFGGGTKLTVLGGGGSDKTHTCPP |
| | | CPAPELLGGPSVFLFPPKPKDTLMISRTPEVTCVVVDVSHEDPEVKF |
| | | NWYVDGVEVHNAKTKPREEQYASTYRVVSVLTVLHQDWLNGKE |
| | | YKCKVSNKALPAPIEKTISKAKGQPREPQVYTLPPCREEMTKNQVS |
| | | LWCLVKGFYPSDIAVEWESNGQPENNYKTTPPVLDSDGSFFLYSKL |
| | | TVDKSRWQQGNVFSCSVMHEALHNHYTQKSLSLSPGX, |
| | | wherein X is K or absent |
| BQ76 | | |
| SEQ ID NO: | 17A5 VH | EVQLLESGGGLVQPGGSLRLSCAASGFTFSSYAMSWVRQAPGKGL |
| 1123 | | EWVSAISGSGGSTYYADSVKGRFTISRDNSKNTLYLQMNSLRAEDT |
| | | AVYYCAKVAPYFAPFDYWGQGTLVTVSS |
| SEQ ID NO: | 17A5 VL | EIVLTQSPGTLSLSPGERATLSCRASQSVSSSYLAWYQQKPGQAPRL |
| 1124 | | LIYGASSRATGIPDRFSGSGSGTDFTLTISRLEPEDFAVYYCQQYGN |
| | | PPLYTFGQGTKVEIK |
| SEQ ID NO: | Anti-BCMA | EVQLLESGGGLVQPGGSLRLSCAASGFTFSSYAMSWVRQAPGKGL |
| 1125 | heavy chain | EWVSAISGSGGSTYYADSVKGRFTISRDNSKNTLYLQMNSLRAEDT |
| | of BQ76 | AVYYCAKVAPYFAPFDYWGQGTLVTVSSASTKGPSVFPLAPSSKST |
| | | SGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTC |
| | | PPCPAPELLGGPSVFLFPPKPKDTLMISRTPEVTCVVVDVSHEDPEV |
| | | KFNWYVDGVEVHNAKTKPREEQYASTYRVVSVLTVLHQDWLNG |
| | | KEYKCKVSNKALPAPIEKTISKAKGQPREPQVCTLPPSREEMTKNQ |
| | | VSLSCAVKGFYPSDIAVEWESNGQPENNYKTTPPVLDSDGSFFLVS |
| | | KLTVDKSRWQQGNVFSCSVMHEALHNHYTQKSLSLSPGX, |
| | | wherein X is K or absent |
| SEQ ID NO: | Anti-BCMA | EIVLTQSPGTLSLSPGERATLSCRASQSVSSSYLAWYQQKPGQAPRL |
| 1126 | light chain | LIYGASSRATGIPDRFSGSGSGTDFTLTISRLEPEDFAVYYCQQYGN |
| | of BQ76 | PPLYTFGQGTKVEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |
| SEQ ID NO: | Anti-CD3 | EVQLVESGGGLVQPGGSLKLSCAASGFTFNTYAMNWVRQASGKG |
| 1122 | scFv-Fc | LEWVGRIRSKYNNYATYYADSVKDRFTISRDDSKSTLYLQMNSLK |
| | of BQ76 | TEDTAVYYCVRHGNFGNSYVSWFAYWGQGTLVTVSSGGGGSGG |
| | | GGSGGGGSGGGGSQAVVTQEPSLTVSPGGTVTLTCRSSTGAVTTSN |
| | | YANWVQQKPGQAPRGLIGGTNKRAPWTPARFSGSLLGDKAALTLS |
| | | GAQPEDEAEYFCALWYSNLWVFGGGTKLTVLGGGGSDKTHTCPP |
| | | CPAPELLGGPSVFLFPPKPKDTLMISRTPEVTCVVVDVSHEDPEVKF |
| | | NWYVDGVEVHNAKTKPREEQYASTYRVVSVLTVLHQDWLNGKE |
| | | YKCKVSNKALPAPIEKTISKAKGQPREPQVYTLPPCREEMTKNQVS |
| | | LWCLVKGFYPSDIAVEWESNGQPENNYKTTPPVLDSDGSFFLYSKL |
| | | TVDKSRWQQGNVFSCSVMHEALHNHYTQKSLSLSPGX, |
| | | wherein X is K or absent |
| BU76 | | |
| SEQ ID NO: | C11D5 VH | QIQLVQSGPELKKPGETVKISCKASGYTFTDYSINWVKRAPGKGLK |
| 1127 | | WMGWINTETREPAYAYDFRGRFAFSLETSASTAYLQINNLKYEDT |
| | | ATYFCALDYSYAMDYWGQGTSVTVSS |
| SEQ ID NO: | C11D5 | DIVLTQSPPSLAMSLGKRATISCRASESVTILGSHLIHWYQQKPGQP |
| 1128 | | PTLLIQLASNVQTGVPARFSGSGSRTDFTLTIDPVEEDDVAVYYCLQ |
| | | SRTIPRTFGGGTKLEIK |
| SEQ ID NO: | Anti-BCMA | QIQLVQSGPELKKPGETVKISCKASGYTFTDYSINWVKRAPGKGLK |
| 1129 | heavy chain | WMGWINTETREPAYAYDFRGRFAFSLETSASTAYLQINNLKYEDT |
| | of BU76 | ATYFCALDYSYAMDYWGQGTSVTVSSASTKGPSVFPLAPSSKSTS |
| | | GGTAALGCLVKDYFPEPVTVSWNSGALTSGVHTFPAVLQSSGLYS |
| | | LSSVVTVPSSSLGTQTYICNVNHKPSNTKVDKRVEPKSCDKTHTCP |
| | | PCPAPELLGGPSVFLFPPKPKDTLMISRTPEVTCVVVDVSHEDPEVK |
| | | FNWYVDGVEVHNAKTKPREEQYASTYRVVSVLTVLHQDWLNGK |
| | | EYKCKVSNKALPAPIEKTISKAKGQPREPQVCTLPPSREEMTKNQV |
| | | SLSCAVKGFYPSDIAVEWESNGQPENNYKTTPPVLDSDGSFFLVSK |
| | | LTVDKSRWQQGNVFSCSVMHEALHNHYTQKSLSLSPGX, |
| | | wherein X is K or absent |
| SEQ ID NO: | Anti-BCMA | DIVLTQSPASLAMSLGKRATISCRASESVSVIGAHLIHWYQQKPGQP |
| 1130 | light chain | PKLLIYLASNLETGVPARFSGSGSGTDFTLTIDPVEEDDVAIYSCLQS |
| | of BU76 | RIFPRTFGGGTKLEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |
| SEQ ID NO: | Anti-CD3 | EVQLVESGGGLVQPGGSLKLSCAASGFTFNTYAMNWVRQASGKG |
| 1122 | scFv-Fc | LEWVGRIRSKYNNYATYYADSVKDRFTISRDDSKSTLYLQMNSLK |
| | of BU76 | TEDTAVYYCVRHGNFGNSYVSWFAYWGQGTLVTVSSGGGGSGG |
| | | GGSGGGGSGGGGSQAVVTQEPSLTVSPGGTVTLTCRSSTGAVTTSN |
| | | YANWVQQKPGQAPRGLIGGTNKRAPWTPARFSGSLLGDKAALTLS |
| | | GAQPEDEAEYFCALWYSNLWVFGGGTKLTVLGGGGSDKTHTCPP |
| | | CPAPELLGGPSVFLFPPKPKDTLMISRTPEVTCVVVDVSHEDPEVKF |
| | | NWYVDGVEVHNAKTKPREEQYASTYRVVSVLTVLHQDWLNGKE |
| | | YKCKVSNKALPAPIEKTISKAKGQPREPQVYTLPPCREEMTKNQVS |
| | | LWCLVKGFYPSDIAVEWESNGQPENNYKTTPPVLD |
| | | wherein Xis K or absent |
| EE11 | | |
| SEQ ID NO: | 83A10 VH | EVQLLESGGGLVQPGGSLRLSCAASGFTFSSYAMSWVRQAPGKGL |
| 1131 | | EWVSAISGSGGSTYYADSVKGRFTISRDNSKNTLYLQMNSLRAEDT |
| | | AVYYCAKVLGWFDYWGQGTLVTVSS |
| SEQ ID NO: | 83A10 VL | EIVLTQSPGTLSLSPGERATLSCRASQSVSSSYLAWYQQKPGQAPRL |
| 1132 | | LIYGASSRATGIPDRFSGSGSGTDFTLTISRLEPEDFAVYYCQQYGY |
| | | PPDFTFGQGTKVEIK |
| SEQ ID NO: | Anti-BCMA | EVQLLESGGGLVQPGGSLRLSCAASGFTFSSYAMSWVRQAPGKGL |
| 1133 | scFv-Fc | EWVSAISGSGGSTYYADSVKGRFTISRDNSKNTLYLQMNSLRAEDT |
| | of EE11 | AVYYCAKVLGWFDYWGQGTLVTVSSGGGGSGGGGSGGGGSGGG |
| | | GSEIVLTQSPGTLSLSPGERATLSCRASQSVSSSYLAWYQQKPGQAP |
| | | RLLIYGASSRATGIPDRFSGSGSGTDFTLTISRLEPEDFAVYYCQQY |
| | | GYPPDFTFGQGTKVEIKGGGGSDKTHTCPPCPAPELLGGPSVFLFPP |
| | | KPKDTLMISRTPEVTCVVVAVSHEDPEVKFNWYVDGVEVHNAKT |
| | | KPREEQYNSTYRVVSVLTVLHQDWLNGKEYKCKVSNKALAAPIEK |
| | | TISKAKGQPREPQVYTLPPCRDELTKNQVSLWCLVKGFYPSDIAVE |
| | | WESNGQPENNYKTTPPVLDSDGSFFLYSKLTVDKSRWQQGNVFSC |
| | | SVMHEALHNHYTQKSLSLSPGX, wherein X is K or absent |
| SEQ ID NO: | Anti-CD3 | EVQLVESGGGLVQPGGSLKLSCAASGFTFNTYAMNWVRQASGKG |
| 1134 | scFv-Fc | LEWVGRIRSKYNNYATYYADSVKDRFTISRDDSKSTLYLQMNSLK |
| | of EE11 | TEDTAVYYCVRHGNFGNSYVSWFAYWGQGTLVTVSSGGGGSGG |
| | | GGSGGGGSGGGGSQAVVTQEPSLTVSPGGTVTLTCRSSTGAVTTSN |
| | | YANWVQQKPGQAPRGLIGGTNKRAPWTPARFSGSLLGDKAALTLS |
| | | GAQPEDEAEYFCALWYSNLWVFGGGTKLTVLGGGGSDKTHTCPP |
| | | CPAPELLGGPSVFLFPPKPKDTLMISRTPEVTCVVVAVSHEDPEVKF |
| | | NWYVDGVEVHNAKTKPREEQYNSTYRVVSVLTVLHQDWLNGKE |
| | | YKCKVSNKALAAPIEKTISKAKGQPREPQVCTLPPSRDELTKNQVS |
| | | LSCAVKGFYPSDIAVEWESNGQPENNYKTTPPVLDSDGSFFLVSKL |
| | | TVDKSRWQQGNVFSCSVMHEALHNHYTQKSLSLSPGX, |
| | | wherein X is K or absent |

XU68

| | | |
|---|---|---|
| SEQ ID NO: | Anti-gH | EVQLVESGGGLVKPGGSLRLSCAASGFTFSPYSVFWVRQAPGKGL |
| 1135 | scFv-Fc | EWVSSINTDSTYKYYADSVKGRFTISRDNAENSIFLQMNSLRAEDT |
| | of XU68 | AVYYCARDRSYYAFSSGSLSDYYYGLDVWGQGTLVTVSSGGGGS |
| | | GGGGSGGGGSGGGGSDIVMTQSPLSLSVTPGEPASISCRSSQSLLHT |
| | | NLYNYLDWYVQKPGQSPQLLIYLASNRASGVPDRFSGSGSGTDFTL |
| | | KISRVETEDVGVYYCMQALQIPRTFGQGTKLEIKGGGGSDKTHTCP |
| | | PCPAPELLGGPSVFLFPPKPKDTLMISRTPEVTCVVVAVSHEDPEVK |
| | | FNWYVDGVEVHNAKTKPREEQYNSTYRVVSVLTVLHQDWLNGK |
| | | EYKCKVSNKALAAPIEKTISKAKGQPREPQVYTLPPCRDELTKNQV |
| | | SLWCLVKGFYPSDIAVEWESNGQPENNYKTTPPVLDSDGSFFLYSK |
| | | LTVDKSRWQQGNVFSCSVMHEALHNHYTQKSLSLSPGX, |
| | | wherein X is K or absent |
| SEQ ID NO: | Anti-CD3 | EVQLVESGGGLVQPGGSLKLSCAASGFTFNKYAMNWVRQAPGKG |
| 1136 | scFv-Fc | LEWVARIRSKYNNYATYYADSVKDRFTISRDDSKNTAYLQMNNL |
| | of XU68 | KTEDTAVYYCVRHGNFGNSYISYWAYWGQGTLVTVSSGGGGSGG |
| | | GGSGGGGSGGGGSQTVVTQEPSLTVSPGGTVTLTCGSSTGAVTSG |
| | | NYPNWVQQKPGQAPRGLIGGTKFLAPGTPARFSGSLLGGKAALTL |
| | | SGVQPEDEAEYYCVLWYSNRWVFGGGTKLTVLGGGGSDKTHTCP |
| | | PCPAPELLGGPSVFLFPPKPKDTLMISRTPEVTCVVVAVSHEDPEVK |
| | | FNWYVDGVEVHNAKTKPREEQYNSTYRVVSVLTVLHQDWLNGK |
| | | EYKCKVSNKALAAPIEKTISKAKGQPREPQVCTLPPSRDELTKNQV |
| | | SLSCAVKGFYPSDIAVEWESNGQPENNYKTTPPVLDSDGSFFLVSK |
| | | LTVDKSRWQQGNVFSCSVMHEALHNHYTQKSLSLSPGX, |
| | | wherein X is K or absent |
| EM90 | | |
| SEQ ID NO: | Common light | QAVVTQEPSLTVSPGGTVTLTCGSSTGAVTTSNYANWVQEKPGQA |
| 1153 | chain of EM90 | FRGLIGGTNKRAPGTPARFSGSLLGGKAALTLSGAQPEDEAEYYCA |
| | | LWYSNLWVFGGGTKLTVLGQPKAAPSVTLFPPSSEELQANKATLV |
| | | CLISDFYPGAVTVAWKADSSPVKAGVETTTPSKQSNNKYAASSYLS |
| | | LTPEQWKSHRSYSCQVTHEGSTVEKTVAPTECS |
| SEQ ID NO: | Anti-BCMA | EVQLVESGGGLVKPGGSLRLSCAASGFTFSNSGMIWVRQAPGKGL |
| 1154 | heavy chain | EWVGHIRSKTDGGTTDYAAPVKGRFTISRDDSKNTLYLQMNSLKT |
| | of EM90 | EDTAVYYCTTGGSGSFDYWGQGTLVTVSSASTKGPSVFPLAPSSKS |
| | | TSGGTAALGCLVKDYFPEPVTVSWNSGALTSGVHTFPAVLQSSGL |
| | | YSLSSVVTVPSSSLGTQTYICNVNHKPSNTKVDKRVEPKSCDKTHT |
| | | CPPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLN |
| | | GKEYKCKVSNKALPAPIEKTISKAKGQPREPQVCTLPPSREEMTKN |
| | | QVSLSCAVKGFYPSDIAVEWESNGQPENNYKTTPPVLDSDGSFFLV |
| | | SKLTVDKSRWQQGNVFSCSVMHEALHNHYTQKSLSLSPGX, |
| | | wherein X is K or absent |
| SEQ ID NO: | Anti-BCMA, | EVQLVESGGGLVKPGGSLRLSCAASGFTFSNSGMIWVRQAPGKGL |
| 1155 | anti-CD3 | EWVGHIRSKTDGGTTDYAAPVKGRFTISRDDSKNTLYLQMNSLKT |
| | heavy chain | EDTAVYYCTTGGSGSFDYWGQGTLVTVSSASTKGPSVFPLAPSSKS |
| | of EM90 | TSGGTAALGCLVKDYFPEPVTVSWNSGALTSGVHTFPAVLQSSGL |
| | | YSLSSVVTVPSSSLGTQTYICNVNHKPSNTKVDKRVEPKSCDGGGG |
| | | SGGGGSEVQLLESGGGLVQPGGSLRLSCAASGFTFSTYAMNWVRQ |
| | | APGKGLEWVSRIRSKYNNYATYYADSVKGRFTISRDDSKNTLYLQ |
| | | MNSLRAEDTAVYYCVRHGNFGNSYVSWFAYWGQGTLVTVSSAST |
| | | KGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVD |
| | | KRVEPKSCDKTHTCPPCPAPEAAGGPSVFLFPPKPKDTLMISRTPEV |
| | | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQV |
| | | YTLPPCREEMTKNQVSLWCLVKGFYPSDIAVEWESNGQPENNYKT |
| | | TPPVLDSDGSFFLYSKLTVDKSRWQQGNVFSCSVMHEALHNHYTQ |
| | | KSLSLSPGX,wherein X is K or absent |
| FP31 | | |
| SEQ ID NO: | Anti-CD3 | DIVMTQSPDSLAVSLGERATINCKSSQSLFNVRSRKNYLAWYQQKP |
| 1157 | light chain | GQPPKLLISWASTRESGVPDRFSGSGSGTDFTLTISSLQAEDVAVYY |
| | of FP31 | CKQSYDLFTFGSGTKLEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| SEQ ID NO: | Anti-BCMA | EIVLTQSPGTLSLSPGERATLSCRASQSVSSSYLAWYQQKPGQAPRL |
| 1158 | light chain | LMYDASIRATGIPDRFSGSGSGTDFTLTISRLEPEDFAVYYCQQYQS |
| | of FP31 | WPLTFGQGTKVEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |
| SEQ ID NO: | Anti-BCMA | EVQLLESGGGLVQPGGSLRLSCAASGFTFSSYPMSWVRQAPGKGL |
| 1159 | heavy chain | EWVSAIGGSGGSLPYADSVKGRFTISRDNSKNTLYLQMNSLRAEDT |
| | of FP31 | AVYYCARYWPMDIWGQGTLVTVSSASTKGPSVFPLAPCSRSTSEST |
| | | AALGCLVKDYFPEPVTVSWNSGALTSGVHTFPAVLQSSGLYSLSSV |
| | | VTVPSSNFGTQTYTCNVDHKPSNTKVDKTVERKCEVECPECPAPPV |
| | | AGPSVFLFPPKPKDTLMISRTPEVTCVVVAVSHEDPEVQFNWYVDG |
| | | VEVHNAKTKPREEQFNSTFRVVSVLTVVHQDWLNGKEYKCKVSN |
| | | KGLPSSIEKTISKTKGQPREPQVYTLPPSREEMTKNQVSLTCEVKGF |
| | | YPSDISVEWESNGQPENNYKTTPPMLDSDGSFFLYSKLTVDKSRWQ |
| | | QGNVFSCSVMHEALHNHYTQKSLSLSPGK |
| SEQ ID NO: | Anti-CD3 | EVQLVESGGGLVQPGGSLRLSCAASGFTFSDYYMTWVRQAPGKGL |
| 1160 | heavy chain | EWVAFIRNRARGYTSDHNASVKGRFTISRDNAKNSLYLQMNSLRA |
| | of FP31 | EDTAVYYCARDRPSYYVLDYWGQGTTVTVSSASTKGPSVFPLAPC |
| | | SRSTSESTAALGCLVKDYFPEPVTVSWNSGALTSGVHTFPAVLQSS |
| | | GLYSLSSVVTVPSSNFGTQTYTCNVDHKPSNTKVDKTVERKCRVR |
| | | CPRCPAPPVAGPSVFLFPPKPKDTLMISRTPEVTCVVVAVSHEDPEV |
| | | QFNWYVDGVEVHNAKTKPREEQFNSTFRVVSVLTVVHQDWLNGK |
| | | EYKCKVSNKGLPSSIEKTISKTKGQPREPQVYTLPPSREEMTKNQVS |
| | | LTCLVKGFYPSDISVEWESNGQPENNYKTTPPMLDSDGSFFLYSRL |
| | | TVDKSRWQQGNVFSCSVMHEALHNHYTQKSLSLSPGK |

TABLE 2
Amino acid and nucleic acid sequences of exemplary anti-BCMA VHs,
VLs, and scFv domains

| | | |
|---|---|---|
| | SEQ | |
| Name/ | ID | |
| Description | NO: | Sequence |

139109

| | | |
|---|---|---|
| 139109-aa | 49 | EVQLVESGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSDIQLTQSPSSLSASVGDRVTITC |
| | | RASQSISSYLNWYQQKPGKAPKLLIYAASSLQSGVPSRFSGSGSGTDFTLTISS |
| | | LQPEDFATYYCQQSYSTPYTFGQGTKVEIK |
| 139109-nt | 64 | GAAGTGCAATTGGTGGAATCAGGGGGAGGACTTGTGCAGCCTGGAGGAT |
| ScFv domain | | CGCTGAGACTGTCATGTGCCGTGTCCGGCTTTGCCCTGTCCAACCACGGGA |
| | | TGTCCTGGGTCCGCCGCGCGCCTGGAAAGGGCCTCGAATGGGTGTCGGGT |
| | | ATTGTGTACAGCGGTAGCACCTACTATGCCGCATCCGTGAAGGGGAGATT |
| | | CACCATCAGCCGGGACAACTCCAGGAACACTCTGTACCTCCAAATGAATT |
| | | CGCTGAGGCCAGAGGACACTGCCATCTACTACTGCTCCGCGCATGGCGGA |
| | | GAGTCCGACGTCTGGGGACAGGGGACCACCGTGACCGTGTCTAGCGCGTC |
| | | CGGCGGAGGCGGCAGCGGGGGTCGGGCATCAGGGGGCGGCGGATCGGAC |
| | | ATCCAGCTCACCCAGTCCCCGAGCTCGCTGTCCGCCTCCGTGGGAGATCG |
| | | GGTCACCATCACGTGCCGCGCCAGCCAGTCGATTTCCTCCTACCTGAACTG |
| | | GTACCAACAGAAGCCCGGAAAAGCCCCGAAGCTTCTCATCTACGCCGCCT |
| | | CGAGCCTGCAGTCAGGAGTGCCCTCACGGTTCTCCGGCTCCGGTTCCGGT |
| | | ACTGATTTCACCCTGACCATTTCCTCCCTGCAACCGGAGGACTTCGCTACT |
| | | TACTACTGCCAGCAGTCGTACTCCACCCCCTACACTTTCGGACAAGGCACC |
| | | AAGGTCGAAATCAAG |
| 139109-aa | 79 | EVQLVESGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139109-aa | 94 | DIQLTQSPSSLSASVGDRVTITCRASQSISSYLNWYQQKPGKAPKLLIYAASSL |
| VL | | QSGVPSRFSGSGSGTDFTLTISSLQPEDFATYYCQQSYSTPYTFGQGTKVEIK |

139103

| | | |
|---|---|---|
| 139103-aa | 39 | QVQLVESGGGLVQPGRSLRLSCAASGFTFSNYAMSWVRQAPGKGLGWVSGI |
| ScFv domain | | SRSGENTYYADSVKGRFTISRDNSKNTLYLQMNSLRDEDTAVYYCARSPAHY |
| | | YGGMDVWGQGTTVTVSSASGGGGSGGRASGGGGSDIVLTQSPGTLSLSPGE |
| | | RATLSCRASQSISSSFLAWYQQKPGQAPRLLIYGASRRATGIPDRFSGSGSGTD |
| | | FTLTISRLEPEDSAVYYCQQYHSSPSWTFGQGTKLEIK |
| 139103-nt | 54 | CAAGTGCAACTCGTGGAATCTGGTGGAGGACTCGTGCAACCCGGAAGATC |
| ScFv domain | | GCTTAGACTGTCGTGTGCCGCCAGCGGGTTCACTTTCTCGAACTACGCGAT |
| | | GTCCTGGGTCCGCCAGGCACCCGGAAAGGGACTCGGTTGGGTGTCCGGCA |
| | | TTTCCCGGTCCGGCGAAAATACCTACTACGCCGACTCCGTGAAGGGCCGC |
| | | TTCACCATCTCAAGGGACAACAGCAAAAACACCCTGTACTTGCAAATGAA |
| | | CTCCCTGCGGGATGAAGATACAGCCGTGTACTATTGCGCCCGGTCGCCTG |
| | | CCCATTACTACGGCGGAATGGACGTCTGGGGACAGGGAACCACTGTGACT |
| | | GTCAGCAGCGCGTCGGGTGGCGGCGGCTCAGGGGGTCGGGCCTCCGGGG |
| | | GGGGAGGGTCCGACATCGTGCTGACCCAGTCCCCGGGAACCCTGAGCCTG |
| | | AGCCCGGGAGAGCGCGCGACCCTGTCATGCCGGGCATCCCAGAGCATTAG |
| | | CTCCTCCTTTCTCGCCTGGTATCAGCAGAAGCCCGGACAGGCCCCGAGGC |
| | | TGCTGATCTACGGCGCTAGCAGAAGGGCTACCGGAATCCCAGACCGGTTC |
| | | TCCGGCTCCGGTTCCGGGACCGATTTCACCCTTACTATCTCGCGCCTGGAA |
| | | CCTGAGGACTCCGCCGTCTACTACTGCCAGCAGTACCACTCATCCCCGTCG |
| | | TGGACGTTCGGACAGGGCACCAAGCTGGAGATTAAG |
| 139103-aa | 69 | QVQLVESGGGLVQPGRSLRLSCAASGFTFSNYAMSWVRQAPGKGLGWVSGI |
| VH | | SRSGENTYYADSVKGRFTISRDNSKNTLYLQMNSLRDEDTAVYYCARSPAHY |
| | | YGGMDVWGQGTTVTVSS |
| 139103-aa | 84 | DIVLTQSPGTLSLSPGERATLSCRASQSISSSFLAWYQQKPGQAPRLLIYGASR |
| VL | | RATGIPDRFSGSGSGTDFTLTISRLEPEDSAVYYCQQYHSSPSWTFGQGTKLEI |
| | | K |
| 139105 | | |
| 139105-aa | 40 | QVQLVESGGGLVQPGRSLRLSCAASGFTFDDYAMHWVRQAPGKGLEWVSGI |
| ScFv domain | | SWNSGSIGYADSVKGRFTISRDNAKNSLYLQMNSLRAEDTALYYCSVHSFLA |
| | | YWGQGTLVTVSSASGGGGSGGRASGGGGSDIVMTQTPLSLPVTPGEPASISCR |
| | | SSQSLLHSNGYNYLDWYLQKPGQSPQLLIYLGSNRASGVPDRFSGSGSGTDFT |
| | | LKISRVEAEDVGVYYCMQALQTPYTFGQGTKVEIK |
| 139105-nt | 55 | CAAGTGCAACTCGTCGAATCCGGTGGAGGTCTGGTCCAACCTGGTAGAAG |
| ScFv domain | | CCTGAGACTGTCGTGTGCGGCCAGCGGATTCACCTTTGATGACTATGCTAT |
| | | GCACTGGGTGCGGCAGGCCCCAGGAAAGGGCCTGGAATGGGTGTCGGGA |
| | | ATTAGCTGGAACTCCGGGTCCATTGGCTACGCCGACTCCGTGAAGGGCCG |
| | | CTTCACCATCTCCCGCGACAACGCAAAGAACTCCCTGTACTTGCAAATGA |
| | | ACTCGCTCAGGGCTGAGGATACCGCGCTGTACTACTGCTCCGTGCATTCCT |
| | | TCCTGGCCTACTGGGGACAGGGAACTCTGGTCACCGTGTCGAGCGCCTCC |
| | | GGCGGCGGGGGCTCGGGTGGACGGGCCTCGGGCGGAGGGGGGTCCGACA |
| | | TCGTGATGACCCAGACCCCGCTGAGCTTGCCCGTGACTCCCGGAGAGCCT |
| | | GCATCCATCTCCTGCCGGTCATCCCAGTCCCTTCTCCACTCCAACGGATAC |
| | | AACTACCTCGACTGGTACCTCCAGAAGCCGGGACAGAGCCCTCAGCTTCT |
| | | GATCTACCTGGGGTCAAATAGAGCCTCAGGAGTGCCGGATCGGTTCAGCG |
| | | GATCTGGTTCGGGAACTGATTTCACTCTGAAGATTTCCCGCGTGGAAGCC |
| | | GAGGACGTGGGCGTCTACTACTGTATGCAGGCGCTGCAGACCCCCTATAC |
| | | CTTCGGCCAAGGGACGAAAGTGGAGATCAAG |
| 139105-aa | 70 | QVQLVESGGGLVQPGRSLRLSCAASGFTFDDYAMHWVRQAPGKGLEWVSGI |
| VH | | SWNSGSIGYADSVKGRFTISRDNAKNSLYLQMNSLRAEDTALYYCSVHSFLA |
| | | YWGQGTLVTVSS |
| 139105-aa | 85 | DIVMTQTPLSLPVTPGEPASISCRSSQSLLHSNGYNYLDWYLQKPGQSPQLLIY |
| VL | | LGSNRASGVPDRFSGSGSGTDFTLKISRVEAEDVGVYYCMQALQTPYTFGQG |
| | | TKVEIK |
| 139111 | | |
| 139111-aa | 41 | EVQLLESGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSDIVMTQTPLSLSVTPGQPASISC |
| | | KSSQSLLRNDGKTPLYWYLQKAGQPPQLLIYEVSNRFSGVPDRFSGSGSGTDF |
| | | TLKISRVEAEDVGAYYCMQNIQFPSFGGGTKLEIK |
| 139111-nt | 56 | GAAGTGCAATTGTTGGAATCTGGAGGAGGACTTGTGCAGCCTGGAGGATC |
| ScFv domain | | ACTGAGACTTTCGTGTGCGGTGTCAGGCTTCGCCCTGAGCAACCACGGCA |
| | | TGAGCTGGGTGCGGAGAGCCCCGGGGAAGGGTCTGGAATGGGTGTCCGG |
| | | GATCGTCTACTCCGGTTCAACTTACTACGCCGCAAGCGTGAAGGGTCGCTT |
| | | CACCATTTCCCGCGATAACTCCCGGAACACCCTGTACCTCCAAATGAACTC |
| | | CCTGCGGCCCGAGGACACCGCCATCTACTACTGTTCCGCGCATGGAGGAG |
| | | AGTCCGATGTCTGGGGACAGGGCACTACCGTGACCGTGTCGAGCGCCTCG |
| | | GGGGGAGGAGGCTCCGGCGGTCGCGCCTCCGGGGGGGGTGGCAGCGACA |
| | | TTGTGATGACGCAGACTCCACTCTCGCTGTCCGTGACCCCGGGACAGCCC |
| | | GCGTCCATCTCGTGCAAGAGCTCCCAGAGCCTGCTGAGGAACGACGGAAA |
| | | GACTCCTCTGTATTGGTACCTCCAGAAGGCTGGACAGCCCCCGCAACTGC |
| | | TCATCTACGAAGTGTCAAATCGCTTCTCCGGGGTGCCGGATCGGTTTTCCG |
| | | GCTCGGGATCGGGCACCGACTTCACCCTGAAAATCTCCAGGGTCGAGGCC |
| | | GAGGACGTGGGAGCCTACTACTGCATGCAAAACATCCAGTTCCCTTCCTT |
| | | CGGCGGCGGCACAAAGCTGGAGATTAAG |
| 139111-aa | 71 | EVQLLESGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139111-aa | 86 | DIVMTQTPLSLSVTPGQPASISCKSSQSLLRNDGKTPLYWYLQKAGQPPQLLI |
| VL | | YEVSNRFSGVPDRFSGSGSGTDFTLKISRVEAEDVGAYYCMQNIQFPSFGGGT |
| | | KLEIK |
| 139100 | | |
| 139100-aa | 42 | QVQLVQSGAEVRKTGASVKVSCKASGYIFDNFGINWVRQAPGQGLEWMGW |
| ScFv domain | | INPKNNNTNYAQKFQGRVTITADESTNTAYMEVSSLRSEDTAVYYCARGPYY |
| | | YQSYMDVWGQGTMVTVSSASGGGGSGGRASGGGGSDIVMTQTPLSLPVTPG |
| | | EPASISCRSSQSLLHSNGYNYLNWYLQKPGQSPQLLIYLGSKRASGVPDRFSG |
| | | SGSGTDFTLHITRVGAEDVGVYYCMQALQTPYTFGQGTKLEIK |
| 139100-nt | 57 | CAAGTCCAACTCGTCCAGTCCGGCGCAGAAGTCAGAAAAACCGGTGCTAG |
| ScFv domain | | CGTGAAAGTGTCCTGCAAGGCCTCCGGCTACATTTTCGATAACTTCGGAAT |
| | | CAACTGGGTCAGACAGGCCCCGGGCCAGGGGCTGGAATGGATGGGATGG |
| | | ATCAACCCCAAGAACAACAACACCAACTACGCACAGAAGTTCCAGGGCC |
| | | GCGTGACTATCACCGCCGATGAATCGACCAATACCGCCTACATGGAGGTG |
| | | TCCTCCCTGCGGTCGGAGGACACTGCCGTGTATTACTGCGCGAGGGGCCC |
| | | ATACTACTACCAAAGCTACATGGACGTCTGGGGACAGGGAACCATGGTGA |
| | | CCGTGTCATCCGCCTCCGGTGGTGGAGGCTCCGGGGGGCGGGCTTCAGGA |
| | | GGCGGAGGAAGCGATATTGTGATGACCCAGACTCCGCTTAGCCTGCCCGT |
| | | GACTCCTGGAGAACCGGCCTCCATTTCCTGCCGGTCCTCGCAATCACTCCT |
| | | GCATTCCAACGGTTACAACTACCTGAATTGGTACCTCCAGAAGCCTGGCC |
| | | AGTCGCCCCAGTTGCTGATCTATCTGGGCTCGAAGCGCGCCTCCGGGGTG |
| | | CCTGACCGGTTTAGCGGATCTGGGAGCGGCACGGACTTCACTCTCCACAT |
| | | CACCCGCGTGGGAGCGGAGGACGTGGGAGTGTACTACTGTATGCAGGCGC |
| | | TGCAGACTCCGTACACATTCGGACAGGGCACCAAGCTGGAGATCAAG |
| 139100-aa | 72 | QVQLVQSGAEVRKTGASVKVSCKASGYIFDNFGINWVRQAPGQGLEWMGW |
| VH | | INPKNNNTNYAQKFQGRVTITADESTNTAYMEVSSLRSEDTAVYYCARGPYY |
| | | YQSYMDVWGQGTMVTVSS |
| 139100-aa | 87 | DIVMTQTPLSLPVTPGEPASISCRSSQSLLHSNGYNYLNWYLQKPGQSPQLLIY |
| VL | | LGSKRASGVPDRFSGSGSGTDFTLHITRVGAEDVGVYYCMQALQTPYTFGQG |
| | | TKLEIK |

139101

| | | |
|---|---|---|
| 139101-aa | 43 | QVQLQESGGGLVQPGGSLRLSCAASGFTFSSDAMTWVRQAPGKGLEWVSVI |
| ScFv domain | | SGSGGTTYYADSVKGRFTISRDNSKNTLYLQMNSLRAEDTAVYYCAKLDSSG |
| | | YYYARGPRYWGQGTLVTVSSASGGGGSGGRASGGGGSDIQLTQSPSSLSASV |
| | | GDRVTITCRASQSISSYLNWYQQKPGKAPKLLIYGASTLASGVPARFSGSGSG |
| | | THFTLTINSLQSEDSATYYCQQSYKRASFGQGTKVEIK |
| 139101-nt | 58 | CAAGTGCAACTTCAAGAATCAGGCGGAGGACTCGTGCAGCCCGGAGGAT |
| ScFv domain | | CATTGCGGCTCTCGTGCGCCGCCTCGGGCTTCACCTTCTCGAGCGACGCCA |
| | | TGACCTGGGTCCGCCAGGCCCCGGGGAAGGGGCTGGAATGGGTGTCTGTG |
| | | ATTTCCGGCTCCGGGGGAACTACGTACTACGCCGATTCCGTGAAAGGTCG |
| | | CTTCACTATCTCCCGGGACAACAGCAAGAACACCCTTTATCTGCAAATGA |
| | | ATTCCCTCCGCGCCGAGGACACCGCCGTGTACTACTGCGCCAAGCTGGAC |
| | | TCCTCGGGCTACTACTATGCCCGGGGTCCGAGATACTGGGGACAGGGAAC |
| | | CCTCGTGACCGTGTCCTCCGCGTCCGGCGGAGGAGGGTCGGGAGGGCGGG |
| | | CCTCCGGCGGCGGCGGTTCGGACATCCAGCTGACCCAGTCCCCATCCTCA |
| | | CTGAGCGCAAGCGTGGGCGACAGAGTCACCATTACATGCAGGGCGTCCCA |
| | | GAGCATCAGCTCCTACCTGAACTGGTACCAACAGAAGCCTGGAAAGGCTC |
| | | CTAAGCTGTTGATCTACGGGGCTTCGACCCTGGCATCCGGGGTGCCCGCG |
| | | AGGTTTAGCGGAAGCGGTAGCGGCACTCACTTCACTCTGACCATTAACAG |
| | | CCTCCAGTCCGAGGATTCAGCCACTTACTACTGTCAGCAGTCCTACAAGC |
| | | GGGCCAGCTTCGGACAGGGCACTAAGGTCGAGATCAAG |
| 139101-aa | 73 | QVQLQESGGGLVQPGGSLRLSCAASGFTFSSDAMTWVRQAPGKGLEWVSVI |
| VH | | SGSGGTTYYADSVKGRFTISRDNSKNTLYLQMNSLRAEDTAVYYCAKLDSSG |
| | | YYYARGPRYWGQGTLVTVSS |
| 139101-aa | 88 | DIQLTQSPSSLSASVGDRVTITCRASQSISSYLNWYQQKPGKAPKLLIYGASTL |
| VL | | ASGVPARFSGSGSGTHFTLTINSLQSEDSATYYCQQSYKRASFGQGTKVEIK |

139102

| | | |
|---|---|---|
| 139102-aa | 44 | QVQLVQSGAEVKKPGASVKVSCKASGYTFSNYGITWVRQAPGQGLEWMGW |
| ScFv domain | | ISAYNGNTNYAQKFQGRVTMTRNTSISTAYMELSSLRSEDTAVYYCARGPYY |
| | | YYMDVWGKGTMVTVSSASGGGGSGGRASGGGGSEIVMTQSPLSLPVTPGEP |
| | | ASISCRSSQSLLYSNGYNYVDWYLQKPGQSPQLLIYLGSNRASGVPDRFSGSG |
| | | SGTDFKLQISRVEAEDVGIYYCMQGRQFPYSFGQGTKVEIK |
| 139102-nt | 59 | CAAGTCCAACTGGTCCAGAGCGGTGCAGAAGTGAAGAAGCCCGGAGCGA |
| ScFv domain | | GCGTGAAAGTGTCCTGCAAGGCTTCCGGGTACACCTTCTCCAACTACGGC |
| | | ATCACTTGGGTGCGCCAGGCCCCGGGACAGGGCCTGGAATGGATGGGGTG |
| | | GATTTCCGCGTACAACGGCAATACGAACTACGCTCAGAAGTTCCAGGGTA |
| | | GAGTGACCATGACTAGGAACACCTCCATTTCCACCGCCTACATGGAACTG |
| | | TCCTCCCTGCGGAGCGAGGACACCGCCGTGTACTATTGCGCCCGGGGACC |
| | | ATACTACTACTACATGGATGTCTGGGGGAAGGGGACTATGGTCACCGTGT |
| | | CATCCGCCTCGGGAGGCGGCGGATCAGGAGGACGCGCCTCTGGTGGTGGA |
| | | GGATCGGAGATCGTGATGACCCAGAGCCCTCTCTCCTTGCCCGTGACTCCT |
| | | GGGGAGCCCGCATCCATTTCATGCCGGAGCTCCCAGTCACTTCTCTACTCC |
| | | AACGGCTATAACTACGTGGATTGGTACCTCCAAAAGCCGGGCCAGAGCCC |
| | | GCAGCTGCTGATCTACCTGGGCTCGAACAGGGCCAGCGGAGTGCCTGACC |
| | | GGTTCTCCGGGTCGGGAAGCGGGACCGACTTCAAGCTGCAAATCTCGAGA |
| | | GTGGAGGCCGAGGACGTGGGAATCTACTACTGTATGCAGGGCCGCCAGTT |
| | | TCCGTACTCGTTCGGACAGGGCACCAAAGTGGAAATCAAG |
| 139102-aa | 74 | QVQLVQSGAEVKKPGASVKVSCKASGYTFSNYGITWVRQAPGQGLEWMGW |
| VH | | ISAYNGNTNYAQKFQGRVTMTRNTSISTAYMELSSLRSEDTAVYYCARGPYY |
| | | YYMDVWGKGTMVTVSS |
| 139102-aa | 89 | EIVMTQSPLSLPVTPGEPASISCRSSQSLLYSNGYNYVDWYLQKPGQSPQLLIY |
| VL | | LGSNRASGVPDRFSGSGSGTDFKLQISRVEAEDVGIYYCMQGRQFPYSFGQGT |
| | | KVEIK |

139104

| | | |
|---|---|---|
| 139104-aa | 45 | EVQLLETGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSEIVLTQSPATLSVSPGESATLSCR |
| | | ASQSVSSNLAWYQQKPGQAPRLLIYGASTRASGIPDRFSGSGSGTDFTLTISSL |
| | | QAEDVAVYYCQQYGSSLTFGGGTKVEIK |
| 139104-nt | 60 | GAAGTGCAATTGCTCGAAACTGGAGGAGGTCTGGTGCAACCTGGAGGATC |
| ScFv domain | | ACTTCGCCTGTCCTGCGCCGTGTCGGGCTTTGCCCTGTCCAACCATGGAAT |
| | | GAGCTGGGTCCGCCGCGCGCCGGGGAAGGGCCTCGAATGGGTGTCCGGC |
| | | ATCGTCTACTCCGGCTCCACCTACTACGCCGCGTCCGTGAAGGGCCGGTTC |
| | | ACGATTTCACGGGACAACTCGCGGAACACCCTGTACCTCCAAATGAATTC |
| | | CCTTCGGCCGGAGGATACTGCCATCTACTACTGCTCCGCCCACGGTGGCG |
| | | AATCCGACGTCTGGGGCCAGGGAACCACCGTGACCGTGTCCAGCGCGTCC |
| | | GGGGGAGGAGGAAGCGGGGGTAGAGCATCGGGTGGAGGCGGATCAGAG |
| | | ATCGTGCTGACCCAGTCCCCCGCCACCTTGAGCGTGTCACCAGGAGAGTC |
| | | CGCCACCCTGTCATGCCGCGCCAGCCAGTCCGTGTCCTCCAACCTGGCTTG |
| | | GTACCAGCAGAAGCCGGGGCAGGCCCCTAGACTCCTGATCTATGGGGCGT |
| | | CGACCCGGGCATCTGGAATTCCCGATAGGTTCAGCGGATCGGGCTCGGGC |
| | | ACTGACTTCACTCTGACCATCTCCTCGCTGCAAGCCGAGGACGTGGCTGTG |
| | | TACTACTGTCAGCAGTACGGAAGCTCCCTGACTTTCGGTGGCGGGACCAA |
| | | AGTCGAGATTAAG |
| 139104-aa | 75 | EVQLLETGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139104-aa | 90 | EIVLTQSPATLSVSPGESATLSCRASQSVSSNLAWYQQKPGQAPRLLIYGASTR |
| VL | | ASGIPDRFSGSGSGTDFTLTISSLQAEDVAVYYCQQYGSSLTFGGGTKVEIK |
| 139106 | | |
| 139106-aa | 46 | EVQLVETGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSEIVMTQSPATLSVSPGERATLSC |
| | | RASQSVSSKLAWYQQKPGQAPRLLMYGASIRATGIPDRFSGSGSGTEFTLTISS |
| | | LEPEDFAVYYCQQYGSSSWTFGQGTKVEIK |
| 139106-nt | 61 | GAAGTGCAATTGGTGGAAACTGGAGGAGGACTTGTGCAACCTGGAGGAT |
| ScFv domain | | CATTGAGACTGAGCTGCGCAGTGTCGGGATTCGCCCTGAGCAACCATGGA |
| | | ATGTCCTGGGTCAGAAGGGCCCCTGGAAAAGGCCTCGAATGGGTGTCAGG |
| | | GATCGTGTACTCCGGTTCCACTTACTACGCCGCCTCCGTGAAGGGGCGCTT |
| | | CACTATCTCACGGGATAACTCCCGCAATACCCTGTACCTCCAAATGAACA |
| | | GCCTGCGGCCGGAGGATACCGCCATCTACTACTGTTCCGCCCACGGTGGA |
| | | GAGTCTGACGTCTGGGGCCAGGGAACTACCGTGACCGTGTCCTCCGCGTC |
| | | CGGCGGTGGAGGGAGCGGCGGCCGCGCCAGCGGCGGCGGAGGCTCCGAG |
| | | ATCGTGATGACCCAGAGCCCCGCTACTCTGTCGGTGTCGCCCGGAGAAAG |
| | | GGCGACCCTGTCCTGCCGGGCGTCGCAGTCCGTGAGCAGCAAGCTGGCTT |
| | | GGTACCAGCAGAAGCCGGGCCAGGCACCACGCCTGCTTATGTACGGTGCC |
| | | TCCATTCGGGCCACCGGAATCCCGGACCGGTTCTCGGGGTCGGGGTCCGG |
| | | TACCGAGTTCACACTGACCATTTCCTCGCTCGAGCCCGAGGACTTTGCCGT |
| | | CTATTACTGCCAGCAGTACGGCTCCTCCTCATGGACGTTCGGCCAGGGGA |
| | | CCAAGGTCGAAATCAAG |
| 139106-aa | 76 | EVQLVETGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139106-aa | 91 | EIVMTQSPATLSVSPGERATLSCRASQSVSSKLAWYQQKPGQAPRLLMYGASI |
| VL | | RATGIPDRFSGSGSGTEFTLTISSLEPEDFAVYYCQQYGSSSWTFGQGTKVEIK |

139107

| | | |
|---|---|---|
| 139107-aa | 47 | EVQLVETGGGVVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSEIVLTQSPGTLSLSPGERATLSCR |
| | | ASQSVGSTNLAWYQQKPGQAPRLLIYDASNRATGIPDRFSGGGSGTDFTLTIS |
| | | RLEPEDFAVYYCQQYGSSPPWTFGQGTKVEIK |
| 139107-nt | 62 | GAAGTGCAATTGGTGGAGACTGGAGGAGGAGTGGTGCAACCTGGAGGAA |
| ScFv domain | | GCCTGAGACTGTCATGCGCGGTGTCGGGCTTCGCCCTCTCCAACCACGGA |
| | | ATGTCCTGGGTCCGCCGGGCCCCTGGGAAAGGACTTGAATGGGTGTCCGG |
| | | CATCGTGTACTCGGGTTCCACCTACTACGCGGCCTCAGTGAAGGGCCGGT |
| | | TTACTATTAGCCGCGACAACTCCAGAAACACACTGTACCTCCAAATGAAC |
| | | TCGCTGCGGCCGGAAGATACCGCTATCTACTACTGCTCCGCCCATGGGGG |
| | | AGAGTCGGACGTCTGGGGACAGGGCACCACTGTCACTGTGTCCAGCGCTT |
| | | CCGGCGGTGGTGGAAGCGGGGGACGGGCCTCAGGAGGCGGTGGCAGCGA |
| | | GATTGTGCTGACCCAGTCCCCCGGGACCCTGAGCCTGTCCCCGGGAGAAA |
| | | GGGCCACCCTCTCCTGTCGGGCATCCCAGTCCGTGGGGTCTACTAACCTTG |
| | | CATGGTACCAGCAGAAGCCCGGCCAGGCCCCTCGCCTGCTGATCTACGAC |
| | | GCGTCCAATAGAGCCACCGGCATCCCGGATCGCTTCAGCGGAGGCGGATC |
| | | GGGCACCGACTTCACCCTCACCATTTCAAGGCTGGAACCGGAGGACTTCG |
| | | CCGTGTACTACTGCCAGCAGTATGGTTCGTCCCCACCCTGGACGTTCGGCC |
| | | AGGGGACTAAGGTCGAGATCAAG |
| 139107-aa | 77 | EVQLVETGGGVVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139107-aa | 92 | EIVLTQSPGTLSLSPGERATLSCRASQSVGSTNLAWYQQKPGQAPRLLIYDAS |
| VL | | NRATGIPDRFSGGGSGTDFTLTISRLEPEDFAVYYCQQYGSSPPWTFGQGTKV |
| | | EIK |

139108

| | | |
|---|---|---|
| 139108-aa | 48 | QVQLVESGGGLVKPGGSLRLSCAASGFTFSDYYMSWIRQAPGKGLEWVSYIS |
| ScFv domain | | SSGSTIYYADSVKGRFTISRDNAKNSLYLQMNSLRAEDTAVYYCARESGDGM |
| | | DVWGQGTTVTVSSASGGGGSGGRASGGGGSDIQMTQSPSSLSASVGDRVTIT |
| | | CRASQSISSYLNWYQQKPGKAPKLLIYAASSLQSGVPSRFSGSGSGTDFTLTIS |
| | | SLQPEDFATYYCQQSYTLAFGQGTKVDIK |
| 139108-nt | 63 | CAAGTGCAACTCGTGGAATCTGGTGGAGGACTCGTGAAACCTGGAGGATC |
| ScFv domain | | ATTGAGACTGTCATGCGCGGCCTCGGGATTCACGTTCTCCGATTACTACAT |
| | | GAGCTGGATTCGCCAGGCTCCGGGGAAGGGACTGGAATGGGTGTCCTACA |
| | | TTTCCTCATCCGGCTCCACCATCTACTACGCGGACTCCGTGAAGGGGAGAT |
| | | TCACCATTAGCCGCGATAACGCCAAGAACAGCCTGTACCTTCAGATGAAC |
| | | TCCCTGCGGGCTGAAGATACTGCCGTCTACTACTGCGCAAGGGAGAGCGG |
| | | AGATGGGATGGACGTCTGGGGACAGGGTACCACTGTGACCGTGTCGTCGG |
| | | CCTCCGGCGGAGGGGGTTCGGGTGGAAGGGCCAGCGGCGGCGGAGGCAG |
| | | CGACATCCAGATGACCCAGTCCCCCTCATCGCTGTCCGCCTCCGTGGGCG |
| | | ACCGCGTCACCATCACATGCCGGGCCTCACAGTCGATCTCCTCCTACCTCA |
| | | ATTGGTATCAGCAGAAGCCCGGAAAGGCCCCTAAGCTTCTGATCTACGCA |
| | | GCGTCCTCCCTGCAATCCGGGGTCCCATCTCGGTTCTCCGGCTCGGGCAGC |
| | | GGTACCGACTTCACTCTGACCATCTCGAGCCTGCAGCCGGAGGACTTCGC |
| | | CACTTACTACTGTCAGCAAAGCTACACCCTCGCGTTTGGCCAGGGCACCA |
| | | AAGTGGACATCAAG |
| 139108-aa | 78 | QVQLVESGGGLVKPGGSLRLSCAASGFTFSDYYMSWIRQAPGKGLEWVSYIS |
| VH | | SSGSTIYYADSVKGRFTISRDNAKNSLYLQMNSLRAEDTAVYYCARESGDGM |
| | | DVWGQGTTVTVSS |
| 139108-aa | 93 | DIQMTQSPSSLSASVGDRVTITCRASQSISSYLNWYQQKPGKAPKLLIYAASSL |
| VL | | QSGVPSRFSGSGSGTDFTLTISSLQPEDFATYYCQQSYTLAFGQGTKVDIK |

139110

| | | |
|---|---|---|
| 139110-aa | 50 | QVQLVQSGGGLVKPGGSLRLSCAASGFTFSDYYMSWIRQAPGKGLEWVSYIS |
| ScFv domain | | SSGNTIYYADSVKGRFTISRDNAKNSLYLQMNSLRAEDTAVYYCARSTMVRE |
| | | DYWGQGTLVTVSSASGGGGSGGRASGGGGSDIVLTQSPLSLPVTLGQPASISC |
| | | KSSESLVHNSGKTYLNWFHQRPGQSPRRLIYEVSNRDSGVPDRFTGSGSGTDF |
| | | TLKISRVEAEDVGVYYCMQGTHWPGTFGQGTKLEIK |
| 139110-nt | 65 | CAAGTGCAACTGGTGCAAAGCGGAGGAGGATTGGTCAAACCCGGAGGAA |
| ScFv domain | | GCCTGAGACTGTCATGCGCGGCCTCTGGATTCACCTTCTCCGATTACTACA |
| | | TGTCATGGATCAGACAGGCCCCGGGGAAGGGCCTCGAATGGGTGTCCTAC |
| | | ATCTCGTCCTCCGGGAACACCATCTACTACGCCGACAGCGTGAAGGGCCG |
| | | CTTTACCATTTCCCGCGACAACGCAAAGAACTCGCTGTACCTTCAGATGA |
| | | ATTCCCTGCGGGCTGAAGATACCGCGGTGTACTATTGCGCCCGGTCCACT |
| | | ATGGTCCGGGAGGACTACTGGGGACAGGGCACACTCGTGACCGTGTCCAG |
| | | CGCGAGCGGGGGTGGAGGCAGCGGTGGACGCGCCTCCGGCGGCGGCGGT |
| | | TCAGACATCGTGCTGACTCAGTCGCCCCTGTCGCTGCCGGTCACCCTGGGC |
| | | CAACCGGCCTCAATTAGCTGCAAGTCCTCGGAGAGCCTGGTGCACAACTC |
| | | AGGAAAGACTTACCTGAACTGGTTCCATCAGCGGCCTGGACAGTCCCCAC |
| | | GGAGGCTCATCTATGAAGTGTCCAACAGGGATTCGGGGGTGCCCGACCGC |
| | | TTCACTGGCTCCGGGTCCGGCACCGACTTCACCTTGAAAATCTCCAGAGTG |
| | | GAAGCCGAGGACGTGGGCGTGTACTACTGTATGCAGGGTACCCACTGGCC |
| | | TGGAACCTTTGGACAAGGAACTAAGCTCGAGATTAAG |
| 139110-aa | 80 | QVQLVQSGGGLVKPGGSLRLSCAASGFTFSDYYMSWIRQAPGKGLEWVSYIS |
| VH | | SSGNTIYYADSVKGRFTISRDNAKNSLYLQMNSLRAEDTAVYYCARSTMVRE |
| | | DYWGQGTLVTVSS |
| 139110-aa | 95 | DIVLTQSPLSLPVTLGQPASISCKSSESLVHNSGKTYLNWFHQRPGQSPRRLIY |
| VL | | EVSNRDSGVPDRFTGSGSGTDFTLKISRVEAEDVGVYYCMQGTHWPGTFGQ |
| | | GTKLEIK |

139112

| | | |
|---|---|---|
| 139112-aa | 51 | QVQLVESGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSDIRLTQSPSPLSASVGDRVTITC |
| | | QASEDINKFLNWYHQTPGKAPKLLIYDASTLQTGVPSRFSGSGSGTDFTLTINS |
| | | LQPEDIGTYYCQQYESLPLTFGGGTKVEIK |
| 139112-nt | 66 | CAAGTGCAACTCGTGGAATCTGGTGGAGGACTCGTGCAACCCGGTGGAAG |
| ScFv domain | | CCTTAGGCTGTCGTGCGCCGTCAGCGGGTTTGCTCTGAGCAACCATGGAA |
| | | TGTCCTGGGTCCGCCGGGCACCGGGAAAAGGGCTGGAATGGGTGTCCGGC |
| | | ATCGTGTACAGCGGGTCAACCTATTACGCCGCGTCCGTGAAGGGCAGATT |
| | | CACTATCTCAAGAGACAACAGCCGGAACACCCTGTACTTGCAAATGAATT |
| | | CCCTGCGCCCCGAGGACACCGCCATCTACTACTGCTCCGCCCACGGAGGA |
| | | GAGTCGGACGTGTGGGGCCAGGGAACGACTGTGACTGTGTCCAGCGCATC |
| | | AGGAGGGGGTGGTTCGGGCGGCCGGGCCTCGGGGGGAGGAGGTTCCGAC |
| | | ATTCGGCTGACCCAGTCCCCGTCCCCACTGTCGGCCTCCGTCGGCGACCGC |
| | | GTGACCATCACTTGTCAGGCGTCCGAGGACATTAACAAGTTCCTGAACTG |
| | | GTACCACCAGACCCCTGGAAAGGCCCCCAAGCTGCTGATCTACGATGCCT |
| | | CGACCCTTCAAACTGGAGTGCCTAGCCGGTTCTCCGGGTCCGGCTCCGGC |
| | | ACTGATTTCACTCTGACCATCAACTCATTGCAGCCGGAAGATATCGGGAC |
| | | CTACTATTGCCAGCAGTACGAATCCCTCCCGCTCACATTCGGCGGGGGAA |
| | | CCAAGGTCGAGATTAAG |
| 139112-aa | 81 | QVQLVESGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139112-aa | 96 | DIRLTQSPSPLSASVGDRVTITCQASEDINKFLNWYHQTPGKAPKLLIYDASTL |
| VL | | QTGVPSRFSGSGSGTDFTLTINSLQPEDIGTYYCQQYESLPLTFGGGTKVEIK |

139113

| | | |
|---|---|---|
| 139113-aa | 52 | EVQLVETGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSETTLTQSPATLSVSPGERATLSC |
| | | RASQSVGSNLAWYQQKPGQGPRLLIYGASTRATGIPARFSGSGSGTEFTLTISS |
| | | LQPEDFAVYYCQQYNDWLPVTFGQGTKVEIK |
| 139113-nt | 67 | GAAGTGCAATTGGTGGAAACTGGAGGAGGACTTGTGCAACCTGGAGGAT |
| ScFv domain | | CATTGCGGCTCTCATGCGCTGTCTCCGGCTTCGCCCTGTCAAATCACGGGA |
| | | TGTCGTGGGTCAGACGGGCCCCGGGAAAGGGTCTGGAATGGGTGTCGGG |
| | | GATTGTGTACAGCGGCTCCACCTACTACGCCGCTTCGGTCAAGGGCCGCTT |
| | | CACTATTTCACGGGACAACAGCCGCAACACCCTCTATCTGCAAATGAACT |
| | | CTCTCCGCCCGGAGGATACCGCCATCTACTACTGCTCCGCACACGGCGGC |
| | | GAATCCGACGTGTGGGGACAGGGAACCACTGTCACCGTGTCGTCCGCATC |
| | | CGGTGGCGGAGGATCGGGTGGCCGGGCCTCCGGGGGCGGCGGCAGCGAG |
| | | ACTACCCTGACCCAGTCCCCTGCCACTCTGTCCGTGAGCCCGGGAGAGAG |
| | | AGCCACCCTTAGCTGCCGGGCCAGCCAGAGCGTGGGCTCCAACCTGGCCT |
| | | GGTACCAGCAGAAGCCAGGACAGGGTCCCAGGCTGCTGATCTACGGAGC |
| | | CTCCACTCGCGCGACCGGCATCCCCGCGAGGTTCTCCGGGTCGGGTTCCG |
| | | GGACCGAGTTCACCCTGACCATCTCCTCCCTCCAACCGGAGGACTTCGCG |
| | | GTGTACTACTGTCAGCAGTACAACGATTGGCTGCCCGTGACATTTGGACA |
| | | GGGGACGAAGGTGGAAATCAAA |
| 139113-aa | 82 | EVQLVETGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139113-aa | 97 | ETTLTQSPATLSVSPGERATLSCRASQSVGSNLAWYQQKPGQGPRLLIYGAST |
| VL | | RATGIPARFSGSGSGTEFTLTISSLQPEDFAVYYCQQYNDWLPVTFGQGTKVEI |
| | | K |

139114

| | | |
|---|---|---|
| 139114-aa | 53 | EVQLVESGGGLVQPGGSLRLSCAVSGFALSNHGMSWVRRAPGKGLEWVSGI |
| ScFv domain | | VYSGSTYYAASVKGRFTISRDNSRNTLYLQMNSLRPEDTAIYYCSAHGGESD |
| | | VWGQGTTVTVSSASGGGGSGGRASGGGGSEIVLTQSPGTLSLSPGERATLSCR |
| | | ASQSIGSSSLAWYQQKPGQAPRLLMYGASSRASGIPDRFSGSGSGTDFTLTISR |
| | | LEPEDFAVYYCQQYAGSPPFTFGQGTKVEIK |
| 139114-nt | 68 | GAAGTGCAATTGGTGGAATCTGGTGGAGGACTTGTGCAACCTGGAGGATC |
| ScFv domain | | ACTGAGACTGTCATGCGCGGTGTCCGGTTTTGCCCTGAGCAATCATGGGA |
| | | TGTCGTGGGTCCGGCGCGCCCCCGGAAAGGGTCTGGAATGGGTGTCGGGT |
| | | ATCGTCTACTCCGGGAGCACTTACTACGCCGCGAGCGTGAAGGGCCGCTT |
| | | CACCATTTCCCGCGATAACTCCCGCAACACCCTGTACTTGCAAATGAACTC |
| | | GCTCCGGCCTGAGGACACTGCCATCTACTACTGCTCCGCACACGGAGGAG |
| | | AATCCGACGTGTGGGGCCAGGGAACTACCGTGACCGTCAGCAGCGCCTCC |
| | | GGCGGCGGGGGCTCAGGCGGACGGGCTAGCGGCGGCGGTGGCTCCGAGA |
| | | TCGTGCTGACCCAGTCGCCTGGCACTCTCTCGCTGAGCCCCGGGGAAAGG |
| | | GCAACCCTGTCCTGTCGGGCCAGCCAGTCCATTGGATCATCCTCCCTCGCC |
| | | TGGTATCAGCAGAAACCGGGACAGGCTCCGCGGCTGCTTATGTATGGGGC |
| | | CAGCTCAAGAGCCTCCGGCATTCCCGACCGGTTCTCCGGGTCCGGTTCCG |
| | | GCACCGATTTCACCCTGACTATCTCGAGGCTGGAGCCAGAGGACTTCGCC |
| | | GTGTACTACTGCCAGCAGTACGCGGGGTCCCCGCCGTTCACGTTCGGACA |
| | | GGGAACCAAGGTCGAGATCAAG |
| 139114-aa | 83 | EVQLVESGGGLVQPGGSLRLSCAVSGFALSNHGMSWVRRAPGKGLEWVSGI |
| VH | | VYSGSTYYAASVKGRFTISRDNSRNTLYLQMNSLRPEDTAIYYCSAHGGESD |
| | | VWGQGTTVTVSS |
| 139114-aa | 98 | EIVLTQSPGTLSLSPGERATLSCRASQSIGSSSLAWYQQKPGQAPRLLMYGASS |
| VL | | RASGIPDRFSGSGSGTDFTLTISRLEPEDFAVYYCQQYAGSPPFTFGQGTKVEI |
| | | K |

149362

| | | |
|---|---|---|
| 149362-aa | 129 | QVQLQESGPGLVKPSETLSLTCTVSGGSISSSYYYWGWIRQPPGKGLEWIGSI |
| ScFv domain | | YYSGSAYYNPSLKSRVTISVDTSKNQFSLRLSSVTAADTAVYYCARHWQEWP |
| | | DAFDIWGQGTMVTVSSGGGGSGGGGSGGGGSETTLTQSPAFMSATPGDKVII |
| | | SCKASQDIDDAMNWYQQKPGEAPLFIIQSATSPVPGIPPRFSGSGFGTDFSLTIN |
| | | NIESEDAAYYFCLQHDNFPLTFGQGTKLEIK |
| 149362-nt | 150 | CAAGTGCAGCTTCAGGAAAGCGGACCGGGCCTGGTCAAGCCATCCGAAA |
| ScFv domain | | CTCTCTCCCTGACTTGCACTGTGTCTGGCGGTTCCATCTCATCGTCGTACTA |
| | | CTACTGGGGCTGGATTAGGCAGCCGCCCGGAAAGGGACTGGAGTGGATC |
| | | GGAAGCATCTACTATTCCGGCTCGGCGTACTACAACCCTAGCCTCAAGTC |
| | | GAGAGTGACCATCTCCGTGGATACCTCCAAGAACCAGTTTTCCCTGCGCCT |
| | | GAGCTCCGTGACCGCCGCTGACACCGCCGTGTACTACTGTGCTCGGCATT |
| | | GGCAGGAATGGCCCGATGCCTTCGACATTTGGGGCCAGGGCACTATGGTC |
| | | ACTGTGTCATCCGGGGGTGGAGGCAGCGGGGGAGGAGGGTCCGGGGGGG |
| | | GAGGTTCAGAGACAACCTTGACCCAGTCACCCGCATTCATGTCCGCCACT |
| | | CCGGGAGACAAGGTCATCATCTCGTGCAAAGCGTCCCAGGATATCGACGA |
| | | TGCCATGAATTGGTACCAGCAGAAGCCTGGCGAAGCGCCGCTGTTCATTA |
| | | TCCAATCCGCAACCTCGCCCGTGCCTGGAATCCCACCGCGGTTCAGCGGC |
| | | AGCGGTTTCGGAACCGACTTTTCCCTGACCATTAACAACATTGAGTCCGA |
| | | GGACGCCGCCTACTACTTCTGCCTGCAACACGACAACTTCCCTCTCACGTT |
| | | CGGCCAGGGAACCAAGCTGGAAATCAAG |
| 149362-aa VH | 171 | QVQLQESGPGLVKPSETLSLTCTVSGGSISSSYYYWGWIRQPPGKGLEWIGSI |
| | | YYSGSAYYNPSLKSRVTISVDTSKNQFSLRLSSVTAADTAVYYCARHWQEWP |
| | | DAFDIWGQGTMVTVSS |
| 149362-aa VL | 192 | ETTLTQSPAFMSATPGDKVIISCKASQDIDDAMNWYQQKPGEAPLFIIQSATSP |
| | | VPGIPPRFSGSGFGTDFSLTINNIESEDAAYYFCLQHDNFPLTFGQGTKLEIK |

149363

| | | |
|---|---|---|
| 149363-aa | 130 | VNLRESGPALVKPTQTLTLTCTFSGFSLRTSGMCVSWIRQPPGKALEWLARID |
| ScFv domain | | WDEDKFYSTSLKTRLTISKDTSDNQVVLRMTNMDPADTATYYCARSGAGGT |
| | | SATAFDIWGPGTMVTVSSGGGGSGGGGSGGGGSDIQMTQSPSSLSASVGDRV |
| | | TITCRASQDIYNNLAWFQLKPGSAPRSLMYAANKSQSGVPSRFSGSASGTDFT |
| | | LTISSLQPEDFATYYCQHYYRFPYSFGQGTKLEIK |
| 149363-nt | 151 | CAAGTCAATCTGCGCGAATCCGGCCCCGCCTTGGTCAAGCCTACCCAGAC |
| ScFv domain | | CCTCACTCTGACCTGTACTTTCTCCGGCTTCTCCCTGCGGACTTCCGGGAT |
| | | GTGCGTGTCCTGGATCAGACAGCCTCCGGGAAAGGCCCTGGAGTGGCTCG |
| | | CTCGCATTGACTGGGATGAGGACAAGTTCTACTCCACCTCACTCAAGACC |
| | | AGGCTGACCATCAGCAAAGATACCTCTGACAACCAAGTGGTGCTCCGCAT |
| | | GACCAACATGGACCCAGCCGACACTGCCACTTACTACTGCGCGAGGAGCG |
| | | GAGCGGGCGGAACCTCCGCCACCGCCTTCGATATTTGGGGCCCGGGTACC |
| | | ATGGTCACCGTGTCAAGCGGAGGAGGGGGGTCCGGGGGCGGCGGTTCCG |
| | | GGGGAGGCGGATCGGACATTCAGATGACTCAGTCACCATCGTCCCTGAGC |
| | | GCTAGCGTGGGCGACAGAGTGACAATCACTTGCCGGGCATCCCAGGACAT |
| | | CTATAACAACCTTGCGTGGTTCCAGCTGAAGCCTGGTTCCGCACCGCGGTC |
| | | ACTTATGTACGCCGCCAACAAGAGCCAGTCGGGAGTGCCGTCCCGGTTTT |
| | | CCGGTTCGGCCTCGGGAACTGACTTCACCCTGACGATCTCCAGCCTGCAA |
| | | CCCGAGGATTTCGCCACCTACTACTGCCAGCACTACTACCGCTTTCCCTAC |
| | | TCGTTCGGACAGGGAACCAAGCTGGAAATCAAG |
| 149363-aa VH | 172 | QVNLRESGPALVKPTQTLTLTCTFSGFSLRTSGMCVSWIRQPPGKALEWLARI |
| | | DWDEDKFYSTSLKTRLTISKDTSDNQVVLRMTNMDPADTATYYCARSGAGG |
| | | TSATAFDIWGPGTMVTVSS |
| 149363-aa VL | 193 | DIQMTQSPSSLSASVGDRVTITCRASQDIYNNLAWFQLKPGSAPRSLMYAAN |
| | | KSQSGVPSRFSGSASGTDFTLTISSLQPEDFATYYCQHYYRFPYSFGQGTKLEI |
| | | K |

149364

| | | |
|---|---|---|
| 149364-aa | 131 | EVQLVESGGGLVKPGGSLRLSCAASGFTFSSYSMNWVRQAPGKGLEWVSSIS |
| ScFv domain | | SSSSYIYYADSVKGRFTISRDNAKNSLYLQMNSLRAEDTAVYYCAKTIAAVY |
| | | AFDIWGQGTTVTVSSGGGGSGGGGSGGGGSEIVLTQSPLSLPVTPEEPASISCR |
| | | SSQSLLHSNGYNYLDWYLQKPGQSPQLLIYLGSNRASGVPDRFSGSGSGTDFT |
| | | LKISRVEAEDVGVYYCMQALQTPYTFGQGTKLEIK |
| 149364-nt | 152 | GAAGTGCAGCTTGTCGAATCCGGGGGGGGACTGGTCAAGCCGGGCGGAT |
| ScFv domain | | CACTGAGACTGTCCTGCGCCGCGAGCGGCTTCACGTTCTCCTCCTACTCCA |
| | | TGAACTGGGTCCGCCAAGCCCCCGGGAAGGGACTGGAATGGGTGTCCTCT |
| | | ATCTCCTCGTCGTCGTCCTACATCTACTACGCCGACTCCGTGAAGGGAAGA |
| | | TTCACCATTTCCCGCGACAACGCAAAGAACTCACTGTACTTGCAAATGAA |
| | | CTCACTCCGGGCCGAAGATACTGCTGTGTACTATTGCGCCAAGACTATTGC |
| | | CGCCGTCTACGCTTTCGACATCTGGGGCCAGGGAACCACCGTGACTGTGT |
| | | CGTCCGGTGGTGGTGGCTCGGGCGGAGGAGGAAGCGGCGGCGGGGGGTC |
| | | CGAGATTGTGCTGACCCAGTCGCCACTGAGCCTCCCTGTGACCCCCGAGG |
| | | AACCCGCCAGCATCAGCTGCCGGTCCAGCCAGTCCCTGCTCCACTCCAAC |
| | | GGATACAATTACCTCGATTGGTACCTTCAGAAGCCTGGACAAAGCCCGCA |
| | | GCTGCTCATCTACTTGGGATCAAACCGCGCGTCAGGAGTGCCTGACCGGT |
| | | TCTCCGGCTCGGGCAGCGGTACCGATTTCACCCTGAAAATCTCCAGGGTG |
| | | GAGGCAGAGGACGTGGGAGTGTATTACTGTATGCAGGCGCTGCAGACTCC |
| | | GTACACATTTGGGCAGGGCACCAAGCTGGAGATCAAG |
| 149364-aa VH | 173 | EVQLVESGGGLVKPGGSLRLSCAASGFTFSSYSMNWVRQAPGKGLEWVSSIS |
| | | SSSSYIYYADSVKGRFTISRDNAKNSLYLQMNSLRAEDTAVYYCAKTIAAVY |
| | | AFDIWGQGTTVTVSS |
| 149364-aa VL | 194 | EIVLTQSPLSLPVTPEEPASISCRSSQSLLHSNGYNYLDWYLQKPGQSPQLLIYL |
| | | GSNRASGVPDRFSGSGSGTDFTLKISRVEAEDVGVYYCMQALQTPYTFGQGT |
| | | KLEIK |

149365

| | | |
|---|---|---|
| 149365-aa | 132 | EVQLVESGGGLVKPGGSLRLSCAASGFTFSDYYMSWIRQAPGKGLEWVSYIS |
| ScFv domain | | SSGSTIYYADSVKGRFTISRDNAKNSLYLQMNSLRAEDTAVYYCARDLRGAF |
| | | DIWGQGTMVTVSSGGGGSGGGGSGGGGSSYVLTQSPSVSAAPGYTATISCGG |
| | | NNIGTKSVHWYQQKPGQAPLLVIRDDSVRPSKIPGRFSGSNSGNMATLTISGV |
| | | QAGDEADFYCQVWDSDSEHVVFGGGTKLTVL |
| 149365-nt | 153 | GAAGTCCAGCTCGTGGAGTCCGGCGGAGGCCTTGTGAAGCCTGGAGGTTC |
| ScFv domain | | GCTGAGACTGTCCTGCGCCGCCTCCGGCTTCACCTTCTCCGACTACTACAT |
| | | GTCCTGGATCAGACAGGCCCCGGGAAAGGGCCTGGAATGGGTGTCCTACA |
| | | TCTCGTCATCGGGCAGCACTATCTACTACGCGGACTCAGTGAAGGGGCGG |
| | | TTCACCATTTCCCGGGATAACGCGAAGAACTCGCTGTATCTGCAAATGAA |
| | | CTCACTGAGGGCCGAGGACACCGCCGTGTACTACTGCGCCCGCGATCTCC |
| | | GCGGGGCATTTGACATCTGGGGACAGGGAACCATGGTCACAGTGTCCAGC |
| | | GGAGGGGGAGGATCGGGTGGCGGAGGTTCCGGGGGTGGAGGCTCCTCCT |
| | | ACGTGCTGACTCAGAGCCCAAGCGTCAGCGCTGCGCCCGGTTACACGGCA |
| | | ACCATCTCCTGTGGCGGAAACAACATTGGGACCAAGTCTGTGCACTGGTA |
| | | TCAGCAGAAGCCGGGCCAAGCTCCCCTGTTGGTGATCCGCGATGACTCCG |
| | | TGCGGCCTAGCAAAATTCCGGGACGGTTCTCCGGCTCCAACAGCGGCAAT |
| | | ATGGCCACTCTCACCATCTCGGGAGTGCAGGCCGGAGATGAAGCCGACTT |
| | | CTACTGCCAAGTCTGGGACTCAGACTCCGAGCATGTGGTGTTCGGGGGCG |
| | | GAACCAAGCTGACTGTGCTC |
| 149365-aa VH | 174 | EVQLVESGGGLVKPGGSLRLSCAASGFTFSDYYMSWIRQAPGKGLEWVSYIS |
| | | SSGSTIYYADSVKGRFTISRDNAKNSLYLQMNSLRAEDTAVYYCARDLRGAF |
| | | DIWGQGTMVTVSS |
| 149365-aa VL | 195 | SYVLTQSPSVSAAPGYTATISCGGNNIGTKSVHWYQQKPGQAPLLVIRDDSVR |
| | | PSKIPGRFSGSNSGNMATLTISGVQAGDEADFYCQVWDSDSEHVVFGGGTKL |
| | | TVL |

149366

| | | |
|---|---|---|
| 149366-aa | 133 | QVQLVQSGAEVKKPGASVKVSCKPSGYTVTSHYIHWVRRAPGQGLEWMGM |
| ScFv domain | | INPSGGVTAYSQTLQGRVTMTSDTSSSTVYMELSSLRSEDTAMYYCAREGSG |
| | | SGWYFDFWGRGTLVTVSSGGGGSGGGGSGGGGSSYVLTQPPSVSVSPGQTA |
| | | SITCSGDGLSKKYVSWYQQKAGQSPVVLISRDKERPSGIPDRFSGSNSADTAT |
| | | LTISGTQAMDEADYYCQAWDDTTVVFGGGTKLTVL |
| 149366-nt | 154 | CAAGTGCAGCTGGTGCAGAGCGGGGCCGAAGTCAAGAAGCCGGGAGCCT |
| ScFv domain | | CCGTGAAAGTGTCCTGCAAGCCTTCGGGATACACCGTGACCTCCCACTAC |
| | | ATTCATTGGGTCCGCCGCGCCCCCGGCCAAGGACTCGAGTGGATGGGCAT |
| | | GATCAACCCTAGCGGCGGAGTGACCGCGTACAGCCAGACGCTGCAGGGA |
| | | CGCGTGACTATGACCTCGGATACCTCCTCCTCCACCGTCTATATGGAACTG |
| | | TCCAGCCTGCGGTCCGAGGATACCGCCATGTACTACTGCGCCCGGGAAGG |
| | | ATCAGGCTCCGGGTGGTATTTCGACTTCTGGGGAAGAGGCACCCTCGTGA |
| | | CTGTGTCATCTGGGGGAGGGGGTTCCGGTGGTGGCGGATCGGGAGGAGGC |
| | | GGTTCATCCTACGTGCTGACCCAGCCACCCTCCGTGTCCGTGAGCCCCGGC |
| | | CAGACTGCATCGATTACATGTAGCGGCGACGGCCTCTCCAAGAAATACGT |
| | | GTCGTGGTACCAGCAGAAGGCCGGACAGAGCCCGGTGGTGCTGATCTCAA |
| | | GAGATAAGGAGCGGCCTAGCGGAATCCCGGACAGGTTCTCGGGTTCCAAC |
| | | TCCGCGGACACTGCTACTCTGACCATCTCGGGGACCCAGGCTATGGACGA |
| | | AGCCGATTACTACTGCCAAGCCTGGGACGACACTACTGTCGTGTTTGGAG |
| | | GGGGCACCAAGTTGACCGTCCTT |
| 149366-aa VH | 175 | QVQLVQSGAEVKKPGASVKVSCKPSGYTVTSHYIHWVRRAPGQGLEWMGM |
| | | INPSGGVTAYSQTLQGRVTMTSDTSSSTVYMELSSLRSEDTAMYYCAREGSG |
| | | SGWYFDFWGRGTLVTVSS |
| 149366-aa VL | 196 | SYVLTQPPSVSVSPGQTASITCSGDGLSKKYVSWYQQKAGQSPVVLISRDKER |
| | | PSGIPDRFSGSNSADTATLTISGTQAMDEADYYCQAWDDTTVVFGGGTKLTV |
| | | L |

149367

| | | |
|---|---|---|
| 149367-aa | 134 | QVQLQESGPGLVKPSQTLSLTCTVSGGSISSGGYYWSWIRQHPGKGLEWIGYI |
| ScFv domain | | YYSGSTYYNPSLKSRVTISVDTSKNQFSLKLSSVTAADTAVYYCARAGIAARL |
| | | RGAFDIWGQGTMVTVSSGGGGSGGGGSGGGGSDIVMTQSPSSVSASVGDRVI |
| | | ITCRASQGIRNWLAWYQQKPGKAPNLLIYAASNLQSGVPSRFSGSGSGADFTL |
| | | TISSLQPEDVATYYCQKYNSAPFTFGPGTKVDIK |
| 149367-nt | 155 | CAAGTGCAGCTTCAGGAGAGCGGCCCGGGACTCGTGAAGCCGTCCCAGAC |
| ScFv domain | | CCTGTCCCTGACTTGCACCGTGTCGGGAGGAAGCATCTCGAGCGGAGGCT |
| | | ACTATTGGTCGTGGATTCGGCAGCACCCTGGAAAGGGCCTGGAATGGATC |
| | | GGCTACATCTACTACTCCGGCTCGACCTACTACAACCCATCGCTGAAGTCC |
| | | AGAGTGACAATCTCAGTGGACACGTCCAAGAATCAGTTCAGCCTGAAGCT |
| | | CTCTTCCGTGACTGCGGCCGACACCGCCGTGTACTACTGCGCACGCGCTG |
| | | GAATTGCCGCCCGGCTGAGGGGTGCCTTCGACATTTGGGGACAGGGCACC |
| | | ATGGTCACCGTGTCCTCCGGCGGCGGAGGTTCCGGGGGTGGAGGCTCAGG |
| | | AGGAGGGGGGTCCGACATCGTCATGACTCAGTCGCCCTCAAGCGTCAGCG |
| | | CGTCCGTCGGGGACAGAGTGATCATCACCTGTCGGGCGTCCCAGGGAATT |
| | | CGCAACTGGCTGGCCTGGTATCAGCAGAAGCCCGGAAAGGCCCCCAACCT |
| | | GTTGATCTACGCCGCCTCAAACCTCCAATCCGGGGTGCCGAGCCGCTTCA |
| | | GCGGCTCCGGTTCGGGTGCCGATTTCACTCTGACCATCTCCTCCCTGCAAC |
| | | CTGAAGATGTGGCTACCTACTACTGCCAAAAGTACAACTCCGCACCTTTTA |
| | | CTTTCGGACCGGGGACCAAAGTGGACATTAAG |
| 149367-aa VH | 176 | QVQLQESGPGLVKPSQTLSLTCTVSGGSISSGGYYWSWIRQHPGKGLEWIGYI |
| | | YYSGSTYYNPSLKSRVTISVDTSKNQFSLKLSSVTAADTAVYYCARAGIAARL |
| | | RGAFDIWGQGTMVTVSS |
| 149367-aa VL | 197 | DIVMTQSPSSVSASVGDRVIITCRASQGIRNWLAWYQQKPGKAPNLLIYAASN |
| | | LQSGVPSRFSGSGSGADFTLTISSLQPEDVATYYCQKYNSAPFTFGPGTKVDIK |

149368

| | | |
|---|---|---|
| 149368-aa | 135 | QVQLVQSGAEVKKPGSSVKVSCKASGGTFSSYAISWVRQAPGQGLEWMGGII |
| ScFv domain | | PIFGTANYAQKFQGRVTITADESTSTAYMELSSLRSEDTAVYYCARRGGYQL |
| | | LRWDVGLLRSAFDIWGQGTMVTVSSGGGGSGGGGSGGGGSSYVLTQPPSVS |
| | | VAPGQTARITCGGNNIGSKSVHWYQQKPGQAPVLVLYGKNNRPSGVPDRFS |
| | | GSRSGTTASLTITGAQAEDEADYYCSSRDSSGDHLRVFGTGTKVTVL |
| 149368-nt | 156 | CAAGTGCAGCTGGTCCAGTCGGGCGCCGAGGTCAAGAAGCCCGGGAGCT |
| ScFv domain | | CTGTGAAAGTGTCCTGCAAGGCCTCCGGGGGCACCTTTAGCTCCTACGCC |
| | | ATCTCCTGGGTCCGCCAAGCACCGGGTCAAGGCCTGGAGTGGATGGGGGG |
| | | AATTATCCCTATCTTCGGCACTGCCAACTACGCCCAGAAGTTCCAGGGAC |
| | | GCGTGACCATTACCGCGGACGAATCCACCTCCACCGCTTATATGGAGCTG |
| | | TCCAGCTTGCGCTCGGAAGATACCGCCGTGTACTACTGCGCCCGGAGGGG |
| | | TGGATACCAGCTGCTGAGATGGGACGTGGGCCTCCTGCGGTCGGCGTTCG |
| | | ACATCTGGGGCCAGGGCACTATGGTCACTGTGTCCAGCGGAGGAGGCGGA |
| | | TCGGGAGGCGGCGGATCAGGGGGAGGCGGTTCCAGCTACGTGCTTACTCA |
| | | ACCCCCTTCGGTGTCCGTGGCCCCGGGACAGACCGCCAGAATCACTTGCG |
| | | GAGGAAACAACATTGGGTCCAAGAGCGTGCATTGGTACCAGCAGAAGCC |
| | | AGGACAGGCCCCTGTGCTGGTGCTCTACGGGAAGAACAATCGGCCCAGCG |
| | | GAGTGCCGGACAGGTTCTCGGGTTCACGCTCCGGTACAACCGCTTCACTG |
| | | ACTATCACCGGGGCCCAGGCAGAGGATGAAGCGGACTACTACTGTTCCTC |
| | | CCGGGATTCATCCGGCGACCACCTCCGGGTGTTCGGAACCGGAACGAAGG |
| | | TCACCGTGCTG |
| 149368-aa VH | 177 | QVQLVQSGAEVKKPGSSVKVSCKASGGTFSSYAISWVRQAPGQGLEWMGGII |
| | | PIFGTANYAQKFQGRVTITADESTSTAYMELSSLRSEDTAVYYCARRGGYQL |
| | | LRWDVGLLRSAFDIWGQGTMVTVSS |
| 149368-aa VL | 198 | SYVLTQPPSVSVAPGQTARITCGGNNIGSKSVHWYQQKPGQAPVLVLYGKNN |
| | | RPSGVPDRFSGSRSGTTASLTITGAQAEDEADYYCSSRDSSGDHLRVFGTGTK |
| | | VTVL |

149369

| | | |
|---|---|---|
| 149369-aa | 136 | EVQLQQSGPGLVKPSQTLSLTCAISGDSVSSNSAAWNWIRQSPSRGLEWLGRT |
| ScFv domain | | YYRSKWYSFYAISLKSRIIINPDTSKNQFSLQLKSVTPEDTAVYYCARSSPEGL |
| | | FLYWFDPWGQGTLVTVSSGGDGSGGGGSGGGGSSSELTQDPAVSVALGQTIR |
| | | ITCQGDSLGNYYATWYQQKPGQAPVLVIYGTNNRPSGIPDRFSASSSGNTASL |
| | | TITGAQAEDEADYYCNSRDSSGHHLLFGTGTKVTVL |
| 149369-nt | 157 | GAAGTGCAGCTCCAACAGTCAGGACCGGGGCTCGTGAAGCCATCCCAGAC |
| ScFv domain | | CCTGTCCCTGACTTGTGCCATCTCGGGAGATAGCGTGTCATCGAACTCCGC |
| | | CGCCTGGAACTGGATTCGGCAGAGCCCGTCCCGCGGACTGGAGTGGCTTG |
| | | GAAGGACCTACTACCGGTCCAAGTGGTACTCTTTCTACGCGATCTCGCTGA |
| | | AGTCCCGCATTATCATTAACCCTGATACCTCCAAGAATCAGTTCTCCCTCC |
| | | AACTGAAATCCGTCACCCCCGAGGACACAGCAGTGTATTACTGCGCACGG |
| | | AGCAGCCCCGAAGGACTGTTCCTGTATTGGTTTGACCCCTGGGGCCAGGG |
| | | GACTCTTGTGACCGTGTCGAGCGGCGGAGATGGGTCCGGTGGCGGTGGTT |
| | | CGGGGGGCGGCGGATCATCATCCGAACTGACCCAGGACCCGGCTGTGTCC |
| | | GTGGCGCTGGGACAAACCATCCGCATTACGTGCCAGGGAGACTCCCTGGG |
| | | CAACTACTACGCCACTTGGTACCAGCAGAAGCCGGGCCAAGCCCCTGTGT |
| | | TGGTCATCTACGGGACCAACAACAGACCTTCCGGCATCCCCGACCGGTTC |
| | | AGCGCTTCGTCCTCCGGCAACACTGCCAGCCTGACCATCACTGGAGCGCA |
| | | GGCCGAAGATGAGGCCGACTACTACTGCAACAGCAGAGACTCCTCGGGTC |
| | | ATCACCTCTTGTTCGGAACTGGAACCAAGGTCACCGTGCTG |
| 149369-aa VH | 178 | EVQLQQSGPGLVKPSQTLSLTCAISGDSVSSNSAAWNWIRQSPSRGLEWLGRT |
| | | YYRSKWYSFYAISLKSRIIINPDTSKNQFSLQLKSVTPEDTAVYYCARSSPEGL |
| | | FLYWFDPWGQGTLVTVSS |
| 149369-aa VL | 199 | SSELTQDPAVSVALGQTIRITCQGDSLGNYYATWYQQKPGQAPVLVIYGTNN |
| | | RPSGIPDRFSAS SSGNTASLTITGAQAEDEADYYCNSRDSSGHHLLFGTGTKV |
| | | TVL |

BCMA_EBB-C1978-A4

| | | |
|---|---|---|
| BCMA_EBB- | 137 | EVQLVESGGGLVQPGGSLRLSCAASGFTFSSYAMSWVRQAPGKGLEWVSAIS |
| C1978-A4-aa | | GSGGSTYYADSVKGRFTISRDNSKNTLYLQMNSLRAEDTAVYYCAKVEGSG |
| ScFv domain | | SLDYWGQGTLVTVSSGGGGSGGGGSGGGGSEIVMTQSPGTLSLSPGERATLS |
| | | CRASQSVSSAYLAWYQQKPGQPPRLLISGASTRATGIPDRFGGSGSGTDFTLTI |
| | | SRLEPEDFAVYYCQHYGSSFNGSSLFTFGQGTRLEIK |
| BCMA_EBB- | 158 | GAAGTGCAGCTCGTGGAGTCAGGAGGCGGCCTGGTCCAGCCGGGAGGGT |
| C1978-A4-nt | | CCCTTAGACTGTCATGCGCCGCAAGCGGATTCACTTTCTCCTCCTATGCCA |
| ScFv domain | | TGAGCTGGGTCCGCCAAGCCCCCGGAAAGGGACTGGAATGGGTGTCCGCC |
| | | ATCTCGGGGTCTGGAGGCTCAACTTACTACGCTGACTCCGTGAAGGGACG |
| | | GTTCACCATTAGCCGCGACAACTCCAAGAACACCCTCTACCTCCAAATGA |
| | | ACTCCCTGCGGGCCGAGGATACCGCCGTCTACTACTGCGCCAAAGTGGAA |
| | | GGTTCAGGATCGCTGGACTACTGGGGACAGGGTACTCTCGTGACCGTGTC |
| | | ATCGGGCGGAGGAGGTTCCGGCGGTGGCGGCTCCGGCGGCGGAGGGTCG |
| | | GAGATCGTGATGACCCAGAGCCCTGGTACTCTGAGCCTTTCGCCGGGAGA |
| | | AAGGGCCACCCTGTCCTGCCGCGCTTCCCAATCCGTGTCCTCCGCGTACTT |
| | | GGCGTGGTACCAGCAGAAGCCGGGACAGCCCCCTCGGCTGCTGATCAGCG |
| | | GGGCCAGCACCCGGGCAACCGGAATCCCAGACAGATTCGGGGGTTCCGG |
| | | CAGCGGCACAGATTTCACCCTGACTATTTCGAGGTTGGAGCCCGAGGACT |
| | | TTGCGGTGTATTACTGTCAGCACTACGGGTCGTCCTTTAATGGCTCCAGCC |
| | | TGTTCACGTTCGGACAGGGGACCCGCCTGGAAATCAAG |
| BCMA_EBB- | 179 | EVQLVESGGGLVQPGGSLRLSCAASGFTFSSYAMSWVRQAPGKGLEWVSAIS |
| C1978-A4-aa | | GSGGSTYYADSVKGRFTISRDNSKNTLYLQMNSLRAEDTAVYYCAKVEGSG |
| VH | | SLDYWGQGTLVTVSS |
| BCMA_EBB- | 200 | EIVMTQSPGTLSLSPGERATLSCRASQSVSSAYLAWYQQKPGQPPRLLISGAST |
| C1978-A4-aa | | RATGIPDRFGGSGSGTDFTLTISRLEPEDFAVYYCQHYGSSFNGSSLFTFGQGT |
| VL | | RLEIK |

BCMA_EBB-C1978-G1

| | | |
|---|---|---|
| BCMA_EBB- | 138 | EVQLVETGGGLVQPGGSLRLSCAASGITFSRYPMSWVRQAPGKGLEWVSGIS |
| C1978-G1-aa | | DSGVSTYYADSAKGRFTISRDNSKNTLFLQMSSLRDEDTAVYYCVTRAGSEA |
| ScFv domain | | SDIWGQGTMVTVSSGGGGSGGGGSGGGGSEIVLTQSPATLSLSPGERATLSCR |
| | | ASQSVSNSLAWYQQKPGQAPRLLIYDASSRATGIPDRFSGSGSGTDFTLTISRL |
| | | EPEDFAIYYCQQFGTSSGLTFGGGTKLEIK |
| BCMA_EBB- | 159 | GAAGTGCAACTGGTGGAAACCGGTGGCGGCCTGGTGCAGCCTGGAGGAT |
| C1978-G1-nt | | CATTGAGGCTGTCATGCGCGGCCAGCGGTATTACCTTCTCCCGGTACCCCA |
| ScFv domain | | TGTCCTGGGTCAGACAGGCCCCGGGGAAAGGGCTTGAATGGGTGTCCGGG |
| | | ATCTCGGACTCCGGTGTCAGCACTTACTACGCCGACTCCGCCAAGGGACG |
| | | CTTCACCATTTCCCGGGACAACTCGAAGAACACCCTGTTCCTCCAAATGA |
| | | GCTCCCTCCGGGACGAGGATACTGCAGTGTACTACTGCGTGACCCGCGCC |
| | | GGGTCCGAGGCGTCTGACATTTGGGGACAGGGCACTATGGTCACCGTGTC |
| | | GTCCGGCGGAGGGGGCTCGGGAGGCGGTGGCAGCGGAGGAGGAGGGTCC |
| | | GAGATCGTGCTGACCCAATCCCCGGCCACCCTCTCGCTGAGCCCTGGAGA |
| | | AAGGGCAACCTTGTCCTGTCGCGCGAGCCAGTCCGTGAGCAACTCCCTGG |
| | | CCTGGTACCAGCAGAAGCCCGGACAGGCTCCGAGACTTCTGATCTACGAC |
| | | GCTTCGAGCCGGGCCACTGGAATCCCCGACCGCTTTTCGGGGTCCGGCTC |
| | | AGGAACCGATTTCACCCTGACAATCTCACGGCTGGAGCCAGAGGATTTCG |
| | | CCATCTATTACTGCCAGCAGTTCGGTACTTCCTCCGGCCTGACTTTCGGAG |
| | | GCGGCACGAAGCTCGAAATCAAG |
| BCMA_EBB- | 180 | EVQLVETGGGLVQPGGSLRLSCAASGITFSRYPMSWVRQAPGKGLEWVSGIS |
| C1978-G1-aa | | DSGVSTYYADSAKGRFTISRDNSKNTLFLQMSSLRDEDTAVYYCVTRAGSEA |
| VH | | SDIWGQGTMVTVSS |
| BCMA_EBB- | 201 | EIVLTQSPATLSLSPGERATLSCRASQSVSNSLAWYQQKPGQAPRLLIYDASSR |
| C1978-G1-aa | | ATGIPDRFSGSGSGTDFTLTISRLEPEDFAIYYCQQFGTSSGLTFGGGTKLEIK |
| VL | | |

BCMA_EBB-C1979-C1

| | | |
|---|---|---|
| BCMA_EBB- | 139 | QVQLVESGGGLVQPGGSLRLSCAASGFTFSSYAMSWVRQAPGKGLEWVSAIS |
| C1979-C1-aa | | GSGGSTYYADSVKGRFTISRDNAKNSLYLQMNSLRAEDTAIYYCARATYKRE |
| ScFv domain | | LRYYYGMDVWGQGTMVTVSSGGGGSGGGGSGGGGSEIVMTQSPGTVSLSP |
| | | GERATLSCRASQSVSSSFLAWYQQKPGQAPRLLIYGASSRATGIPDRFSGSGS |
| | | GTDFTLTISRLEPEDSAVYYCQQYHSSPSWTFGQGTRLEIK |
| BCMA_EBB- | 160 | CAAGTGCAGCTCGTGGAATCGGGTGGCGGACTGGTGCAGCCGGGGGGCTC |
| C1979-C1-nt | | ACTTAGACTGTCCTGCGCGGCCAGCGGATTCACTTTCTCCTCCTACGCCAT |
| ScFv domain | | GTCCTGGGTCAGACAGGCCCCTGGAAAGGGCCTGGAATGGGTGTCCGCAA |
| | | TCAGCGGCAGCGGCGGCTCGACCTATTACGCGGATTCAGTGAAGGGCAGA |
| | | TTCACCATTTCCCGGGACAACGCCAAGAACTCCTTGTACCTTCAAATGAAC |
| | | TCCCTCCGCGCGGAAGATACCGCAATCTACTACTGCGCTCGGGCCACTTA |
| | | CAAGAGGGAACTGCGCTACTACTACGGGATGGACGTCTGGGGCCAGGGA |
| | | ACCATGGTCACCGTGTCCAGCGGAGGAGGAGGATCGGGAGGAGGCGGTA |
| | | GCGGGGGTGGAGGGTCGGAGATCGTGATGACCCAGTCCCCCGGCACTGTG |
| | | TCGCTGTCCCCCGGCGAACGGGCCACCCTGTCATGTCGGGCCAGCCAGTC |
| | | AGTGTCGTCAAGCTTCCTCGCCTGGTACCAGCAGAAACCGGGACAAGCTC |
| | | CCCGCCTGCTGATCTACGGAGCCAGCAGCCGGGCCACCGGTATTCCTGAC |
| | | CGGTTCTCCGGTTCGGGGTCCGGGACCGACTTTACTCTGACTATCTCTCGC |
| | | CTCGAGCCAGAGGACTCCGCCGTGTATTACTGCCAGCAGTACCACTCCTC |
| | | CCCGTCCTGGACGTTCGGACAGGGCACAAGGCTGGAGATTAAG |
| BCMA_EBB- | 181 | QVQLVESGGGLVQPGGSLRLSCAASGFTFSSYAMSWVRQAPGKGLEWVSAIS |
| C1979-C1-aa | | GSGGSTYYADSVKGRFTISRDNAKNSLYLQMNSLRAEDTAIYYCARATYKRE |
| VH | | LRYYYGMDVWGQGTMVTVSS |
| BCMA_EBB- | 202 | EIVMTQSPGTVSLSPGERATLSCRASQSVSSSFLAWYQQKPGQAPRLLIYGAS |
| C1979-C1-aa | | SRATGIPDRFSGSGSGTDFTLTISRLEPEDSAVYYCQQYHSSPSWTFGQGTRLE |
| VL | | IK |

BCMA_EBB-C1978-C7

| | | |
|---|---|---|
| BCMA_EBB- | 140 | EVQLVETGGGLVQPGGSLRLSCAASGFTFSSYAMSWVRQAPGKGLEWVSAIS |
| C1978-C7-aa | | GSGGSTYYADSVKGRFTISRDNSKNTLYLQMNTLKAEDTAVYYCARATYKR |
| ScFv domain | | ELRYYYGMDVWGQGTTVTVSSGGGGSGGGGSGGGGSEIVLTQSPSTLSLSPG |
| | | ESATLSCRASQSVSTTFLAWYQQKPGQAPRLLIYGSSNRATGIPDRFSGSGSGT |
| | | DFTLTIRRLEPEDFAVYYCQQYHSSPSWTFGQGTKVEIK |
| BCMA_EBB- | 161 | GAGGTGCAGCTTGTGGAAACCGGTGGCGGACTGGTGCAGCCCGGAGGAA |
| C1978-C7-nt | | GCCTCAGGCTGTCCTGCGCCGCGTCCGGCTTCACCTTCTCCTCGTACGCCA |
| ScFv domain | | TGTCCTGGGTCCGCCAGGCCCCCGGAAAGGGCCTGGAATGGGTGTCCGCC |
| | | ATCTCTGGAAGCGGAGGTTCCACGTACTACGCGGACAGCGTCAAGGGAAG |
| | | GTTCACAATCTCCCGCGATAATTCGAAGAACACTCTGTACCTTCAAATGA |
| | | ACACCCTGAAGGCCGAGGACACTGCTGTGTACTACTGCGCACGGGCCACC |
| | | TACAAGAGAGAGCTCCGGTACTACTACGGAATGGACGTCTGGGGCCAGG |
| | | GAACTACTGTGACCGTGTCCTCGGGAGGGGGTGGCTCCGGGGGGGGCGGC |
| | | TCCGGCGGAGGCGGTTCCGAGATTGTGCTGACCCAGTCACCTTCAACTCT |
| | | GTCGCTGTCCCCGGGAGAGAGCGCTACTCTGAGCTGCCGGGCCAGCCAGT |
| | | CCGTGTCCACCACCTTCCTCGCCTGGTATCAGCAGAAGCCGGGGCAGGCA |
| | | CCACGGCTCTTGATCTACGGGTCAAGCAACAGAGCGACCGGAATTCCTGA |
| | | CCGCTTCTCGGGGAGCGGTTCAGGCACCGACTTCACCCTGACTATCCGGC |
| | | GCCTGGAACCCGAAGATTTCGCCGTGTATTACTGTCAACAGTACCACTCCT |
| | | CGCCGTCCTGGACCTTTGGCCAAGGAACCAAAGTGGAAATCAAG |
| BCMA_EBB- | 182 | EVQLVETGGGLVQPGGSLRLSCAASGFTFSSYAMSWVRQAPGKGLEWVSAIS |
| C1978-C7-aa | | GSGGSTYYADSVKGRFTISRDNSKNTLYLQMNTLKAEDTAVYYCARATYKR |
| VH | | ELRYYYGMDVWGQGTTVTVSS |
| BCMA_EBB- | 203 | EIVLTQSPSTLSLSPGESATLSCRASQSVSTTFLAWYQQKPGQAPRLLIYGSSN |
| C1978-C7-aa | | RATGIPDRFSGSGSGTDFTLTIRRLEPEDFAVYYCQQYHSSPSWTFGQGTKVEI |
| VL | | K |

BCMA_EBB-C1978-D10

| | | |
|---|---|---|
| BCMA_EBB- | 141 | EVQLVETGGGLVQPGRSLRLSCAASGFTFDDYAMHWVRQAPGKGLEWVSGI |
| C1978-D10- | | SWNSGSIGYADSVKGRFTISRDNAKNSLYLQMNSLRDEDTAVYYCARVGKA |
| aa | | VPDVWGQGTTVTVSSGGGGSGGGGSGGGGSDIVMTQTPSSLSASVGDRVTIT |
| ScFv domain | | CRASQSISSYLNWYQQKPGKAPKLLIYAASSLQSGVPSRFSGSGSGTDFTLTIS |
| | | SLQPEDFATYYCQQSYSTPYSFGQGTRLEIK |
| BCMA_EBB- | 162 | GAAGTGCAGCTCGTGGAAACTGGAGGTGGACTCGTGCAGCCTGGACGGTC |
| C1978-D10-nt | | GCTGCGGCTGAGCTGCGCTGCATCCGGCTTCACCTTCGACGATTATGCCAT |
| ScFv domain | | GCACTGGGTCAGACAGGCGCCAGGGAAGGGACTTGAGTGGGTGTCCGGT |
| | | ATCAGCTGGAATAGCGGCTCAATCGGATACGCGGACTCCGTGAAGGGAA |
| | | GGTTCACCATTTCCCGCGACAACGCCAAGAACTCCCTGTACTTGCAAATG |
| | | AACAGCCTCCGGGATGAGGACACTGCCGTGTACTACTGCGCCCGCGTCGG |
| | | AAAAGCTGTGCCCGACGTCTGGGGCCAGGGAACCACTGTGACCGTGTCCA |
| | | GCGGCGGGGGTGGATCGGGCGGTGGAGGGTCCGGTGGAGGGGGCTCAGA |
| | | TATTGTGATGACCCAGACCCCCTCGTCCCTGTCCGCCTCGGTCGGCGACCG |
| | | CGTGACTATCACATGTAGAGCCTCGCAGAGCATCTCCAGCTACCTGAACT |
| | | GGTATCAGCAGAAGCCGGGGAAGGCCCCGAAGCTCCTGATCTACGCGGC |
| | | ATCATCACTGCAATCGGGAGTGCCGAGCCGGTTTTCCGGGTCCGGCTCCG |
| | | GCACCGACTTCACGCTGACCATTTCTTCCCTGCAACCCGAGGACTTCGCCA |
| | | CTTACTACTGCCAGCAGTCCTACTCCACCCCTTACTCCTTCGGCCAAGGAA |
| | | CCAGGCTGGAAATCAAG |
| BCMA_EBB- | 183 | EVQLVETGGGLVQPGRSLRLSCAASGFTFDDYAMHWVRQAPGKGLEWVSGI |
| C1978-D10-aa | | SWNSGSIGYADSVKGRFTISRDNAKNSLYLQMNSLRDEDTAVYYCARVGKA |
| VH | | VPDVWGQGTTVTVSS |
| BCMA_EBB- | 204 | DIVMTQTPSSLSASVGDRVTITCRASQSISSYLNWYQQKPGKAPKLLIYAASSL |
| C1978-D10-aa VL | | QSGVPSRFSGSGSGTDFTLTISSLQPEDFATYYCQQSYSTPYSFGQGTRLEIK |

BCMA_EBB-C1979-C12

| | | |
|---|---|---|
| BCMA_EBB- | 142 | EVQLVESGGGLVQPGRSLRLSCTASGFTFDDYAMHWVRQRPGKGLEWVASI |
| C1979-C12-aa | | NWKGNSLAYGDSVKGRFAISRDNAKNTVFLQMNSLRTEDTAVYYCASHQG |
| ScFv domain | | VAYYNYAMDVWGRGTLVTVSSGGGGSGGGGSGGGGSEIVLTQSPGTLSLSP |
| | | GERATLSCRATQSIGSSFLAWYQQRPGQAPRLLIYGASQRATGIPDRFSGRGS |
| | | GTDFTLTISRVEPEDSAVYYCQHYESSPSWTFGQGTKVEIK |
| BCMA_EBB- | 163 | GAAGTGCAGCTCGTGGAGAGCGGGGGAGGATTGGTGCAGCCCGGAAGGT |
| C1979-C12-nt | | CCCTGCGGCTCTCCTGCACTGCGTCTGGCTTCACCTTCGACGACTACGCGA |
| ScFv domain | | TGCACTGGGTCAGACAGCGCCCGGGAAAGGGCCTGGAATGGGTCGCCTCA |
| | | ATCAACTGGAAGGGAAACTCCCTGGCCTATGGCGACAGCGTGAAGGGCC |
| | | GCTTCGCCATTTCGCGCGACAACGCCAAGAACACCGTGTTTCTGCAAATG |
| | | AATTCCCTGCGGACCGAGGATACCGCTGTGTACTACTGCGCCAGCCACCA |
| | | GGGCGTGGCATACTATAACTACGCCATGGACGTGTGGGGAAGAGGGACG |
| | | CTCGTCACCGTGTCCTCCGGGGGCGGTGGATCGGGTGGAGGAGGAAGCGG |
| | | TGGCGGGGGCAGCGAAATCGTGCTGACTCAGAGCCCGGGAACTCTTTCAC |
| | | TGTCCCCGGGAGAACGGGCCACTCTCTCGTGCCGGGCCACCCAGTCCATC |
| | | GGCTCCTCCTTCCTTGCCTGGTACCAGCAGAGGCCAGGACAGGCGCCCCG |
| | | CCTGCTGATCTACGGTGCTTCCCAACGCGCCACTGGCATTCCTGACCGGTT |
| | | CAGCGGCAGAGGGTCGGGAACCGATTTCACACTGACCATTTCCCGGGTGG |
| | | AGCCCGAAGATTCGGCAGTCTACTACTGTCAGCATTACGAGTCCTCCCCTT |
| | | CATGGACCTTCGGTCAAGGGACCAAAGTGGAGATCAAG |
| BCMA_EBB- | 184 | EVQLVESGGGLVQPGRSLRLSCTASGFTFDDYAMHWVRQRPGKGLEWVASI |
| C1979-C12-aa | | NWKGNSLAYGDSVKGRFAISRDNAKNTVFLQMNSLRTEDTAVYYCASHQG |
| VH | | VAYYNYAMDVWGRGTLVTVSS |
| BCMA_EBB- | 205 | EIVLTQSPGTLSLSPGERATLSCRATQSIGSSFLAWYQQRPGQAPRLLIYGASQ |
| C1979-C12-aa | | RATGIPDRFSGRGSGTDFTLTISRVEPEDSAVYYCQHYESSPSWTFGQGTKVEI |
| VL | | K |

BCMA_EBB-C1980-G4

| | | |
|---|---|---|
| BCMA_EBB- | 143 | EVQLVESGGGLVQPGGSLRLSCAASGFTFSSYAMSWVRQAPGKGLEWVSAIS |
| C1980-G4-aa | | GSGGSTYYADSVKGRFTISRDNSKNTLYLQMNSLRAEDTAVYYCAKVVRDG |
| ScFv domain | | MDVWGQGTTVTVSSGGGGSGGGGSGGGGSEIVLTQSPATLSLSPGERATLSC |
| | | RASQSVSSSYLAWYQQKPGQAPRLLIYGASSRATGIPDRFSGNGSGTDFTLTIS |
| | | RLEPEDFAVYYCQQYGSPPRFTFGPGTKVDIK |
| BCMA_EBB- | 164 | GAGGTGCAGTTGGTCGAAAGCGGGGGCGGGCTTGTGCAGCCTGGCGGATC |
| C1980-G4-nt | | ACTGCGGCTGTCCTGCGCGGCATCAGGCTTCACGTTTTCTTCCTACGCCAT |
| ScFv domain | | GTCCTGGGTGCGCCAGGCCCCTGGAAAGGGACTGGAATGGGTGTCCGCGA |
| | | TTTCGGGGTCCGGCGGGAGCACCTACTACGCCGATTCCGTGAAGGGCCGC |
| | | TTCACTATCTCGCGGGACAACTCCAAGAACACCCTCTACCTCCAAATGAA |
| | | TAGCCTGCGGGCCGAGGATACCGCCGTCTACTATTGCGCTAAGGTCGTGC |
| | | GCGACGGAATGGACGTGTGGGGACAGGGTACCACCGTGACAGTGTCCTCG |
| | | GGGGGAGGCGGTAGCGGCGGAGGAGGAAGCGGTGGTGGAGGTTCCGAGA |
| | | TTGTGCTGACTCAATCACCCGCGACCCTGAGCCTGTCCCCCGGCGAAAGG |
| | | GCCACTCTGTCCTGTCGGGCCAGCCAATCAGTCTCCTCCTCGTACCTGGCC |
| | | TGGTACCAGCAGAAGCCAGGACAGGCTCCGAGACTCCTTATCTATGGCGC |
| | | ATCCTCCCGCGCCACCGGAATCCCGGATAGGTTCTCGGGAAACGGATCGG |
| | | GGACCGACTTCACTCTCACCATCTCCCGGCTGGAACCGGAGGACTTCGCC |
| | | GTGTACTACTGCCAGCAGTACGGCAGCCCGCCTAGATTCACTTTCGGCCCC |
| | | GGCACCAAAGTGGACATCAAG |
| BCMA_EBB- | 185 | EVQLVESGGGLVQPGGSLRLSCAASGFTFSSYAMSWVRQAPGKGLEWVSAIS |
| C1980-G4-aa | | GSGGSTYYADSVKGRFTISRDNSKNTLYLQMNSLRAEDTAVYYCAKVVRDG |
| VH | | MDVWGQGTTVTVSS |
| BCMA_EBB- | 206 | EIVLTQSPATLSLSPGERATLSCRASQSVSSSYLAWYQQKPGQAPRLLIYGASS |
| C1980-G4-aa | | RATGIPDRFSGNGSGTDFTLTISRLEPEDFAVYYCQQYGSPPRFTFGPGTKVDI |
| VL | | K |

BCMA_EBB-C1980-D2

| | | |
|---|---|---|
| BCMA_EBB- | 144 | EVQLLESGGGLVQPGGSLRLSCAASGFTFSSYAMSWVRQAPGKGLEWVSAIS |
| C1980-D2-aa | | GSGGSTYYADSVKGRFTISRDNSKNTLYLQMNSLRAEDTAVYYCAKIPQTGT |
| ScFv domain | | FDYWGQGTLVTVSSGGGGSGGGGSGGGGSEIVLTQSPGTLSLSPGERATLSC |
| | | RASQSVSSSYLAWYQQRPGQAPRLLIYGASSRATGIPDRFSGSGSGTDFTLTIS |
| | | RLEPEDFAVYYCQHYGSSPSWTFGQGTRLEIK |
| BCMA_EBB- | 165 | GAAGTGCAGCTGCTGGAGTCCGGCGGTGGATTGGTGCAACCGGGGGGATC |
| C1980-D2-nt | | GCTCAGACTGTCCTGTGCGGCGTCAGGCTTCACCTTCTCGAGCTACGCCAT |
| ScFv domain | | GTCATGGGTCAGACAGGCCCCTGGAAAGGGTCTGGAATGGGTGTCCGCCA |
| | | TTTCCGGGAGCGGGGGATCTACATACTACGCCGATAGCGTGAAGGGCCGC |
| | | TTCACCATTTCCCGGGACAACTCCAAGAACACTCTCTATCTGCAAATGAAC |
| | | TCCCTCCGCGCTGAGGACACTGCCGTGTACTACTGCGCCAAAATCCCTCA |
| | | GACCGGCACCTTCGACTACTGGGGACAGGGGACTCTGGTCACCGTCAGCA |
| | | GCGGTGGCGGAGGTTCGGGGGGAGGAGGAAGCGGCGGCGGAGGGTCCGA |
| | | GATTGTGCTGACCCAGTCACCCGGCACTTTGTCCCTGTCGCCTGGAGAAA |
| | | GGGCCACCCTTTCCTGCCGGGCATCCCAATCCGTGTCCTCCTCGTACCTGG |
| | | CCTGGTACCAGCAGAGGCCCGGACAGGCCCCACGGCTTCTGATCTACGGA |
| | | GCAAGCAGCCGCGCGACCGGTATCCCGGACCGGTTTTCGGGCTCGGGCTC |
| | | AGGAACTGACTTCACCCTCACCATCTCCCGCCTGGAACCCGAAGATTTCG |
| | | CTGTGTATTACTGCCAGCACTACGGCAGCTCCCCGTCCTGGACGTTCGGCC |
| | | AGGGAACTCGGCTGGAGATCAAG |
| BCMA_EBB- | 186 | EVQLLESGGGLVQPGGSLRLSCAASGFTFSSYAMSWVRQAPGKGLEWVSAIS |
| C1980-D2-aa | | GSGGSTYYADSVKGRFTISRDNSKNTLYLQMNSLRAEDTAVYYCAKIPQTGT |
| VH | | FDYWGQGTLVTVSS |
| BCMA_EBB- | 207 | EIVLTQSPGTLSLSPGERATLSCRASQSVSSSYLAWYQQRPGQAPRLLIYGASS |
| C1980-D2-aa VL | | RATGIPDRFSGSGSGTDFTLTISRLEPEDFAVYYCQHYGSSPSWTFGQGTRLEI |
| | | K |

BCMA_EBB-C1978-A10

| | | |
|---|---|---|
| BCMA_EBB- | 145 | EVQLVETGGGLVQPGGSLRLSCAASGFTFSSYAMSWVRQAPGKGLEWVSAIS |
| C1978-A10-aa | | GSGGSTYYADSVKGRFTMSRENDKNSVFLQMNSLRVEDTGVYYCARANYK |
| ScFv domain | | RELRYYYGMDVWGQGTMVTVSSGGGGSGGGGSGGGGSEIVMTQSPGTLSLS |
| | | PGESATLSCRASQRVASNYLAWYQHKPGQAPSLLISGASSRATGVPDRFSGSG |
| | | SGTDFTLAISRLEPEDSAVYYCQHYDSSPSWTFGQGTKVEIK |
| BCMA_EBB- | 166 | GAAGTGCAACTGGTGGAAACCGGTGGAGGACTCGTGCAGCCTGGCGGCA |
| C1978-A10-nt | | GCCTCCGGCTGAGCTGCGCCGCTTCGGGATTCACCTTTTCCTCCTACGCGA |
| ScFv domain | | TGTCTTGGGTCAGACAGGCCCCCGGAAAGGGGCTGGAATGGGTGTCAGCC |
| | | ATCTCCGGCTCCGGCGGATCAACGTACTACGCCGACTCCGTGAAAGGCCG |
| | | GTTCACCATGTCGCGCGAGAATGACAAGAACTCCGTGTTCCTGCAAATGA |
| | | ACTCCCTGAGGGTGGAGGACACCGGAGTGTACTATTGTGCGCGCGCCAAC |
| | | TACAAGAGAGAGCTGCGGTACTACTACGGAATGGACGTCTGGGGACAGG |
| | | GAACTATGGTGACCGTGTCATCCGGTGGAGGGGGAAGCGGCGGTGGAGG |
| | | CAGCGGGGGCGGGGGTTCAGAAATTGTCATGACCCAGTCCCCGGGAACTC |
| | | TTTCCCTCTCCCCCGGGGAATCCGCGACTTTGTCCTGCCGGGCCAGCCAGC |
| | | GCGTGGCCTCGAACTACCTCGCATGGTACCAGCATAAGCCAGGCCAAGCC |
| | | CCTTCCCTGCTGATTTCCGGGGCTAGCAGCCGCGCCACTGGCGTGCCGGAT |
| | | AGGTTCTCGGGAAGCGGCTCGGGTACCGATTTCACCCTGGCAATCTCGCG |
| | | GCTGGAACCGGAGGATTCGGCCGTGTACTACTGCCAGCACTATGACTCAT |
| | | CCCCCTCCTGGACATTCGGACAGGGCACCAAGGTCGAGATCAAG |
| BCMA_EBB- | 187 | EVQLVETGGGLVQPGGSLRLSCAASGFTFSSYAMSWVRQAPGKGLEWVSAIS |
| C1978-A10-aa | | GSGGSTYYADSVKGRFTMSRENDKNSVFLQMNSLRVEDTGVYYCARANYK |
| VH | | RELRYYYGMDVWGQGTMVTVSS |
| BCMA_EBB- | 208 | EIVMTQSPGTLSLSPGESATLSCRASQRVASNYLAWYQHKPGQAPSLLISGAS |
| C1978-A10-aa | | SRATGVPDRFSGSGSGTDFTLAISRLEPEDSAVYYCQHYDSSPSWTFGQGTKV |
| VL | | EIK |

BCMA_EBB-C1978-D4

| | | |
|---|---|---|
| BCMA_EBB- | 146 | EVQLLETGGGLVQPGGSLRLSCAASGFSFSSYAMSWVRQAPGKGLEWVSAIS |
| C1978-D4-aa | | GSGGSTYYADSVKGRFTISRDNSKNTLYLQMNSLRAEDTAVYYCAKALVGA |
| ScFv domain | | TGAFDIWGQGTLVTVSSGGGGSGGGGSGGGGSEIVLTQSPGTLSLSPGERATL |
| | | SCRASQSLSSNFLAWYQQKPGQAPGLLIYGASNWATGTPDRFSGSGSGTDFT |
| | | LTITRLEPEDFAVYYCQYYGTSPMYTFGQGTKVEIK |
| BCMA_EBB- | 167 | GAAGTGCAGCTGCTCGAAACCGGTGGAGGGCTGGTGCAGCCAGGGGGCT |
| C1978-D4-nt | | CCCTGAGGCTTTCATGCGCCGCTAGCGGATTCTCCTTCTCCTCTTACGCCA |
| ScFv domain | | TGTCGTGGGTCCGCCAAGCCCCTGGAAAAGGCCTGGAATGGGTGTCCGCG |
| | | ATTTCCGGGAGCGGAGGTTCGACCTATTACGCCGACTCCGTGAAGGGCCG |
| | | CTTTACCATCTCCCGGGATAACTCCAAGAACACTCTGTACCTCCAAATGAA |
| | | CTCGCTGAGAGCCGAGGACACCGCCGTGTATTACTGCGCGAAGGCGCTGG |
| | | TCGGCGCGACTGGGGCATTCGACATCTGGGGACAGGGAACTCTTGTGACC |
| | | GTGTCGAGCGGAGGCGGCGGCTCCGGCGGAGGAGGGAGCGGGGGCGGTG |
| | | GTTCCGAAATCGTGTTGACTCAGTCCCCGGGAACCCTGAGCTTGTCACCCG |
| | | GGGAGCGGGCCACTCTCTCCTGTCGCGCCTCCCAATCGCTCTCATCCAATT |
| | | TCCTGGCCTGGTACCAGCAGAAGCCCGGACAGGCCCCGGGCCTGCTCATC |
| | | TACGGCGCTTCAAACTGGGCAACGGGAACCCCTGATCGGTTCAGCGGAAG |
| | | CGGATCGGGTACTGACTTTACCCTGACCATCACCAGACTGGAACCGGAGG |
| | | ACTTCGCCGTGTACTACTGCCAGTACTACGGCACCTCCCCCATGTACACAT |
| | | TCGGACAGGGTACCAAGGTCGAGATTAAG |
| BCMA_EBB- | 188 | EVQLLETGGGLVQPGGSLRLSCAASGFSFSSYAMSWVRQAPGKGLEWVSAIS |
| C1978-D4-aa VH | | GSGGSTYYADSVKGRFTISRDNSKNTLYLQMNSLRAEDTAVYYCAKALVGA |
| | | TGAFDIWGQGTLVTVSS |
| BCMA_EBB- | 209 | EIVLTQSPGTLSLSPGERATLSCRASQSLSSNFLAWYQQKPGQAPGLLIYGASN |
| C1978-D4-aa VL | | WATGTPDRFSGSGSGTDFTLTITRLEPEDFAVYYCQYYGTSPMYTFGQGTKV |
| | | EIK |

BCMA_EBB-C1980-A2

| | | |
|---|---|---|
| BCMA_EBB- | 147 | EVQLLESGGGLVQPGGSLRLSCAASGFTFSSYAMSWVRQAPGKGLEWVSAIS |
| C1980-A2-aa | | GSGGSTYYADSVKGRFTISRDNSKNTLYLQMNSLRAEDTAVYYCVLWFGEG |
| ScFv domain | | FDPWGQGTLVTVSSGGGGSGGGGSGGGGSDIVLTQSPLSLPVTPGEPASISCR |
| | | SSQSLLHSNGYNYLDWYLQKPGQSPQLLIYLGSNRASGVPDRFSGSGSGTDFT |
| | | LKISRVEAEDVGVYYCMQALQTPLTFGGGTKVDIK |
| BCMA_EBB- | 168 | GAAGTGCAGCTGCTTGAGAGCGGTGGAGGTCTGGTGCAGCCCGGGGGATC |
| C1980-A2-nt | | ACTGCGCCTGTCCTGTGCCGCGTCCGGTTTCACTTTCTCCTCGTACGCCAT |
| ScFv domain | | GTCGTGGGTCAGACAGGCACCGGGAAAGGGACTGGAATGGGTGTCAGCC |
| | | ATTTCGGGTTCGGGGGGCAGCACCTACTACGCTGACTCCGTGAAGGGCCG |
| | | GTTCACCATTTCCCGCGACAACTCCAAGAACACCTTGTACCTCCAAATGA |
| | | ACTCCCTGCGGGCCGAAGATACCGCCGTGTATTACTGCGTGCTGTGGTTCG |
| | | GAGAGGGATTCGACCCGTGGGGACAAGGAACACTCGTGACTGTGTCATCC |
| | | GGCGGAGGCGGCAGCGGTGGCGGCGGTTCCGGCGGCGGCGGATCTGACA |
| | | TCGTGTTGACCCAGTCCCCTCTGAGCCTGCCGGTCACTCCTGGCGAACCAG |
| | | CCAGCATCTCCTGCCGGTCGAGCCAGTCCCTCCTGCACTCCAATGGGTACA |
| | | ACTACCTCGATTGGTATCTGCAAAAGCCGGGCCAGAGCCCCCAGCTGCTG |
| | | ATCTACCTTGGGTCAAACCGCGCTTCCGGGGTGCCTGATAGATTCTCCGGG |
| | | TCCGGGAGCGGAACCGACTTTACCCTGAAAATCTCGAGGGTGGAGGCCGA |
| | | GGACGTCGGAGTGTACTACTGCATGCAGGCGCTCCAGACTCCCCTGACCT |
| | | TCGGAGGAGGAACGAAGGTCGACATCAAGA |
| BCMA_EBB- | 189 | EVQLLESGGGLVQPGGSLRLSCAASGFTFSSYAMSWVRQAPGKGLEWVSAIS |
| C1980-A2-aa | | GSGGSTYYADSVKGRFTISRDNSKNTLYLQMNSRAEDTAVYYCVLWFGEG |
| VH | | FDPWGQGTLVTVSS |
| BCMA_EBB- | 210 | DIVLTQSPLSLPVTPGEPASISCRSSQSLLHSNGYNYLDWYLQKPGQSPQLLIY |
| C1980-A2-aa | | LGSNRASGVPDRFSGSGSGTDFTLKISRVEAEDVGVYYCMQALQTPLTFGGG |
| VL | | TKVDIK |

BCMA_EBB-C1981-C3

| | | |
|---|---|---|
| BCMA_EBB- | 148 | QVQLVESGGGLVQPGGSLRLSCAASGFTFSSYAMSWVRQAPGKGLEWVSAIS |
| C1981-C3-aa | | GSGGSTYYADSVKGRFTISRDNSKNTLYLQMNSLRAEDTAVYYCAKVGYDS |
| ScFv domain | | SGYYRDYYGMDVWGQGTTVTVSSGGGGSGGGGSGGGGSEIVLTQSPGTLSL |
| | | SPGERATLSCRASQSVSSSYLAWYQQKPGQAPRLLIYGTSSRATGISDRFSGSG |
| | | SGTDFTLTISRLEPEDFAVYYCQHYGNSPPKFTFGPGTKLEIK |
| BCMA_EBB- | 169 | CAAGTGCAGCTCGTGGAGTCAGGCGGAGGACTGGTGCAGCCCGGGGGCT |
| C1981-C3-nt | | CCCTGAGACTTTCCTGCGCGGCATCGGGTTTTACCTTCTCCTCCTATGCTAT |
| ScFv domain | | GTCCTGGGTGCGCCAGGCCCCGGGAAAGGGACTGGAATGGGTGTCCGCA |
| | | ATCAGCGGTAGCGGGGGCTCAACATACTACGCCGACTCCGTCAAGGGTCG |
| | | CTTCACTATTTCCCGGGACAACTCCAAGAATACCCTGTACCTCCAAATGAA |
| | | CAGCCTCAGGGCCGAGGATACTGCCGTGTACTACTGCGCCAAAGTCGGAT |
| | | ACGATAGCTCCGGTTACTACCGGGACTACTACGGAATGGACGTGTGGGGA |
| | | CAGGGCACCACCGTGACCGTGTCAAGCGGCGGAGGCGGTTCAGGAGGGG |
| | | GAGGCTCCGGCGGTGGAGGGTCCGAAATCGTCCTGACTCAGTCGCCTGGC |
| | | ACTCTGTCGTTGTCCCCGGGGGAGCGCGCTACCCTGTCGTGTCGGGCGTCG |
| | | CAGTCCGTGTCGAGCTCCTACCTCGCGTGGTACCAGCAGAAGCCCGGACA |
| | | GGCCCCTAGACTTCTGATCTACGGCACTTCTTCACGCGCCACCGGGATCAG |
| | | CGACAGGTTCAGCGGCTCCGGCTCCGGGACCGACTTCACCCTGACCATTA |
| | | GCCGGCTGGAGCCTGAAGATTTCGCCGTGTATTACTGCCAACACTACGGA |
| | | AACTCGCCGCCAAAGTTCACGTTCGGACCCGGAACCAAGCTGGAAATCAA |
| | | G |
| BCMA_EBB- | 190 | QVQLVESGGGLVQPGGSLRLSCAASGFTFSSYAMSWVRQAPGKGLEWVSAIS |
| C1981-C3-aa | | GSGGSTYYADSVKGRFTISRDNSKNTLYLQMNSLRAEDTAVYYCAKVGYDS |
| VH | | SGYYRDYYGMDVWGQGTTVTVSS |
| BCMA_EBB- | 211 | EIVLTQSPGTLSLSPGERATLSCRASQSVSSSYLAWYQQKPGQAPRLLIYGTSS |
| C1981-C3-aa | | RATGISDRFSGSGSGTDFTLTISRLEPEDFAVYYCQHYGNSPPKFTFGPGTKLEI |
| VL | | K |

BCMA_EBB-C1978-G4

| | | |
|---|---|---|
| BCMA_EBB- | 149 | EVQLVESGGGLVQPGGSLRLSCAASGFTFSSYAMSWVRQAPGKGLEWVSAIS |
| C1978-G4-aa | | GSGGSTYYADSVKGRFTISRDNSKNTLYLQMNSLRAEDTAVYYCAKMGWSS |
| ScFv domain | | GYLGAFDIWGQGTTVTVSSGGGGSGGGGSGGGGSEIVLTQSPGTLSLSPGER |
| | | ATLSCRASQSVASSFLAWYQQKPGQAPRLLIYGASGRATGIPDRFSGSGSGTD |
| | | FTLTISRLEPEDFAVYYCQHYGGSPRLTFGGGTKVDIK |
| BCMA_EBB- | 170 | GAAGTCCAACTGGTGGAGTCCGGGGGAGGGCTCGTGCAGCCCGGAGGCA |
| C1978-G4-nt | | GCCTTCGGCTGTCGTGCGCCGCCTCCGGGTTCACGTTCTCATCCTACGCGA |
| ScFv domain | | TGTCGTGGGTCAGACAGGCACCAGGAAAGGGACTGGAATGGGTGTCCGC |
| | | CATTAGCGGCTCCGGCGGTAGCACCTACTATGCCGACTCAGTGAAGGGAA |
| | | GGTTCACTATCTCCCGCGACAACAGCAAGAACACCCTGTACCTCCAAATG |
| | | AACTCTCTGCGGGCCGAGGATACCGCGGTGTACTATTGCGCCAAGATGGG |
| | | TTGGTCCAGCGGATACTTGGGAGCCTTCGACATTTGGGGACAGGGCACTA |
| | | CTGTGACCGTGTCCTCCGGGGGTGGCGGATCGGGAGGCGGCGGCTCGGGT |
| | | GGAGGGGGTTCCGAAATCGTGTTGACCCAGTCACCGGGAACCCTCTCGCT |
| | | GTCCCCGGGAGAACGGGCTACACTGTCATGTAGAGCGTCCCAGTCCGTGG |
| | | CTTCCTCGTTCCTGGCCTGGTACCAGCAGAAGCCGGGACAGGCACCCCGC |
| | | CTGCTCATCTACGGAGCCAGCGGCCGGGCGACCGGCATCCCTGACCGCTT |
| | | CTCCGGTTCCGGCTCGGGCACCGACTTTACTCTGACCATTAGCAGGCTTGA |
| | | GCCCGAGGATTTTGCCGTGTACTACTGCCAACACTACGGGGGGAGCCCTC |
| | | GCCTGACCTTCGGAGGCGGAACTAAGGTCGATATCAAAA |
| BCMA_EBB- | 191 | EVQLVESGGGLVQPGGSLRLSCAASGFTFSSYAMSWVRQAPGKGLEWVSAIS |
| C1978-G4-aa VH | | GSGGSTYYADSVKGRFTISRDNSKNTLYLQMNSLRAEDTAVYYCAKMGWSS |
| | | GYLGAFDIWGQGTTVTVSS |
| BCMA_EBB- | 212 | EIVLTQSPGTLSLSPGERATLSCRASQSVASSFLAWYQQKPGQAPRLLIYGASG |
| C1978-G4-aa VL | | RATGIPDRFSGSGSGTDFTLTISRLEPEDFAVYYCQHYGGSPRLTFGGGTKVDI |
| | | K |

TABLE 3
Heavy chain variable domain CDRs according to the Kabat
numbering scheme (Kabat et al. (1991), “Sequences of
Proteins of Immunological Interest,” 5th Ed. Public
Health Service, National Institutes of Health, Bethesda,
MD) “ID” stands for the respective SEQ ID NO for each CDR.

| Candidate | HCDR1 | ID | HCDR2 | ID | HCDR3 | ID |
|---|---|---|---|---|---|---|
| 139109 | NHGMS | 394 | GIVYSGSTYYAASVKG | 434 | HGGESDV | 474 |
| 139103 | NYAMS | 384 | GISRSGENTYYADSVKG | 424 | SPAHYYGGMDV | 464 |
| 139105 | DYAMH | 385 | GISWNSGSIGYADSVKG | 425 | HSFLAY | 465 |
| 139111 | NHGMS | 386 | GIVYSGSTYYAASVKG | 426 | HGGESDV | 466 |
| 139100 | NFGIN | 387 | WINPKNNNTNYAQKFQG | 427 | GPYYYQSYMDV | 467 |
| 139101 | SDAMT | 388 | VISGSGGTTYYADSVKG | 428 | LDSSGYYYARGPRY | 468 |
| 139102 | NYGIT | 389 | WISAYNGNTNYAQKFQG | 429 | GPYYYYMDV | 469 |
| 139104 | NHGMS | 390 | GIVYSGSTYYAASVKG | 430 | HGGESDV | 470 |
| 139106 | NHGMS | 391 | GIVYSGSTYYAASVKG | 431 | HGGESDV | 471 |
| 139107 | NHGMS | 392 | GIVYSGSTYYAASVKG | 432 | HGGESDV | 472 |
| 139108 | DYYMS | 393 | YISSSGSTIYYADSVKG | 433 | ESGDGMDV | 473 |
| 139110 | DYYMS | 395 | YISSSGNTIYYADSVKG | 435 | STMVREDY | 475 |
| 139112 | NHGMS | 396 | GIVYSGSTYYAASVKG | 436 | HGGESDV | 476 |
| 139113 | NHGMS | 397 | GIVYSGSTYYAASVKG | 437 | HGGESDV | 477 |
| 139114 | NHGMS | 398 | GIVYSGSTYYAASVKG | 438 | HGGESDV | 478 |
| 149362 | SSYYYWG | 399 | SIYYSGSAYYNPSLKS | 439 | HWQEWPDAFDI | 479 |
| 149363 | TSGMCVS | 400 | RIDWDEDKFYSTSLKT | 440 | SGAGGTSATAFDI | 480 |
| 149364 | SYSMN | 401 | SISSSSSYIYYADSVKG | 441 | TIAAVYAFDI | 481 |
| 149365 | DYYMS | 402 | YISSSGSTIYYADSVKG | 442 | DLRGAFDI | 482 |
| 149366 | SHYIH | 403 | MINPSGGVTAYSQTLQG | 443 | EGSGSGWYFDF | 483 |
| 149367 | SGGYYWS | 404 | YIYYSGSTYYNPSLKS | 444 | AGIAARLRGAFDI | 484 |
| 149368 | SYAIS | 405 | GIIPIFGTANYAQKFQG | 445 | RGGYQLLRWDVGLLR | 485 |
| | | | | | SAFDI | |
| 149369 | SNSAAWN | 406 | RTYYRSKWYSFYAISLKS | 446 | SSPEGLFLYWFDP | 486 |
| BCMA_EBB- | SYAMS | 407 | AISGSGGSTYYADSVKG | 447 | VEGSGSLDY | 487 |
| C1978-A4 | | | | | | |
| BCMA_EBB- | RYPMS | 408 | GISDSGVSTYYADSAKG | 448 | RAGSEASDI | 488 |
| C1978-G1 | | | | | | |
| BCMA_EBB- | SYAMS | 409 | AISGSGGSTYYADSVKG | 449 | ATYKRELRYYYGMDV | 489 |
| C1979-C1 | | | | | | |
| BCMA_EBB- | SYAMS | 410 | AISGSGGSTYYADSVKG | 450 | ATYKRELRYYYGMDV | 490 |
| C1978-C7 | | | | | | |
| BCMA_EBB- | DYAMH | 411 | GISWNSGSIGYADSVKG | 451 | VGKAVPDV | 491 |
| C1978-D10 | | | | | | |
| BCMA_EBB- | DYAMH | 412 | SINWKGNSLAYGDSVKG | 452 | HQGVAYYNYAMDV | 492 |
| C1979-C12 | | | | | | |
| BCMA_EBB- | SYAMS | 413 | AISGSGGSTYYADSVKG | 453 | VVRDGMDV | 493 |
| C1980-G4 | | | | | | |
| BCMA_EBB- | SYAMS | 414 | AISGSGGSTYYADSVKG | 454 | IPQTGTFDY | 494 |
| C1980-D2 | | | | | | |
| BCMA_EBB- | SYAMS | 415 | AISGSGGSTYYADSVKG | 455 | ANYKRELRYYYGMD | 495 |
| C1978-A10 | | | | | V | |
| BCMA_EBB- | SYAMS | 416 | AISGSGGSTYYADSVKG | 456 | ALVGATGAFDI | 496 |
| C1978-D4 | | | | | | |
| BCMA_EBB- | SYAMS | 417 | AISGSGGSTYYADSVKG | 457 | WFGEGFDP | 497 |
| C1980-A2 | | | | | | |
| BCMA_EBB- | SYAMS | 418 | AISGSGGSTYYADSVKG | 458 | VGYDSSGYYRDYYGM | 498 |
| C1981-C3 | | | | | DV | |
| BCMA_EBB- | SYAMS | 419 | AISGSGGSTYYADSVKG | 459 | MGWSSGYLGAFDI | 499 |
| C1978-G4 | | | | | | |
| A7D12.2 | NFGMN | 420 | WINTYTGESYFADDFKG | 460 | GEIYYGYDGGFAY | 500 |
| C11D5.3 | DYSIN | 421 | WINTETREPAYAYDFRG | 461 | DYSYAMDY | 501 |
| C12A3.2 | HYSMN | 422 | RINTESGVPIYADDFKG | 462 | DYL YSLDF | 502 |
| C13F12.1 | HYSMN | 423 | RINTETGEPLYADDFKG | 463 | DYLYSCDY | 503 |

TABLE 4
Light chain variable domain CDRs according to the Kabat
numbering scheme (Kabat et al. (1991), “Sequences of
Proteins of Immunological Interest,” 5th Ed. Public
Health Service, National Institutes of Health, Bethesda,
MD) “ID” stands for the respective SEQ ID NO for each CDR.

| Candidate | LCDR1 | ID | LCDR2 | ID | LCDR3 | ID |
|---|---|---|---|---|---|---|
| 139109 | RASQSISSYLN | 514 | AASSLQS | 554 | QQSYSTPYT | 594 |
| 139103 | RASQSISSSFLA | 504 | GASRRAT | 544 | QQYHSSPSWT | 584 |
| 139105 | RSSQSLLHSNGYNYLD | 505 | LGSNRAS | 545 | MQALQTPYT | 585 |
| 139111 | KSSQSLLRNDGKTPLY | 506 | EVSNRFS | 546 | MQNIQFPS | 586 |
| 139100 | RSSQSLLHSNGYNYLN | 507 | LGSKRAS | 547 | MQALQTPYT | 587 |
| 139101 | RASQSISSYLN | 508 | GASTLAS | 548 | QQSYKRAS | 588 |
| 139102 | RSSQSLLYSNGYNYVD | 509 | LGSNRAS | 549 | MQGRQFPYS | 589 |
| 139104 | RASQSVSSNLA | 510 | GASTRAS | 550 | QQYGSSLT | 590 |
| 139106 | RASQSVSSKLA | 511 | GASIRAT | 551 | QQYGSSSWT | 591 |
| 139107 | RASQSVGSTNLA | 512 | DASNRAT | 552 | QQYGSSPPWT | 592 |
| 139108 | RASQSISSYLN | 513 | AASSLQS | 553 | QQSYTLA | 593 |
| 139110 | KSSESLVHNSGKTYLN | 515 | EVSNRDS | 555 | MQGTHWPGT | 595 |
| 139112 | QASEDINKFLN | 516 | DASTLQT | 556 | QQYESLPLT | 596 |
| 139113 | RASQSVGSNLA | 517 | GASTRAT | 557 | QQYNDWLPVT | 597 |
| 139114 | RASQSIGSSSLA | 518 | GASSRAS | 558 | QQYAGSPPFT | 598 |
| 149362 | KASQDIDDAMN | 519 | SATSPVP | 559 | LQHDNFPLT | 599 |
| 149363 | RASQDIYNNLA | 520 | AANKSQS | 560 | QHYYRFPYS | 600 |
| 149364 | RSSQSLLHSNGYNYLD | 521 | LGSNRAS | 561 | MQALQTPYT | 601 |
| 149365 | GGNNIGTKSVH | 522 | DDSVRPS | 562 | QVWDSDSEHVV | 602 |
| 149366 | SGDGLSKKYVS | 523 | RDKERPS | 563 | QAWDDTTVV | 603 |
| 149367 | RASQGIRNWLA | 524 | AASNLQS | 564 | QKYNSAPFT | 604 |
| 149368 | GGNNIGSKSVH | 525 | GKNNRPS | 565 | SSRDSSGDHLRV | 605 |
| 149369 | QGDSLGNYYAT | 526 | GTNNRPS | 566 | NSRDSSGHHLL | 606 |
| BCMAEBB- | RASQSVSSAYLA | 527 | GASTRAT | 567 | QHYGSSFNGSSLFT | 607 |
| C1978-A4 | | | | | | |
| BCMAEBB- | RASQSVSNSLA | 528 | DASSRAT | 568 | QQFGTSSGLT | 608 |
| C1978-G1 | | | | | | |
| BCMAEBB- | RASQSVSSSFLA | 529 | GASSRAT | 569 | QQYHSSPSWT | 609 |
| C1979-C1 | | | | | | |
| BCMAEBB- | RASQSVSTTFLA | 530 | GSSNRAT | 570 | QQYHSSPSWT | 610 |
| C1978-C7 | | | | | | |
| BCMAEBB- | RASQSISSYLN | 531 | AASSLQS | 571 | QQSYSTPYS | 611 |
| C1978-D10 | | | | | | |
| BCMAEBB- | RATQSIGSSFLA | 532 | GASQRAT | 572 | QHYESSPSWT | 612 |
| C1979-C12 | | | | | | |
| BCMAEBB- | RASQSVSSSYLA | 533 | GASSRAT | 573 | QQYGSPPRFT | 613 |
| C1980-G4 | | | | | | |
| BCMAEBB- | RASQSVSSSYLA | 534 | GASSRAT | 574 | QHYGSSPSWT | 614 |
| C1980-D2 | | | | | | |
| BCMAEBB- | RASQRVASNYLA | 535 | GASSRAT | 575 | QHYDSSPSWT | 615 |
| C1978-A10 | | | | | | |
| BCMAEBB- | RASQSLSSNFLA | 536 | GASNWAT | 576 | QYYGTSPMYT | 616 |
| C1978-D4 | | | | | | |
| BCMAEBB- | RSSQSLLHSNGYNYLD | 537 | LGSNRAS | 577 | MQALQTPLT | 617 |
| C1980-A2 | | | | | | |
| BCMAEBB- | RASQSVSSSYLA | 538 | GTSSRAT | 578 | QHYGNSPPKFT | 618 |
| C1981-C3 | | | | | | |
| BCMAEBB- | RASQSVASSFLA | 539 | GASGRAT | 579 | QHYGGSPRLT | 619 |
| C1978-G4 | | | | | | |
| A7D12.2 | RASQDVNTAVS | 540 | SASYRYT | 580 | QQHYSTPWT | 620 |
| C11D5.3 | RASESVSVIGAHLIH | 541 | LASNLET | 581 | LQSRIFPRT | 621 |
| C12A3.2 | RASESVTILGSHLIY | 542 | LASNVQT | 582 | LQSRTIPRT | 622 |
| C13F12.1 | RASESVTILGSHLIY | 543 | LASNVQT | 583 | LQSRTIPRT | 623 |

TABLE 5
Heavy chain variable domain CDRs according to the
Chothia numbering scheme (A1-Lazikani et al., (1997)
JMB 273,927-948) “ID” stands for the respective SEQ
ID NO for each CDR.

| Candidate | HCDR1 | ID | HCDR2 | ID | HCDR3 | ID |
|---|---|---|---|---|---|---|
| 139109 | GFALSNH | 634 | VYSGS | 674 | HGGESDV | 714 |
| 139103 | GFTFSNY | 624 | SRSGEN | 664 | SPAHYYGGMDV | 704 |
| 139105 | GFTFDDY | 625 | SWNSGS | 665 | HSFLAY | 705 |
| 139111 | GFALSNH | 626 | VYSGS | 666 | HGGESDV | 706 |
| 139100 | GYIFDNF | 627 | NPKNNN | 667 | GPYYYQSYMDV | 707 |
| 139101 | GFTFSSD | 628 | SGSGGT | 668 | LDSSGYYYARGPRY | 708 |
| 139102 | GYTFSNY | 629 | SAYNGN | 669 | GPYYYYMDV | 709 |
| 139104 | GFALSNH | 630 | VYSGS | 670 | HGGESDV | 710 |
| 139106 | GFALSNH | 631 | VYSGS | 671 | HGGESDV | 711 |
| 139107 | GFALSNH | 632 | VYSGS | 672 | HGGESDV | 712 |
| 139108 | GFTFSDY | 633 | SSSGST | 673 | ESGDGMDV | 713 |
| 139110 | GFTFSDY | 635 | SSSGNT | 675 | STMVREDY | 715 |
| 139112 | GFALSNH | 636 | VYSGS | 676 | HGGESDV | 716 |
| 139113 | GFALSNH | 637 | VYSGS | 677 | HGGESDV | 717 |
| 139114 | GFALSNH | 638 | VYSGS | 678 | HGGESDV | 718 |
| 149362 | GGSISSSYY | 639 | YYSGS | 679 | HWQEWPDAFDI | 719 |
| 149363 | GFSLRTSGM | 640 | DWDED | 680 | SGAGGTSATAFDI | 720 |
| 149364 | GFTFSSY | 641 | SSSSSY | 681 | TIAAVYAFDI | 721 |
| 149365 | GFTFSDY | 642 | SSSGST | 682 | DLRGAFDI | 722 |
| 149366 | GYTVTSH | 643 | NPSGGV | 683 | EGSGSGWYFDF | 723 |
| 149367 | GGSISSGGY | 644 | YYSGS | 684 | AGIAARLRGAFDI | 724 |
| 149368 | GGTFSSY | 645 | IPIFGT | 685 | RGGYQLLRWDVGLL | 725 |
| | | | | | RSAFDI | |
| 149369 | GDSVSSNSA | 646 | YYRSKWY | 686 | SSPEGLFLYWFDP | 726 |
| BCMA_EBB- | GFTFSSY | 647 | SGSGGS | 687 | VEGSGSLDY | 727 |
| C1978-A4 | | | | | | |
| BCMA_EBB- | GITFSRY | 648 | SDSGVS | 688 | RAGSEASDI | 728 |
| C1978-G1 | | | | | | |
| BCMA_EBB- | GFTFSSY | 649 | SGSGGS | 689 | ATYKRELRYYYGMDV | 729 |
| C1979-C1 | | | | | | |
| BCMA_EBB- | GFTFSSY | 650 | SGSGGS | 690 | ATYKRELRYYYGMDV | 730 |
| C1978-C7 | | | | | | |
| BCMA_EBB- | GFTFDDY | 651 | SWNSGS | 691 | VGKAVPDV | 731 |
| C1978-D10 | | | | | | |
| BCMA_EBB- | GFTFDDY | 652 | NWKGNS | 692 | HQGVAYYNYAMDV | 732 |
| C1979-C12 | | | | | | |
| BCMA_EBB- | GFTFSSY | 653 | SGSGGS | 693 | VVRDGMDV | 733 |
| C1980-G4 | | | | | | |
| BCMA_EBB- | GFTFSSY | 654 | SGSGGS | 694 | IPQTGTFDY | 734 |
| C1980-D2 | | | | | | |
| BCMA_EBB- | GFTFSSY | 655 | SGSGGS | 695 | ANYKRELRYYYGMDV | 735 |
| C1978-A10 | | | | | | |
| BCMA_EBB- | GFSFSSY | 656 | SGSGGS | 696 | AL VGATGAFDI | 736 |
| C1978-D4 | | | | | | |
| BCMA_EBB- | GFTFSSY | 657 | SGSGGS | 697 | WFGEGFDP | 737 |
| C1980-A2 | | | | | | |
| BCMA_EBB- | GFTFSSY | 658 | SGSGGS | 698 | VGYDSSGYYRDYYGM | 738 |
| C1981-C3 | | | | | DV | |
| BCMA_EBB- | GFTFSSY | 659 | SGSGGS | 699 | MGWSSGYLGAFDI | 739 |
| C1978-G4 | | | | | | |
| A7D12.2 | GYTFTNF | 660 | NTYTGE | 700 | GEIYYGYDGGFAY | 740 |
| C11D5.3 | GYTFTDY | 661 | NTETRE | 701 | DYSYAMDY | 741 |
| C12A3.2 | GYTFRHY | 662 | NTESGV | 702 | DYL YSLDF | 742 |
| C13F12.1 | GYTFTHY | 663 | NTETGE | 703 | DYL YSCDY | 743 |

TABLE 6
Light chain variable domain CDRs according to the
Chothia numbering scheme (A1-Lazikani et al., (1997)
JMB 273,927-948) “ID” stands for the respective SEQ
ID NO for each CDR.

| Candidate | LCDR1 | ID | LCDR2 | ID | LCDR3 | ID |
|---|---|---|---|---|---|---|
| 139109 | SQSISSY | 754 | AAS | 794 | SYSTPY | 834 |
| 139103 | SQSISSSF | 744 | GAS | 784 | YHSSPSW | 824 |
| 139105 | SQSLLHSNGYNY | 745 | LGS | 785 | ALQTPY | 825 |
| 139111 | SQSLLRNDGKTP | 746 | EVS | 786 | NIQFP | 826 |
| 139100 | SQSLLHSNGYNY | 747 | LGS | 787 | ALQTPY | 827 |
| 139101 | SQSISSY | 748 | GAS | 788 | SYKRA | 828 |
| 139102 | SQSLLYSNGYNY | 749 | LGS | 789 | GRQFPY | 829 |
| 139104 | SQSVSSN | 750 | GAS | 790 | YGSSL | 830 |
| 139106 | SQSVSSK | 751 | GAS | 791 | YGSSSW | 831 |
| 139107 | SQSVGSTN | 752 | DAS | 792 | YGSSPPW | 832 |
| 139108 | SQSISSY | 753 | AAS | 793 | SYTL | 833 |
| 139110 | SESLVHNSGKTY | 755 | EVS | 795 | GTHWPG | 835 |
| 139112 | SEDINKF | 756 | DAS | 796 | YESLPL | 836 |
| 139113 | SQSVGSN | 757 | GAS | 797 | YNDWLPV | 837 |
| 139114 | SQSIGSSS | 758 | GAS | 798 | YAGSPPF | 838 |
| 149362 | SQDIDDA | 759 | SAT | 799 | HDNFPL | 839 |
| 149363 | SQDIYNN | 760 | AAN | 800 | YYRFPY | 840 |
| 149364 | SQSLLHSNGYNY | 761 | LGS | 801 | ALQTPY | 841 |
| 149365 | NNIGTKS | 762 | DDS | 802 | WDSDSEHV | 842 |
| 149366 | DGLSKKY | 763 | RDK | 803 | WDDTTV | 843 |
| 149367 | SQGIRNW | 764 | AAS | 804 | YNSAPF | 844 |
| 149368 | NNIGSKS | 765 | GKN | 805 | RDSSGDHLR | 845 |
| 149369 | DSLGNYY | 766 | GTN | 806 | RDSSGHHL | 846 |
| BCMA_EBB- | SQSVSSAY | 767 | GAS | 807 | YGSSFNGSSLF | 847 |
| C1978-A4 | | | | | | |
| BCMA_EBB- | SQSVSNS | 768 | DAS | 808 | FGTSSGL | 848 |
| C1978-G1 | | | | | | |
| BCMA_EBB- | SQSVSSSF | 769 | GAS | 809 | YHSSPSW | 849 |
| C1979-C1 | | | | | | |
| BCMA_EBB- | SQSVSTTF | 770 | GSS | 810 | YHSSPSW | 850 |
| C1978-C7 | | | | | | |
| BCMA_EBB- | SQSISSY | 771 | AAS | 811 | SYSTPY | 851 |
| C1978-D10 | | | | | | |
| BCMA_EBB- | TQSIGSSF | 772 | GAS | 812 | YESSPSW | 852 |
| C1979-C12 | | | | | | |
| BCMA_EBB- | SQSVSSSY | 773 | GAS | 813 | YGSPPRF | 853 |
| C1980-G4 | | | | | | |
| BCMA_EBB- | SQSVSSSY | 774 | GAS | 814 | YGSSPSW | 854 |
| C1980-D2 | | | | | | |
| BCMA_EBB- | SQRVASNY | 775 | GAS | 815 | YDSSPSW | 855 |
| C1978-A10 | | | | | | |
| BCMA_EBB- | SQSLSSNF | 776 | GAS | 816 | YGTSPMY | 856 |
| C1978-D4 | | | | | | |
| BCMA_EBB- | SQSLLHSNGYNY | 777 | LGS | 817 | ALQTPL | 857 |
| C1980-A2 | | | | | | |
| BCMA_EBB- | SQSVSSSY | 778 | GTS | 818 | YGNSPPKF | 858 |
| C1981-C3 | | | | | | |
| BCMA_EBB- | SQSVASSF | 779 | GAS | 819 | YGGSPRL | 859 |
| C1978-G4 | | | | | | |
| A7D12.2 | SQDVNTA | 780 | SAS | 820 | HYSTPW | 860 |
| C11D5.3 | SESVSVIGAHL | 781 | LAS | 821 | SRIFPR | 861 |
| C12A3.2 | SESVTILGSHL | 782 | LAS | 822 | SRTIPR | 862 |
| C13F12.1 | SESVTILGSHL | 783 | LAS | 823 | SRTIPR | 863 |

TABLE 7
Heavy chain variable domain CDRs according to a combination of the
Kabat numbering scheme (Kabat et al. (1991), “Sequences of Proteins
of Immunological Interest,” 5th Ed. Public Health Service, National
Institutes of Health, Bethesda, MD) and the Chothia numbering scheme
(A1-Lazikani et al., (1997) JMB 273,927-948). “ID” stands for the
respective SEQ ID NO for each CDR.

| Candidate | HCDR1 | ID | HCDR2 | ID | HCDR3 | ID |
|---|---|---|---|---|---|---|
| 139109 | GFALSNHGMS | 874 | GIVYSGSTYYAASVKG | 914 | HGGESDV | 954 |
| 139103 | GFTFSNYAMS | 864 | GISRSGENTYYADSVKG | 904 | SPAHYYGGMDV | 944 |
| 139105 | GFTFDDYAMH | 865 | GISWNSGSIGYADSVKG | 905 | HSFLAY | 945 |
| 139111 | GFALSNHGMS | 866 | GIVYSGSTYYAASVKG | 906 | HGGESDV | 946 |
| 139100 | GYIFDNFGIN | 867 | WINPKNNNTNYAQKFQG | 907 | GPYYYQSYMDV | 947 |
| 139101 | GFTFSSDAMT | 868 | VISGSGGTTYYADSVKG | 908 | LDSSGYYYARGPRY | 948 |
| 139102 | GYTFSNYGIT | 869 | WISAYNGNTNYAQKFQG | 909 | GPYYYYMDV | 949 |
| 139104 | GFALSNHGMS | 870 | GIVYSGSTYYAASVKG | 910 | HGGESDV | 950 |
| 139106 | GFALSNHGMS | 871 | GIVYSGSTYYAASVKG | 911 | HGGESDV | 951 |
| 139107 | GFALSNHGMS | 872 | GIVYSGSTYYAASVKG | 912 | HGGESDV | 952 |
| 139108 | GFTFSDYYMS | 873 | YISSSGSTIYYADSVKG | 913 | ESGDGMDV | 953 |
| 139110 | GFTFSDYYMS | 875 | YISSSGNTIYYADSVKG | 915 | STMVREDY | 955 |
| 139112 | GFALSNHGMS | 876 | GIVYSGSTYYAASVKG | 916 | HGGESDV | 956 |
| 139113 | GFALSNHGMS | 877 | GIVYSGSTYYAASVKG | 917 | HGGESDV | 957 |
| 139114 | GFALSNHGMS | 878 | GIVYSGSTYYAASVKG | 918 | HGGESDV | 958 |
| 149362 | GGSISSSYYYWG | 879 | SIYYSGSAYYNPSLKS | 919 | HWQEWPDAFDI | 959 |
| 149363 | GFSLRTSGMCVS | 880 | RIDWDEDKFYSTSLKT | 920 | SGAGGTSATAFDI | 960 |
| 149364 | GFTFSSYSMN | 881 | SISSSSSYIYYADSVKG | 921 | TIAAVYAFDI | 961 |
| 149365 | GFTFSDYYMS | 882 | YISSSGSTIYYADSVKG | 922 | DLRGAFDI | 962 |
| 149366 | GYTVTSHYIH | 883 | MINPSGGVTAYSQTLQG | 923 | EGSGSGWYFDF | 963 |
| 149367 | GGSISSGGYYWS | 884 | YIYYSGSTYYNPSLKS | 924 | AGIAARLRGAFDI | 964 |
| 149368 | GGTFSSYAIS | 885 | GIIPIFGTANYAQKFQG | 925 | RGGYQLLRWDVGLL | 965 |
| | | | | | RSAFDI | |
| 149369 | GDSVSSNSAAWN | 886 | RTYYRSKWYSFYAISLKS | 926 | SSPEGLFLYWFDP | 966 |
| BCMA_EBB- | GFTFSSYAMS | 887 | AISGSGGSTYYADSVKG | 927 | VEGSGSLDY | 967 |
| C1978-A4 | | | | | | |
| BCMA_EBB- | GITFSRYPMS | 888 | GISDSGVSTYYADSAKG | 928 | RAGSEASDI | 968 |
| C1978-G1 | | | | | | |
| BCMA_EBB- | GFTFSSYAMS | 889 | AISGSGGSTYYADSVKG | 929 | ATYKRELRYYYGM | 969 |
| C1979-C1 | | | | | DV | |
| BCMA_EBB- | GFTFSSYAMS | 890 | AISGSGGSTYYADSVKG | 930 | ATYKRELRYYYGM | 970 |
| C1978-C7 | | | | | DV | |
| BCMA_EBB- | GFTFDDYAMH | 891 | GISWNSGSIGYADSVKG | 931 | VGKAVPDV | 971 |
| C1978-D10 | | | | | | |
| BCMA_EBB- | GFTFDDYAMH | 892 | SINWKGNSLAYGDSVKG | 932 | HQGVAYYNYAMDV | 972 |
| C1979-C12 | | | | | | |
| BCMA_EBB- | GFTFSSYAMS | 893 | AISGSGGSTYYADSVKG | 933 | VVRDGMDV | 973 |
| C1980-G4 | | | | | | |
| BCMA_EBB- | GFTFSSYAMS | 894 | AISGSGGSTYYADSVKG | 934 | IPQTGTFDY | 974 |
| C1980-D2 | | | | | | |
| BCMA_EBB- | GFTFSSYAMS | 895 | AISGSGGSTYYADSVKG | 935 | ANYKRELRYYYGMD | 975 |
| C1978-A10 | | | | | V | |
| BCMA_EBB- | GFSFSSYAMS | 896 | AISGSGGSTYYADSVKG | 936 | ALVGATGAFDI | 976 |
| C1978-D4 | | | | | | |
| BCMA_EBB- | GFTFSSYAMS | 897 | AISGSGGSTYYADSVKG | 937 | WFGEGFDP | 977 |
| C1980-A2 | | | | | | |
| BCMA_EBB- | GFTFSSYAMS | 898 | AISGSGGSTYYADSVKG | 938 | VGYDSSGYYRDYYG | 978 |
| C1981-C3 | | | | | MDV | |
| BCMA_EBB- | GFTFSSYAMS | 899 | AISGSGGSTYYADSVKG | 939 | MGWSSGYLGAFDI | 979 |
| C1978-G4 | | | | | | |
| A7D12.2 | GYTFTNFGMN | 900 | WINTYTGESYFADDFKG | 940 | GEIYYGYDGGFAY | 980 |
| C11D5.3 | GYTFTDYSIN | 901 | WINTETREPAYAYDFRG | 941 | DYSYAMDY | 981 |
| C12A3.2 | GYTFRHYSMN | 902 | RINTESGVPIYADDFKG | 942 | DYLYSLDF | 982 |
| C13F12.1 | GYTFTHYSMN | 903 | RINTETGEPLYADDFKG | 943 | DYLYSCDY | 983 |

TABLE 8
Light chain variable domain CDRs according to a combination of
the Kabat numbering scheme (Kabat et al. (1991), “Sequences of
Proteins of Immunological Interest,” 5th Ed. Public Health
Service, National Institutes of Health, Bethesda, MD) and the
Chothia numbering scheme (A1-Lazikani et al., (1997) JMB
273,927-948). “ID” stands for the respective SEQ ID NO for
each CDR.

| Candidate | LCDR1 | ID | LCDR2 | ID | LCDR3 | ID |
|---|---|---|---|---|---|---|
| 139109 | RASQSISSYLN | 994 | AASSLOS | 1034 | QQSYSTPYT | 1074 |
| 139103 | RASQSISSSFLA | 984 | GASRRAT | 1024 | QQYHSSPSWT | 1064 |
| 139105 | RSSQSLLHSNGYNYLD | 985 | LGSNRAS | 1025 | MQALQTPYT | 1065 |
| 139111 | KSSQSLLRNDGKTPLY | 986 | EVSNRFS | 1026 | MQNIQFPS | 1066 |
| 139100 | RSSQSLLHSNGYNYLN | 987 | LGSKRAS | 1027 | MQALQTPYT | 1067 |
| 139101 | RASQSISSYLN | 988 | GASTLAS | 1028 | QQSYKRAS | 1068 |
| 139102 | RSSQSLLYSNGYNYVD | 989 | LGSNRAS | 1029 | MQGRQFPYS | 1069 |
| 139104 | RASQSVSSNLA | 990 | GASTRAS | 1030 | QQYGSSLT | 1070 |
| 139106 | RASQSVSSKLA | 991 | GASIRAT | 1031 | QQYGSSSWT | 1071 |
| 139107 | RASQSVGSTNLA | 992 | DASNRAT | 1032 | QQYGSSPPWT | 1072 |
| 139108 | RASQSISSYLN | 993 | AASSLQS | 1033 | QQSYTLA | 1073 |
| 139110 | KSSESLVHNSGKTYLN | 995 | EVSNRDS | 1035 | MQGTHWPGT | 1075 |
| 139112 | QASEDINKFLN | 996 | DASTLQT | 1036 | QQYESLPLT | 1076 |
| 139113 | RASQSVGSNLA | 997 | GASTRAT | 1037 | QQYNDWLPVT | 1077 |
| 139114 | RASQSIGSSSLA | 998 | GASSRAS | 1038 | QQYAGSPPFT | 1078 |
| 149362 | KASQDIDDAMN | 999 | SATSPVP | 1039 | LQHDNFPLT | 1079 |
| 149363 | RASQDIYNNLA | 1000 | AANKSQS | 1040 | QHYYRFPYS | 1080 |
| 149364 | RSSQSLLHSNGYNYLD | 1001 | LGSNRAS | 1041 | MQALQTPYT | 1081 |
| 149365 | GGNNIGTKSVH | 1002 | DDSVRPS | 1042 | QVWDSDSEHVV | 1082 |
| 149366 | SGDGLSKKYVS | 1003 | RDKERPS | 1043 | QAWDDTTVV | 1083 |
| 149367 | RASQGIRNWLA | 1004 | AASNLQS | 1044 | QKYNSAPFT | 1084 |
| 149368 | GGNNIGSKSVH | 1005 | GKNNRPS | 1045 | SSRDSSGDHLRV | 1085 |
| 149369 | QGDSLGNYYAT | 1006 | GTNNRPS | 1046 | NSRDSSGHHLL | 1086 |
| BCMA_EBB- | RASQSVSSAYLA | 1007 | GASTRAT | 1047 | QHYGSSFNGSSL | 1087 |
| C1978-A4 | | | | | FT | |
| BCMA_EBB- | RASQSVSNSLA | 1008 | DASSRAT | 1048 | QQFGTSSGLT | 1088 |
| C1978-G1 | | | | | | |
| BCMA_EBB- | RASQSVSSSFLA | 1009 | GASSRAT | 1049 | QQYHSSPSWT | 1089 |
| C1979-C1 | | | | | | |
| BCMA_EBB- | RASQSVSTTFLA | 1010 | GSSNRAT | 1050 | QQYHSSPSWT | 1090 |
| C1978-C7 | | | | | | |
| BCMA_EBB- | RASQSISSYLN | 1011 | AASSLQS | 1051 | QQSYSTPYS | 1091 |
| C1978-D10 | | | | | | |
| BCMA_EBB- | RATQSIGSSFLA | 1012 | GASQRAT | 1052 | QHYESSPSWT | 1092 |
| C1979-C12 | | | | | | |
| BCMA_EBB- | RASQSVSSSYLA | 1013 | GASSRAT | 1053 | QQYGSPPRFT | 1093 |
| C1980-G4 | | | | | | |
| BCMA_EBB- | RASQSVSSSYLA | 1014 | GASSRAT | 1054 | QHYGSSPSWT | 1094 |
| C1980-D2 | | | | | | |
| BCMA_EBB- | RASQRVASNYLA | 1015 | GASSRAT | 1055 | QHYDSSPSWT | 1095 |
| C1978-A10 | | | | | | |
| BCMA_EBB- | RASQSLSSNFLA | 1016 | GASNWAT | 1056 | QYYGTSPMYT | 1096 |
| C1978-D4 | | | | | | |
| BCMA_EBB- | RSSQSLLHSNGYNYLD | 1017 | LGSNRAS | 1057 | MQALQTPLT | 1097 |
| C1980-A2 | | | | | | |
| BCMA_EBB- | RASQSVSSSYLA | 1018 | GTSSRAT | 1058 | QHYGNSPPKFT | 1098 |
| C1981-C3 | | | | | | |
| BCMA_EBB- | RASQSVASSFLA | 1019 | GASGRAT | 1059 | QHYGGSPRLT | 1099 |
| C1978-G4 | | | | | | |
| A7D12.2 | RASQDVNTAVS | 1020 | SASYRYT | 1060 | QQHYSTPWT | 1100 |
| C11D5.3 | RASESVSVIGAHLIH | 1021 | LASNLET | 1061 | LQSRIFPRT | 1101 |
| C12A3.2 | RASESVTILGSHLIY | 1022 | LASNVQT | 1062 | LQSRTIPRT | 1102 |
| C13F12.1 | RASESVTILGSHLIY | 1023 | LASNVQT | 1063 | LQSRTIPRT | 1103 |

TABLE 9
Amino acid sequences of additional exemplary
anti-BCMA VHs, VLs, and scFv domains

| | | SEQ ID |
|---|---|---|
| Name | Sequence | NO: |
| A7D12.2 | QIQLVQSGPDLKKPGETVKLSCKASGYTFTNFGMNWVKQAPGKGFKWMA | 255 |
| VH | WINTYTGESYFADDFKGRFAFSVETSATTAYLQINNLKTEDTATYFCAR | |
| | GEIYYGYDGGFAYWGQGTLVTVSA | |
| A7D12.2 | DVVMTQSHRFMSTSVGDRVSITCRASQDVNTAVSWYQQKPGQSPKLLIF | 259 |
| VL | SASYRYTGVPDRFTGSGSGADFTLTISSVQAEDLAVYYCQQHYSTPWTF | |
| | GGGTKLDIK | |
| A7D12.2 | QIQLVQSGPDLKKPGETVKLSCKASGYTFTNFGMNWVKQAPGKGFKWMA | 263 |
| scFv | WINTYTGESYFADDFKGRFAFSVETSATTAYLQINNLKTEDTATYFCAR | |
| domain | GEIYYGYDGGFAYWGQGTLVTVSAGGGGSGGGGSGGGGSDVVMTQSHRF | |
| | MSTSVGDRVSITCRASQDVNTAVSWYQQKPGQSPKLLIFSASYRYTGVP | |
| | DRFTGSGSGADFTLTISSVQAEDLAVYYCQQHYSTPWTFGGGTKLDIK | |
| C11D5.3 | QIQLVQSGPELKKPGETVKISCKASGYTFTDYSINWVKRAPGKGLKWMG | 256 |
| VH | WINTETREPAYAYDFRGRFAFSLETSASTAYLQINNLKYEDTATYFCAL | |
| | DYSYAMDYWGQGTSVTVSS | |
| C11D5.3 | DIVLTQSPASLAMSLGKRATISCRASESVSVIGAHLIHWYQQKPGQPPK | 260 |
| VL | LLIYLASNLETGVPARFSGSGSGTDFTLTIDPVEEDDVAIYSCLQSRIF | |
| | PRTFGGGTKLEIK | |
| C11D5.3 | QIQLVQSGPELKKPGETVKISCKASGYTFTDYSINWVKRAPGKGLKWMG | 264 |
| scFv | WINTETREPAYAYDFRGRFAFSLETSASTAYLQINNLKYEDTATYFCAL | |
| domain | DYSYAMDYWGQGTSVTVSSGGGGSGGGGSGGGGSQIQLVQSGPELKKPG | |
| | ETVKISCKASGYTFTDYSINWVKRAPGKGLKWMGWINTETREPAYAYDF | |
| | RGRFAFSLETSASTAYLQINNLKYEDTATYFCALDYSYAMDYWGQGTSV | |
| | TVSS | |
| C12A3.2 | QIQLVQSGPELKKPGETVKISCKASGYTFRHYSMNWVKQAPGKGLKWMG | 257 |
| VH | RINTESGVPIYADDFKGRFAFSVETSASTAYLVINNLKDEDTASYFCSN | |
| | DYLYSLDFWGQGTALTVSS | |
| C12A3.2 | DIVLTQSPPSLAMSLGKRATISCRASESVTILGSHLIYWYQQKPGQPPT | 261 |
| VL | LLIQLASNVQTGVPARFSGSGSRTDFTLTIDPVEEDDVAVYYCLQSRTI | |
| | PRTFGGGTKLEIK | |
| C12A3.2 | QIQLVQSGPELKKPGETVKISCKASGYTFRHYSMNWVKQAPGKGLKWMG | 265 |
| scFv | RINTESGVPIYADDFKGRFAFSVETSASTAYLVINNLKDEDTASYFCSN | |
| domain | DYLYSLDFWGQGTALTVSSGGGGSGGGGSGGGGSDIVLTQSPPSLAMSL | |
| | GKRATISCRASESVTILGSHLIYWYQQKPGQPPTLLIQLASNVQTGVPA | |
| | RFSGSGSRTDFTLTIDPVEEDDVAVYYCLQSRTIPRTFGGGTKLEIK | |
| C13F12.1 | QIQLVQSGPELKKPGETVKISCKASGYTFTHYSMNWVKQAPGKGLKWMG | 258 |
| VH | RINTETGEPLYADDFKGRFAFSLETSASTAYLVINNLKNEDTATFFCSN | |
| | DYLYSCDYWGQGTTLTVSS | |
| C13F12.1 | DIVLTQSPPSLAMSLGKRATISCRASESVTILGSHLIYWYQQKPGQPPT | 262 |
| VL | LLIQLASNVQTGVPARFSGSGSRTDFTLTIDPVEEDDVAVYYCLQSRTI | |
| | PRTFGGGTKLEIK | |
| C13F12.1 | QIQLVQSGPELKKPGETVKISCKASGYTFTHYSMNWVKQAPGKGLKWMG | 266 |
| scFv | RINTETGEPLYADDFKGRFAFSLETSASTAYLVINNLKNEDTATFFCSN | |
| domain | DYLYSCDYWGQGTTLTVSSGGGGSGGGGSGGGGSDIVLTQSPPSLAMSL | |
| | GKRATISCRASESVTILGSHLIYWYQQKPGQPPTLLIQLASNVQTGVPA | |
| | RFSGSGSRTDFTLTIDPVEEDDVAVYYCLQSRTIPRTFGGGTKLEIK | |

Additional exemplary BCMA-targeting sequences that can be used in the anti-BCMA antibody molecule (e.g., a monospecific or multispecific anti-BCMA antibody molecule) or the anti-BCMA antibody-drug conjugate are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, US 2015/0232557, herein incorporated by reference in their entirety.

In one embodiment, the anti-BCMA antibody molecule (e.g., a monospecific or multispecific anti-BCMA antibody molecule) or the anti-BCMA antibody-drug conjugate comprises one or more (e.g., all three) of VHCDR1, VHCDR2, and VHCDR3 of an anti-BCMA binding domain described herein, and/or one or more (e.g., all three) of VLCDR1, VLCDR2, and a VLCDR3 of an anti-BCMA binding domain described herein, e.g., an anti-BCMA binding domain described in Tables 2, 9, 3-8, and 1. In some embodiments, the CDRs are according to the Kabat definition. In some embodiments, the CDRs are according to the Chothia definition. In some embodiments, the CDRs are according to the combined CDR definitions of both Kabat and Chothia. In some embodiments, the CDRs are according to the IMGT definition.

In one embodiment, the anti-BCMA antibody molecule (e.g., a monospecific or multispecific anti-BCMA antibody molecule) or the anti-BCMA antibody-drug conjugate comprises a heavy chain complementary determining region 1 (VHCDR1), a VHCDR2, and a VHCDR3 of any anti-BMCA heavy chain binding domain amino acid sequence listed in Tables 2, 9, 3, 5, 7, and 1, or a sequence at least about 85%, 90%, 95%, 99% or more identical thereto, and/or having one, two, three or more substitutions, insertions or deletions, e.g., conserved substitutions.

In one embodiment, the anti-BCMA antibody molecule (e.g., a monospecific or multispecific anti-BCMA antibody molecule) or the anti-BCMA antibody-drug conjugate comprises a light chain variable region (VL) comprising a light chain complementarity determining region 1 (VLCDR1), a VLCDR2, and a VLCDR3 of any anti-BMCA light chain binding domain amino acid sequences listed in Tables 2, 9, 4, 6, 8, and 1, or a sequence at least about 85%, 90%, 95%, 99% or more identical thereto, and/or having one, two, three or more substitutions, insertions or deletions, e.g., conserved substitutions.

In certain embodiments, the anti-BCMA antibody molecule (e.g., a monospecific or multispecific anti-BCMA antibody molecule) or the anti-BCMA antibody-drug conjugate comprises one, two or three CDRs from the heavy chain variable region (e.g., HCDR1, HCDR2 and/or HCDR3), provided in Table 3; and/or one, two or three CDRs from the light chain variable region (e.g., LCDR1, LCDR2 and/or LCDR3) of provided in Table 4; or a sequence substantially identical (e.g., 95-99% identical, or up to 20, 15, 10, 8, 6, 5, 4, 3, 2, or 1 amino acid changes, e.g., substitutions (e.g., conservative substitutions)) to any of the aforesaid sequences.

In certain embodiments, the anti-BCMA antibody molecule (e.g., a monospecific or multispecific anti-BCMA antibody molecule) or the anti-BCMA antibody-drug conjugate comprises one, two or three CDRs from the heavy chain variable region (e.g., HCDR1, HCDR2 and/or HCDR3), provided in Table 5; and/or one, two or three CDRs from the light chain variable region (e.g., LCDR1, LCDR2 and/or LCDR3) of provided in Table 6; or a sequence substantially identical (e.g., 95-99% identical, or up to 20, 15, 10, 8, 6, 5, 4, 3, 2, or 1 amino acid changes, e.g., substitutions (e.g., conservative substitutions)) to any of the aforesaid sequences.

In certain embodiments, the anti-BCMA antibody molecule (e.g., a monospecific or multispecific anti-BCMA antibody molecule) or the anti-BCMA antibody-drug conjugate comprises one, two or three CDRs from the heavy chain variable region (e.g., HCDR1, HCDR2 and/or HCDR3), provided in Table 7; and/or one, two or three CDRs from the light chain variable region (e.g., LCDR1, LCDR2 and/or LCDR3) of provided in Table 8; or a sequence substantially identical (e.g., 95-99% identical, or up to 20, 15, 10, 8, 6, 5, 4, 3, 2, or 1 amino acid changes, e.g., substitutions (e.g., conservative substitutions)) to any of the aforesaid sequences.

In certain embodiments, the anti-BCMA antibody molecule (e.g., a monospecific or multispecific anti-BCMA antibody molecule) or the anti-BCMA antibody-drug conjugate comprises:
(1) one, two, or three light chain (LC) CDRs chosen from one of the following:
   (i) a LC CDR1 of SEQ ID NO: 504, LC CDR2 of SEQ ID NO: 544, or LC CDR3 of SEQ ID NO: 584;
   (ii) a LC CDR1 of SEQ ID NO: 514, LC CDR2 of SEQ ID NO: 554, or LC CDR3 of SEQ ID NO: 594;
   (iii) a LC CDR1 of SEQ ID NO: 516, LC CDR2 of SEQ ID NO: 556, or LC CDR3 of SEQ ID NO: 596; or
   (iv) a LC CDR1 of SEQ ID NO: 518, LC CDR2 of SEQ ID NO: 558, or LC CDR3 of SEQ ID NO: 598, and/or
(2) one, two, or three heavy chain (HC) CDRs from one of the following:
   (i) a HC CDR1 of SEQ ID NO: 384, HC CDR2 of SEQ ID NO: 424, or HC CDR3 of SEQ ID NO: 464;
   (ii) a HC CDR1 of SEQ ID NO: 394, HC CDR2 of SEQ ID NO: 434, or HC CDR3 of SEQ ID NO: 474;
   (iii) a HC CDR1 of SEQ ID NO: 396, HC CDR2 of SEQ ID NO: 436, or HC CDR3 of SEQ ID NO: 476; or
   (iv) a HC CDR1 of SEQ ID NO: 398, HC CDR2 of SEQ ID NO: 438, or HC CDR3 of SEQ ID NO: 478.

In certain embodiments, the anti-BCMA antibody molecule (e.g., a monospecific or multispecific anti-BCMA antibody molecule) or the anti-BCMA antibody-drug conjugate comprises:
(1) one, two, or three light chain (LC) CDRs chosen from one of the following:
(i) a LC CDR1 of SEQ ID NO: 744, LC CDR2 of SEQ ID NO: 784, or LC CDR3 of SEQ ID NO: 824;
(ii) a LC CDR1 of SEQ ID NO: 754, LC CDR2 of SEQ ID NO: 794, or LC CDR3 of SEQ ID NO: 834;
(iii) a LC CDR1 of SEQ ID NO: 756, LC CDR2 of SEQ ID NO: 796, or LC CDR3 of SEQ ID NO: 836; or
(iv) a LC CDR1 of SEQ ID NO: 758, LC CDR2 of SEQ ID NO: 798, or LC CDR3 of SEQ ID NO: 838; and/or
(2) one, two, or three heavy chain (HC) CDRs chosen from one of the following:
(i) a HC CDR1 of SEQ ID NO: 624, HC CDR2 of SEQ ID NO: 664, or HC CDR3 of SEQ ID NO: 704;
(ii) a HC CDR1 of SEQ ID NO: 634, HC CDR2 of SEQ ID NO: 674, or HC CDR3 of SEQ ID NO: 714;
(iii) a HC CDR1 of SEQ ID NO: 636, HC CDR2 of SEQ ID NO: 676, or HC CDR3 of SEQ ID NO: 716; or
(iv) a HC CDR1 of SEQ ID NO: 638, HC CDR2 of SEQ ID NO: 678, or HC CDR3 of SEQ ID NO: 718.

In certain embodiments, the anti-BCMA antibody molecule (e.g., a monospecific or multispecific anti-BCMA antibody molecule) or the anti-BCMA antibody-drug conjugate comprises:
(1) one, two, or three light chain (LC) CDRs chosen from one of the following:
   (i) a LC CDR1 of SEQ ID NO: 984 LC CDR2 of SEQ ID NO: 1024, or LC CDR3 of SEQ ID NO: 1064;
   (ii) a LC CDR1 of SEQ ID NO: 994, LC CDR2 of SEQ ID NO: 1034, or LC CDR3 of SEQ ID NO: 1074;
   (iii) a LC CDR1 of SEQ ID NO: 996, LC CDR2 of SEQ ID NO: 1036, or LC CDR3 of SEQ ID NO: 1076; or
   (iv) a LC CDR1 of SEQ ID NO: 998, LC CDR2 of SEQ ID NO: 1038, or LC CDR3 of SEQ ID NO: 1078; and/or
(2) one, two, or three heavy chain (HC) CDRs chosen from one of the following:
   (i) a HC CDR1 of SEQ ID NO: 864, HC CDR2 of SEQ ID NO: 904, or HC CDR3 of SEQ ID NO: 944;
   (ii) a HC CDR1 of SEQ ID NO: 874, HC CDR2 of SEQ ID NO: 914, or HC CDR3 of SEQ ID NO: 954;
   (iii) a HC CDR1 of SEQ ID NO: 876, HC CDR2 of SEQ ID NO: 916, or HC CDR3 of SEQ ID NO: 956;
   (iv) a HC CDR1 of SEQ ID NO: 878, HC CDR2 of SEQ ID NO: 918, or HC CDR3 of SEQ ID NO: 958.

In one embodiment, the anti-BCMA antibody molecule (e.g., a monospecific or multispecific anti-BCMA antibody molecule) or the anti-BCMA antibody-drug conjugate comprises a heavy chain variable region described herein (e.g., in Tables 2, 9, and 1) and/or a light chain variable region described herein (e.g., in Tables 2, 9, and 1). In one embodiment, the anti-BCMA antibody molecule (e.g., a monospecific or multispecific anti-BCMA antibody molecule) or the anti-BCMA antibody-drug conjugate comprises a heavy chain variable region described herein (e.g., in Tables 2, 9, and 1), e.g., at least two heavy chain variable regions described herein (e.g., in Tables 2, 9, and 1). In one embodiment, the anti-BCMA antibody molecule (e.g., a monospecific or multispecific anti-BCMA antibody molecule) or the anti-BCMA antibody-drug conjugate comprises a light chain variable region described herein (e.g., in Tables 2, 9, and 1), e.g., at least two light chain variable regions described herein (e.g., in Tables 2, 9, and 1). In an embodiment, the anti-BCMA antibody molecule (e.g., a monospecific or multispecific anti-BCMA antibody molecule) or the anti-BCMA antibody-drug conjugate comprises: a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region described herein (e.g., in Tables 2, 9, and 1), or a sequence with 95-99% identity with an amino acid sequence described herein (e.g., in Tables 2, 9, and 1); and/or a light chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region described herein (e.g., in Tables 2, 9, and 1), or a sequence with 95-99% identity to an amino acid sequence described herein (e.g., in Tables 2, 9, and 1). In one embodiment, the anti-BCMA antibody molecule (e.g., a monospecific or multispecific anti-BCMA antibody molecule) or the anti-BCMA antibody-drug conjugate comprises an scFv comprising a heavy chain variable region and a light chain variable region of an amino acid sequence described herein (e.g., in Tables 2, 9, and 1).

In one embodiment, the anti-BCMA antibody molecule (e.g., a monospecific or multispecific anti-BCMA antibody molecule) or the anti-BCMA antibody-drug conjugate comprises a heavy chain variable region described herein (e.g., in Table 2) and/or a light chain variable region described herein (e.g., in Table 2). In one embodiment, the anti-BCMA antibody molecule (e.g., a monospecific or multispecific anti-BCMA antibody molecule) or the anti-BCMA antibody-drug conjugate comprises a heavy chain variable region provided in SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a light chain variable region provided in SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a heavy chain variable region provided in SEQ ID NO: 171, SEQ ID NO: 172, SEQ ID NO: 173, SEQ ID NO: 174, SEQ ID NO: 175, SEQ ID NO: 176, SEQ ID NO: 177, or SEQ ID NO: 178, and/or a light chain variable region provided in SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199. In one embodiment, the anti-BCMA antibody molecule (e.g., a monospecific or multispecific anti-BCMA antibody molecule) or the anti-BCMA antibody-drug conjugate comprises: a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, or a sequence with 95-99% identity thereof; and/or a light chain variable region comprising an amino acid sequence having at least one, two or three modifications (e.g., substitutions, e.g., conservative substitutions) but not more than 30, 20 or modifications (e.g., substitutions, e.g., conservative substitutions) of an amino acid sequence of a light chain variable region provided in SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 171 and a VL comprising the amino acid sequence of SEQ ID NO: 192. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 172 and a VL comprising the amino acid sequence of SEQ ID NO: 193. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 173 and a VL comprising the amino acid sequence of SEQ ID NO: 194. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 174 and a VL comprising the amino acid sequence of SEQ ID NO: 195. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 175 and a VL comprising the amino acid sequence of SEQ ID NO: 196. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 176 and a VL comprising the amino acid sequence of SEQ ID NO: 197. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 177 and a VL comprising the amino acid sequence of SEQ ID NO: 198.

In one embodiment, the anti-BCMA antibody molecule (e.g., a monospecific or multispecific anti-BCMA antibody molecule) or the anti-BCMA antibody-drug conjugate comprises a heavy chain variable region described herein (e.g., in Table 9) and/or a light chain variable region described herein (e.g., in Table 9). In one embodiment, the anti-BCMA antibody molecule (e.g., a monospecific or multispecific anti-BCMA antibody molecule) or the anti-BCMA antibody-drug conjugate comprises a heavy chain variable region provided in SEQ ID NO: 255, SEQ ID NO: 256, SEQ ID NO: 257, SEQ ID NO: 258, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in SEQ ID NO: 255, SEQ ID NO: 256, SEQ ID NO: 257, SEQ ID NO: 258, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a light chain variable region provided in SEQ ID NO: 259, SEQ ID NO: 260, SEQ ID NO: 261, SEQ ID NO: 262, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in SEQ ID NO: 259, SEQ ID NO: 260, SEQ ID NO: 261, SEQ ID NO: 262, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a heavy chain variable region provided in SEQ ID NO: 255, SEQ ID NO: 256, SEQ ID NO: 257, or SEQ ID NO: 258, and/or a light chain variable region provided in SEQ ID NO: 259, SEQ ID NO: 260, SEQ ID NO: 261, or SEQ ID NO: 262. In one embodiment, the anti-BCMA antibody molecule (e.g., a monospecific or multispecific anti-BCMA antibody molecule) or the anti-BCMA antibody-drug conjugate comprises: a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in SEQ ID NO: 255, SEQ ID NO: 256, SEQ ID NO: 257, SEQ ID NO: 258, or a sequence with 95-99% identity thereof; and/or a light chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in SEQ ID NO: 259, SEQ ID NO: 260, SEQ ID NO: 261, SEQ ID NO: 262, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 255 and a VL comprising the amino acid sequence of SEQ ID NO: 259. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 256 and a VL comprising the amino acid sequence of SEQ ID NO: 260. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 257 and a VL comprising the amino acid sequence of SEQ ID NO: 261. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 258 and a VL comprising the amino acid sequence of SEQ ID NO: 262.

In one embodiment, the anti-BCMA antibody molecule (e.g., a monospecific or multispecific anti-BCMA antibody molecule) or the anti-BCMA antibody-drug conjugate comprises a heavy chain variable region described herein (e.g., in Table 1) and/or a light chain variable region described herein (e.g., in Table 1). In one embodiment, the anti-BCMA antibody molecule (e.g., a monospecific or multispecific anti-BCMA antibody molecule) or the anti-BCMA antibody-drug conjugate comprises a heavy chain variable region provided in SEQ ID NO: 1118, SEQ ID NO: 1123, SEQ ID NO: 1127, SEQ ID NO: 1131, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in SEQ ID NO: 1118, SEQ ID NO: 1123, SEQ ID NO: 1127, SEQ ID NO: 1131, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a light chain variable region provided in SEQ ID NO: 1119, SEQ ID NO: 1124, SEQ ID NO: 1128, SEQ ID NO: 1132, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in SEQ ID NO: 1119, SEQ ID NO: 1124, SEQ ID NO: 1128, SEQ ID NO: 1132, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a heavy chain variable region provided in SEQ ID NO: 1118, SEQ ID NO: 1123, SEQ ID NO: 1127, or SEQ ID NO: 1131, and/or a light chain variable region provided in SEQ ID NO: 1119, SEQ ID NO: 1124, SEQ ID NO: 1128, or SEQ ID NO: 1132. In one embodiment, the anti-BCMA antibody molecule (e.g., a monospecific or multispecific anti-BCMA antibody molecule) or the anti-BCMA antibody-drug conjugate comprises: a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in SEQ ID NO: 1118, SEQ ID NO: 1123, SEQ ID NO: 1127, SEQ ID NO: 1131, or a sequence with 95-99% identity thereof; and/or a light chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in SEQ ID NO: 1119, SEQ ID NO: 1124, SEQ ID NO: 1128, SEQ ID NO: 1132, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 1118 and a VL comprising the amino acid sequence of SEQ ID NO: 1119. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 1123 and a VL comprising the amino acid sequence of SEQ ID NO: 1124. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 1127 and a VL comprising the amino acid sequence of SEQ ID NO: 1128. In one embodiment, the anti-BCMA antibody molecule (e.g., a monospecific or multispecific anti-BCMA antibody molecule) or the anti-BCMA antibody-drug conjugate comprises a VH comprising the amino acid sequence of SEQ ID NO: 1131 and a VL comprising the amino acid sequence of SEQ ID NO: 1132.

In one embodiment, the anti-BCMA antibody molecule (e.g., a monospecific or multispecific anti-BCMA antibody molecule) or the anti-BCMA antibody-drug conjugate comprises a heavy chain described herein (e.g., in Table 1) and/or a light chain described herein (e.g., in Table 1). In one embodiment, the anti-BCMA antibody molecule (e.g., a monospecific or multispecific anti-BCMA antibody molecule) or the anti-BCMA antibody-drug conjugate comprises a heavy chain provided in SEQ ID NO: 1120, SEQ ID NO: 1125, SEQ ID NO: 1129, SEQ ID NO: 1133, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain provided in SEQ ID NO: 1120, SEQ ID NO: 1125, SEQ ID NO: 1129, SEQ ID NO: 1133, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a light chain provided in SEQ ID NO: 1121, SEQ ID NO: 1126, SEQ ID NO: 1130, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain provided in SEQ ID NO: 1121, SEQ ID NO: 1126, SEQ ID NO: 1130, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a heavy chain provided in SEQ ID NO: 1120, SEQ ID NO: 1125, SEQ ID NO: 1129, or SEQ ID NO: 1133, and/or a light chain provided in SEQ ID NO: 1121, SEQ ID NO: 1126, or SEQ ID NO: 1130. In one embodiment, the anti-BCMA antibody molecule (e.g., a monospecific or multispecific anti-BCMA antibody molecule) or the anti-BCMA antibody-drug conjugate comprises: a heavy chain comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain provided in SEQ ID NO: 1120, SEQ ID NO: 1125, SEQ ID NO: 1129, SEQ ID NO: 1133, or a sequence with 95-99% identity thereof; and/or a light chain comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain provided in SEQ ID NO: 1121, SEQ ID NO: 1126, SEQ ID NO: 1130, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises a heavy chain comprising the amino acid sequence of SEQ ID NO: 1120 and a light chain comprising the amino acid sequence of SEQ ID NO: 1121. In one embodiment, the anti-BCMA antibody molecule (e.g., a monospecific or multispecific anti-BCMA antibody molecule) or the anti-BCMA antibody-drug conjugate comprises a heavy chain comprising the amino acid sequence of SEQ ID NO: 1125 and a light chain comprising the amino acid sequence of SEQ ID NO: 1126. In one embodiment, the anti-BCMA antibody molecule (e.g., a monospecific or multispecific anti-BCMA antibody molecule) or the anti-BCMA antibody-drug conjugate comprises a heavy chain comprising the amino acid sequence of SEQ ID NO: 1129 and a light chain comprising the amino acid sequence of SEQ ID NO: 1130.

In one embodiment, the anti-BCMA antibody molecule (e.g., a monospecific or multispecific anti-BCMA antibody molecule) or the anti-BCMA antibody-drug conjugate comprises an scFv described herein (e.g., in Tables 2 and 9). In one embodiment, the anti-BCMA antibody molecule (e.g., a monospecific or multispecific anti-BCMA antibody molecule) or the anti-BCMA antibody-drug conjugate comprises an scFv provided in SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO:131, SEQ ID NO: 132, SEQ ID NO: 133, SEQ ID NO: 134, SEQ ID NO: 135, SEQ ID NO:136, SEQ ID NO: 137, SEQ ID NO:138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 263, SEQ ID NO: 264, SEQ ID NO: 265, SEQ ID NO: 266, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of an scFv provided in SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO:130, SEQ ID NO:131, SEQ ID NO: 132, SEQ ID NO:133, SEQ ID NO: 134, SEQ ID NO: 135, SEQ ID NO:136, SEQ ID NO: 137, SEQ ID NO:138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 263, SEQ ID NO: 264, SEQ ID NO: 265, SEQ ID NO: 266, or a sequence with 95-99% identity thereof. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 39. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 40. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 41. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 42. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 43. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 44. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 45. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 46. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 47. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 48. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 49. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 50. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 51. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 52. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 53. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 129. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 130. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 131. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 132. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 133. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 134. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 135. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 136. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 137. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 138. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 139. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 140. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 141. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 142. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 143. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 144. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 145. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 146. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 147. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 148. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 149. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 263. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 264. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 265. In one embodiment, the anti-BCMA antibody molecule (e.g., a monospecific or multispecific anti-BCMA antibody molecule) or the anti-BCMA antibody-drug conjugate comprises the scFv provided in SEQ ID NO: 266.

In one embodiment, the anti-BCMA antibody molecule (e.g., a monospecific or multispecific anti-BCMA antibody molecule) or the anti-BCMA antibody-drug conjugate comprises an scFv that is a humanized variant of an scFv domain of an antibody or antibody fragment described in PCT Publication No. WO 2012/163805, U.S. Pat. No. 7,083,785, EP Patent No. 1975231B1, or PCT Publication No. WO 13/154760 (the contents of each are hereby incorporated by reference in their entireties), which disclose antibodies or scFv fragments of murine origin that specifically binds to human BCMA. Humanization of these mouse antibodies and/or scFvs may be desired for the clinical setting, where the mouse-specific residues may induce a human-anti-mouse antigen (HAMA) response in patients who receive the BCMA-targeting treatment.

Also included in the invention is a nucleotide sequence that encodes the polypeptide of each of the scFv fragments selected from the group consisting of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 263, SEQ ID NO: 264, SEQ ID NO: 265, and SEQ ID NO: 266.

In one embodiment, the present invention encompasses a recombinant nucleic acid construct comprising a nucleic acid molecule encoding an anti-BCMA binding domain. In one embodiment, the nucleic acid molecule is selected from the group consisting of SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, and SEQ ID NO: 170.

In one aspect, the anti-BCMA binding domain is a fragment, e.g., a single chain variable fragment (scFv). In one aspect, the anti-BCMA binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a BCMA protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:25). In one embodiment, the linker can be (Gly₄Ser)₄ (SEQ ID NO:27) or (Gly₄Ser)₃ (SEQ ID NO:28). Variation in the linker length may retain or enhance activity, giving rise to superior efficacy in activity studies.

In one aspect, the BCMA-targeting agent is a recombinant non-antibody protein that binds to BCMA. In one embodiment, the recombinant non-antibody protein that binds to BCMA comprises a BCMA ligand. In one embodiment, the recombinant non-antibody protein that binds to BCMA comprises the amino acid sequence of B-cell activating factor (BAFF) or fragment thereof. In one embodiment, the recombinant non-antibody protein that binds to BCMA comprises the amino acid sequence of a proliferation-inducing ligand (APRIL) or fragment thereof. The exemplary amino acid sequences of BAFF, APRIL, or fragment thereof are shown in Table 10. In one embodiment, the BCMA-targeting agent comprises an amino acid sequence selected from the group consisting of SEQ ID NOs: 1137-1147, or any fragment thereof. In one embodiment, the BCMA-targeting agent comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence selected from the group consisting of SEQ ID NOs: 1137-1147, or a sequence with 95-99% identity thereof, or any fragment thereof.

In one embodiment, the recombinant non-antibody protein that binds to BCMA is linked, e.g., via a linker, to a drug moiety. In one embodiment, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor. In one embodiment, the linker is chosen from a cleavable linker, a non-cleavable linker, a hydrophilic linker, a procharged linker, or a dicarboxylic acid based linker.

TABLE 10
Exemplary amino acid sequences of BAFF, APRIL,
or fragment thereof

| SEQ ID | | |
|---|---|---|
| NO | Description | Sequence |
| 1137 | Exemplary BAFF | MDDSTEREQSRLTSCLKKREEMKLKECVSILPRKESPSVRSSKD |
| | isoform 1 | GKLLAATLLLALLSCCLTVVSFYQVAALQGDLASLRAELQGHHA |
| | (Q9Y275-1) | EKLPAGAGAPKAGLEEAPAVTAGLKIFEPPAPGEGNSSQNSRNK |
| | | RAVQGPEETVTQDCLQLIADSETPTIQKGSYTFVPWLLSFKRGS |
| | | ALEEKENKILVKETGYFFIYGQVLYTDKTYAMGHLIQRKKVHVF |
| | | GDELSLVTLFRCIQNMPETLPNNSCYSAGIAKLEEGDELQLAIP |
| | | RENAQISLDGDVTFFGALKLL |
| 1138 | Exemplary BAFF | MDDSTEREQSRLTSCLKKREEMKLKECVSILPRKESPSVRSSKD |
| | isoform 2 | GKLLAATLLLALLSCCLTVVSFYQVAALQGDLASLRAELQGHHA |
| | (Q9Y275-2) | EKLPAGAGAPKAGLEEAPAVTAGLKIFEPPAPGEGNSSQNSRNK |
| | | RAVQGPEETGSYTFVPWLLSFKRGSALEEKENKILVKETGYFFI |
| | | YGQVLYTDKTYAMGHLIQRKKVHVFGDELSLVTLFRCIQNMPET |
| | | LPNNSCYSAGIAKLEEGDELQLAIPRENAQISLDGDVTFFGALK |
| | | LL |
| 1139 | Exemplary BAFF | MDDSTEREQSRLTSCLKKREEMKLKECVSILPRKESPSVRSSKD |
| | isoform 3 | GKLLAATLLLALLSCCLTVVSFYQVAALQGDLASLRAELQGHHA |
| | (Q9Y275-3) | EKLPAGAGAPKAGLEEAPAVTAGLKIFEPPAPGEGNSSQNSRNK |
| | | RAVQGPEETVTQDCLQLIADSETPTIQKGFIY |
| 1140 | Exemplary BAFF | QVAALQGDLASLRAELQGHHAEKLPAGAGAPKAGLEEAPAVTAG |
| | extracellular | LKIFEPPAPGEGNSSQNSRNKRAVQGPEETVTQDCLQLIADSET |
| | domain | PTIQKGSYTFVPWLLSFKRGSALEEKENKILVKETGYFFIYGQV |
| | | LYTDKTYAMGHLIQRKKVHVFGDELSLVTLFRCIQNMPETLPNN |
| | | SCYSAGIAKLEEGDELQLAIPRENAQISLDGDVTFFGALKLL |
| 1141 | Exemplary | AVQGPEETVTQDCLQLIADSETPTIQKGSYTFVPWLLSFKRGSA |
| | soluble BAFF | LEEKENKILVKETGYFFIYGQVLYTDKTYAMGHLIQRKKVHVFG |
| | | DELSLVTLFRCIQNMPETLPNNSCYSAGIAKLEEGDELQLAIPR |
| | | ENAQISLDGDVTFFGALKLL |
| 1142 | Exemplary | MPASSPFLLAPKGPPGNMGGPVREPALSVALWLSWGAALGAVAC |
| | APRIL isoform | AMALLTQQTELQSLRREVSRLQGTGGPSQNGEGYPWQSLPEQSS |
| | alpha | DALEAWENGERSRKRRAVLTQKQKKQHSVLHLVPINATSKDDSD |
| | (O75888-1) | VTEVMWQPALRRGRGLQAQGYGVRIQDAGVYLLYSQVLFQDVTF |
| | | TMGQVVSREGQGRQETLFRCIRSMPSHPDRAYNSCYSAGVFHLH |
| | | QGDILSVIIPRARAKLNLSPHGTFLGFVKL |
| 1143 | Exemplary | MPASSPFLLAPKGPPGNMGGPVREPALSVALWLSWGAALGAVAC |
| | APRIL isoform | AMALLTQQTELQSLRREVSRLQGTGGPSQNGEGYPWQSLPEQSS |
| | beta | DALEAWENGERSRKRRAVLTQKQKNDSDVTEVMWQPALRRGRGL |
| | (O75888-2) | QAQGYGVRIQDAGVYLLYSQVLFQDVTFTMGQVVSREGQGRQET |
| | | LFRCIRSMPSHPDRAYNSCYSAGVFHLHQGDILSVIIPRARAKL |
| | | NLSPHGTFLGFVKL |
| 1144 | Exemplary | MPASSPFLLAPKGPPGNMGGPVREPALSVALWLSWGAALGAVAC |
| | APRIL isoform | AMALLTQQTELQSLRREVSRLQGTGGPSQNGEGYPWQSLPEQSS |
| | gamma | DALEAWENGERSRKRRAVLTQKQKKQHSVLHLVPINATSKDDSD |
| | (O75888-3) | VTEVMWQPALRRGRGLQAQGYGVRIQDAGVYLLYSQVLFQDVTF |
| | | TMGQVVSREGQGRQETLFRCIRSMPSHPDRAYNSCYSAGVFHLH |
| | | QGDILSVIIPRARAKLNLSPHGTFLGL |
| 1145 | Exemplary | MPASSPFLLAPKGPPGNMGGPVREPALSVALWLSWGAALGAVAC |
| | APRIL | AMALLTQQTELQSLRREVSRLQGTGGPSQNGEGYPWQSLPEQHS |
| | isoform 4 | VLHLVPINATSKDDSDVTEVMWQPALRRGRGLQAQGYGVRIQDA |
| | (O75888-4) | GVYLLYSQVLFQDVTFTMGQVVSREGQGRQETLFRCIRSMPSHP |
| | | DRAYNSCYSAGVFHLHQGDILSVIIPRARAKLNLSPHGTFLGFV |
| | | KL |
| 1146 | Exemplary | MGGPVREPALSVALWLSWGAALGAVACAMALLTQQTELQSLRRE |
| | APRIL | VSRLQGTGGPSQNGEGYPWQSLPEQHSVLHLVPINATSKDDSDV |
| | isoform 5 | TEVMWQPALRRGRGLQAQGYGVRIQDAGVYLLYSQVLFQDVTFT |
| | (O75888-5) | MGQVVSREGQGRQETLFRCIRSMPSHPDRAYNSCYSAGVFHLHQ |
| | | GDILSVIIPRARAKLNLSPHGTFLGFVKL |
| 1147 | Exemplary | AVLTQKQKKQHSVLHLVPINATSKDDSDVTEVMWQPALRRGRGL |
| | APRIL fragment | QAQGYGVRIQDAGVYLLYSQVLFQDVTFTMGQVVSREGQGRQET |
| | | LFRCIRSMPSHPDRAYNSCYSAGVFHLHQGDILSVIIPRARAKL |
| | | NLSPHGTFLGFVKL |

### Nucleic Acids

The invention also features nucleic acids comprising nucleotide sequences that encode the BCMA-targeting agent, e.g., an anti-BCMA antibody molecule or a recombinant non-antibody protein that binds to BCMA, as described herein.

For example, the invention features a first and second nucleic acid encoding heavy and light chain variable regions, respectively, of an anti-BCMA antibody molecule chosen from one or more of the antibody molecules disclosed herein. The nucleic acid can comprise a nucleotide sequence as set forth in Table 2, or a sequence substantially identical thereto (e.g., a sequence at least about 85%, 90%, 95%, 99% or more identical thereto, or which differs by no more than 3, 6, 15, 30, or 45 nucleotides from the sequences shown in Table 2).

In certain embodiments, the nucleic acid can comprise a nucleotide sequence encoding at least one, two, or three CDRs or hypervariable loops from a heavy chain variable region having an amino acid sequence as set forth in Table 2, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in Table 2, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in Table 2, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in Table 2, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein).

In one embodiment, the invention features a nucleic acid encoding a recombinant non-antibody protein that binds to BCMA, e.g., a recombinant non-antibody protein having an amino acid sequence disclosed in Table 10, or any fragment thereof.

In another aspect, the application features host cells and vectors containing the nucleic acids described herein. The nucleic acids may be present in a single vector or separate vectors present in the same host cell or separate host cell, as described in more detail herein below.

### Vectors

Further provided herein are vectors comprising nucleotide sequences encoding a BCMA-targeting agent described herein. In one embodiment, the vectors comprise nucleotides encoding an antibody molecule described herein. In one embodiment, the vectors comprise nucleotides encoding a recombinant non-antibody protein that binds to BCMA describe herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the BCMA-targeting agent produced are known to those skilled in the art, and may be varied or optimized depending upon the specific expression vector and mammalian host cell employed, based upon the present description.

### Cells

The invention also provides host cells comprising a nucleic acid encoding a BCMA-targeting agent as described herein.

In one embodiment, the host cells are genetically engineered to comprise nucleic acids encoding the BCMA-targeting agent.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Pharmaceutical Compositions and Kits

In some aspects, this disclosure provides compositions, e.g., pharmaceutically acceptable compositions, which include a BCMA-targeting agent (e.g., an anti-BCMA antibody molecule or a recombinant non-antibody protein that binds to BCMA) described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some embodiments, the BCMA-targeting agent is administered by intravenous infusion or injection. In certain embodiments, the BCMA-targeting agent is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high protein concentration. Sterile injectable solutions can be prepared by incorporating the active compound (e.g., a BCMA-targeting agent) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The BCMA-targeting agents (e.g., an anti-BCMA antibody molecule or a recombinant non-antibody protein that binds to BCMA) can be administered by a variety of methods. Several are known in the art, and for many therapeutic applications, an appropriate route/mode of administration is intravenous injection or infusion. In an embodiment, the BCMA-targeting agents (e.g., an anti-BCMA antibody molecule or a recombinant non-antibody protein that binds to BCMA) can be administered by intravenous infusion at a rate of more than 20 mg/min, e.g., 20-40 mg/min. In an embodiment, the BCMA-targeting agents (e.g., an anti-BCMA antibody molecule or a recombinant non-antibody protein that binds to BCMA) can be administered by intravenous infusion at a rate of greater than or equal to 40 mg/min to reach a dose of about 35 to 440 mg/m², about 70 to 310 mg/m², or about 110 to 130 mg/m². In an embodiment, the BCMA-targeting agents (e.g., an anti-BCMA antibody molecule or a recombinant non-antibody protein that binds to BCMA) can be administered by intravenous infusion at a rate of less than 10 mg/min, e.g., less than or equal to 5 mg/min to reach a dose of about 1 to 100 mg/m², about 5 to 50 mg/m², about 7 to 25 mg/m², or about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a BCMA-targeting agent (e.g., an anti-BCMA antibody molecule or a recombinant non-antibody protein that binds to BCMA) can be orally administered, for example, with an inert diluent or an assimilable edible carrier. The BCMA-targeting agent (e.g., an anti-BCMA antibody molecule or a recombinant non-antibody protein that binds to BCMA) (and other ingredients, if desired) may also be enclosed in a hard or soft shell gelatin capsule, compressed into tablets, or incorporated directly into the subject's diet. For oral therapeutic administration, the BCMA-targeting agent (e.g., an anti-BCMA antibody molecule or a recombinant non-antibody protein that binds to BCMA) may be incorporated with excipients and used in the form of ingestible tablets, buccal tablets, troches, capsules, elixirs, suspensions, syrups, wafers, and the like. To administer a BCMA-targeting agent (e.g., an anti-BCMA antibody molecule or a recombinant non-antibody protein that binds to BCMA) by other than parenteral administration, it may be necessary to coat the compound with, or co-administer the compound with, a material to prevent its inactivation. Therapeutic compositions can also be administered with medical devices known in the art.

Dosage regimens are adjusted to provide the desired response (e.g., a therapeutic response). For example, a single bolus may be administered, several divided doses may be administered overtime or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a BCMA-targeting agent (e.g., an anti-BCMA antibody molecule or a recombinant non-antibody protein that binds to BCMA) is 0.1-30 mg/kg, more preferably 1-25 mg/kg. Dosages and therapeutic regimens of the BCMA-targeting agent (e.g., an anti-BCMA antibody molecule or a recombinant non-antibody protein that binds to BCMA) can be determined by a skilled artisan. In certain embodiments, the BCMA-targeting agent (e.g., an anti-BCMA antibody molecule or a recombinant non-antibody protein that binds to BCMA) is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the BCMA-targeting agent (e.g., an anti-BCMA antibody molecule or a recombinant non-antibody protein that binds to BCMA) is administered at a dose from about 10 to 20 mg/kg every other week. The BCMA-targeting agent (e.g., an anti-BCMA antibody molecule or a recombinant non-antibody protein that binds to BCMA) can be administered by intravenous infusion at a rate of more than 20 mg/min, e.g., 20-40 mg/min, e.g., greater than or equal to 40 mg/min to reach a dose of about 35 to 440 mg/m², about 70 to 310 mg/m², or about 110 to 130 mg/m². In embodiments, the infusion rate of about 110 to 130 mg/m² achieves a level of about 3 mg/kg. In other embodiments, the BCMA-targeting agent (e.g., an anti-BCMA antibody molecule or a recombinant non-antibody protein that binds to BCMA) can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or about 10 mg/m². In some embodiments, the BCMA-targeting agent (e.g., an anti-BCMA antibody molecule or a recombinant non-antibody protein that binds to BCMA) is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of a BCMA-targeting agent (e.g., an anti-BCMA antibody molecule or a recombinant non-antibody protein that binds to BCMA) of the invention. A “therapeutically effective amount” refers to an amount effective, at dosages and for periods of time necessary, to achieve the desired therapeutic result. A therapeutically effective amount of the BCMA-targeting agent (e.g., an anti-BCMA antibody molecule or a recombinant non-antibody protein that binds to BCMA) may vary according to factors such as the disease state, age, sex, and weight of the individual, and the ability of the BCMA-targeting agent (e.g., an anti-BCMA antibody molecule or a recombinant non-antibody protein that binds to BCMA) to elicit a desired response in the individual. A therapeutically effective amount is also one in which any toxic or detrimental effects of the BCMA-targeting agent (e.g., an anti-BCMA antibody molecule or a recombinant non-antibody protein that binds to BCMA) are outweighed by the therapeutically beneficial effects. A “therapeutically effective dosage” preferably inhibits a measurable parameter by at least about 20%, more preferably by at least about 40%, even more preferably by at least about 60%, and still more preferably by at least about 80% relative to untreated subjects. The measurable parameter may be, e.g., tumor growth rate or pathogen growth rate. The ability of a BCMA-targeting agent (e.g., an anti-BCMA antibody molecule or a recombinant non-antibody protein that binds to BCMA) to inhibit a measurable parameter can be evaluated in an animal model system predictive of efficacy in the corresponding human disease. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention is a kit comprising a BCMA-targeting agent (e.g., an anti-BCMA antibody molecule or a recombinant non-antibody protein that binds to BCMA) described herein. The kit can include one or more other elements including: instructions for use; other reagents, e.g., a label, a therapeutic agent, or an agent useful for chelating, or otherwise coupling, a BCMA-targeting agent (e.g., an anti-BCMA antibody molecule or a recombinant non-antibody protein that binds to BCMA) to a label or therapeutic agent, or a radioprotective composition; devices or other materials for preparing the BCMA-targeting agent (e.g., an anti-BCMA antibody molecule or a recombinant non-antibody protein that binds to BCMA) for administration; pharmaceutically acceptable carriers; and devices or other materials for administration to a subject.

### Therapeutic Application

### BCMA Associated Diseases and or Disorders

In one aspect, the invention provides methods for treating a disease associated with BCMA expression. In one aspect, the invention provides methods for treating a disease wherein part of the tumor is negative for BCMA and part of the tumor is positive for BCMA. For example, the composition comprising a BCMA-targeting agent and a GSI is useful for treating subjects that have undergone treatment for a disease associated with elevated expression of BCMA, wherein the subject that has undergone treatment for elevated levels of BCMA exhibits a disease associated with elevated levels of BCMA. In embodiments, disclosed herein is a method for treating subjects that have undergone treatment for a disease associated with expression of BCMA, comprising administering to the subject an effective amount of: (i) a BCMA-targeting agent (e.g., an anti-BCMA antibody molecule or a recombinant non-antibody protein that binds to BCMA), and (ii) a GSI, wherein the subject that has undergone treatment related to expression of BCMA exhibits a disease associated with expression of BCMA.

In one embodiment, the invention provides methods for treating a disease wherein BCMA is expressed on both normal cells and cancers cells, but is expressed at lower levels on normal cells. In one embodiment, the method further comprises selecting a BCMA-targeting agent of the invention that binds with an affinity that allows the BCMA-targeting agent to bind and kill the cancer cells expressing BCMA but less than 30%, 25%, 20%, 15%, 10%, 5% or less of the normal cells expressing BCMA are killed, e.g., as determined by an assay described herein. For example, a killing assay such as flow cytometry based on Cr51 CTL can be used. In one embodiment, the BCMA-targeting agent has an antigen binding domain that has a binding affinity K_{D} of 10⁻⁴ M to 10⁻⁸ M, e.g., 10⁻⁵ M to 10⁻⁷ M, e.g., 10⁻⁶ M or 10⁻⁷ M, for the target antigen. In one embodiment, the BCMA antigen binding domain has a binding affinity that is at least five-fold, 10-fold, 20-fold, 30-fold, 50-fold, 100-fold or 1,000-fold less than a reference antibody, e.g., an antibody described herein.

In one aspect, the invention pertains to a method of inhibiting growth of a BCMA-expressing tumor cell, comprising contacting the tumor cell with a BCMA-targeting agent (e.g., an anti-BCMA antibody molecule or a recombinant non-antibody protein that binds to BCMA) and a GSI of the present invention such that the growth of the tumor cell is inhibited.

In one aspect, the invention pertains to a method of treating cancer in a subject. The method comprises administering to the subject a BCMA-targeting agent (e.g., an anti-BCMA antibody molecule or a recombinant non-antibody protein that binds to BCMA) and a GSI such that the cancer is treated in the subject. An example of a cancer that is treatable by the BCMA-targeting agent and GSI of the present invention is a cancer associated with expression of BCMA.

Generally, disclosed herein is a method of treating and/or preventing a disease that arises in individuals who are immunocompromised, comprising administering a BCMA-targeting agent (e.g., an anti-BCMA antibody molecule or a recombinant non-antibody protein that binds to BCMA) and a GSI. In particular, disclosed herein is a method of treating diseases, disorders and conditions associated with expression of BCMA, comprising administering a BCMA-targeting agent (e.g., an anti-BCMA antibody molecule or a recombinant non-antibody protein that binds to BCMA) and a GSI. In certain aspects, disclosed herein is a method of treating patients at risk for developing diseases, disorders and conditions associated with expression of BCMA, comprising administering a BCMA-targeting agent (e.g., an anti-BCMA antibody molecule or a recombinant non-antibody protein that binds to BCMA) and a GSI. Thus, the present invention provides methods for the treatment or prevention of diseases, disorders and conditions associated with expression of BCMA comprising administering to a subject in need thereof, a therapeutically effective amount of (i) a BCMA-targeting agent (e.g., an anti-BCMA antibody molecule or a recombinant non-antibody protein that binds to BCMA), and (ii) a gamma secretase inhibitor (GSI).

In one aspect, disclosed herein is a method of treating a proliferative disease such as a cancer or malignancy or a precancerous condition such as a myelodysplasia, a myelodysplastic syndrome or a preleukemia, comprising administering a BCMA-targeting agent (e.g., an anti-BCMA antibody molecule or a recombinant non-antibody protein that binds to BCMA) and a GSI. In one aspect, the cancer is a hematological cancer. Hematological cancer conditions are the types of cancer such as leukemia and malignant lymphoproliferative conditions that affect blood, bone marrow and the lymphatic system. In one aspect, the hematological cancer is a leukemia. An example of a disease or disorder associated with BCMA is multiple myeloma (also known as MM) (See Claudio et al., Blood. 2002, 100(6):2175-86; and Novak et al., Blood. 2004, 103(2):689-94). Multiple myeloma, also known as plasma cell myeloma or Kahler's disease, is a cancer characterized by an accumulation of abnormal or malignant plasma B-cells in the bone marrow. Frequently, the cancer cells invade adjacent bone, destroying skeletal structures and resulting in bone pain and fractures. Most cases of myeloma also feature the production of a paraprotein (also known as M proteins or myeloma proteins), which is an abnormal immunoglobulin produced in excess by the clonal proliferation of the malignant plasma cells. Blood serum paraprotein levels of more than 30 g/L is diagnostic of multiple myeloma, according to the diagnostic criteria of the International Myeloma Working Group (IMWG) (See Kyle et al. (2009), Leukemia. 23:3-9). Other symptoms or signs of multiple myeloma include reduced kidney function or renal failure, bone lesions, anemia, hypercalcemia, and neurological symptoms.

Criteria for distinguishing multiple myeloma from other plasma cell proliferative disorders have been established by the International Myeloma Working Group (See Kyle et al. (2009), Leukemia. 23:3-9). All three of the following criteria must be met:
Clonal bone marrow plasma cells ≥10%
Presence of serum and/or urinary monoclonal protein (except in patients with true non-secretory multiple myeloma)
Evidence of end-organ damage attributable to the underlying plasma cell proliferative disorder, specifically:
   Hypercalcemia: serum calcium ≥11.5 mg/100 ml
   Renal insufficienty: serum creatinine >1.73 mmol/l
   Anemia: normochromic, normocytic with a hemoglobin value of >2 g/100 ml below the lower limit of normal, or a hemoglobin value <10 g/100 ml
   Bone lesions: lytic lesions, severe osteopenia, or pathologic fractures.

Other plasma cell proliferative disorders that can be treated by the compositions and methods described herein include, but are not limited to, asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

Two staging systems are used in the staging of multiple myeloma: the International Staging System (ISS) (See Greipp et al. (2005), J. Clin. Oncol. 23 (15):3412-3420) and the Durie-Salmon Staging system (DSS) (See Durie et al. (1975), Cancer 36 (3): 842-854). The two staging systems are summarized in the table below:

TABLE 11

| | International Staging System | | Durie-Salmon Staging System | |
|---|---|---|---|---|
| | | Median | | Median |
| Stage | Criteria | survival | Criteria | survival* |
| I | β₂M <3.5 mg/l and | 62 | All of the following: | IA: 62 |
| | serum albumin ≥3.5 | months | Hemoglobin level >10 | months |
| | g/dL | | g/dL Serum calcium, | IB: 22 |
| | | | normal or <12 mg/dL | months |
| | | | Bone x-ray, normal or | |
| | | | 1 plasmacytoma only | |
| | | | Low monoclonal | |
| | | | protein production | |
| | | | (IgG <5 g/dL, IgA <3 | |
| | | | g/dL, Bence Jones | |
| | | | protein <4 g/dL per | |
| | | | 24 hours | |
| II | Neither stage I or | 44 | Neither stage I or | IIA: 58 |
| | stage III | months | stage III | months |
| | | | | IIB: 354 |
| | | | | months |
| III | β₂M ≥5.5 mg/l | 29 | One or more of the | IIIA: 45 |
| | | months | following: | months |
| | | | Hemogloblin | IIIB: 24 |
| | | | level <8.5 g/dL Serum | months |
| | | | calcium, normal | |
| | | | or >12 mg/dL | |
| | | | Advanced osteolytic | |
| | | | lesions High | |
| | | | monoclonal protein | |
| | | | production (IgG >7 | |
| | | | g/dL, IgA >5 g/dL, | |
| | | | Bence Jones | |
| | | | protein >12 g/dL per | |
| | | | 24 hours | |

*The Durie-Salmon Staging system also includes a subclassification that designates the status of renal function. The designation of “A” or “B” is added after the stage number, wherein “A” indicates relatively normal renal function (serum creatinine value <2.0 mg/dL), and B indicates abnormal renal function (serum creatinine value >2.0 mg/dL).

Standard treatment for multiple myeloma and associated diseases includes chemotherapy, stem cell transplant (autologous or allogeneic), radiation therapy, and other drug therapies. Frequently used anti-myeloma drugs include alkylating agents (e.g., bendamustine, cyclophosphamide and melphalan), proteasome inhibitors (e.g., bortezomib), corticosteroids (e.g., dexamethasone and prednisone), and immunomodulators (e.g., thalidomide and lenalidomide or Revlimid®), or any combination thereof. Biphosphonate drugs are also frequently administered in combination with the standard anti-MM treatments to prevent bone loss. Patients older than 65-70 years of age are unlikely candidates for stem cell transplant. In some cases, double-autologous stem cell transplants are options for patients less than 60 years of age with suboptimal response to the first transplant. The compositions and methods of the present invention may be administered in combination with any of the currently prescribed treatments for multiple myeloma.

Another example of a disease or disorder associated with BCMA is Hodgkin's lymphoma and non-Hodgkin's lymphoma (See Chiu et al., Blood. 2007, 109(2):729-39; He et al., J Immunol. 2004, 172(5):3268-79).

Hodgkin's lymphoma (HL), also known as Hodgkin's disease, is a cancer of the lymphatic system that originates from white blood cells, or lymphocytes. The abnormal cells that comprise the lymphoma are called Reed-Sternberg cells. In Hodgkin's lymphoma, the cancer spreads from one lymph node group to another. Hodgkin's lymphoma can be subclassified into four pathologic subtypes based upon Reed-Sternberg cell morphology and the cell composition around the Reed-Sternberg cells (as determined through lymph node biopsy): nodular sclerosing HL, mixed-cellularity subtype, lymphocyte-rich or lymphocytic predominance, lymphocyte depleted. Some Hodgkin's lymphoma can also be nodular lymphocyte predominant Hodgkin's lymphoma, or can be unspecified. Symptoms and signs of Hodgkin's lymphoma include painless swelling in the lymph nodes in the neck, armpits, or groin, fever, night sweats, weight loss, fatigue, itching, or abdominal pain.

Non-Hodgkin's lymphoma (NHL) comprises a diverse group of blood cancers that include any kind of lymphoma other than Hodgkin's lymphoma. Subtypes of non-Hodgkin's lymphoma are classified primarily by cell morphology, chromosomal aberrations, and surface markers. NHL subtypes (or NHL-associated cancers) include B cell lymphomas such as, but not limited to, Burkitt's lymphoma, B-cell chronic lymphocytic leukemia (B-CLL), B-cell prolymphocytic leukemia (B-PLL), chronic lymphocytic leukemia (CLL), diffuse large B-cell lymphoma (DLBCL) (e.g., intravascular large B-cell lymphoma and primary mediastinal B-cell lymphoma), follicular lymphoma (e.g., follicle center lymphoma, follicular small cleaved cell), hair cell leukemia, high grade B-cell lymphoma (Burkitt's like), lymphoplasmacytic lymphoma (Waldenstrom's macroglublinemia), mantle cell lymphoma, marginal zone B-cell lymphomas (e.g., extranodal marginal zone B-cell lymphoma or mucosa-associated lymphoid tissue (MALT) lymphoma, nodal marginal zone B-cell lymphoma, and splenic marginal zone B-cell lymphoma), plasmacytoma/myeloma, precursor B-lymphoblastic leukemia/lymphoma (PB-LBL/L), primary central nervous system (CNS) lymphoma, primary intraocular lymphoma, small lymphocytic lymphoma (SLL); and T cell lymphomas, such as, but not limited to, anaplastic large cell lymphoma (ALCL), adult T-cell lymphoma/leukemia (e.g., smoldering, chronic, acute and lymphomatous), angiocentric lymphoma, angioimmunoblastic T-cell lymphoma, cutaneous T-cell lymphomas (e.g., mycosis fungoides, Sezary syndrome, etc.), extranodal natural killer/T-cell lymphoma (nasal-type), enteropathy type intestinal T-cell lymphoma, large granular lymphocyte leukemia, precursor T-lymphoblastic lymphoma/leukemia (T-LBL/L), T-cell chronic lymphocytic leukemia/prolymphocytic leukemia (T-CLL/PLL), and unspecified peripheral T-cell lymphoma. Symptoms and signs of Hodgkin's lymphoma include painless swelling in the lymph nodes in the neck, armpits, or groin, fever, night sweats, weight loss, fatigue, itching, abdominal pain, coughing, or chest pain.

The staging is the same for both Hodgkin's and non-Hodgkin's lymphoma, and refers to the extent of spread of the cancer cells within the body. In stage I, the lymphoma cells are in one lymph node group. In stage II, lymphoma cells are present in at least two lymph node groups, but both groups are on the same side of the diaphragm, or in one part of a tissue or organ and the lymph nodes near that organ on the same side of the diaphragm. In stage III, lymphoma cells are in lymph nodes on both sides of the diaphragm, or in one part of a tissue or organ near these lymph node groups or in the spleen. In stage IV, lymphoma cells are found in several parts of at least one organ or tissue, or lymphoma cells are in an organ and in lymph nodes on the other side of the diaphragm. In addition to the Roman numeral staging designation, the stages of can also be described by letters A, B, E, and S, wherein A refers to patients without symptoms, B refers to patients with symptoms, E refers to patients in which lymphoma is found in tissues outside the lymph system, and S refers to patients in which lymphoma is found in the spleen.

Hodgkin's lymphoma is commonly treated with radiation therapy, chemotherapy, or hematopoietic stem cell transplantation. The most common therapy for non-Hodgkin's lymphoma is R-CHOP, which consists of four different chemotherapies (cyclophosphamide, doxorubicin, vincristine, and prenisolone) and rituximab (Rituxan®). Other therapies commonly used to treat NHL include other chemotherapeutic agents, radiation therapy, stem cell transplantation (autologous or allogeneic bone marrow transplantation), or biological therapy, such as immunotherapy. Other examples of biological therapeutic agents include, but are not limited to, rituximab (Rituxan®), tositumomab (Bexxar®), epratuzumab (LymphoCide®), and alemtuzumab (MabCampath®). The compositions and methods of the present invention may be administered in combination with any of the currently prescribed treatments for Hodgkin's lymphoma or non-Hodgkin's lymphoma.

BCMA expression has also been associated with Waldenstrom's macroglobulinemia (WM), also known as lymphoplasmacytic lymphoma (LPL). (See Elsawa et al., Blood. 2006, 107(7):2882-8). Waldenstrom's macroglobulinemia was previously considered to be related to multiple myeloma, but has more recently been classified as a subtype of non-Hodgkin's lymphoma. WM is characterized by uncontrolled B-cell lymphocyte proliferation, resulting in anemia and production of excess amounts of paraprotein, or immunoglobulin M (IgM), which thickens the blood and results in hyperviscosity syndrome. Other symptoms or signs of WM include fever, night sweats, fatigue, anemia, weight loss, lymphadenopathy or splenomegaly, blurred vision, dizziness, nose bleeds, bleeding gums, unusual bruises, renal impairment or failure, amyloidosis, or peripheral neuropathy.

Standard treatment for WM consists of chemotherapy, specifically with rituximab (Rituxan®). Other chemotherapeutic drugs can be used in combination, such as chlorambucil (Leukeran®), cyclophosphamide (Neosar®), fludarabine (Fludara®), cladribine (Leustatin®), vincristine, and/or thalidomide. Corticosteriods, such as prednisone, can also be administered in combination with the chemotherapy. Plasmapheresis, or plasma exchange, is commonly used throughout treatment of the patient to alleviate some symptoms by removing the paraprotein from the blood. In some cases, stem cell transplantation is an option for some patients.

Another example of a disease or disorder associated with BCMA expression is brain cancer. Specifically, expression of BCMA has been associated with astrocytoma or glioblastoma (See Deshayes et al, Oncogene. 2004, 23(17):3005-12, Pelekanou et al., PLoS One. 2013, 8(12):e83250). Astrocytomas are tumors that arise from astrocytes, which are a type of glial cell in the brain. Glioblastoma (also known as glioblastoma multiforme or GBM) is the most malignant form of astrocytoma, and is considered the most advanced stage of brain cancer (stage IV). There are two variants of glioblastoma: giant cell glioblastoma and gliosarcoma. Other astrocytomas include juvenile pilocytic astrocytoma (JPA), fibrillary astrocytoma, pleomorphic xantroastrocytoma (PXA), desembryoplastic neuroepithelial tumor (DNET), and anaplastic astrocytoma (AA).

Symptoms or signs associated with glioblastoma or astrocytoma include increased pressure in the brain, headaches, seizures, memory loss, changes in behavior, loss in movement or sensation on one side of the body, language dysfunction, cognitive impairments, visual impairment, nausea, vomiting, and weakness in the arms or legs.

Surgical removal of the tumor (or resection) is the standard treatment for removal of as much of the glioma as possible without damaging or with minimal damage to the normal, surrounding brain. Radiation therapy and/or chemotherapy are often used after surgery to suppress and slow recurrent disease from any remaining cancer cells or satellite lesions. Radiation therapy includes whole brain radiotherapy (conventional external beam radiation), targeted three-dimensional conformal radiotherapy, and targeted radionuclides. Chemotherapeutic agents commonly used to treat glioblastoma include temozolomide, gefitinib or erlotinib, and cisplatin. Angiogenesis inhibitors, such as Bevacizumab (Avastin®), are also commonly used in combination with chemotherapy and/or radiotherapy.

Supportive treatment is also frequently used to relieve neurological symptoms and improve neurologic function, and is administered in combination any of the cancer therapies described herein. The primary supportive agents include anticonvulsants and corticosteroids. Thus, the compositions and methods of the present invention may be used in combination with any of the standard or supportive treatments to treat a glioblastoma or astrocytoma.

Non-cancer related diseases and disorders associated with BCMA expression can also be treated by the compositions and methods disclosed herein. Examples of non-cancer related diseases and disorders associated with BCMA expression include, but are not limited to: viral infections; e.g., HIV, fungal infections, e.g., C. neoformans; irritable bowel disease; ulcerative colitis, and disorders related to mucosal immunity.

The BCMA-targeting agent (e.g., an anti-BCMA antibody molecule or a recombinant non-antibody protein that binds to BCMA) and GSI of the present invention may be administered either alone, or as a pharmaceutical composition in combination with diluents and/or with other components such as IL-2 or other cytokines or cell populations.

The present invention provides for compositions and methods for treating cancer. In one aspect, the cancer is a hematologic cancer including but not limited to a leukemia or a lymphoma. In one aspect, disclosed herein are methods of treating cancers and malignancies including, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further diseases associated with BCMA expression include, but are not limited to, e.g., atypical and/or non-classical cancers, malignancies, precancerous conditions or proliferative diseases expressing BCMA.

In embodiments, a composition described herein can be used to treat a disease including but not limited to a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

In embodiments, a composition described herein can be used to treat a disease including but not limited to a cancer, e.g., a cancer described herein, e.g., a prostate cancer (e.g., castrate-resistant or therapy-resistant prostate cancer, or metastatic prostate cancer), pancreatic cancer, or lung cancer.

The present invention also provides methods for inhibiting the proliferation or reducing a BCMA-expressing cell population, the methods comprising contacting a population of cells comprising a BMCA-expressing cell with a BCMA-targeting agent (e.g., an anti-BCMA antibody molecule or a recombinant non-antibody protein that binds to BCMA) and a GSI. In a specific aspect, the present invention provides methods for inhibiting the proliferation or reducing the population of cancer cells expressing BCMA, the methods comprising contacting the BCMA-expressing cancer cell population with a BCMA-targeting agent (e.g., an anti-BCMA antibody molecule or a recombinant non-antibody protein that binds to BCMA) and a GSI. In one aspect, the present invention provides methods for inhibiting the proliferation or reducing the population of cancer cells expressing BCMA, the methods comprising contacting the BMCA-expressing cancer cell population with a BCMA-targeting agent (e.g., an anti-BCMA antibody molecule or a recombinant non-antibody protein that binds to BCMA) and a GSI. In certain aspects, the methods reduce the quantity, number, amount or percentage of cells and/or cancer cells by at least 25%, at least 30%, at least 40%, at least 50%, at least 65%, at least 75%, at least 85%, at least 95%, or at least 99% in a subject with or an animal model for myeloid leukemia or another cancer associated with BCMA-expressing cells relative to a negative control. In one aspect, the subject is a human.

The present invention also provides methods for preventing, treating and/or managing a disease associated with BCMA-expressing cells (e.g., a hematologic cancer or atypical cancer expressing BCMA), the methods comprising administering to a subject in need a BCMA-targeting agent (e.g., an anti-BCMA antibody molecule or a recombinant non-antibody protein that binds to BCMA) and a GSI. In one aspect, the subject is a human. Non-limiting examples of disorders associated with BCMA-expressing cells include viral or fungal infections, and disorders related to mucosal immunity.

The present invention also provides methods for preventing, treating and/or managing a disease associated with BCMA-expressing cells, the methods comprising administering to a subject in need a BCMA-targeting agent (e.g., an anti-BCMA antibody molecule or a recombinant non-antibody protein that binds to BCMA) and a GSI. In one aspect, the subject is a human.

The present invention provides methods for preventing relapse of cancer associated with BCMA-expressing cells, the methods comprising administering to a subject in need thereof a BCMA-targeting agent (e.g., an anti-BCMA antibody molecule or a recombinant non-antibody protein that binds to BCMA) and a GSI. In one aspect, the methods comprise administering to the subject in need thereof an effective amount of a BCMA-targeting agent (e.g., an anti-BCMA antibody molecule or a recombinant non-antibody protein that binds to BCMA) and a GSI in combination with an effective amount of a third therapy.

### Combination Therapies

A BCMA-targeting agent (e.g., an anti-BCMA antibody molecule or a recombinant non-antibody protein that binds to BCMA), e.g., as disclosed herein, may be used in combination with a GSI, e.g., as disclosed herein, and/or with other known agents and therapies.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A BCMA-targeting agent (e.g., an anti-BCMA antibody molecule or a recombinant non-antibody protein that binds to BCMA) and a GSI can be administered simultaneously, in the same or in separate compositions, or sequentially. For sequential administration, the GSI can be administered first, and the BCMA-targeting agent (e.g., an anti-BCMA antibody molecule or a recombinant non-antibody protein that binds to BCMA) can be administered second, or the order of administration can be reversed.

The BCMA-targeting agent (e.g., an anti-BCMA antibody molecule or a recombinant non-antibody protein that binds to BCMA), GSI, and/or other therapeutic agents, procedures or modalities can be administered during periods of active disorder, or during a period of remission or less active disease. The BCMA-targeting agent (e.g., an anti-BCMA antibody molecule or a recombinant non-antibody protein that binds to BCMA) can be administered before the other treatment, concurrently with the treatment, post-treatment, or during remission of the disorder.

When administered in combination, the BCMA-targeting agent (e.g., an anti-BCMA antibody molecule or a recombinant non-antibody protein that binds to BCMA), GSI, and/or the additional agent (e.g., a third agent), or all, can be administered in an amount or dose that is higher, lower or the same than the amount or dosage of each agent used individually, e.g., as a monotherapy. In certain embodiments, the administered amount or dosage of the BCMA-targeting agent (e.g., an anti-BCMA antibody molecule or a recombinant non-antibody protein that binds to BCMA), GSI, and/or the additional agent (e.g., a third agent), or all, is lower (e.g., at least 20%, at least 30%, at least 40%, or at least 50%) than the amount or dosage of each agent used individually, e.g., as a monotherapy. In other embodiments, the amount or dosage of the BCMA-targeting agent (e.g., an anti-BCMA antibody molecule or a recombinant non-antibody protein that binds to BCMA), GSI, and/or the additional agent (e.g., a third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a BCMA-targeting agent (e.g., an anti-BCMA antibody molecule or a recombinant non-antibody protein that binds to BCMA) and/or GSI described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, irradiation, and peptide vaccine, such as that described in Izumoto et al. 2008 J Neurosurg 108:963-971.

In certain instances, a BCMA-targeting agent (e.g., an anti-BCMA antibody molecule or a recombinant non-antibody protein that binds to BCMA) and/or GSI of the present invention are combined with other therapeutic agents, such as other anti-cancer agents, anti-allergic agents, anti-nausea agents (or anti-emetics), pain relievers, cytoprotective agents, and combinations thereof. In one embodiment, a BCMA-targeting agent (e.g., an anti-BCMA antibody molecule or a recombinant non-antibody protein that binds to BCMA) and/or GSI described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab, obinutuzumab, ofatumumab, daratumumab, elotuzumab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (CytoxanR or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (AdriamycinR, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the compositions of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (TemodarR and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (BusulfexR and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 383), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In embodiments, a BCMA-targeting agent and a GSI described herein are administered to a subject in combination with fludarabine, cyclophosphamide, and/or rituximab. In embodiments, a BCMA-targeting agent and a GSI described herein are administered to a subject in combination with fludarabine, cyclophosphamide, and rituximab (FCR). In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the fludarabine is administered at a dosage of about 10-50 mg/m² (e.g., about 10-15, 15-20, 20-25, 25-30, 30-35, 35-40, 40-45, or 45-50 mg/m²), e.g., intravenously. In embodiments, the cyclophosphamide is administered at a dosage of about 200-300 mg/m² (e.g., about 200-225, 225-250, 250-275, or 275-300 mg/m²), e.g., intravenously. In embodiments, the rituximab is administered at a dosage of about 400-600 mg/m2 (e.g., 400-450, 450-500, 500-550, or 550-600 mg/m²), e.g., intravenously.

In embodiments, a BCMA-targeting agent and a GSI described herein are administered to a subject in combination with bendamustine and rituximab. In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the bendamustine is administered at a dosage of about 70-110 mg/m2 (e.g., 70-80, 80-90, 90-100, or 100-110 mg/m2), e.g., intravenously. In embodiments, the rituximab is administered at a dosage of about 400-600 mg/m2 (e.g., 400-450, 450-500, 500-550, or 550-600 mg/m²), e.g., intravenously.

In embodiments, a BCMA-targeting agent and a GSI described herein are administered to a subject in combination with rituximab, cyclophosphamide, doxorubicine, vincristine, and/or a corticosteroid (e.g., prednisone). In embodiments, a BCMA-targeting agent and a GSI described herein are administered to a subject in combination with rituximab, cyclophosphamide, doxorubicine, vincristine, and prednisone (R-CHOP). In embodiments, the subject has diffuse large B-cell lymphoma (DLBCL). In embodiments, the subject has nonbulky limited-stage DLBCL (e.g., comprises a tumor having a size/diameter of less than 7 cm). In embodiments, the subject is treated with radiation in combination with the R-CHOP. For example, the subject is administered R-CHOP (e.g., 1-6 cycles, e.g., 1, 2, 3, 4, 5, or 6 cycles of R-CHOP), followed by radiation. In some cases, the subject is administered R-CHOP (e.g., 1-6 cycles, e.g., 1, 2, 3, 4, 5, or 6 cycles of R-CHOP) following radiation.

In embodiments, a BCMA-targeting agent and a GSI described herein are administered to a subject in combination with etoposide, prednisone, vincristine, cyclophosphamide, doxorubicin, and/or rituximab. In embodiments, a BCMA-targeting agent and a GSI described herein are administered to a subject in combination with etoposide, prednisone, vincristine, cyclophosphamide, doxorubicin, and rituximab (EPOCH-R). In embodiments, a BCMA-targeting agent and a GSI described herein are administered to a subject in combination with dose-adjusted EPOCH-R (DA-EPOCH-R). In embodiments, the subject has a B cell lymphoma, e.g., a Myc-rearranged aggressive B cell lymphoma.

In embodiments, a BCMA-targeting agent and a GSI described herein are administered to a subject in combination with rituximab and/or lenalidomide. Lenalidomide ((RS)-3-(4-Amino-1-oxo 1,3-dihydro-2H-isoindol-2-yl)piperidine-2,6-dione) is an immunomodulator. In embodiments, a BCMA-targeting agent and a GSI described herein are administered to a subject in combination with rituximab and lenalidomide. In embodiments, the subject has follicular lymphoma (FL) or mantle cell lymphoma (MCL). In embodiments, the subject has FL and has not previously been treated with a cancer therapy. In embodiments, lenalidomide is administered at a dosage of about 10-20 mg (e.g., 10-15 or 15-20 mg), e.g., daily. In embodiments, rituximab is administered at a dosage of about 350-550 mg/m² (e.g., 350-375, 375-400, 400-425, 425-450, 450-475, or 475-500 mg/m²), e.g., intravenously.

In embodiments, a BCMA-targeting agent and a GSI described herein are administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+ HL. In embodiments, the subject has undergone an autologous stem cell transplant (ASCT). In embodiments, the subject has not undergone an ASCT. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In embodiments, a BCMA-targeting agent and a GSI described herein are administered to a subject in combination with brentuximab and dacarbazine or in combination with brentuximab and bendamustine. Dacarbazine is an alkylating agent with a chemical name of 5-(3,3-Dimethyl-1-triazenyl)imidazole-4-carboxamide. Bendamustine is an alkylating agent with a chemical name of 4-[5-[Bis(2-chloroethyl)amino]-1-methylbenzimidazol-2-yl]butanoic acid. In embodiments, the subject has Hodgkin's lymphoma (HL). In embodiments, the subject has not previously been treated with a cancer therapy. In embodiments, the subject is at least 60 years of age, e.g., 60, 65, 70, 75, 80, 85, or older. In embodiments, dacarbazine is administered at a dosage of about 300-450 mg/m² (e.g., about 300-325, 325-350, 350-375, 375-400, 400-425, or 425-450 mg/m²), e.g., intravenously. In embodiments, bendamustine is administered at a dosage of about 75-125 mg/m2 (e.g., 75-100 or 100-125 mg/m², e.g., about 90 mg/m²), e.g., intravenously. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In some embodiments, a BCMA-targeting agent and a GSI described herein are administered to a subject in combination with a CD20 inhibitor, e.g., an anti-CD20 antibody (e.g., an anti-CD20 mono- or bispecific antibody) or a fragment thereof. Exemplary anti-CD20 antibodies include but are not limited to rituximab, ofatumumab, ocrelizumab, veltuzumab, obinutuzumab, TRU-015 (Trubion Pharmaceuticals), ocaratuzumab, and Pro131921 (Genentech). See, e.g., Lim et al. Haematologica. 95.1(2010):135-43.

In some embodiments, the anti-CD20 antibody comprises rituximab. Rituximab is a chimeric mouse/human monoclonal antibody IgG1 kappa that binds to CD20 and causes cytolysis of a CD20 expressing cell, e.g., as described in accessdata.fda.gov/drugsatfda_docs/label/2010/103705s53111bl.pdf. In embodiments, a BCMA-targeting agent and a GSI described herein are administered to a subject in combination with rituximab. In embodiments, the subject has CLL or SLL.

In some embodiments, rituximab is administered intravenously, e.g., as an intravenous infusion. For example, each infusion provides about 500-2000 mg (e.g., about 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, or 1900-2000 mg) of rituximab. In some embodiments, rituximab is administered at a dose of 150 mg/m² to 750 mg/m², e.g., about 150-175 mg/m², 175-200 mg/m², 200-225 mg/m², 225-250 mg/m², 250-300 mg/m², 300-325 mg/m², 325-350 mg/m², 350-375 mg/m², 375-400 mg/m², 400-425 mg/m², 425-450 mg/m², 450-475 mg/m², 475-500 mg/m², 500-525 mg/m², 525-550 mg/m², 550-575 mg/m², 575-600 mg/m², 600-625 mg/m², 625-650 mg/m², 650-675 mg/m², or 675-700 mg/m², where m² indicates the body surface area of the subject. In some embodiments, rituximab is administered at a dosing interval of at least 4 days, e.g., 4, 7, 14, 21, 28, 35 days, or more. For example, rituximab is administered at a dosing interval of at least 0.5 weeks, e.g., 0.5, 1, 2, 3, 4, 5, 6, 7, 8 weeks, or more. In some embodiments, rituximab is administered at a dose and dosing interval described herein for a period of time, e.g., at least 2 weeks, e.g., at least 2, 3, 4, 5, 6, 7, 8, 9, 10, 11, 12, 13, 14, 15, 16, 17, 18, 19, 20 weeks, or greater. For example, rituximab is administered at a dose and dosing interval described herein for a total of at least 4 doses per treatment cycle (e.g., at least 4, 5, 6, 7, 8, 9, 10, 11, 12, 13, 14, 15, 16, or more doses per treatment cycle).

In some embodiments, the anti-CD20 antibody comprises ofatumumab. Ofatumumab is an anti-CD20 IgG1κ human monoclonal antibody with a molecular weight of approximately 149 kDa. For example, ofatumumab is generated using transgenic mouse and hybridoma technology and is expressed and purified from a recombinant murine cell line (NS0). See, e.g., accessdata.fda.gov/drugsatfda_docs/label/2009/1253261bl.pdf; and Clinical Trial Identifier number NCT01363128, NCT01515176, NCT01626352, and NCT01397591. In embodiments, a BCMA-targeting agent and a GSI described herein are administered to a subject in combination with ofatumumab. In embodiments, the subject has CLL or SLL.

In some embodiments, ofatumumab is administered as an intravenous infusion. For example, each infusion provides about 150-3000 mg (e.g., about 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1200, 1200-1400, 1400-1600, 1600-1800, 1800-2000, 2000-2200, 2200-2400, 2400-2600, 2600-2800, or 2800-3000 mg) of ofatumumab. In embodiments, ofatumumab is administered at a starting dosage of about 300 mg, followed by 2000 mg, e.g., for about 11 doses, e.g., for 24 weeks. In some embodiments, ofatumumab is administered at a dosing interval of at least 4 days, e.g., 4, 7, 14, 21, 28, 35 days, or more. For example, ofatumumab is administered at a dosing interval of at least 1 week, e.g., 1, 2, 3, 4, 5, 6, 7, 8, 9, 10, 11, 12, 24, 26, 28, 20, 22, 24, 26, 28, 30 weeks, or more. In some embodiments, ofatumumab is administered at a dose and dosing interval described herein for a period of time, e.g., at least 1 week, e.g., 1, 2, 3, 4, 5, 6, 7, 8, 9, 10, 11, 12, 13, 14, 15, 16, 17, 18, 19, 20, 22, 24, 26, 28, 30, 40, 50, 60 weeks or greater, or 1, 2, 3, 4, 5, 6, 7, 8, 9, 10, 11, 12 months or greater, or 1, 2, 3, 4, 5 years or greater. For example, ofatumumab is administered at a dose and dosing interval described herein for a total of at least 2 doses per treatment cycle (e.g., at least 2, 3, 4, 5, 6, 7, 8, 9, 10, 11, 12, 13, 14, 15, 16, 18, 20, or more doses per treatment cycle).

In some cases, the anti-CD20 antibody comprises ocrelizumab. Ocrelizumab is a humanized anti-CD20 monoclonal antibody, e.g., as described in Clinical Trials Identifier Nos. NCT00077870, NCT01412333, NCT00779220, NCT00673920, NCT01194570, and Kappos et al. Lancet. 19.378(2011):1779-87.

In some cases, the anti-CD20 antibody comprises veltuzumab. Veltuzumab is a humanized monoclonal antibody against CD20. See, e.g., Clinical Trial Identifier No. NCT00547066, NCT00546793, NCT01101581, and Goldenberg et al. Leuk Lymphoma. 51(5)(2010):747-55.

In some cases, the anti-CD20 antibody comprises GA101. GA101 (also called obinutuzumab or R05072759) is a humanized and glyco-engineered anti-CD20 monoclonal antibody. See, e.g., Robak. Curr. Opin. Investig. Drugs. 10.6(2009):588-96; Clinical Trial Identifier Numbers: NCT01995669, NCT01889797, NCT02229422, and NCT01414205; and accessdata.fda.gov/drugsatfda_docs/label/2013/125486s0001bl.pdf.

In some cases, the anti-CD20 antibody comprises AME-133v. AME-133v (also called LY2469298 or ocaratuzumab) is a humanized IgG1 monoclonal antibody against CD20 with increased affinity for the FcγRIIIa receptor and an enhanced antibody dependent cellular cytotoxicity (ADCC) activity compared with rituximab. See, e.g., Robak et al. BioDrugs 25.1(2011):13-25; and Forero-Torres et al. Clin Cancer Res. 18.5(2012):1395-403.

In some cases, the anti-CD20 antibody comprises PRO131921. PRO131921 is a humanized anti-CD20 monoclonal antibody engineered to have better binding to FcγRIIIa and enhanced ADCC compared with rituximab. See, e.g., Robak et al. BioDrugs 25.1(2011):13-25; and Casulo et al. Clin Immunol. 154.1(2014):37-46; and Clinical Trial Identifier No. NCT00452127.

In some cases, the anti-CD20 antibody comprises TRU-015. TRU-015 is an anti-CD20 fusion protein derived from domains of an antibody against CD20. TRU-015 is smaller than monoclonal antibodies, but retains Fc-mediated effector functions. See, e.g., Robak et al. BioDrugs 25.1(2011):13-25. TRU-015 contains an anti-CD20 single-chain variable fragment (scFv) linked to human IgG1 hinge, CH2, and CH3 domains but lacks CH1 and CL domains.

In some embodiments, an anti-CD20 antibody described herein is conjugated or otherwise bound to a therapeutic agent, e.g., a chemotherapeutic agent (e.g., cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein.

In embodiments, a BCMA-targeting agent and a GSI described herein are administered to a subject in combination with a B-cell lymphoma 2 (BCL-2) inhibitor (e.g., venetoclax, also called ABT-199 or GDC-0199); and/or rituximab. In embodiments, a BCMA-targeting agent and a GSI described herein are administered to a subject in combination with venetoclax and rituximab. Venetoclax is a small molecule that inhibits the anti-apoptotic protein, BCL-2. The structure of venetoclax (4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) is shown below.

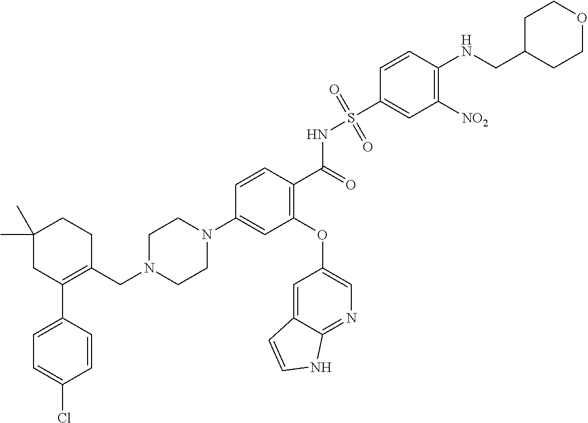 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In embodiments, the subject has CLL. In embodiments, the subject has relapsed CLL, e.g., the subject has previously been administered a cancer therapy. In embodiments, venetoclax is administered at a dosage of about 15-600 mg (e.g., 15-20, 20-50, 50-75, 75-100, 100-200, 200-300, 300-400, 400-500, or 500-600 mg), e.g., daily. In embodiments, rituximab is administered at a dosage of about 350-550 mg/m2 (e.g., 350-375, 375-400, 400-425, 425-450, 450-475, or 475-500 mg/m2), e.g., intravenously, e.g., monthly.

Without being bound by theory, it is believed that in some cancers, B cells (e.g., B regulatory cells) can suppress T cells. Further, it is believed that a combination of oxiplatin and the B cell depleting agent may reduce tumor size and/or eliminate tumors in a subject. In some embodiments, a BCMA-targeting agent and a GSI described herein are administered in combination with a B cell depleting agent (e.g., a CD19 CAR-expressing cell, a CD20 CAR-expressing cell, rituximab, ocrelizumab, epratuzumab, or belimumab) and oxiplatin. In embodiments, the cancer cell can be CD19 negative or CD19 positive; or BCMA negative or BMCA positive. In embodiments, a BCMA-targeting agent and a GSI described herein are administered in combination with a B cell depleting agent and oxiplatin to treat a cancer, e.g., a cancer described herein, e.g., solid cancer, e.g., prostate cancer, pancreatic cancer, or lung cancer.

In embodiments, a BCMA-targeting agent described herein may deplete B cells (e.g., B cells having a plasma cell-like phenotype, e.g., that express BCMA, CD19, and/or CD20) in a subject. In embodiments, the B cell can be CD19 negative or CD19 positive; or BCMA negative or BMCA positive. In some embodiments, a BCMA-targeting agent and a GSI described herein are administered in combination with oxiplatin. In embodiments, a BCMA-targeting agent and a GSI described herein are administered in combination with oxiplatin to treat a cancer, e.g., solid cancer, e.g., prostate cancer, pancreatic cancer, or lung cancer. In some embodiments, a BCMA-targeting agent and a GSI described herein are administered in combination with an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In some embodiments, the oncolytic virus is a virus, e.g., recombinant oncolytic virus, described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1, incorporated herein by reference in its entirety. In embodiments, the recombinant oncolytic virus, e.g., oncolytic NDV, comprises a pro-apoptotic protein (e.g., apoptin), a cytokine (e.g., GM-CSF, interferon-gamma, interleukin-2 (IL-2), tumor necrosis factor-alpha), an immunoglobulin (e.g., an antibody against ED-B firbonectin), tumor associated antigen, a bispecific adapter protein (e.g., bispecific antibody or antibody fragment directed against NDV HN protein and a T cell co-stimulatory receptor, such as CD3 or CD28; or fusion protein between human IL-2 and single chain antibody directed against NDV HN protein). See, e.g., Zamarin et al. Future Microbiol. 7.3(2012):347-67, incorporated herein by reference in its entirety. In some embodiments, the oncolytic virus is a chimeric oncolytic NDV described in U.S. Pat. No. 8,591,881 B2, US 2012/0122185 A1, or US 2014/0271677 A1, each of which is incorporated herein by reference in their entireties.

In some embodiments, the oncolytic virus comprises a conditionally replicative adenovirus (CRAd), which is designed to replicate exclusively in cancer cells. See, e.g., Alemany et al. Nature Biotechnol. 18(2000):723-27. In some embodiments, an oncolytic adenovirus comprises one described in Table 1 on page 725 of Alemany et al., incorporated herein by reference in its entirety.

Exemplary oncolytic viruses include but are not limited to the following:
Group B Oncolytic Adenovirus (ColoAd1) (PsiOxus Therapeutics Ltd.) (see, e.g., Clinical Trial Identifier: NCT02053220);
ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);
VCN-01, which is a genetically modified oncolytic human adenovirus encoding human PH20 hyaluronidase (VCN Biosciences, S.L.) (see, e.g., Clinical Trial Identifiers: NCT02045602 and NCT02045589);
Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Catalá d'Oncologia) (see, e.g., Clinical Trial Identifier: NCT01864759);
Celyvir, which comprises bone marrow-derived autologous mesenchymal stem cells (MSCs) infected with ICOVIR5, an oncolytic adenovirus (Hospital Infantil Universitario Nino Jesns, Madrid, Spain/Ramon Alemany) (see, e.g., Clinical Trial Identifier: NCT01844661);
CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); or
DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmucosally, orally, intranasally, or via pulmonary administration.

In an embodiment, a BCMA-targeting agent and a GSI described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to administration of a BCMA-targeting agent described herein reduces the number of unwanted immune cells (e.g., Tregs) in the tumor microenvironment and reduces the subject's risk of relapse. In one embodiment, a BCMA-targeting agent and a GSI described herein are administered to a subject in combination with a molecule targeting GITR and/or modulating GITR functions, such as a GITR agonist and/or a GITR antibody molecule that depletes regulatory T cells (Tregs). In embodiments, a BCMA-targeting agent and a GSI described herein are administered to a subject in combination with cyclophosphamide. In one embodiment, the GITR binding molecules and/or molecules modulating GITR functions (e.g., GITR agonist and/or Treg depleting GITR antibodies) are administered prior to administration of the BCMA-targeting agent. In embodiments, cyclophosphamide is administered to the subject prior to administration of the BCMA-targeting agent. In embodiments, cyclophosphamide and an anti-GITR antibody are administered to the subject prior to administration of the BCMA-targeting agent. In one embodiment, the subject has cancer (e.g., a solid cancer or a hematological cancer such as multiple myeloma, ALL or CLL). In an embodiment, the subject has CLL. In embodiments, the subject has multiple myeloma. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No. WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a BCMA-targeting agent and a GSI described herein are administered to a subject in combination with an mTOR inhibitor, e.g., an mTOR inhibitor described herein, e.g., a rapalog such as everolimus.

In one embodiment, a BCMA-targeting agent and a GSI described herein are administered to a subject in combination with a GITR agonist, e.g., a GITR agonist described herein.

In one embodiment, a BCMA-targeting agent and a GSI described herein are administered to a subject in combination with a protein tyrosine phosphatase inhibitor, e.g., a protein tyrosine phosphatase inhibitor described herein. In one embodiment, the protein tyrosine phosphatase inhibitor is an SHP-1 inhibitor, e.g., an SHP-1 inhibitor described herein, such as, e.g., sodium stibogluconate. In one embodiment, the protein tyrosine phosphatase inhibitor is an SHP-2 inhibitor.

In one embodiment, a BCMA-targeting agent and a GSI described herein can be used in combination with a kinase inhibitor. In one embodiment, the kinase inhibitor is a CDK4 inhibitor, e.g., a CDK4 inhibitor described herein, e.g., a CDK4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo [3,4-d]pyrimidine. The MNK inhibitor can be, e.g., a MNK1a, MNK1b, MNK2a and/or MNK2b inhibitor. In one embodiment, the kinase inhibitor is a dual PI3K/mTOR inhibitor described herein, such as, e.g., PF-04695102. In one embodiment, the kinase inhibitor is a DGK inhibitor, e.g., a DGK inhibitor described herein, such as, e.g., DGKinh1 (D5919) or DGKinh2 (D5794).

In one embodiment, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In one embodiment, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In one embodiment, 1, 2, 3, 4, 5, 6, 7, 8, 9, 10, 11, 12 or more cycles of palbociclib are administered.

In embodiments, a BCMA-targeting agent and a GSI described herein are administered to a subject in combination with a cyclin-dependent kinase (CDK) 4 or 6 inhibitor, e.g., a CDK4 inhibitor or a CDK6 inhibitor described herein. In embodiments, a BCMA-targeting agent and a GSI described herein are administered to a subject in combination with a CDK4/6 inhibitor (e.g., an inhibitor that targets both CDK4 and CDK6), e.g., a CDK4/6 inhibitor described herein. In an embodiment, the subject has MCL. MCL is an aggressive cancer that is poorly responsive to currently available therapies, i.e., essentially incurable. In many cases of MCL, cyclin D1 (a regulator of CDK4/6) is expressed (e.g., due to chromosomal translocation involving immunoglobulin and Cyclin D1 genes) in MCL cells. Thus, without being bound by theory, it is thought that MCL cells are highly sensitive to CDK4/6 inhibition with high specificity (i.e., minimal effect on normal immune cells). CDK4/6 inhibitors alone have had some efficacy in treating MCL, but have only achieved partial remission with a high relapse rate. An exemplary CDK4/6 inhibitor is LEE011 (also called ribociclib), the structure of which is shown below.

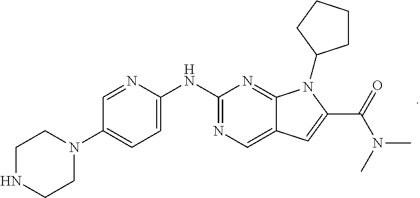 [SMILES: CN(C)C(=O)c1cc2cnc(Nc3ccc(N4CCNCC4)cn3)nc2n1C1CCCC1]

Without being bound by theory, it is believed that administration of a BCMA-targeting agent and a GSI described herein with a CDK4/6 inhibitor (e.g., LEE011 or other CDK4/6 inhibitor described herein) can achieve higher responsiveness, e.g., with higher remission rates and/or lower relapse rates, e.g., compared to a CDK4/6 inhibitor alone.

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In a preferred embodiment, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765). In embodiments, a BCMA-targeting agent and a GSI described herein are administered to a subject in combination with a BTK inhibitor (e.g., ibrutinib). In embodiments, a BCMA-targeting agent and a GSI described herein are administered to a subject in combination with ibrutinib (also called PCI-32765). The structure of ibrutinib (1-[(3R)-3-[4-Amino-3-(4-phenoxyphenyl)-1H-pyrazolo[3,4-d]pyrimidin-1-yl]piperidin-1-yl]prop-2-en-1-one) is shown below.

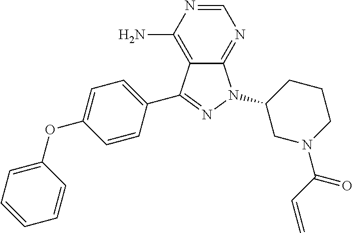 [SMILES: C=CC(=O)N1CCC[C@@H](n2nc(-c3ccc(Oc4ccccc4)cc3)c3c(N)ncnc32)C1]

In embodiments, the subject has CLL, mantle cell lymphoma (MCL), or small lymphocytic lymphoma (SLL). For example, the subject has a deletion in the short arm of chromosome 17 (del(17p), e.g., in a leukemic cell). In other examples, the subject does not have a del(17p). In embodiments, the subject has relapsed CLL or SLL, e.g., the subject has previously been administered a cancer therapy (e.g., previously been administered one, two, three, or four prior cancer therapies). In embodiments, the subject has refractory CLL or SLL. In other embodiments, the subject has follicular lymphoma, e.g., relapse or refractory follicular lymphoma. In some embodiments, ibrutinib is administered at a dosage of about 300-600 mg/day (e.g., about 300-350, 350-400, 400-450, 450-500, 500-550, or 550-600 mg/day, e.g., about 420 mg/day or about 560 mg/day), e.g., orally. In embodiments, the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of ibrutinib are administered. In some embodiments, ibrutinib is administered in combination with rituximab. See, e.g., Burger et al. (2013) Ibrutinib In Combination With Rituximab (iR) Is Well Tolerated and Induces a High Rate Of Durable Remissions In Patients With High-Risk Chronic Lymphocytic Leukemia (CLL): New, Updated Results Of a Phase II Trial In Patients, Abstract 675 presented at 55^{th} ASH Annual Meeting and Exposition, New Orleans, LA 7-10 December Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses.

In some embodiments of the methods, uses, and compositions herein, the BTK inhibitor is a BTK inhibitor described in International Application WO/2015/079417, which is herein incorporated by reference in its entirety. For instance, in some embodiments, the BTK inhibitor is a compound of formula (I) or a pharmaceutically acceptable salt thereof;

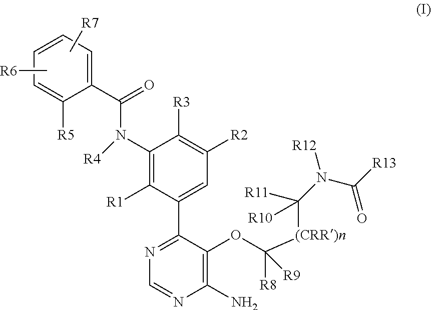

wherein,
R1 is hydrogen, C1-C6 alkyl optionally substituted by hydroxy;
R2 is hydrogen or halogen;
R3 is hydrogen or halogen;
R4 is hydrogen;
R5 is hydrogen or halogen;
or R4 and R5 are attached to each other and stand for a bond, —CH2-, —CH2-CH2-, —CH═CH—, —CH═CH—CH2-; —CH2-CH═CH—; or —CH2-CH2-CH2-;
R6 and R7 stand independently from each other for H, C1-C6 alkyl optionally substituted by hydroxyl, C3-C6 cycloalkyl optionally substituted by halogen or hydroxy, or halogen;
R8, R9, R, R′, R10 and R11 independently from each other stand for H, or C1-C6 alkyl optionally substituted by C1-C6 alkoxy; or any two of R8, R9, R, R′, R10 and R11 together with the carbon atom to which they are bound may form a 3-6 membered saturated carbocyclic ring;
R12 is hydrogen or C1-C6 alkyl optionally substituted by halogen or C1-C6 alkoxy; or R12 and any one of R8, R9, R, R′, R10 or R11 together with the atoms to which they are bound may form a 4, 5, 6 or 7 membered azacyclic ring, which ring may optionally be substituted by halogen, cyano, hydroxyl, C1-C6 alkyl or C1-C6 alkoxy;
n is 0 or 1; and
R13 is C2-C6 alkenyl optionally substituted by C1-C6 alkyl, C1-C6 alkoxy or N,N-di-C1-C6 alkyl amino; C2-C6 alkynyl optionally substituted by C1-C6 alkyl or C1-C6 alkoxy; or C2-C6 alkylenyl oxide optionally substituted by C1-C6 alkyl.

In some embodiments, the BTK inhibitor of Formula I is chosen from: N-(3-(5-((1-Acryloylazetidin-3-yl)oxy)-6-aminopyrimidin-4-yl)-5-fluoro-2-methylphenyl)-4-cyclopropyl-2-fluorobenzamide; (E)-N-(3-(6-Amino-5-((1-(but-2-enoyl)azetidin-3-yl)oxy)pyrimidin-4-yl)-5-fluoro-2-methylphenyl)-4-cyclopropyl-2-fluorobenzamide; N-(3-(6-Amino-5-((1-propioloylazetidin-3-yl)oxy)pyrimidin-4-yl)-5-fluoro-2-methylphenyl)-4-cyclopropyl-2-fluorobenzamide; N-(3-(6-Amino-5-((1-(but-2-ynoyl)azetidin-3-yl)oxy)pyrimidin-4-yl)-5-fluoro-2-methylphenyl)-4-cyclopropyl-2-fluorobenzamide; N-(3-(5-((1-Acryloylpiperidin-4-yl)oxy)-6-aminopyrimidin-4-yl)-5-fluoro-2-methylphenyl)-4-cyclopropyl-2-fluorobenzamide; N-(3-(6-Amino-5-(2-(N-methylacrylamido)ethoxy)pyrimidin-4-yl)-5-fluoro-2-methylphenyl)-4-cyclopropyl-2-fluorobenzamide; (E)-N-(3-(6-Amino-5-(2-(N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylpropiolamido)ethoxy)pyrimidin-4-yl)-5-fluoro-2-methylphenyl)-4-cyclopropyl-2-fluorobenzamide; (E)-N-(3-(6-Amino-5-(2-(4-methoxy-N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylbut-2-ynamido)ethoxy)pyrimidin-4-yl)-5-fluoro-2-methylphenyl)-4-cyclopropyl-2-fluorobenzamide; N-(2-((4-Amino-6-(3-(4-cyclopropyl-2-fluorobenzamido)-5-fluoro-2-methylphenyl)pyrimidin-5-yl)oxy)ethyl)-N-methyloxirane-2-carboxamide; N-(2-((4-Amino-6-(3-(6-cyclopropyl-8-fluoro-1-oxoisoquinolin-2(1H)-yl)phenyl)pyrimidin-5-yl)oxy)ethyl)-N-methylacrylamide; N-(3-(5-(2-Acrylamidoethoxy)-6-aminopyrimidin-4-yl)-5-fluoro-2-methylphenyl)-4-cyclopropyl-2-fluorobenzamide; N-(3-(6-Amino-5-(2-(N-ethylacrylamido)ethoxy)pyrimidin-4-yl)-5-fluoro-2-methylphenyl)-4-cyclopropyl-2-fluorobenzamide; N-(3-(6-Amino-5-(2-(N-(2-fluoroethyl)acrylamido)ethoxy)pyrimidin-4-yl)-5-fluoro-2-methylphenyl)-4-cyclopropyl-2-fluorobenzamide; N-(3-(5-((1-Acrylamidocyclopropyl)methoxy)-6-aminopyrimidin-4-yl)-5-fluoro-2-methylphenyl)-4-cyclopropyl-2-fluorobenzamide; (S)-N-(3-(5-(2-Acrylamidopropoxy)-6-aminopyrimidin-4-yl)-5-fluoro-2-methylphenyl)-4-cyclopropyl-2-fluorobenzamide; (S)-N-(3-(6-Amino-5-(2-(but-2-ynamido)propoxy)pyrimidin-4-yl)-5-fluoro-2-methylphenyl)-4-cyclopropyl-2-fluorobenzamide; (S)-N-(3-(6-Amino-5-(2-(N-methylacrylamido)propoxy)pyrimidin-4-yl)-5-fluoro-2-methylphenyl)-4-cyclopropyl-2-fluorobenzamide; (S)-N-(3-(6-Amino-5-(2-(N-methylbut-2-ynamido)propoxy)pyrimidin-4-yl)-5-fluoro-2-methylphenyl)-4-cyclopropyl-2-fluorobenzamide; N-(3-(6-Amino-5-(3-(N-methylacrylamido)propoxy)pyrimidin-4-yl)-5-fluoro-2-methylphenyl)-4-cyclopropyl-2-fluorobenzamide; (S)-N-(3-(5-((1-Acryloylpyrrolidin-2-yl)methoxy)-6-aminopyrimidin-4-yl)-5-fluoro-2-methylphenyl)-4-cyclopropyl-2-fluorobenzamide; (S)-N-(3-(6-Amino-5-((1-(but-2-ynoyl)pyrrolidin-2-yl)methoxy)pyrimidin-4-yl)-5-fluoro-2-methylphenyl)-4-cyclopropyl-2-fluorobenzamide; (S)-2-(3-(5-((1-Acryloylpyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(2-((4-Amino-6-(3-(6-cyclopropyl-1-oxo-3,4-dihydroisoquinolin-2(1H)-yl)-5-fluoro-2-(hydroxymethyl)phenyl)pyrimidin-5-yl)oxy)ethyl)-N-methylacrylamide; N-(3-(5-(((2S,4R)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; 2-(3-(5-(((2S,4R)-1-Acryloyl-4-methoxypyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(3-(5-(((2S,4S)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4S)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; N-(3-(5-(((2S,4R)-1-Acryloyl-4-fluoropyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-fluoropyrrolidin-2-yl)methoxy)pyrimidin-4-yl)-5-fluoro-2-methylphenyl)-4-cyclopropyl-2-fluorobenzamide; (S)-N-(3-(5-((1-Acryloylazetidin-2-yl)methoxy)-6-aminopyrimidin-4-yl)-5-fluoro-2-methylphenyl)-4-cyclopropyl-2-fluorobenzamide; (S)-N-(3-(6-Amino-5-((1-propioloylazetidin-2-yl)methoxy)pyrimidin-4-yl)-5-fluoro-2-methylphenyl)-4-cyclopropyl-2-fluorobenzamide; (S)-2-(3-(5-((1-Acryloylazetidin-2-yl)methoxy)-6-aminopyrimidin-4-yl)-5-fluoro-2-(hydroxymethyl)phenyl)-6-cyclopropyl-3,4-dihydroisoquinolin-1(2H)-one; (R)-N-(3-(5-((1-Acryloylazetidin-2-yl)methoxy)-6-aminopyrimidin-4-yl)-5-fluoro-2-methylphenyl)-4-cyclopropyl-2-fluorobenzamide; (R)-N-(3-(5-((1-Acryloylpiperidin-3-yl)methoxy)-6-aminopyrimidin-4-yl)-5-fluoro-2-methylphenyl)-4-cyclopropyl-2-fluorobenzamide; N-(3-(5-(((2R,3S)-1-Acryloyl-3-methoxypyrrolidin-2-yl)methoxy)-6-aminopyrimidin-4-yl)-5-fluoro-2-methylphenyl)-4-cyclopropyl-2-fluorobenzamide; N-(3-(5-(((2S,4R)-1-Acryloyl-4-cyanopyrrolidin-2-yl)methoxy)-6-aminopyrimidin-4-yl)-5-fluoro-2-methylphenyl)-4-cyclopropyl-2-fluorobenzamide; or N-(3-(5-(((2S,4S)-1-Acryloyl-4-cyanopyrrolidin-2-yl)methoxy)-6-aminopyrimidin-4-yl)-5-fluoro-2-methylphenyl)-4-cyclopropyl-2-fluorobenzamide.

Unless otherwise provided, the chemical terms used above in describing the BTK inhibitor of Formula I are used according to their meanings as set out in International Application WO/2015/079417, which is herein incorporated by reference in its entirety.

In one embodiment, the kinase inhibitor is an mTOR inhibitor selected from temsirolimus; ridaforolimus (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 383), inner salt (SF1126); and XL765.

In one embodiment, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of rapamycin are administered. In one embodiment, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In one embodiment, 1, 2, 3, 4, 5, 6, 7, 8, 9, 10, 11, 12 or more cycles of everolimus are administered.

In one embodiment, the kinase inhibitor is an MNK inhibitor selected from CGP052088; 4-amino-3-(p-fluorophenylamino)-pyrazolo [3,4-d]pyrimidine (CGP57380); cercosporamide; ETC-1780445-2; and 4-amino-5-(4-fluoroanilino)-pyrazolo [3,4-d]pyrimidine.

In embodiments, a BCMA-targeting agent and a GSI described herein are administered to a subject in combination with a phosphoinositide 3-kinase (PI3K) inhibitor (e.g., a PI3K inhibitor described herein, e.g., idelalisib or duvelisib) and/or rituximab. In embodiments, a BCMA-targeting agent and a GSI described herein are administered to a subject in combination with idelalisib and rituximab. In embodiments, a BCMA-targeting agent and a GSI described herein are administered to a subject in combination with duvelisib and rituximab. Idelalisib (also called GS-1101 or CAL-101; Gilead) is a small molecule that blocks the delta isoform of PI3K. The structure of idelalisib (5-Fluoro-3-phenyl-2-[(1S)-1-(7H-purin-6-ylamino)propyl]-4(3H)-quinazolinone) is shown below.

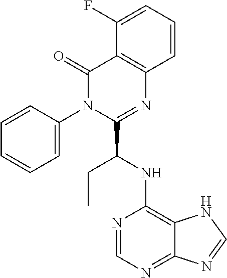 [SMILES: CC[C@H](Nc1ncnc2nc[nH]c12)c1nc2cccc(F)c2c(=O)n1-c1ccccc1]

Duvelisib (also called IPI-145; Infinity Pharmaceuticals and Abbvie) is a small molecule that blocks PI3K-δ,γ. The structure of duvelisib (8-Chloro-2-phenyl-3-[(1S)-1-(9H-purin-6-ylamino)ethyl]-1(2H)-isoquinolinone) is shown below.

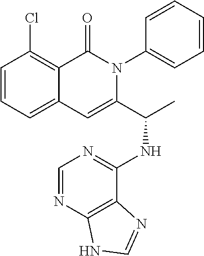 [SMILES: C[C@H](Nc1ncnc2[nH]cnc12)c1cc2cccc(Cl)c2c(=O)n1-c1ccccc1]

In embodiments, the subject has CLL. In embodiments, the subject has relapsed CLL, e.g., the subject has previously been administered a cancer therapy (e.g., previously been administered an anti-CD20 antibody or previously been administered ibrutinib). For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the subject has a deletion in the long arm of chromosome 11 (del(11 q)). In other embodiments, the subject does not have a del(11 q). In embodiments, idelalisib is administered at a dosage of about 100-400 mg (e.g., 100-125, 125-150, 150-175, 175-200, 200-225, 225-250, 250-275, 275-300, 325-350, 350-375, or 375-400 mg), e.g., BID. In embodiments, duvelisib is administered at a dosage of about 15-100 mg (e.g., about 15-25, 25-50, 50-75, or 75-100 mg), e.g., twice a day. In embodiments, rituximab is administered at a dosage of about 350-550 mg/m² (e.g., 350-375, 375-400, 400-425, 425-450, 450-475, or 475-500 mg/m²), e.g., intravenously.

In embodiments, a BCMA-targeting agent and a GSI described herein are administered to a subject in combination with an anaplastic lymphoma kinase (ALK) inhibitor. Exemplary ALK kinases include but are not limited to crizotinib (Pfizer), ceritinib (Novartis), alectinib (Chugai), brigatinib (also called AP26113; Ariad), entrectinib (Ignyta), PF-06463922 (Pfizer), TSR-OIl (Tesaro) (see, e.g., Clinical Trial Identifier No. NCT02048488), CEP-37440 (Teva), and X-396 (Xcovery). In some embodiments, the subject has a solid cancer, e.g., a solid cancer described herein, e.g., lung cancer.

The chemical name of crizotinib is 3-[(1R)-1-(2,6-dichloro-3-fluorophenyl)ethoxy]-5-(1-piperidin-4-ylpyrazol-4-yl)pyridin-2-amine. The chemical name of ceritinib is 5-Chloro-N²-[2-isopropoxy-5-methyl-4-(4-piperidinyl)phenyl]-N⁴-[2-(isopropylsulfonyl)phenyl]-2,4-pyrimidinediamine. The chemical name of alectinib is 9-ethyl-6,6-dimethyl-8-(4-morpholinopiperidin-1-yl)-11-oxo-6,11-dihydro-5H-benzo[b]carbazole-3-carbonitrile. The chemical name ofbrigatinib is 5-Chloro-N²-{4-[4-(dimethylamino)-1-piperidinyl]-2-methoxyphenyl}-N⁴-[2-(dimethylphosphoryl)phenyl]-2,4-pyrimidinediamine. The chemical name of entrectinib is N-(5-(3,5-difluorobenzyl)-1H-indazol-3-yl)-4-(4-methylpiperazin-1-yl)-2-((tetrahydro-2H-pyran-4-yl)amino)benzamide. The chemical name of PF-06463922 is (10R)-7-Amino-12-fluoro-2,10,16-trimethyl-15-oxo-10,15,16,17-tetrahydro-2H-8,4-(metheno)pyrazolo[4,3-h][2,5,11]-benzoxadiazacyclotetradecine-3-carbonitrile. The chemical structure of CEP-37440 is (S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide. The chemical name of X-396 is (R)-6-amino-5-(1-(2,6-dichloro-3-fluorophenyl)ethoxy)-N-(4-(4-methylpiperazine-1-carbonyl)phenyl)pyridazine-3-carboxamide.

In one embodiment, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present invention may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In one aspect, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In one embodiment, a BCMA-targeting agent and a GSI described herein are administered to a subject in combination with a biphosphonate, e.g., Pamidronate (Aredia®); Zoledronic acid or Zoledronate (Zometa®, Zomera®, Aclasta®, or Reclast®); Alendronate (Fosamax®); Risedronate (Actonel®); Ibandronate (Boniva®); Clondronate (Bonefos®); Etidronate (Didronel®); Tiludronate (Skelid®); Pamidronate (Aredia®); Neridronate (Nerixia®); Strontiun ranelate (Protelos®, or Protos®); and Teriparatide (Forteo®).

In one embodiment, a BCMA-targeting agent and a GSI described herein are administered to a subject in combination with a corticosteroid, e.g., dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

In one embodiment, a BCMA-targeting agent and a GSI described herein are administered to a subject in combination with an immunomodulator, e.g., Afutuzumab (available from Roche®); Pegfilgrastim (Neulasta®); Lenalidomide (CC-5013, Revlimid®); Thalidomide (Thalomid®), Actimid (CC4047); and IRX-2 (mixture of human cytokines including interleukin 1, interleukin 2, and interferon γ, CAS 951209-71-5, available from IRX Therapeutics.

In one embodiment, a BCMA-targeting agent and a GSI described herein are administered to a subject in combination with a proteasome inhibitor, e.g., Bortezomib (Velcade®); Ixazomib citrate (MLN9708, CAS 1201902-80-8); Danoprevir (RG7227, CAS 850876-88-9); Ixazomib (MLN2238, CAS 1072833-77-2); and (S)-N-[(phenylmethoxy)carbonyl]-L-leucyl-N-(1-formyl-3-methylbutyl)-L-Leucinamide (MG-132, CAS 133407-82-6).

In one embodiment, a BCMA-targeting agent and a GSI described herein are administered to a subject in combination with a vascular endothelial growth factor (VEGF) receptor, e.g., Bevacizumab (Avastin®), axitinib (Inlyta®); Brivanib alaninate (BMS-582664, (S)-((R)-1-(4-(4-Fluoro-2-methyl-1H-indol-5-yloxy)-5-methylpyrrolo[2,1-J][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aa,5p,6aa)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

In one embodiment, a BCMA-targeting agent and a GSI described herein are administered to a subject in combination with a CD20 antibody or a conjugate thereof, e.g., Rituximab (Riuxan® and MabThera®); and Tositumomab (Bexxar®); and Ofatumumab (Arzerra®), Ibritumomab tiuxetan (Zevalin®); and Tositumomab.

In one embodiment, a BCMA-targeting agent and a GSI described herein are administered to a subject in combination with an anticonvulsant, e.g., Anticonvulsants (antiepileptic or antiseizure drugs): aldehydes, e.g., paraldehyde; aromatic allylic alcohols, e.g., stiripentol (Diacomit®); barbiturates, e.g., phenobarbital (Luminal®), methylphenobarbital (Mebaral®), barbexaclone (Maliasin®), benzodiazepines, e.g., clobazam (Onfi®), clonazepam (Klonopin®), clorazepate (Tranxene® and Novo-Clopate®), diazepam (Valium®, Lembrol®, Diastat®), midazolam (Versed®), lorazepam (Ativan® and Orfidal®), nitrazepam (Alodorm®, Arem®, Insoma®), temazepam (Restoril®, Normison®), nimetzepam (Erimin®), bromides, e.g., potassium bromide; carbamates, e.g., felbamate (Felbatol®); carboxamides, e.g., carbamazepine (Tegretol®, Equetro®), oxcarbazepine (Trileptal®, Oxcarb®), eslicarbazepine acetate (Aptiom®); fatty acids, e.g., valproates (valproic acid, sodium valproate, divalproex sodium), vigabatrin (Sabril®), progabide (Gabren®), tiagabine (Gabitril®); fructose derivatives, e.g., topiramate (Topamax®); GABA analogs, e.g., gabapentin (Neurontin®), pregabalin (Lyrica®); hydantoins, e.g., ethotoin (Peganone®), phenytoin (Dilantin®), mephenytoin (Mesantoin®), fosphenytoin (Cerebyx®, Prodilantin®); oxazolidinediones, e.g., paramethadione (Paradione®), trimethadione (Tridione®); propionates, e.g., beclamide (Choracon®, Hibicon®, Posedrine®); pyrimidinediones, e.g., primidone (Mysoline®); pyrrolidines, e.g., brivaracetam, levetiracetam, seletracetam (Keppra®); succinimides, e.g., ethosuximide (Zarontin®), phensuximide (Milontin®), mesuximide (Celontin®, Petinutin®); sulfonamides, e.g., acetazolamide (Diamox®), sultiame (Ospolot®), methazolamide (Neptazane®), zonisamide (Zonegran®); triazines, e.g., lamotrigine (Lamictal®); ureas, e.g., pheneturide, phenacemide (Phenurone®); valproylamides (amide derivaties of valproate), e.g., valpromide (Depamide®), valnoctamide; AMPA receptor antagonist, e.g., perampanel (Fycompa®).

In embodiments, a BCMA-targeting agent and a GSI described herein are administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by theory, it is thought that a decrease in L-tryptophan (e.g., catalyzed by IDO) results in an immunosuppressive milieu by inducing T-cell anergy and apoptosis. Thus, without being bound by theory, it is thought that an IDO inhibitor can enhance the efficacy of a CAR-expressing cell described herein, e.g., by decreasing the suppression or death of a CAR-expressing immune cell. In embodiments, the subject has a solid tumor, e.g., a solid tumor described herein, e.g., prostatic, colorectal, pancreatic, cervical, gastric, ovarian, head, or lung cancer. Exemplary inhibitors of IDO include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255)

In embodiments, a BCMA-targeting agent and a GSI described herein are administered to a subject in combination with a modulator of myeloid-derived suppressor cells (MDSCs). MDSCs accumulate in the periphery and at the tumor site of many solid tumors. These cells suppress T cell responses. Without being bound by theory, it is thought that administration of a MDSC modulator enhances the efficacy of a BCMA-targeting agent described herein. In an embodiment, the subject has a solid tumor, e.g., a solid tumor described herein, e.g., glioblastoma. Exemplary modulators of MDSCs include but are not limited to MCS 110 and BLZ945. MCS 110 is a monoclonal antibody (mAb) against macrophage colony-stimulating factor (M-CSF). See, e.g., Clinical Trial Identifier No. NCT00757757. BLZ945 is a small molecule inhibitor of colony stimulating factor 1 receptor (CSF1R). See, e.g., Pyonteck et al. Nat. Med. 19(2013):1264-72. The structure of BLZ945 is shown below.

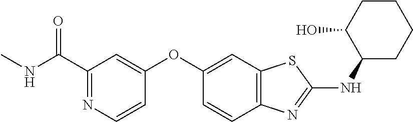 [SMILES: CNC(=O)c1cc(Oc2ccc3nc(N[C@@H]4CCCC[C@H]4O)sc3c2)ccn1]

In some embodiments, a BCMA-targeting agent and a GSI described herein are administered to a subject in combination with a interleukin-15 (IL-15) polypeptide, a interleukin-15 receptor alpha (IL-15Ra) polypeptide, or a combination of both a IL-15 polypeptide and a IL-15Ra polypeptide e.g., hetIL-(Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311, incorporated herein by reference. In embodiments, het-IL-15 is administered subcutaneously. In embodiments, the subject has a cancer, e.g., solid cancer, e.g., melanoma or colon cancer. In embodiments, the subject has a metastatic cancer.

In some embodiment, the subject is administered a corticosteroid, such as, e.g., methylprednisolone, hydrocortisone, among others.

In some embodiments, the subject is administered a vasopressor, such as, e.g., norepinephrine, dopamine, phenylephrine, epinephrine, vasopressin, or a combination thereof.

In an embodiment, the subject can be administered an antipyretic agent. In an embodiment, the subject can be administered an analgesic agent.

In one embodiment, the subject can be administered an agent which inhibits an inhibitory molecule, e.g., the agent is a checkpoint inhibitor. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta.

In one embodiment, a BCMA-targeting agent and a GSI described herein are administered to a subject in combination with an agent, e.g., an antibody or antibody fragment that binds to an inhibitory molecule. In an embodiment, the agent is an antibody or antibody fragment that binds to PD1, PD-L1, PD-L2 or CTLA4 (e.g., Ipilimumab (also referred to as MDX-010 and MDX-101, and marketed as Yervoy®; Bristol-Myers Squibb); or Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

PD-1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD-1, PD-L1 and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD-1 with PD-L1. Antibodies, antibody fragments, and other inhibitors of PD-1, PD-L1 and PD-L2 are available in the art and may be used combination with a BCMA-targeting agent and a GSI described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. MED14736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.S70 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

TIM3 (T cell immunoglobulin-3) also negatively regulates T cell function, particularly in IFN-g-secreting CD4+ T helper 1 and CD8+ T cytotoxic 1 cells, and plays a critical role in T cell exhaustion. Inhibition of the interaction between TIM3 and its ligands, e.g., galectin-9 (Gal9), phosphotidylserine (PS), and HMGB1, can increase immune response. Antibodies, antibody fragments, and other inhibitors of TIM3 and its ligands are available in the art and may be used in combination with a BCMA-targeting agent and a GSI described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-1 are disclosed in US20130156774.

In other embodiments, the agent is a CEACAM inhibitor (e.g., CEACAM-1, CEACAM-3, and/or CEACAM-5 inhibitor). In one embodiment, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., amonoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; j1(6):1300-10; Stem et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer an ovarian cancer, and other cancers as described herein.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with a BCMA-targeting agent and a GSI described herein. For example, BMS-986016 (Bristoli-Myers Squib) is a monoclonal antibody that targets LAG3. IMP701 (Immutep) is an antagonist LAG3 antibody and IMP73I (Inmnutep and GlaxoSmiithKline) is a depleting LAG3 antibody. Other LAG3 inhibitors include IMP32I (Immutep), which is a recombinant fusion protein of a soluble portion of LAG3 and Ig that binds to MHC class II molecules and activates antigen presenting cells (APC). Other antibodies are disclosed, e.g., in WO2010/019570.

In one embodiment, the agent which enhances activity of a BCMA-targeting agent and a GSI described herein is miR-17-92.

In one embodiment, the agent which enhances activity of a BCMA-targeting agent and a GSI described herein is a cytokine. Cytokines have important functions related to T cell expansion, differentiation, survival, and homeostatis. Cytokines that can be administered to the subject receiving a BCMA-targeting agent and a GSI described herein include: IL-2, IL-4, IL-7, IL-9, IL-15, IL-18, and IL-21, or a combination thereof. In preferred embodiments, the cytokine administered is IL-7, IL-15, or IL-21, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days.

In embodiments, the cytokine is administered in combination with a BCMA-targeting agent and a GSI. The cytokine can be administered simultaneously or concurrently with the BCMA-targeting agent, e.g., administered on the same day. The cytokine may be prepared in the same pharmaceutical composition as the BCMA-targeting agent, or may be prepared in a separate pharmaceutical composition. Alternatively, the cytokine can be administered shortly after administration of the BCMA-targeting agent, e.g., 1 day, 2 days, 3 days, 4 days, 5 days, 6 days, or 7 days after administration of the BCMA-targeting agent. In embodiments where the cytokine is administered in a dosing regimen that occurs over more than one day, the first day of the cytokine dosing regimen can be on the same day as administration of the BCMA-targeting agent, or the first day of the cytokine dosing regimen can be 1 day, 2 days, 3 days, 4 days, 5 days, 6 days, or 7 days after administration of the BCMA-targeting agent. In one embodiment, on the first day, the BCMA-targeting agent is administered to the subject, and on the second day, a cytokine is administered once a day for the next 7 days. In a preferred embodiment, the cytokine to be administered in combination with the BCMA-targeting agent is IL-7, IL-15, or IL-21.

In other embodiments, the cytokine is administered a period of time after administration of the BCMA-targeting agent, e.g., at least 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of the BCMA-targeting agent. In one embodiment, the cytokine is administered after assessment of the subject's response to the BCMA-targeting agent. For example, the subject is administered the BCMA-targeting agent according to the dosage and regimens described herein. The response of the subject to the BCMA-targeting agent is assessed at 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of the BCMA-targeting agent, using any of the methods described herein, including inhibition of tumor growth, reduction of circulating tumor cells, or tumor regression. Subjects that do not exhibit a sufficient response to the BCMA-targeting agent can be administered a cytokine. Administration of the cytokine to the subject that has sub-optimal response to the BCMA-targeting agent improves the efficacy or anti-cancer activity of the BCMA-targeting agent. In a preferred embodiment, the cytokine administered after administration of the BCMA-targeting agent is IL-7.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the molecules of the present invention and practice the claimed methods. The following working examples specifically point out various aspects of the present invention, and are not to be construed as limiting in any way the remainder of the disclosure.

### Example 1

### Summary

In this example, the effect of gamma secretase inhibitors (GSIs) on BCMA cell surface expression, BCMA shedding, and CD3×BCMA bispecific antibody activity and potency in vitro was examined. Treatment of BCMA-expressing cells with a GSI prevented shedding of BCMA into cell culture supernatants, which in turn increased cell surface expression of BCMA in vitro. Treating BCMA-expressing cells with a GSI also enhanced potency and activity of BCMA-targeted therapies. In an in vitro redirected T cell cytotoxicity (RTCC) assay, CD3×BCMA bispecific antibodies efficiently killed several multiple myeloma cell lines, and the potency of these antibodies were significantly enhanced upon GSI treatment. This finding supports extending this therapy enhancement to other BCMA-targeted therapies, including but not limited to antibody-drug conjugates (ADCs), antibodies that induce antibody-dependent cellular cytotoxicity (ADCC) or complement-dependent cytotoxicity (CDC), and CAR-T therapies.

### Materials and Methods

Five BCMA×CD3 bispecific antibodies, ER26, BQ76, BU76, EE11, and EM90 were generated. The sequence information of these antibodies is disclosed in Table 1.

The bispecific antibodies ER26, BQ76, and BU76 comprise an anti-BCMA Fab linked to a first human IgG1 Fc domain, paired with an anti-CD3 scFv similarly linked to a second human IgG1 Fc domain (FIG. 1A). The anti-BCMA Fab of the bispecific antibody ER26 was generated based on the variable region sequences of the antibody J6M0 disclosed in U.S. Pat. No. 9,273,141, herein incorporated by reference in its entirety. The anti-BCMA Fab of the bispecific antibody BQ76 was generated based on the variable region sequences of the antibody 17A5 disclosed in WO 2016/020332, herein incorporated by reference in its entirety. The anti-BCMA Fab of the bispecific antibody BU76 was generated based on the variable region sequences of the antibody C1 ID5 disclosed in WO 2010/104949, herein incorporated by reference in its entirety. As shown in FIG. 1A, the antibodies ER26, BQ76, and BU76 all have three polypeptide chains: a first chain comprising a VH, CH1, and a first Fc domain; a second chain comprising VL and CL; and a third chain comprising an scFv and a second Fc domain. In ER26, the first, second, and third chains comprise the amino acid sequences of SEQ ID NOs: 1120, 1121, and 1122, respectively. In BQ76, the first, second, and third chains comprise the amino acid sequences of SEQ ID NOs: 1125, 1126, and 1122, respectively. In BU76, the first, second, and third chains comprise the amino acid sequences of SEQ ID NOs: 1129, 1130, and 1122, respectively.

The bispecific antibody EE 11 comprises a first chain comprising an anti-BCMA scFv linked to a first human IgG1 Fc domain, paired with a second chain comprising an anti-CD3 scFv linked to a second human IgG1 Fc domain (FIG. 1B). The anti-BCMA scFv of the bispecific antibody EE11 was generated based on the variable region sequences of the antibody 83A10 disclosed in WO 2016/020332, herein incorporated by reference in its entirety. The first and second chains of EE 11 comprise the amino acid sequences of SEQ ID NOs: 1133 and 1134, respectively.

The bispecific antibody EM90 comprises a first anti-BCMA Fab, linked to an anti-CD3 Fab, which is in turn linked to a first Fc domain; paired with a second anti-BCMA Fab linked to a second Fc domain (FIG. 1D). The anti-BCMA Fab and anti-CD3 Fab of the bispecific antibody EM90 were generated based on the sequence of the antibody pSCHLI372 disclosed in WO 2016/079177, herein incorporated by reference in its entirety. The three light chains of EM90 share the same amino acid sequence of SEQ ID NO: 1153. EM90 comprises two heavy chains: a first heavy chain comprising a first VH, a first CH1, a second VH, a second CH1, and a first Fc domain; and a second heavy chain comprising a third VH, a third CH1, and a second Fc domain. These two heavy chains of EM90 comprise the amino acid sequences of SEQ ID NOs: 1155 and 1154, respectively.

An gH (glycoprotein H)×CD3 bispecific antibody, named XU68, was also generated using the configuration shown in FIG. 1B and used as a negative control. XU68 comprises a first chain comprising an anti-gH scFv linked to a first Fc domain, paired with a second chain comprising an anti-CD3 scFv linked to a second Fc domain. The first and second chains of XU68 comprise the amino acid sequences of SEQ ID NOs: 1135 and 1136, respectively.

Antibody dose titrations for in vitro cell based assays represented here range from 0.1 μM to 300 nM.

Peripheral blood mononuclear cells (PBMCs) were isolated from the blood of healthy human donors using a Ficoll-Paque PLUS (GE Healthcare #17-1440-02) density gradient. T-cells (pan) were isolated from the PBMC fraction by negative selection (Miltenyi #130-096-535, 130-041-407, 130-042-401). These isolated T-cells were activated for expansion with a 3× ratio of Human T-Activator CD3/CD28 Dynabeads (Gibco #11132D) for nine days, magnetically debeaded and stored as frozen aliquots in liquid nitrogen. Frozen aliquots were thawed, counted and used immediately in T-cell killing assays at an E:T ratio of 3:1 with target cancer cell lines.

Target human cancer cell lines expressing various levels of human BCMA (NCI-H929^{[High]}, MM1S^{[Med]} and U266B1^{[low]}) were all transduced to constitutively express luciferase, which is used to measure cell viability/survival with the BrightGlo reagent (Promega #E2650). Target cells were plated at 30,000 cells per well in a 96 well plate (Costar #3904) together with thawed T-cells and a serial dilution of various BCMA×CD3 bispecific antibodies, all in media containing RPMI/1640, 10% FBS, 2 mM L-glutamine, 0.1 mM Non-essential amino acids, 1 mM Sodium pyruvate, 10 mM HEPES, and 0.055 mM 2-mercaptoethanol (Gibco #22400089, 16140, 25030-081, 11140-050, 11360-070, 15630-080, and 21985-023 respectively). These treatments were also compared in the presence or absence of either of two GSIs: DAPT [1.0 μM](Enzo Life Sciences, #ALX-270-416-M005), or LY-411,575 [0.1 μM] (Sigma, #SML0506). The assay was incubated at 37° C. and 5% CO₂ for 20-24 hours, followed by measurements of target cell viability (BrightGlo, Promega #E2650), following vendor supplied protocols.

BCMA expressing cell lines were also treated overnight with and without GSIs. Cell culture supernatants were collected and quantified for levels of soluble BCMA by ELISA (R&D Systems, #DY193). Additionally, remaining cells were pelleted, stained with a PE conjugated BCMA antibody (clone 19F2, Biolegend #357504) and analyzed by flow cytometry for BCMA cell surface expression levels.

### Results

The level of soluble (shed) BCMA in cell culture supernatants was measured by ELISA following 24-hour treatment with or without 0.5 μM DAPT. GSI treatment decreased levels of soluble BCMA in cultures to levels below the assay detection limit (Table 12).

TABLE 12
Gamma secretase inhibitor DAPT inhibits
shedding of BCMA in vitro

| ng/mL BMCA | NCI-H929 | MM1S | RPM-8226 |
|---|---|---|---|
| Untreated | 137.5 | 40.8 | 49.8 |
| 0.5 μM DAPT | <0.03 | <0.03 | <0.03 |

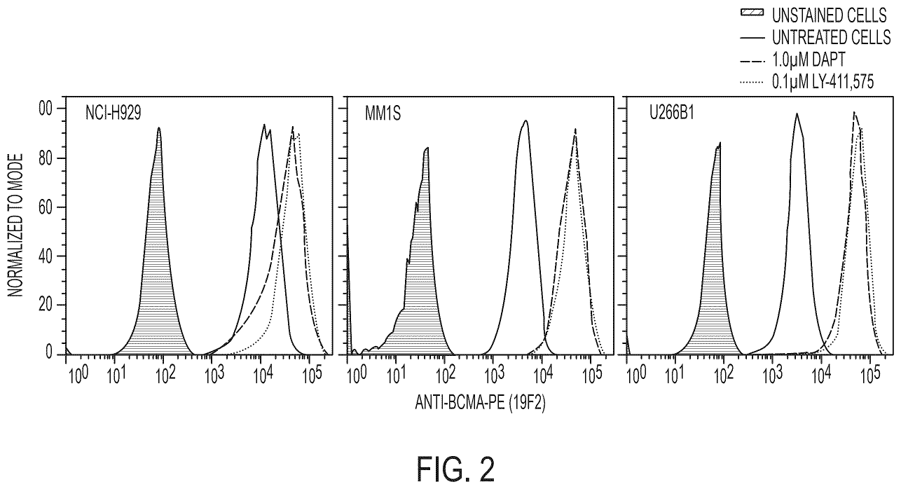

BCMA cell surface expression level was determined by flow cytometry on cell lines with or without GSI treatment. As shown in FIG. 2, GSI treatment resulted in a 3-15 fold increase in cell surface expression of BCMA compared to non-treated cells (3 fold in NCI-H929, 10 fold in MMIS, and fold in U266B1).

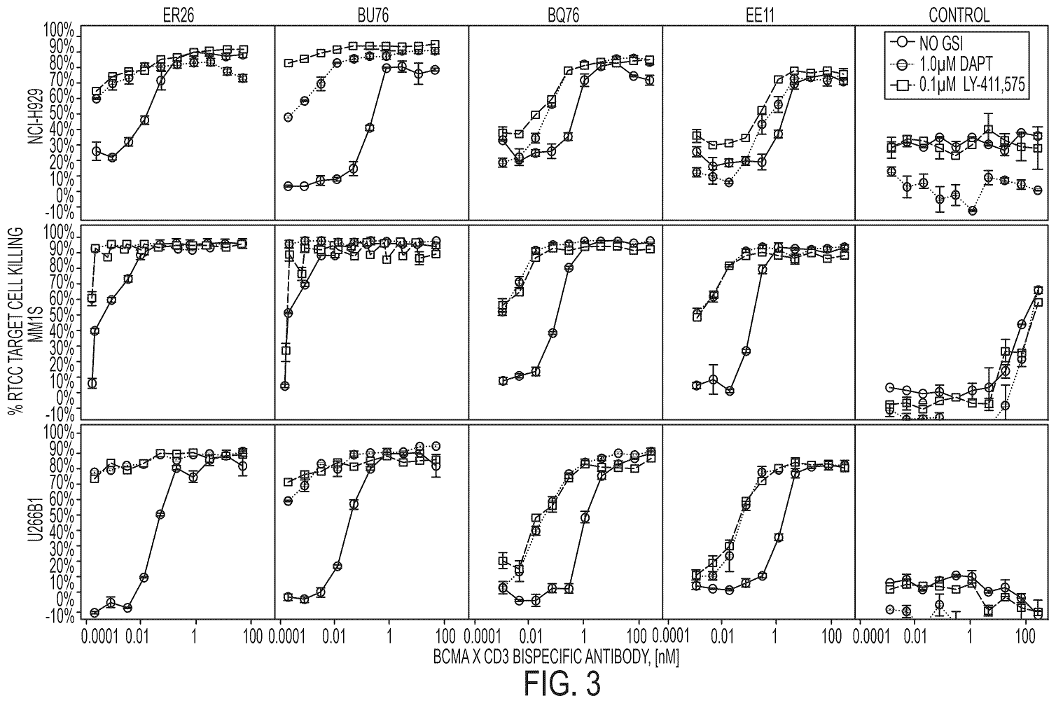

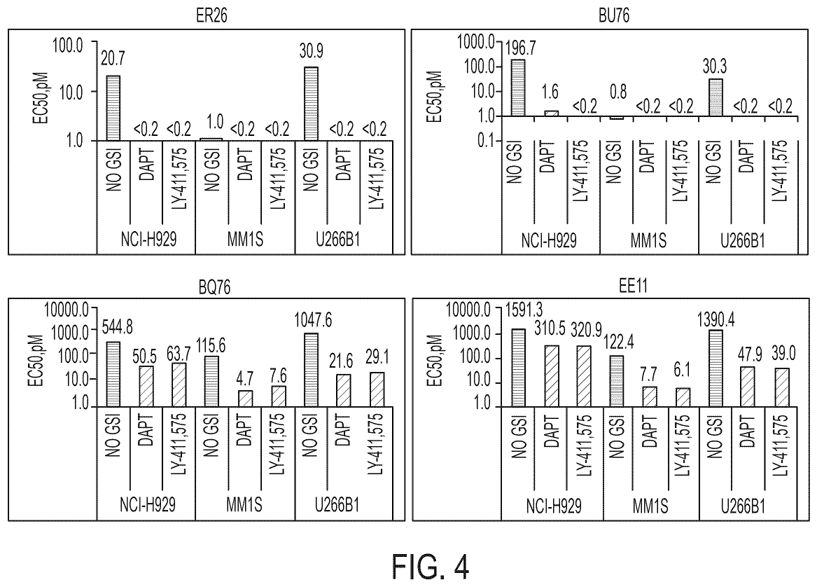

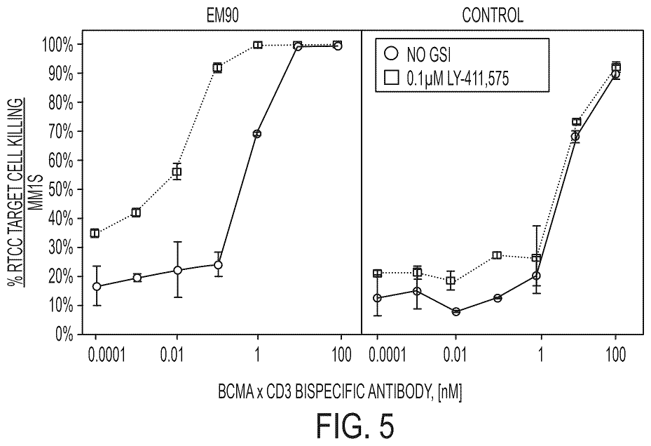

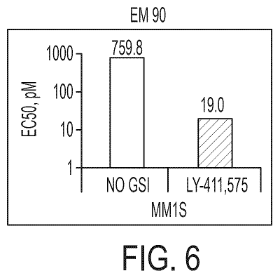

Redirected T-cell cytotoxicity (RTCC) killing assays were used to show that treatment of BCMA-expressing cell lines with GSIs enhanced the potency of BCMA×CD3 bispecific antibodies in vitro (FIGS. 3 and 5), showing significantly lower EC50 values for the individual bispecific antibodies (FIGS. 4 and 6).

### Discussion

Based on the data shown, BCMA×CD3 bispecific antibodies are capable of re-directing T-cells to kill BCMA-expressing tumor cells in vitro, and this antibody specific killing is significantly enhanced by GSI treatment.

Table 12 shows the efficiency of DAPT at preventing the shedding of BCMA from the cell surface into the culture supernatant. This would be expected to enhance BCMA-directed therapies by removing a potential drug sink: binding of the therapies to soluble BCMA may neutralize their ability to bind and target cancer cells. FIG. 2 demonstrates that treatment of BCMA-expressing multiple myeloma cell lines with two different GSIs, DAPT and LY-411,575, strongly enhances cell surface expression of BCMA. Increased cell surface expression of BCMA provides more potential antibody binding events, thereby enhancing T cell engagement and target cell killing.

FIGS. 3 and 5 confirm that GSI treatment of three different BCMA-expressing cell lines does in fact significantly enhance the potency of BCMA×CD3 bispecific antibodies in an in vitro re-directed T cell cytotoxicity assay. Five different BCMA×CD3 bispecific antibodies were all enhanced by treatment with either of two different GSIs: DAPT, or LY-411,575. FIGS. 4 and 6 summarize EC50 values from FIGS. 3 and 5 to demonstrate that EM90 was enhanced by more than 40-fold upon GSI treatment, ER26 and BU76 were enhanced by more than 100-fold, and BQ76 and EE 11 were enhanced by 10-fold or more. Without wishing to be bound by theory, this difference in the level of enhancement may be attributed to variations in affinities of these antibodies to BCMA, or the mechanism by which the GSI enhances activity, whether by removing the soluble BCMA neutralizing sink, by increasing BCMA cell surface expression, by altering the conformation and/or posttranslational modification of the BCMA ECD, or a combination of any of the three factors.

Taken together, these data strongly support the use of GSIs in combination with BCMA-targeted therapies, including but not limited to BCMA×CD3 bispecific antibodies, ADCC-competent antibodies, antibody drug conjugates, and chimeric antigen receptor T cell therapy.

### Example 2

In this example, the effect of gamma secretase inhibitors on BCMA cell surface expression and BCMA shedding was investigated in vitro. Treatment of BCMA expressing cells with a gamma secretase inhibitor (GSI) prevented shedding of the receptor into cell culture supernatants, which in turn increased cell surface expression of the receptor in vitro.

### Materials and Methods

Target human cancer cell lines expressing various levels of human BCMA (NCI-H929^{[High]}, MM1S^{[Med]}, U266B1^{[low]}) were all transduced to constitutively express luciferase, which is used to measure cell viability/survival with the BrightGlo reagent (Promega E2650). Target cells were plated 30,000 cells per well in a 96 well plate (Costar 3904) together with serial dilutions of a panel of gamma secretase inhibitors (Table 13). The plate was incubated at 37° C./5% CO₂ for 72 hours, followed by measurements of cell viability (BrightGlo, Promega #E2650) according to vendor protocol. In a replicate set of assay plates, cell culture supernatants were collected by centrifugation and quantified for levels of soluble BCMA by ELISA (R&D Systems, DY193), and the remaining cells were stained with a PE conjugated BCMA antibody (clone 19F2, Biolegend 357504) and analyzed by flow cytometry for BCMA cell surface expression levels.

TABLE 13
Gamma secretase inhibitors

| Gamma Secretase | | | |
|---|---|---|---|
| Inhibitor (GSI) | Developer | Supplier | item # |
| LY-450139 | Lilly/Elan | Novartis | NVP-ANJ-889 |
| (Semangacestat) | | | |
| PF-5212362 | Pfizer (Wyeth) | Novartis | NVP-CGX-400 |
| (Begacestat, | | | |
| GSI-953) | | | |
| BMS-708163 | BMS | Novartis | NVP-CHG-662 |
| (Avagacestat) | | | |
| LY-411575 | Lilly | Sigma | SML0506-5mg |
| PF-3084014 | Pfizer | Novartis | NVP-CHE-743 |
| (Nirogacestat) | | | |
| BMS-299897 | BMS | R&D Systems | 2870/10 |
| DAPT | Lilly | Enzo | ALX-270-416- |
| | | | M005 |
| RO4929097 | Roche | Selleck | S1575-5MG |
| MK-0752 | Merck | Novartis | NVP-LJR-137 |

### Results

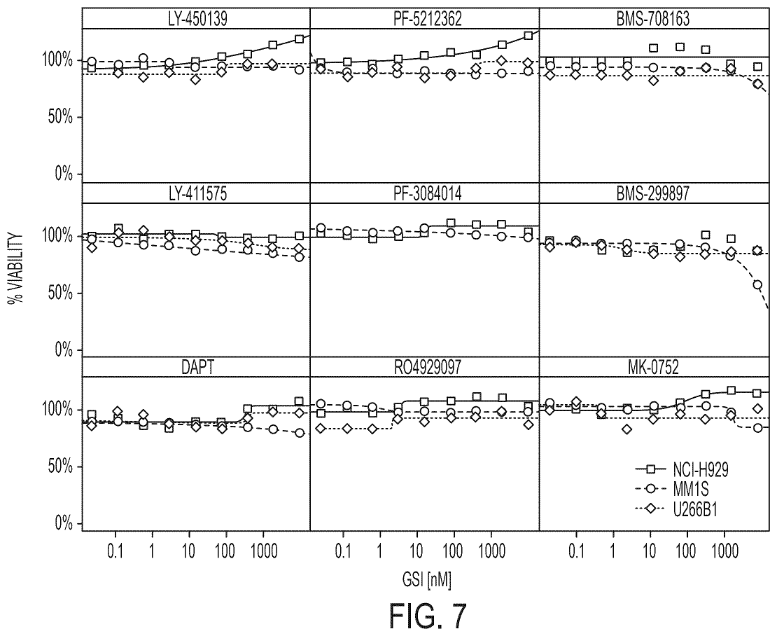

Luminescence values obtained from the BrightGlo assay were normalized to percent viability by taking the ratio of values from treated over untreated and plotted in Spotfire. All the GSI compounds were well tolerated at all the concentrations used (FIG. 7).

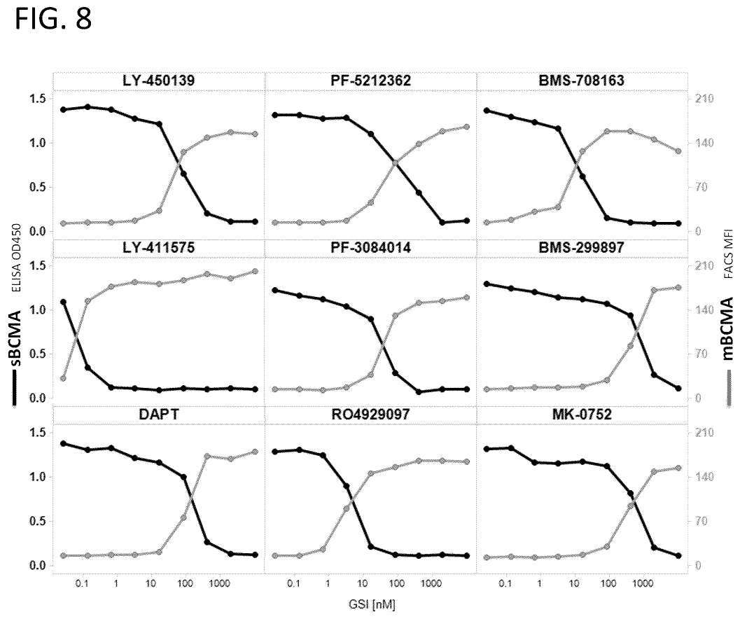

Raw OD450 values from the BCMA ELISA were plotted together with the mean fluorescent intensity values from flow cytometry analysis of cells stained with anti-BCMA antibody, and are shown for MM1S cells treated with the various GSIs in FIG. 8. Table 14 summarizes the EC50 results of all the GSIs on the three BCMA-expressing cell lines.

TABLE 14
EC50 [nM] summary of GSI inhibition of shed/soluble
BCMA, and enhancement of membrane BCMA expression.

| | H929 | | MM1S | | U266B1 | |
|---|---|---|---|---|---|---|
| EC50, nM | sBCMA | mBCMA | sBCMA | mBCMA | sBCMA | mBCMA |
| LY-450139 | 99.9 | 54.8 | 65.0 | 40.7 | 236.0 | 190.4 |
| PF-5212362 | 136.6 | 13.8 | 120.9 | 52.2 | 583.0 | 560.0 |
| BMS-708163 | 2.5 | 3.6 | 13.3 | 7.4 | 29.8 | 32.4 |
| LY-411575 | 0.2 | 0.1 | 0.1 | 0.0 | 0.1 | 0.1 |
| PF-3084014 | 34.8 | 31.6 | 32.2 | 36.6 | 67.2 | 70.4 |
| BMS-299897 | 528.1 | 151.6 | 799.8 | 484.9 | | 1687.7 |
| DAPT | 220.4 | 144.7 | 144.2 | 97.4 | 458.9 | 401.3 |
| RO4929097 | 3.0 | 4.4 | 4.6 | 3.2 | 6.2 | 10.1 |
| MK-0752 | 269.1 | 69.3 | 565.7 | 331.6 | 1056.8 | 1208.3 |

### Discussion

It was demonstrated here that a broad panel of gamma secretase inhibitors, some reaching Phase III clinical trials, are all effective at inhibiting cleavage, and thereby shedding, of BCMA in vitro. This inhibition has been shown to result in increased membrane expression of BCMA in three different multiple myeloma cell lines, ideally maximizing the clinical efficacy of BCMA targeted therapies when used in combination. FIG. 7 suggests all of these inhibitors are well tolerated in vitro at effective concentrations.

Interestingly, all of the inhibitors show a correlation between effective concentrations that inhibit shedding of BCMA and that enhance membrane expression of BCMA. This correlation is exhibited by similar EC50 values in Table 14, as well as the overlapping infection points in FIG. 8. Among the panel of GSIs, efficacious concentrations vary considerably, with LY-411575 being the most potent, and BMS-299897 being the least. Effective concentrations also vary among the different cell lines tested, and may not necessarily be based on level of BCMA expression.

Taken together, these data strongly support the further development and use of gamma secretase inhibitors in combination with BCMA targeted therapies, including but not limited to CD3 bispecific antibodies, chimeric antigen receptor T cell therapy, and antibody drug conjugates.

### Example 3: Effect of GSI Treatment on BCMA Targeted Therapies

### Materials and Methods

### Gamma Secretase Inhibitors

Information of gamma secretase inhibitors tested in this Example is provided in Table 13 of Example 2.

### Anti-BCMA Antibodies

The following antibodies were tested in this Example.

TABLE 15
Anti-BCMA antibodies

| Antibody | SMFID |
|---|---|
| Pfizer_CD3_H2B4_RRRR-Pfizer_BCMA_comboC29_{—} | ZC-03-FP31 |
| EEEE hIgG2a_ASS | |
| RSV × CD3_[30 nM] (negative ctrl) | 25153 |
| Anti-BCMA J6M0 hIgG DLE CGW117 (MMAE) | OE-16-ET44 |
| conjugate | |
| Anti-CD19 hIgG DLE CGW117 (MMAE) conjugate | IB-91-HU40 |

### Human T Cell Isolation

Peripheral blood mononuclear cells (PBMCs) were isolated from the blood of healthy human donors using a Ficoll-Paque PLUS (GE Healthcare #17-1440-02) density gradient. T-cells (pan) were isolated from the PBMC fraction by negative selection (Miltenyi #130-096-535, 130-041-407, 130-042-401) and aliquots stored in liquid nitrogen in CryoStor10 solution (HemaCare #210374).

In vitro Antibody Potency

### RTCC Assay

Target human cancer cell lines which either express BCMA (KMS11) or do not express BCMA (NALM6) were transduced to constitutively express luciferase, which is used to measure cell viability/survival with the BrightGlo reagent (Promega E2650). Target cells were plated 7,500 cells per well in a 384 well plate (Costar 3765). Cells were treated with or without a gamma secretase inhibitor (GSI, comparison of 3 different compounds) for 30 minutes prior to the addition of 22,500 thawed T-cells, and a serial dilution of BCMA×CD3 bispecific or negative control CD3 bispecific antibodies ranging from 100 nM to 0.1 pM. The base assay media (TCM, or T cell Medium) contains RPMI/1640, 10% FBS, 2 mM L-glutamine, 0.1 mM Non-essential amino acids, 1 mM Sodium pyruvate, 10 mM HEPES, 0.055 mM 2-mercaptoethanol (Gibco 22400089, 16140, 25030-081, 11140-050, 11360-070, 15630-080, 21985-023 respectively). The final concentration of all gamma secretase inhibitors was 2 μM. The assay was incubated at 37° C./5% CO₂ for 48 hours, followed by measurements of target cell viability (BrightGlo, Promega #E2650), following vendor supplied protocols. Analysis was done using Graphpad Prism software.

### ADC Killing Assay

Target human cancer cell lines that express BCMA (MM1S and U266B1) or are BCMA negative (NALM6), were added to wells of a 384 well assay plate (Costar #3765), 7,500 cells per well. All of these cell lines were previously engineered to constitutively express firefly luciferase via lentiviral transduction. These cells were pretreated with or without gamma secretase inhibitor LY-411,575 (Sigma #SML0506), 100 nM for 30 minutes. A serial dilution of antibody drug conjugate (ADC) was then added, final concentration range from 100 nM to 0.4 μM. The assay was incubated 37° C. in 5% CO₂ environment for 72 hours. BrightGlo luminescence reagent (Promega #E2650) was used according to vendor supplied protocol to detect surviving cells. Analysis was done using GraphPad Prism software.

### Results

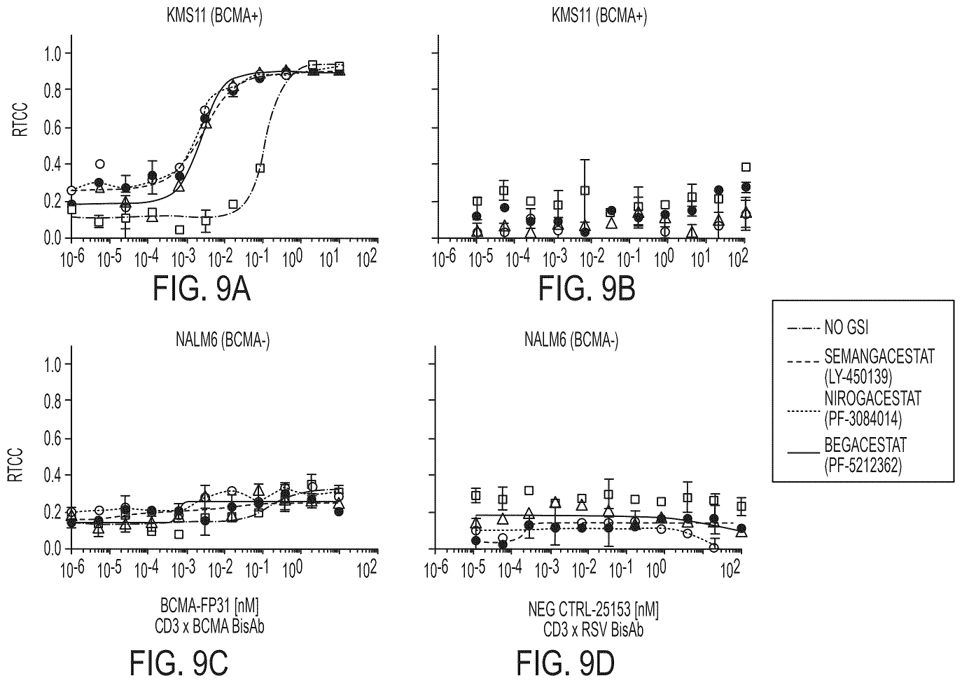

Redirected T cell cytotoxicity assay (RTCC) results indicate that treatment of BCMA expressing target cells (KMS 11, FIG. 9A) with a gamma secretase inhibitor could enhance the potency of BCMA×CD3 bispecific antibody FP31, while the non-targeting RSV×CD3 antibody (25153) had no effect (FIG. 9B), nor were the treatments active on cells that do not express BCMA (NALM6, FIGS. 9C and 9D).

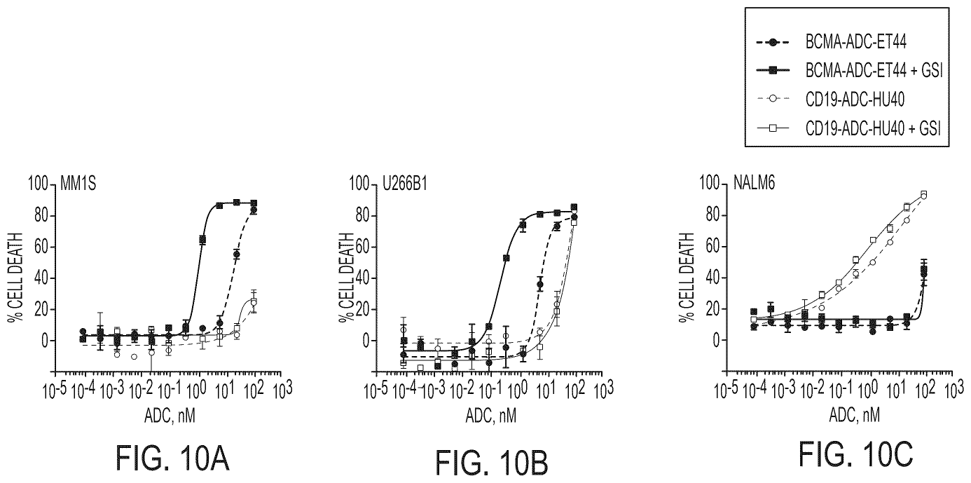

ADC Cell killing assay results show GSI treatment enhanced the potency of BCMA antibody drug conjugates in BCMA expressing cell lines MM1S (FIG. 10A) and U266B1 (FIG. 10B), yet not in a BCMA negative cell line NALM6 (FIG. 10C). The CD19 antibody drug conjugate control showed little to no activity in CD19 negative cell lines MM1S (FIG. 10A) and U266B1 (FIG. 10B). NALM6 cells express CD19, and therefore the control CD19 antibody drug conjugate was active, however its activity was not enhanced by GSI treatment (FIG. 10C).

IC50 values for both RTCC and ADC assays show increased potency for all BCMA antibodies or antibody drug conjugates upon gamma secretase treatment (Table 16).

TABLE 16
IC50 of RTCC and ADC assays

| BCMA + | | | IC50, nM | | | | | |
|---|---|---|---|---|---|---|---|---|
| Target | | BCMA | No | LY- | LY- | PF- | PF- | Fold GSI |
| cell line | Assay | Antibody | GSI | 450139 | 411575 | 5212362 | 3084014 | Enhancement |
| KMS11 | RTCC | BCMA(Pf) × | 0.097 | 0.002 | | 0.002 | 0.002 | 48.3 |
| | | CD3_FP31 | | | | | | |
| MM1S | ADC | BCMA(J6M0) - | 19.40 | | 1.12 | | | 17.4 |
| | | MMAE_ET44 | | | | | | |
| U266B1 | ADC | BCMA(J6M0) - | 6.04 | | 0.26 | | | 23.1 |
| | | MMAE_ET44 | | | | | | |

### Discussion

The studies described in Examples 1-3 demonstrated that treatment with gamma secretase inhibitor (GSI) prevents the cleavage and release of BCMA from the cell membrane, resulting in higher membrane density of BCMA, and less soluble BCMA in the surrounding medium. These studies further demonstrated that GSI treatment enhances the potency of BCMA-targeting therapies. Without wishing to be bound by theory, possible mechanisms may include increased target density on the cell membrane, and/or reduction of the soluble factor that could potentially act to neutralize BCMA-targeting moieties. FIGS. 9A-9D show a GSI induced enhancement of BCMA×CD3 bispecific antibody potency in an in vitro re-directed T-cell cytotoxicity assay. In the BCMA-expressing KMS 11 target cell line, three separate gamma secretase inhibitors similarly improved the potency of the bispecific antibody nearly 50-fold (97 pM to ˜2 pM). FIGS. 10A-10C further demonstrate the benefit of GSI treatment in BCMA-targeting therapies by showing enhanced antibody drug conjugate (ADC) potency in BCMA-expressing cells MM1S and U266B1 by 17 and 23-fold respectively.

Based on these findings, GSI combination with BCMA-targeting therapies could greatly enhance the outcome for patients whose BCMA expression may be too low for efficient targeting, since GSI treatment could increase BCMA expression on the cell surface, allowing more robust targeting and responses to BCMA therapies.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of treating a multiple myeloma, the method comprising administering to a subject in need thereof an effective amount of:
(i) a multispecific antibody molecule that binds to B-cell maturation antigen (BCMA) and CD3, and
(ii) a gamma secretase inhibitor (GSI),
wherein the multispecific antibody molecule comprises:
(a) a first binding moiety that binds BCMA and comprises a heavy chain comprising an amino acid sequence having at least 95% identity to SEQ ID NO: 1159, and a light chain comprising an amino acid sequence having at least 95% identity to SEQ ID NO: 1158; and
(b) a second binding moiety that binds CD3 and comprises a heavy chain comprising an amino acid sequence having at least 95% identity to SEQ ID NO: 1160, and a light chain comprising an amino acid sequence having at least 95% identity to SEQ ID NO: 1157; and
wherein the GSI is LY-450139, PF-5212362, or PF-3084014,
thereby treating the multiple myeloma.

2. The method of claim 1, wherein the heavy chain of the first binding moiety comprises an amino acid sequence having at least 99% identity to SEQ ID NO: 1159; and wherein the light chain of the first binding moiety comprises an amino acid sequence having at least 99% identity to SEQ ID NO: 1158.

3. The method of claim 1, wherein the heavy chain of the second binding moiety comprises an amino acid sequence having at least 99% identity to SEQ ID NO: 1160; and wherein the light chain of the second binding moiety comprises an amino acid sequence having at least 99% identity to SEQ ID NO: 1157.

4. The method of claim 1, wherein the GSI is LY-450139.

5. The method of claim 1, wherein the GSI is PF-5212362.

6. The method of claim 1, wherein the GSI is PF-3084014.

7. The method of claim 1, wherein the multispecific antibody molecule and the GSI are administered simultaneously.

8. The method of claim 1, wherein the multispecific antibody molecule and the GSI are administered sequentially.

9. The method of claim 8, wherein the GSI is administered prior to the administration of the multispecific antibody molecule.

10. The method of claim 1, wherein the multispecific antibody molecule, when bound to BCMA-expressing cells, is capable of inducing antibody-dependent cellular cytotoxicity (ADCC) or complement-dependent cytotoxicity (CDC).

11. The method of claim 10, wherein the multispecific antibody molecule comprises an Fc region comprising at least one mutation that increases the ability of the multispecific antibody molecule to induce ADCC or CDC.

12. The method of claim 1, wherein the multispecific antibody molecule and the GSI are administered in combination with a third therapeutic agent or procedure.

13. The method of claim 12, wherein the third therapeutic agent is:
(i) dexamethasone;
(ii) a PD-1 inhibitor;
(iii) a PD-L1 inhibitor;
(iv) a CTLA-4 inhibitor;
(v) a TIM-3 inhibitor;
(vi) a LAG-3 inhibitor;
(vii) an mTOR inhibitor; or
(viii) an agent that is HetIL-15, an anti-TGFβ antibody, an anti-CD47 antibody, an IDO inhibitor, a STING agonist, a TLR agonist, an immunomodulatory drug (IMiD), or a proteasome inhibitor.

14. The method of claim 1, wherein the multispecific antibody molecule further binds to a third antigen.

15. The method of claim 1, wherein the multispecific antibody molecule comprises an afucosylated Fc region.

16. The method of claim 1, wherein the multispecific antibody molecule is linked to a drug moiety that exerts a cytotoxic or cytostatic activity.

17. The method of claim 16, wherein the multispecific antibody molecule is linked to the drug moiety through a cleavable linker, a non-cleavable linker, a hydrophilic linker, or a dicarboxylic acid-based linker.

18. The method of claim 1, wherein the multispecific antibody molecule is linked to a drug moiety that is a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

19. A method of treating a multiple myeloma, the method comprising administering to a subject in need thereof an effective amount of:
(i) a multispecific antibody molecule that binds to BCMA and CD3, and
(ii) PF-3084014,
wherein the multispecific antibody molecule comprises:
(a) a first binding moiety that binds BCMA and comprises a heavy chain comprising an amino acid sequence having at least 99% identity to SEQ ID NO: 1159, and a light chain comprising an amino acid sequence having at least 99% identity to SEQ ID NO: 1158; and
(b) a second binding moiety that binds CD3 and comprises a heavy chain comprising an amino acid sequence having at least 99% identity to SEQ ID NO: 1160, and a light chain comprising an amino acid sequence having at least 99% identity to SEQ ID NO: 1157, thereby treating the multiple myeloma.

20. A method of treating a multiple myeloma, the method comprising administering to a subject in need thereof an effective amount of:
(i) a multispecific antibody molecule that binds to BCMA and CD3, and
(ii) a GSI,
wherein the GSI is administered prior to the administration of the multispecific antibody molecule;
wherein the multispecific antibody molecule comprises:
(a) a first binding moiety that binds BCMA and comprises a heavy chain comprising an amino acid sequence having at least 99% identity to SEQ ID NO: 1159, and a light chain comprising an amino acid sequence having at least 99% identity to SEQ ID NO: 1158; and
(b) a second binding moiety that binds CD3 and comprises a heavy chain comprising an amino acid sequence having at least 99% identity to SEQ ID NO: 1160, and a light chain comprising an amino acid sequence having at least 99% identity to SEQ ID NO: 1157; and
wherein the GSI is LY-450139, PF-5212362, or PF-3084014.


SEQUENCE LISTING (1160 sequences)

SEQ ID NO: 1-24 - skipped (24 entries)

SEQ ID NO: 25 - protein, 5 aa, synthetic construct
  REGION 1..5: note = source = /note="Description of Artificial Sequence: Synthetic peptide"
GGGGS

SEQ ID NO: 26 - skipped

SEQ ID NO: 27 - protein, 20 aa, synthetic construct
  REGION 1..20: note = source = /note="Description of Artificial Sequence: Synthetic peptide"
GGGGSGGGGSGGGGSGGGGS

SEQ ID NO: 28 - protein, 15 aa, synthetic construct
  REGION 1..15: note = source = /note="Description of Artificial Sequence: Synthetic peptide"
GGGGSGGGGSGGGGS

SEQ ID NO: 29-38 - skipped (10 entries)

SEQ ID NO: 39 - protein, 246 aa, synthetic construct
  REGION 1..246: note = source = /note="Description of Artificial Sequence: Synthetic polypeptide"
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 40 - protein, 244 aa, synthetic construct
  REGION 1..244: note = source = /note="Description of Artificial Sequence: Synthetic polypeptide"
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSVHSFLAYWGQGTLVTVSSASGGGGSGGRASGGGGSDIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 41 - protein, 243 aa, synthetic construct
  REGION 1..243: note = source = /note="Description of Artificial Sequence: Synthetic polypeptide"
EVQLLESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIVMTQTPLSLSVTPGQPASISCKSSQSLLRNDGKTPLYWYLQKAGQPPQLLIYEVSNRFSGVPDRFSGSGSGTDFTLKISRVEAEDVGAYYCMQNIQFPSFGGGTKLEIK

SEQ ID NO: 42 - protein, 249 aa, synthetic construct
  REGION 1..249: note = source = /note="Description of Artificial Sequence: Synthetic polypeptide"
QVQLVQSGAEVRKTGASVKVSCKASGYIFDNFGINWVRQAPGQGLEWMGWINPKNNNTNYAQKFQGRVTITADESTNTAYMEVSSLRSEDTAVYYCARGPYYYQSYMDVWGQGTMVTVSSASGGGGSGGRASGGGGSDIVMTQTPLSLPVTPGEPASISCRSSQSLLHSNGYNYLNWYLQKPGQSPQLLIYLGSKRASGVPDRFSGSGSGTDFTLHITRVGAEDVGVYYCMQALQTPYTFGQGTKLEIK

SEQ ID NO: 43 - protein, 246 aa, synthetic construct
  REGION 1..246: note = source = /note="Description of Artificial Sequence: Synthetic polypeptide"
QVQLQESGGGLVQPGGSLRLSCAASGFTFSSDAMTWVRQAPGKGLEWVSVISGSGGTTYYADSVKGRFTISRDNSKNTLYLQMNSLRAEDTAVYYCAKLDSSGYYYARGPRYWGQGTLVTVSSASGGGGSGGRASGGGGSDIQLTQSPSSLSASVGDRVTITCRASQSISSYLNWYQQKPGKAPKLLIYGASTLASGVPARFSGSGSGTHFTLTINSLQSEDSATYYCQQSYKRASFGQGTKVEIK

SEQ ID NO: 44 - protein, 247 aa, synthetic construct
  REGION 1..247: note = source = /note="Description of Artificial Sequence: Synthetic polypeptide"
QVQLVQSGAEVKKPGASVKVSCKASGYTFSNYGITWVRQAPGQGLEWMGWISAYNGNTNYAQKFQGRVTMTRNTSISTAYMELSSLRSEDTAVYYCARGPYYYYMDVWGKGTMVTVSSASGGGGSGGRASGGGGSEIVMTQSPLSLPVTPGEPASISCRSSQSLLYSNGYNYVDWYLQKPGQSPQLLIYLGSNRASGVPDRFSGSGSGTDFKLQISRVEAEDVGIYYCMQGRQFPYSFGQGTKVEIK

SEQ ID NO: 45 - protein, 238 aa, synthetic construct
  REGION 1..238: note = source = /note="Description of Artificial Sequence: Synthetic polypeptide"
EVQLLETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPATLSVSPGESATLSCRASQSVSSNLAWYQQKPGQAPRLLIYGASTRASGIPDRFSGSGSGTDFTLTISSLQAEDVAVYYCQQYGSSLTFGGGTKVEIK

SEQ ID NO: 46 - protein, 239 aa, synthetic construct
  REGION 1..239: note = source = /note="Description of Artificial Sequence: Synthetic polypeptide"
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVMTQSPATLSVSPGERATLSCRASQSVSSKLAWYQQKPGQAPRLLMYGASIRATGIPDRFSGSGSGTEFTLTISSLEPEDFAVYYCQQYGSSSWTFGQGTKVEIK

SEQ ID NO: 47 - protein, 241 aa, synthetic construct
  REGION 1..241: note = source = /note="Description of Artificial Sequence: Synthetic polypeptide"
EVQLVETGGGVVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSVGSTNLAWYQQKPGQAPRLLIYDASNRATGIPDRFSGGGSGTDFTLTISRLEPEDFAVYYCQQYGSSPPWTFGQGTKVEIK

SEQ ID NO: 48 - protein, 239 aa, synthetic construct
  REGION 1..239: note = source = /note="Description of Artificial Sequence: Synthetic polypeptide"
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARESGDGMDVWGQGTTVTVSSASGGGGSGGRASGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYTLAFGQGTKVDIK

SEQ ID NO: 49 - protein, 239 aa, synthetic construct
  REGION 1..239: note = source = /note="Description of Artificial Sequence: Synthetic polypeptide"
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 50 - protein, 246 aa, synthetic construct
  REGION 1..246: note = source = /note="Description of Artificial Sequence: Synthetic polypeptide"
QVQLVQSGGGLVKPGGSLRLSCAASGFTFSDYYMSWIRQAPGKGLEWVSYISSSGNTIYYADSVKGRFTISRDNAKNSLYLQMNSLRAEDTAVYYCARSTMVREDYWGQGTLVTVSSASGGGGSGGRASGGGGSDIVLTQSPLSLPVTLGQPASISCKSSESLVHNSGKTYLNWFHQRPGQSPRRLIYEVSNRDSGVPDRFTGSGSGTDFTLKISRVEAEDVGVYYCMQGTHWPGTFGQGTKLEIK

SEQ ID NO: 51 - protein, 239 aa, synthetic construct
  REGION 1..239: note = source = /note="Description of Artificial Sequence: Synthetic polypeptide"
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 52 - protein, 240 aa, synthetic construct
  REGION 1..240: note = source = /note="Description of Artificial Sequence: Synthetic polypeptide"
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSETTLTQSPATLSVSPGERATLSCRASQSVGSNLAWYQQKPGQGPRLLIYGASTRATGIPARFSGSGSGTEFTLTISSLQPEDFAVYYCQQYNDWLPVTFGQGTKVEIK

SEQ ID NO: 53 - protein, 241 aa, synthetic construct
  REGION 1..241: note = source = /note="Description of Artificial Sequence: Synthetic polypeptide"
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 54 - DNA, 738 bp, synthetic construct
  misc_feature 1..738: note = source = /note="Description of Artificial Sequence: Synthetic polynucleotide"
  source 1..738: mol_type = other DNA
caagtgcaactcgtggaatctggtggaggactcgtgcaacccggaagatcgcttagactgtcgtgtgccgccagcgggttcactttctcgaactacgcgatgtcctgggtccgccaggcacccggaaagggactcggttgggtgtccggcatttcccggtccggcgaaaatacctactacgccgactccgtgaagggccgcttcaccatctcaagggacaacagcaaaaacaccctgtacttgcaaatgaactccctgcgggatgaagatacagccgtgtactattgcgcccggtcgcctgcccattactacggcggaatggacgtctggggacagggaaccactgtgactgtcagcagcgcgtcgggtggcggcggctcagggggtcgggcctccggggggggagggtccgacatcgtgctgacccagtccccgggaaccctgagcctgagcccgggagagcgcgcgaccctgtcatgccgggcatcccagagcattagctcctcctttctcgcctggtatcagcagaagcccggacaggccccgaggctgctgatctacggcgctagcagaagggctaccggaatcccagaccggttctccggctccggttccgggaccgatttcacccttactatctcgcgcctggaacctgaggactccgccgtctactactgccagcagtaccactcatccccgtcgtggacgttcggacagggcaccaagctggagattaag

SEQ ID NO: 55 - DNA, 732 bp, synthetic construct
  misc_feature 1..732: note = source = /note="Description of Artificial Sequence: Synthetic polynucleotide"
  source 1..732: mol_type = other DNA
caagtgcaactcgtcgaatccggtggaggtctggtccaacctggtagaagcctgagactgtcgtgtgcggccagcggattcacctttgatgactatgctatgcactgggtgcggcaggccccaggaaagggcctggaatgggtgtcgggaattagctggaactccgggtccattggctacgccgactccgtgaagggccgcttcaccatctcccgcgacaacgcaaagaactccctgtacttgcaaatgaactcgctcagggctgaggataccgcgctgtactactgctccgtgcattccttcctggcctactggggacagggaactctggtcaccgtgtcgagcgcctccggcggcgggggctcgggtggacgggcctcgggcggaggggggtccgacatcgtgatgacccagaccccgctgagcttgcccgtgactcccggagagcctgcatccatctcctgccggtcatcccagtcccttctccactccaacggatacaactacctcgactggtacctccagaagccgggacagagccctcagcttctgatctacctggggtcaaatagagcctcaggagtgccggatcggttcagcggatctggttcgggaactgatttcactctgaagatttcccgcgtggaagccgaggacgtgggcgtctactactgtatgcaggcgctgcagaccccctataccttcggccaagggacgaaagtggagatcaag

SEQ ID NO: 56 - DNA, 729 bp, synthetic construct
  misc_feature 1..729: note = source = /note="Description of Artificial Sequence: Synthetic polynucleotide"
  source 1..729: mol_type = other DNA
gaagtgcaattgttggaatctggaggaggacttgtgcagcctggaggatcactgagactttcgtgtgcggtgtcaggcttcgccctgagcaaccacggcatgagctgggtgcggagagccccggggaagggtctggaatgggtgtccgggatcgtctactccggttcaacttactacgccgcaagcgtgaagggtcgcttcaccatttcccgcgataactcccggaacaccctgtacctccaaatgaactccctgcggcccgaggacaccgccatctactactgttccgcgcatggaggagagtccgatgtctggggacagggcactaccgtgaccgtgtcgagcgcctcggggggaggaggctccggcggtcgcgcctccggggggggtggcagcgacattgtgatgacgcagactccactctcgctgtccgtgaccccgggacagcccgcgtccatctcgtgcaagagctcccagagcctgctgaggaacgacggaaagactcctctgtattggtacctccagaaggctggacagcccccgcaactgctcatctacgaagtgtcaaatcgcttctccggggtgccggatcggttttccggctcgggatcgggcaccgacttcaccctgaaaatctccagggtcgaggccgaggacgtgggagcctactactgcatgcaaaacatccagttcccttccttcggcggcggcacaaagctggagattaag

SEQ ID NO: 57 - DNA, 747 bp, synthetic construct
  misc_feature 1..747: note = source = /note="Description of Artificial Sequence: Synthetic polynucleotide"
  source 1..747: mol_type = other DNA
caagtccaactcgtccagtccggcgcagaagtcagaaaaaccggtgctagcgtgaaagtgtcctgcaaggcctccggctacattttcgataacttcggaatcaactgggtcagacaggccccgggccaggggctggaatggatgggatggatcaaccccaagaacaacaacaccaactacgcacagaagttccagggccgcgtgactatcaccgccgatgaatcgaccaataccgcctacatggaggtgtcctccctgcggtcggaggacactgccgtgtattactgcgcgaggggcccatactactaccaaagctacatggacgtctggggacagggaaccatggtgaccgtgtcatccgcctccggtggtggaggctccggggggcgggcttcaggaggcggaggaagcgatattgtgatgacccagactccgcttagcctgcccgtgactcctggagaaccggcctccatttcctgccggtcctcgcaatcactcctgcattccaacggttacaactacctgaattggtacctccagaagcctggccagtcgccccagttgctgatctatctgggctcgaagcgcgcctccggggtgcctgaccggtttagcggatctgggagcggcacggacttcactctccacatcacccgcgtgggagcggaggacgtgggagtgtactactgtatgcaggcgctgcagactccgtacacattcggacagggcaccaagctggagatcaag

SEQ ID NO: 58 - DNA, 738 bp, synthetic construct
  misc_feature 1..738: note = source = /note="Description of Artificial Sequence: Synthetic polynucleotide"
  source 1..738: mol_type = other DNA
caagtgcaacttcaagaatcaggcggaggactcgtgcagcccggaggatcattgcggctctcgtgcgccgcctcgggcttcaccttctcgagcgacgccatgacctgggtccgccaggccccggggaaggggctggaatgggtgtctgtgatttccggctccgggggaactacgtactacgccgattccgtgaaaggtcgcttcactatctcccgggacaacagcaagaacaccctttatctgcaaatgaattccctccgcgccgaggacaccgccgtgtactactgcgccaagctggactcctcgggctactactatgcccggggtccgagatactggggacagggaaccctcgtgaccgtgtcctccgcgtccggcggaggagggtcgggagggcgggcctccggcggcggcggttcggacatccagctgacccagtccccatcctcactgagcgcaagcgtgggcgacagagtcaccattacatgcagggcgtcccagagcatcagctcctacctgaactggtaccaacagaagcctggaaaggctcctaagctgttgatctacggggcttcgaccctggcatccggggtgcccgcgaggtttagcggaagcggtagcggcactcacttcactctgaccattaacagcctccagtccgaggattcagccacttactactgtcagcagtcctacaagcgggccagcttcggacagggcactaaggtcgagatcaag

SEQ ID NO: 59 - DNA, 741 bp, synthetic construct
  misc_feature 1..741: note = source = /note="Description of Artificial Sequence: Synthetic polynucleotide"
  source 1..741: mol_type = other DNA
caagtccaactggtccagagcggtgcagaagtgaagaagcccggagcgagcgtgaaagtgtcctgcaaggcttccgggtacaccttctccaactacggcatcacttgggtgcgccaggccccgggacagggcctggaatggatggggtggatttccgcgtacaacggcaatacgaactacgctcagaagttccagggtagagtgaccatgactaggaacacctccatttccaccgcctacatggaactgtcctccctgcggagcgaggacaccgccgtgtactattgcgcccggggaccatactactactacatggatgtctgggggaaggggactatggtcaccgtgtcatccgcctcgggaggcggcggatcaggaggacgcgcctctggtggtggaggatcggagatcgtgatgacccagagccctctctccttgcccgtgactcctggggagcccgcatccatttcatgccggagctcccagtcacttctctactccaacggctataactacgtggattggtacctccaaaagccgggccagagcccgcagctgctgatctacctgggctcgaacagggccagcggagtgcctgaccggttctccgggtcgggaagcgggaccgacttcaagctgcaaatctcgagagtggaggccgaggacgtgggaatctactactgtatgcagggccgccagtttccgtactcgttcggacagggcaccaaagtggaaatcaag

SEQ ID NO: 60 - DNA, 714 bp, synthetic construct
  misc_feature 1..714: note = source = /note="Description of Artificial Sequence: Synthetic polynucleotide"
  source 1..714: mol_type = other DNA
gaagtgcaattgctcgaaactggaggaggtctggtgcaacctggaggatcacttcgcctgtcctgcgccgtgtcgggctttgccctgtccaaccatggaatgagctgggtccgccgcgcgccggggaagggcctcgaatgggtgtccggcatcgtctactccggctccacctactacgccgcgtccgtgaagggccggttcacgatttcacgggacaactcgcggaacaccctgtacctccaaatgaattcccttcggccggaggatactgccatctactactgctccgcccacggtggcgaatccgacgtctggggccagggaaccaccgtgaccgtgtccagcgcgtccgggggaggaggaagcgggggtagagcatcgggtggaggcggatcagagatcgtgctgacccagtcccccgccaccttgagcgtgtcaccaggagagtccgccaccctgtcatgccgcgccagccagtccgtgtcctccaacctggcttggtaccagcagaagccggggcaggcccctagactcctgatctatggggcgtcgacccgggcatctggaattcccgataggttcagcggatcgggctcgggcactgacttcactctgaccatctcctcgctgcaagccgaggacgtggctgtgtactactgtcagcagtacggaagctccctgactttcggtggcgggaccaaagtcgagattaag

SEQ ID NO: 61 - DNA, 717 bp, synthetic construct
  misc_feature 1..717: note = source = /note="Description of Artificial Sequence: Synthetic polynucleotide"
  source 1..717: mol_type = other DNA
gaagtgcaattggtggaaactggaggaggacttgtgcaacctggaggatcattgagactgagctgcgcagtgtcgggattcgccctgagcaaccatggaatgtcctgggtcagaagggcccctggaaaaggcctcgaatgggtgtcagggatcgtgtactccggttccacttactacgccgcctccgtgaaggggcgcttcactatctcacgggataactcccgcaataccctgtacctccaaatgaacagcctgcggccggaggataccgccatctactactgttccgcccacggtggagagtctgacgtctggggccagggaactaccgtgaccgtgtcctccgcgtccggcggtggagggagcggcggccgcgccagcggcggcggaggctccgagatcgtgatgacccagagccccgctactctgtcggtgtcgcccggagaaagggcgaccctgtcctgccgggcgtcgcagtccgtgagcagcaagctggcttggtaccagcagaagccgggccaggcaccacgcctgcttatgtacggtgcctccattcgggccaccggaatcccggaccggttctcggggtcggggtccggtaccgagttcacactgaccatttcctcgctcgagcccgaggactttgccgtctattactgccagcagtacggctcctcctcatggacgttcggccaggggaccaaggtcgaaatcaag

SEQ ID NO: 62 - DNA, 723 bp, synthetic construct
  misc_feature 1..723: note = source = /note="Description of Artificial Sequence: Synthetic polynucleotide"
  source 1..723: mol_type = other DNA
gaagtgcaattggtggagactggaggaggagtggtgcaacctggaggaagcctgagactgtcatgcgcggtgtcgggcttcgccctctccaaccacggaatgtcctgggtccgccgggcccctgggaaaggacttgaatgggtgtccggcatcgtgtactcgggttccacctactacgcggcctcagtgaagggccggtttactattagccgcgacaactccagaaacacactgtacctccaaatgaactcgctgcggccggaagataccgctatctactactgctccgcccatgggggagagtcggacgtctggggacagggcaccactgtcactgtgtccagcgcttccggcggtggtggaagcgggggacgggcctcaggaggcggtggcagcgagattgtgctgacccagtcccccgggaccctgagcctgtccccgggagaaagggccaccctctcctgtcgggcatcccagtccgtggggtctactaaccttgcatggtaccagcagaagcccggccaggcccctcgcctgctgatctacgacgcgtccaatagagccaccggcatcccggatcgcttcagcggaggcggatcgggcaccgacttcaccctcaccatttcaaggctggaaccggaggacttcgccgtgtactactgccagcagtatggttcgtccccaccctggacgttcggccaggggactaaggtcgagatcaag

SEQ ID NO: 63 - DNA, 717 bp, synthetic construct
  misc_feature 1..717: note = source = /note="Description of Artificial Sequence: Synthetic polynucleotide"
  source 1..717: mol_type = other DNA
caagtgcaactcgtggaatctggtggaggactcgtgaaacctggaggatcattgagactgtcatgcgcggcctcgggattcacgttctccgattactacatgagctggattcgccaggctccggggaagggactggaatgggtgtcctacatttcctcatccggctccaccatctactacgcggactccgtgaaggggagattcaccattagccgcgataacgccaagaacagcctgtaccttcagatgaactccctgcgggctgaagatactgccgtctactactgcgcaagggagagcggagatgggatggacgtctggggacagggtaccactgtgaccgtgtcgtcggcctccggcggagggggttcgggtggaagggccagcggcggcggaggcagcgacatccagatgacccagtccccctcatcgctgtccgcctccgtgggcgaccgcgtcaccatcacatgccgggcctcacagtcgatctcctcctacctcaattggtatcagcagaagcccggaaaggcccctaagcttctgatctacgcagcgtcctccctgcaatccggggtcccatctcggttctccggctcgggcagcggtaccgacttcactctgaccatctcgagcctgcagccggaggacttcgccacttactactgtcagcaaagctacaccctcgcgtttggccagggcaccaaagtggacatcaag

SEQ ID NO: 64 - DNA, 717 bp, synthetic construct
  misc_feature 1..717: note = source = /note="Description of Artificial Sequence: Synthetic polynucleotide"
  source 1..717: mol_type = other DNA
gaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcgcgtccggcggaggcggcagcgggggtcgggcatcagggggcggcggatcggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaag

SEQ ID NO: 65 - DNA, 738 bp, synthetic construct
  misc_feature 1..738: note = source = /note="Description of Artificial Sequence: Synthetic polynucleotide"
  source 1..738: mol_type = other DNA
caagtgcaactggtgcaaagcggaggaggattggtcaaacccggaggaagcctgagactgtcatgcgcggcctctggattcaccttctccgattactacatgtcatggatcagacaggccccggggaagggcctcgaatgggtgtcctacatctcgtcctccgggaacaccatctactacgccgacagcgtgaagggccgctttaccatttcccgcgacaacgcaaagaactcgctgtaccttcagatgaattccctgcgggctgaagataccgcggtgtactattgcgcccggtccactatggtccgggaggactactggggacagggcacactcgtgaccgtgtccagcgcgagcgggggtggaggcagcggtggacgcgcctccggcggcggcggttcagacatcgtgctgactcagtcgcccctgtcgctgccggtcaccctgggccaaccggcctcaattagctgcaagtcctcggagagcctggtgcacaactcaggaaagacttacctgaactggttccatcagcggcctggacagtccccacggaggctcatctatgaagtgtccaacagggattcgggggtgcccgaccgcttcactggctccgggtccggcaccgacttcaccttgaaaatctccagagtggaagccgaggacgtgggcgtgtactactgtatgcagggtacccactggcctggaacctttggacaaggaactaagctcgagattaag

SEQ ID NO: 66 - DNA, 717 bp, synthetic construct
  misc_feature 1..717: note = source = /note="Description of Artificial Sequence: Synthetic polynucleotide"
  source 1..717: mol_type = other DNA
caagtgcaactcgtggaatctggtggaggactcgtgcaacccggtggaagccttaggctgtcgtgcgccgtcagcgggtttgctctgagcaaccatggaatgtcctgggtccgccgggcaccgggaaaagggctggaatgggtgtccggcatcgtgtacagcgggtcaacctattacgccgcgtccgtgaagggcagattcactatctcaagagacaacagccggaacaccctgtacttgcaaatgaattccctgcgccccgaggacaccgccatctactactgctccgcccacggaggagagtcggacgtgtggggccagggaacgactgtgactgtgtccagcgcatcaggagggggtggttcgggcggccgggcctcggggggaggaggttccgacattcggctgacccagtccccgtccccactgtcggcctccgtcggcgaccgcgtgaccatcacttgtcaggcgtccgaggacattaacaagttcctgaactggtaccaccagacccctggaaaggcccccaagctgctgatctacgatgcctcgacccttcaaactggagtgcctagccggttctccgggtccggctccggcactgatttcactctgaccatcaactcattgcagccggaagatatcgggacctactattgccagcagtacgaatccctcccgctcacattcggcgggggaaccaaggtcgagattaag

SEQ ID NO: 67 - DNA, 720 bp, synthetic construct
  misc_feature 1..720: note = source = /note="Description of Artificial Sequence: Synthetic polynucleotide"
  source 1..720: mol_type = other DNA
gaagtgcaattggtggaaactggaggaggacttgtgcaacctggaggatcattgcggctctcatgcgctgtctccggcttcgccctgtcaaatcacgggatgtcgtgggtcagacgggccccgggaaagggtctggaatgggtgtcggggattgtgtacagcggctccacctactacgccgcttcggtcaagggccgcttcactatttcacgggacaacagccgcaacaccctctatctgcaaatgaactctctccgcccggaggataccgccatctactactgctccgcacacggcggcgaatccgacgtgtggggacagggaaccactgtcaccgtgtcgtccgcatccggtggcggaggatcgggtggccgggcctccgggggcggcggcagcgagactaccctgacccagtcccctgccactctgtccgtgagcccgggagagagagccacccttagctgccgggccagccagagcgtgggctccaacctggcctggtaccagcagaagccaggacagggtcccaggctgctgatctacggagcctccactcgcgcgaccggcatccccgcgaggttctccgggtcgggttccgggaccgagttcaccctgaccatctcctccctccaaccggaggacttcgcggtgtactactgtcagcagtacaacgattggctgcccgtgacatttggacaggggacgaaggtggaaatcaaa

SEQ ID NO: 68 - DNA, 723 bp, synthetic construct
  misc_feature 1..723: note = source = /note="Description of Artificial Sequence: Synthetic polynucleotide"
  source 1..723: mol_type = other DNA
gaagtgcaattggtggaatctggtggaggacttgtgcaacctggaggatcactgagactgtcatgcgcggtgtccggttttgccctgagcaatcatgggatgtcgtgggtccggcgcgcccccggaaagggtctggaatgggtgtcgggtatcgtctactccgggagcacttactacgccgcgagcgtgaagggccgcttcaccatttcccgcgataactcccgcaacaccctgtacttgcaaatgaactcgctccggcctgaggacactgccatctactactgctccgcacacggaggagaatccgacgtgtggggccagggaactaccgtgaccgtcagcagcgcctccggcggcgggggctcaggcggacgggctagcggcggcggtggctccgagatcgtgctgacccagtcgcctggcactctctcgctgagccccggggaaagggcaaccctgtcctgtcgggccagccagtccattggatcatcctccctcgcctggtatcagcagaaaccgggacaggctccgcggctgcttatgtatggggccagctcaagagcctccggcattcccgaccggttctccgggtccggttccggcaccgatttcaccctgactatctcgaggctggagccagaggacttcgccgtgtactactgccagcagtacgcggggtccccgccgttcacgttcggacagggaaccaaggtcgagatcaag

SEQ ID NO: 69 - protein, 120 aa, synthetic construct
  REGION 1..120: note = source = /note="Description of Artificial Sequence: Synthetic polypeptide"
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 70 - protein, 115 aa, synthetic construct
  REGION 1..115: note = source = /note="Description of Artificial Sequence: Synthetic polypeptide"
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSVHSFLAYWGQGTLVTVSS

SEQ ID NO: 71 - protein, 115 aa, synthetic construct
  REGION 1..115: note = source = /note="Description of Artificial Sequence: Synthetic polypeptide"
EVQLLESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 72 - protein, 120 aa, synthetic construct
  REGION 1..120: note = source = /note="Description of Artificial Sequence: Synthetic polypeptide"
QVQLVQSGAEVRKTGASVKVSCKASGYIFDNFGINWVRQAPGQGLEWMGWINPKNNNTNYAQKFQGRVTITADESTNTAYMEVSSLRSEDTAVYYCARGPYYYQSYMDVWGQGTMVTVSS

SEQ ID NO: 73 - protein, 123 aa, synthetic construct
  REGION 1..123: note = source = /note="Description of Artificial Sequence: Synthetic polypeptide"
QVQLQESGGGLVQPGGSLRLSCAASGFTFSSDAMTWVRQAPGKGLEWVSVISGSGGTTYYADSVKGRFTISRDNSKNTLYLQMNSLRAEDTAVYYCAKLDSSGYYYARGPRYWGQGTLVTVSS

SEQ ID NO: 74 - protein, 118 aa, synthetic construct
  REGION 1..118: note = source = /note="Description of Artificial Sequence: Synthetic polypeptide"
QVQLVQSGAEVKKPGASVKVSCKASGYTFSNYGITWVRQAPGQGLEWMGWISAYNGNTNYAQKFQGRVTMTRNTSISTAYMELSSLRSEDTAVYYCARGPYYYYMDVWGKGTMVTVSS

SEQ ID NO: 75 - protein, 115 aa, synthetic construct
  REGION 1..115: note = source = /note="Description of Artificial Sequence: Synthetic polypeptide"
EVQLLETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 76 - protein, 115 aa, synthetic construct
  REGION 1..115: note = source = /note="Description of Artificial Sequence: Synthetic polypeptide"
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 77 - protein, 115 aa, synthetic construct
  REGION 1..115: note = source = /note="Description of Artificial Sequence: Synthetic polypeptide"
EVQLVETGGGVVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 78 - protein, 117 aa, synthetic construct
  REGION 1..117: note = source = /note="Description of Artificial Sequence: Synthetic polypeptide"
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARESGDGMDVWGQGTTVTVSS

SEQ ID NO: 79 - protein, 115 aa, synthetic construct
  REGION 1..115: note = source = /note="Description of Artificial Sequence: Synthetic polypeptide"
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 80 - protein, 117 aa, synthetic construct
  REGION 1..117: note = source = /note="Description of Artificial Sequence: Synthetic polypeptide"
QVQLVQSGGGLVKPGGSLRLSCAASGFTFSDYYMSWIRQAPGKGLEWVSYISSSGNTIYYADSVKGRFTISRDNAKNSLYLQMNSLRAEDTAVYYCARSTMVREDYWGQGTLVTVSS

SEQ ID NO: 81 - protein, 115 aa, synthetic construct
  REGION 1..115: note = source = /note="Description of Artificial Sequence: Synthetic polypeptide"
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 82 - protein, 115 aa, synthetic construct; identical to SEQ ID NO: 76
  REGION 1..115: note = source = /note="Description of Artificial Sequence: Synthetic polypeptide"

SEQ ID NO: 83 - protein, 115 aa, synthetic construct; identical to SEQ ID NO: 79
  REGION 1..115: note = source = /note="Description of Artificial Sequence: Synthetic polypeptide"

SEQ ID NO: 84 - protein, 109 aa, synthetic construct
  REGION 1..109: note = source = /note="Description of Artificial Sequence: Synthetic polypeptide"
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 85 - protein, 112 aa, synthetic construct
  REGION 1..112: note = source = /note="Description of Artificial Sequence: Synthetic polypeptide"
DIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 86 - protein, 111 aa, synthetic construct
  REGION 1..111: note = source = /note="Description of Artificial Sequence: Synthetic polypeptide"
DIVMTQTPLSLSVTPGQPASISCKSSQSLLRNDGKTPLYWYLQKAGQPPQLLIYEVSNRFSGVPDRFSGSGSGTDFTLKISRVEAEDVGAYYCMQNIQFPSFGGGTKLEIK

SEQ ID NO: 87 - protein, 112 aa, synthetic construct
  REGION 1..112: note = source = /note="Description of Artificial Sequence: Synthetic polypeptide"
DIVMTQTPLSLPVTPGEPASISCRSSQSLLHSNGYNYLNWYLQKPGQSPQLLIYLGSKRASGVPDRFSGSGSGTDFTLHITRVGAEDVGVYYCMQALQTPYTFGQGTKLEIK

SEQ ID NO: 88 - protein, 106 aa, synthetic construct
  REGION 1..106: note = source = /note="Description of Artificial Sequence: Synthetic polypeptide"
DIQLTQSPSSLSASVGDRVTITCRASQSISSYLNWYQQKPGKAPKLLIYGASTLASGVPARFSGSGSGTHFTLTINSLQSEDSATYYCQQSYKRASFGQGTKVEIK

SEQ ID NO: 89 - protein, 112 aa, synthetic construct
  REGION 1..112: note = source = /note="Description of Artificial Sequence: Synthetic polypeptide"
EIVMTQSPLSLPVTPGEPASISCRSSQSLLYSNGYNYVDWYLQKPGQSPQLLIYLGSNRASGVPDRFSGSGSGTDFKLQISRVEAEDVGIYYCMQGRQFPYSFGQGTKVEIK

SEQ ID NO: 90 - protein, 106 aa, synthetic construct
  REGION 1..106: note = source = /note="Description of Artificial Sequence: Synthetic polypeptide"
EIVLTQSPATLSVSPGESATLSCRASQSVSSNLAWYQQKPGQAPRLLIYGASTRASGIPDRFSGSGSGTDFTLTISSLQAEDVAVYYCQQYGSSLTFGGGTKVEIK

SEQ ID NO: 91 - protein, 107 aa, synthetic construct
  REGION 1..107: note = source = /note="Description of Artificial Sequence: Synthetic polypeptide"
EIVMTQSPATLSVSPGERATLSCRASQSVSSKLAWYQQKPGQAPRLLMYGASIRATGIPDRFSGSGSGTEFTLTISSLEPEDFAVYYCQQYGSSSWTFGQGTKVEIK

SEQ ID NO: 92 - protein, 109 aa, synthetic construct
  REGION 1..109: note = source = /note="Description of Artificial Sequence: Synthetic polypeptide"
EIVLTQSPGTLSLSPGERATLSCRASQSVGSTNLAWYQQKPGQAPRLLIYDASNRATGIPDRFSGGGSGTDFTLTISRLEPEDFAVYYCQQYGSSPPWTFGQGTKVEIK

SEQ ID NO: 93 - protein, 105 aa, synthetic construct
  REGION 1..105: note = source = /note="Description of Artificial Sequence: Synthetic polypeptide"
DIQMTQSPSSLSASVGDRVTITCRASQSISSYLNWYQQKPGKAPKLLIYAASSLQSGVPSRFSGSGSGTDFTLTISSLQPEDFATYYCQQSYTLAFGQGTKVDIK

SEQ ID NO: 94 - protein, 107 aa, synthetic construct
  REGION 1..107: note = source = /note="Description of Artificial Sequence: Synthetic polypeptide"
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 95 - protein, 112 aa, synthetic construct
  REGION 1..112: note = source = /note="Description of Artificial Sequence: Synthetic polypeptide"
DIVLTQSPLSLPVTLGQPASISCKSSESLVHNSGKTYLNWFHQRPGQSPRRLIYEVSNRDSGVPDRFTGSGSGTDFTLKISRVEAEDVGVYYCMQGTHWPGTFGQGTKLEIK

SEQ ID NO: 96 - protein, 107 aa, synthetic construct
  REGION 1..107: note = source = /note="Description of Artificial Sequence: Synthetic polypeptide"
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 97 - protein, 108 aa, synthetic construct
  REGION 1..108: note = source = /note="Description of Artificial Sequence: Synthetic polypeptide"
ETTLTQSPATLSVSPGERATLSCRASQSVGSNLAWYQQKPGQGPRLLIYGASTRATGIPARFSGSGSGTEFTLTISSLQPEDFAVYYCQQYNDWLPVTFGQGTKVEIK

SEQ ID NO: 98 - protein, 109 aa, synthetic construct
  REGION 1..109: note = source = /note="Description of Artificial Sequence: Synthetic polypeptide"
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 99-128 - skipped (30 entries)

SEQ ID NO: 129 - protein, 243 aa, synthetic construct
  REGION 1..243: note = source = /note="Description of Artificial Sequence: Synthetic polypeptide"
QVQLQESGPGLVKPSETLSLTCTVSGGSISSSYYYWGWIRQPPGKGLEWIGSIYYSGSAYYNPSLKSRVTISVDTSKNQFSLRLSSVTAADTAVYYCARHWQEWPDAFDIWGQGTMVTVSSGGGGSGGGGSGGGGSETTLTQSPAFMSATPGDKVIISCKASQDIDDAMNWYQQKPGEAPLFIIQSATSPVPGIPPRFSGSGFGTDFSLTINNIESEDAAYYFCLQHDNFPLTFGQGTKLEIK

SEQ ID NO: 130 - protein, 244 aa, synthetic construct
  REGION 1..244: note = source = /note="Description of Artificial Sequence: Synthetic polypeptide"
VNLRESGPALVKPTQTLTLTCTFSGFSLRTSGMCVSWIRQPPGKALEWLARIDWDEDKFYSTSLKTRLTISKDTSDNQVVLRMTNMDPADTATYYCARSGAGGTSATAFDIWGPGTMVTVSSGGGGSGGGGSGGGGSDIQMTQSPSSLSASVGDRVTITCRASQDIYNNLAWFQLKPGSAPRSLMYAANKSQSGVPSRFSGSASGTDFTLTISSLQPEDFATYYCQHYYRFPYSFGQGTKLEIK

SEQ ID NO: 131 - protein, 246 aa, synthetic construct
  REGION 1..246: note = source = /note="Description of Artificial Sequence: Synthetic polypeptide"
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAKTIAAVYAFDIWGQGTTVTVSSGGGGSGGGGSGGGGSEIVLTQSPLSLPVTPEEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 132 - protein, 240 aa, synthetic construct
  REGION 1..240: note = source = /note="Description of Artificial Sequence: Synthetic polypeptide"
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDLRGAFDIWGQGTMVTVSSGGGGSGGGGSGGGGSSYVLTQSPSVSAAPGYTATISCGGNNIGTKSVHWYQQKPGQAPLLVIRDDSVRPSKIPGRFSGSNSGNMATLTISGVQAGDEADFYCQVWDSDSEHVVFGGGTKLTVL

SEQ ID NO: 133 - protein, 241 aa, synthetic construct
  REGION 1..241: note = source = /note="Description of Artificial Sequence: Synthetic polypeptide"
QVQLVQSGAEVKKPGASVKVSCKPSGYTVTSHYIHWVRRAPGQGLEWMGMINPSGGVTAYSQTLQGRVTMTSDTSSSTVYMELSSLRSEDTAMYYCAREGSGSGWYFDFWGRGTLVTVSSGGGGSGGGGSGGGGSSYVLTQPPSVSVSPGQTASITCSGDGLSKKYVSWYQQKAGQSPVVLISRDKERPSGIPDRFSGSNSADTATLTISGTQAMDEADYYCQAWDDTTVVFGGGTKLTVL

SEQ ID NO: 134 - protein, 245 aa, synthetic construct
  REGION 1..245: note = source = /note="Description of Artificial Sequence: Synthetic polypeptide"
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARAGIAARLRGAFDIWGQGTMVTVSSGGGGSGGGGSGGGGSDIVMTQSPSSVSASVGDRVIITCRASQGIRNWLAWYQQKPGKAPNLLIYAASNLQSGVPSRFSGSGSGADFTLTISSLQPEDVATYYCQKYNSAPFTFGPGTKVDIK

SEQ ID NO: 135 - protein, 253 aa, synthetic construct
  REGION 1..253: note = source = /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGGYQLLRWDVGLLRSAFDIWGQGTMVTVSSGGGGSGGGGSGGGGSSYVLTQPPSVSVAPGQTARITCGGNNIGSKSVHWYQQKPGQAPVLVLYGKNNRPSGVPDRFSGSRSGTTASLTITGAQAEDEADYYCSSRDSSGDHLRVFGTGTKVTVL

SEQ ID NO: 136 - protein, 248 aa, synthetic construct
  REGION 1..248: note = source = /note="Description of Artificial Sequence: Synthetic polypeptide"
EVQLQQSGPGLVKPSQTLSLTCAISGDSVSSNSAAWNWIRQSPSRGLEWLGRTYYRSKWYSFYAISLKSRIIINPDTSKNQFSLQLKSVTPEDTAVYYCARSSPEGLFLYWFDPWGQGTLVTVSSGGDGSGGGGSGGGGSSSELTQDPAVSVALGQTIRITCQGDSLGNYYATWYQQKPGQAPVLVIYGTNNRPSGIPDRFSASSSGNTASLTITGAQAEDEADYYCNSRDSSGHHLLFGTGTKVTVL

SEQ ID NO: 137 - protein, 246 aa, synthetic construct
  REGION 1..246: note = source = /note="Description of Artificial Sequence: Synthetic polypeptide"
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVEGSGSLDYWGQGTLVTVSSGGGGSGGGGSGGGGSEIVMTQSPGTLSLSPGERATLSCRASQSVSSAYLAWYQQKPGQPPRLLISGASTRATGIPDRFGGSGSGTDFTLTISRLEPEDFAVYYCQHYGSSFNGSSLFTFGQGTRLEIK

SEQ ID NO: 138 - protein, 241 aa, synthetic construct
  REGION 1..241: note = source = /note="Description of Artificial Sequence: Synthetic polypeptide"
EVQLVETGGGLVQPGGSLRLSCAASGITFSRYPMSWVRQAPGKGLEWVSGISDSGVSTYYADSAKGRFTISRDNSKNTLFLQMSSLRDEDTAVYYCVTRAGSEASDIWGQGTMVTVSSGGGGSGGGGSGGGGSEIVLTQSPATLSLSPGERATLSCRASQSVSNSLAWYQQKPGQAPRLLIYDASSRATGIPDRFSGSGSGTDFTLTISRLEPEDFAIYYCQQFGTSSGLTFGGGTKLEIK

SEQ ID NO: 139 - protein, 248 aa, synthetic construct
  REGION 1..248: note = source = /note="Description of Artificial Sequence: Synthetic polypeptide"
QVQLVESGGGLVQPGGSLRLSCAASGFTFSSYAMSWVRQAPGKGLEWVSAISGSGGSTYYADSVKGRFTISRDNAKNSLYLQMNSLRAEDTAIYYCARATYKRELRYYYGMDVWGQGTMVTVSSGGGGSGGGGSGGGGSEIVMTQSPGTVSLSPGERATLSCRASQSVSSSFLAWYQQKPGQAPRLLIYGASSRATGIPDRFSGSGSGTDFTLTISRLEPEDSAVYYCQQYHSSPSWTFGQGTRLEIK

SEQ ID NO: 140 - protein, 248 aa, synthetic construct
  REGION 1..248: note = source = /note="Description of Artificial Sequence: Synthetic polypeptide"
EVQLVETGGGLVQPGGSLRLSCAASGFTFSSYAMSWVRQAPGKGLEWVSAISGSGGSTYYADSVKGRFTISRDNSKNTLYLQMNTLKAEDTAVYYCARATYKRELRYYYGMDVWGQGTTVTVSSGGGGSGGGGSGGGGSEIVLTQSPSTLSLSPGESATLSCRASQSVSTTFLAWYQQKPGQAPRLLIYGSSNRATGIPDRFSGSGSGTDFTLTIRRLEPEDFAVYYCQQYHSSPSWTFGQGTKVEIK

SEQ ID NO: 141 - protein, 239 aa, synthetic construct
  REGION 1..239: note = source = /note="Description of Artificial Sequence: Synthetic polypeptide"
EVQLVETGGGLVQPGRSLRLSCAASGFTFDDYAMHWVRQAPGKGLEWVSGISWNSGSIGYADSVKGRFTISRDNAKNSLYLQMNSLRDEDTAVYYCARVGKAVPDVWGQGTTVTVSSGGGGSGGGGSGGGGSDIVMTQTPSSLSASVGDRVTITCRASQSISSYLNWYQQKPGKAPKLLIYAASSLQSGVPSRFSGSGSGTDFTLTISSLQPEDFATYYCQQSYSTPYSFGQGTRLEIK

SEQ ID NO: 142 - protein, 246 aa, synthetic construct
  REGION 1..246: note = source = /note="Description of Artificial Sequence: Synthetic polypeptide"
EVQLVESGGGLVQPGRSLRLSCTASGFTFDDYAMHWVRQRPGKGLEWVASINWKGNSLAYGDSVKGRFAISRDNAKNTVFLQMNSLRTEDTAVYYCASHQGVAYYNYAMDVWGRGTLVTVSSGGGGSGGGGSGGGGSEIVLTQSPGTLSLSPGERATLSCRATQSIGSSFLAWYQQRPGQAPRLLIYGASQRATGIPDRFSGRGSGTDFTLTISRVEPEDSAVYYCQHYESSPSWTFGQGTKVEIK

SEQ ID NO: 143 - protein, 241 aa, synthetic construct
  REGION 1..241: note = source = /note="Description of Artificial Sequence: Synthetic polypeptide"
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVVRDGMDVWGQGTTVTVSSGGGGSGGGGSGGGGSEIVLTQSPATLSLSPGERATLSCRASQSVSSSYLAWYQQKPGQAPRLLIYGASSRATGIPDRFSGNGSGTDFTLTISRLEPEDFAVYYCQQYGSPPRFTFGPGTKVDIK

SEQ ID NO: 144 - protein, 242 aa, synthetic construct
  REGION 1..242: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKIPQTGTFDYWGQGTLVTVSSGGGGSGGGGSGGGGSEIVLTQSPGTLSLSPGERATLSCRASQSVSSSYLAWYQQRPGQAPRLLIYGASSRATGIPDRFSGSGSGTDFTLTISRLEPEDFAVYYCQHYGSSPSWTFGQGTRLEIK

SEQ ID NO: 145 - protein, 248 aa, synthetic construct
  REGION 1..248: note = source = /note="Description of Artificial Sequence: Synthetic polypeptide"
EVQLVETGGGLVQPGGSLRLSCAASGFTFSSYAMSWVRQAPGKGLEWVSAISGSGGSTYYADSVKGRFTMSRENDKNSVFLQMNSLRVEDTGVYYCARANYKRELRYYYGMDVWGQGTMVTVSSGGGGSGGGGSGGGGSEIVMTQSPGTLSLSPGESATLSCRASQRVASNYLAWYQHKPGQAPSLLISGASSRATGVPDRFSGSGSGTDFTLAISRLEPEDSAVYYCQHYDSSPSWTFGQGTKVEIK

SEQ ID NO: 146 - protein, 244 aa, synthetic construct
  REGION 1..244: note = source = /note="Description of Artificial Sequence: Synthetic polypeptide"
EVQLLETGGGLVQPGGSLRLSCAASGFSFSSYAMSWVRQAPGKGLEWVSAISGSGGSTYYADSVKGRFTISRDNSKNTLYLQMNSLRAEDTAVYYCAKALVGATGAFDIWGQGTLVTVSSGGGGSGGGGSGGGGSEIVLTQSPGTLSLSPGERATLSCRASQSLSSNFLAWYQQKPGQAPGLLIYGASNWATGTPDRFSGSGSGTDFTLTITRLEPEDFAVYYCQYYGTSPMYTFGQGTKVEIK

SEQ ID NO: 147 - protein, 244 aa, synthetic construct
  REGION 1..244: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVLWFGEGFDPWGQGTLVTVSSGGGGSGGGGSGGGGSDIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIK

SEQ ID NO: 148 - protein, 251 aa, synthetic construct
  REGION 1..251: note = source = /note="Description of Artificial Sequence: Synthetic polypeptide"
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSSGGGGSGGGGSGGGGSEIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIK

SEQ ID NO: 149 - protein, 246 aa, synthetic construct
  REGION 1..246: note = source = /note="Description of Artificial Sequence: Synthetic polypeptide"
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSSGGGGSGGGGSGGGGSEIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 150 - DNA, 729 bp, synthetic construct
  misc_feature 1..729: note = source = /note="Description of Artificial Sequence: Synthetic polynucleotide"
  source 1..729: mol_type = other DNA
caagtgcagcttcaggaaagcggaccgggcctggtcaagccatccgaaactctctccctgacttgcactgtgtctggcggttccatctcatcgtcgtactactactggggctggattaggcagccgcccggaaagggactggagtggatcggaagcatctactattccggctcggcgtactacaaccctagcctcaagtcgagagtgaccatctccgtggatacctccaagaaccagttttccctgcgcctgagctccgtgaccgccgctgacaccgccgtgtactactgtgctcggcattggcaggaatggcccgatgccttcgacatttggggccagggcactatggtcactgtgtcatccgggggtggaggcagcgggggaggagggtccggggggggaggttcagagacaaccttgacccagtcacccgcattcatgtccgccactccgggagacaaggtcatcatctcgtgcaaagcgtcccaggatatcgacgatgccatgaattggtaccagcagaagcctggcgaagcgccgctgttcattatccaatccgcaacctcgcccgtgcctggaatcccaccgcggttcagcggcagcggtttcggaaccgacttttccctgaccattaacaacattgagtccgaggacgccgcctactacttctgcctgcaacacgacaacttccctctcacgttcggccagggaaccaagctggaaatcaag

SEQ ID NO: 151 - DNA, 735 bp, synthetic construct
  misc_feature 1..735: note = source = /note="Description of Artificial Sequence: Synthetic polynucleotide"
  source 1..735: mol_type = other DNA
caagtcaatctgcgcgaatccggccccgccttggtcaagcctacccagaccctcactctgacctgtactttctccggcttctccctgcggacttccgggatgtgcgtgtcctggatcagacagcctccgggaaaggccctggagtggctcgctcgcattgactgggatgaggacaagttctactccacctcactcaagaccaggctgaccatcagcaaagatacctctgacaaccaagtggtgctccgcatgaccaacatggacccagccgacactgccacttactactgcgcgaggagcggagcgggcggaacctccgccaccgccttcgatatttggggcccgggtaccatggtcaccgtgtcaagcggaggaggggggtccgggggcggcggttccgggggaggcggatcggacattcagatgactcagtcaccatcgtccctgagcgctagcgtgggcgacagagtgacaatcacttgccgggcatcccaggacatctataacaaccttgcgtggttccagctgaagcctggttccgcaccgcggtcacttatgtacgccgccaacaagagccagtcgggagtgccgtcccggttttccggttcggcctcgggaactgacttcaccctgacgatctccagcctgcaacccgaggatttcgccacctactactgccagcactactaccgctttccctactcgttcggacagggaaccaagctggaaatcaag

SEQ ID NO: 152 - DNA, 738 bp, synthetic construct
  misc_feature 1..738: note = source = /note="Description of Artificial Sequence: Synthetic polynucleotide"
  source 1..738: mol_type = other DNA
gaagtgcagcttgtcgaatccggggggggactggtcaagccgggcggatcactgagactgtcctgcgccgcgagcggcttcacgttctcctcctactccatgaactgggtccgccaagcccccgggaagggactggaatgggtgtcctctatctcctcgtcgtcgtcctacatctactacgccgactccgtgaagggaagattcaccatttcccgcgacaacgcaaagaactcactgtacttgcaaatgaactcactccgggccgaagatactgctgtgtactattgcgccaagactattgccgccgtctacgctttcgacatctggggccagggaaccaccgtgactgtgtcgtccggtggtggtggctcgggcggaggaggaagcggcggcggggggtccgagattgtgctgacccagtcgccactgagcctccctgtgacccccgaggaacccgccagcatcagctgccggtccagccagtccctgctccactccaacggatacaattacctcgattggtaccttcagaagcctggacaaagcccgcagctgctcatctacttgggatcaaaccgcgcgtcaggagtgcctgaccggttctccggctcgggcagcggtaccgatttcaccctgaaaatctccagggtggaggcagaggacgtgggagtgtattactgtatgcaggcgctgcagactccgtacacatttgggcagggcaccaagctggagatcaag

SEQ ID NO: 153 - DNA, 720 bp, synthetic construct
  misc_feature 1..720: note = source = /note="Description of Artificial Sequence: Synthetic polynucleotide"
  source 1..720: mol_type = other DNA
gaagtccagctcgtggagtccggcggaggccttgtgaagcctggaggttcgctgagactgtcctgcgccgcctccggcttcaccttctccgactactacatgtcctggatcagacaggccccgggaaagggcctggaatgggtgtcctacatctcgtcatcgggcagcactatctactacgcggactcagtgaaggggcggttcaccatttcccgggataacgcgaagaactcgctgtatctgcaaatgaactcactgagggccgaggacaccgccgtgtactactgcgcccgcgatctccgcggggcatttgacatctggggacagggaaccatggtcacagtgtccagcggagggggaggatcgggtggcggaggttccgggggtggaggctcctcctacgtgctgactcagagcccaagcgtcagcgctgcgcccggttacacggcaaccatctcctgtggcggaaacaacattgggaccaagtctgtgcactggtatcagcagaagccgggccaagctcccctgttggtgatccgcgatgactccgtgcggcctagcaaaattccgggacggttctccggctccaacagcggcaatatggccactctcaccatctcgggagtgcaggccggagatgaagccgacttctactgccaagtctgggactcagactccgagcatgtggtgttcgggggcggaaccaagctgactgtgctc

SEQ ID NO: 154 - DNA, 723 bp, synthetic construct
  misc_feature 1..723: note = source = /note="Description of Artificial Sequence: Synthetic polynucleotide"
  source 1..723: mol_type = other DNA
caagtgcagctggtgcagagcggggccgaagtcaagaagccgggagcctccgtgaaagtgtcctgcaagccttcgggatacaccgtgacctcccactacattcattgggtccgccgcgcccccggccaaggactcgagtggatgggcatgatcaaccctagcggcggagtgaccgcgtacagccagacgctgcagggacgcgtgactatgacctcggatacctcctcctccaccgtctatatggaactgtccagcctgcggtccgaggataccgccatgtactactgcgcccgggaaggatcaggctccgggtggtatttcgacttctggggaagaggcaccctcgtgactgtgtcatctgggggagggggttccggtggtggcggatcgggaggaggcggttcatcctacgtgctgacccagccaccctccgtgtccgtgagccccggccagactgcatcgattacatgtagcggcgacggcctctccaagaaatacgtgtcgtggtaccagcagaaggccggacagagcccggtggtgctgatctcaagagataaggagcggcctagcggaatcccggacaggttctcgggttccaactccgcggacactgctactctgaccatctcggggacccaggctatggacgaagccgattactactgccaagcctgggacgacactactgtcgtgtttggagggggcaccaagttgaccgtcctt

SEQ ID NO: 155 - DNA, 735 bp, synthetic construct
  misc_feature 1..735: note = source = /note="Description of Artificial Sequence: Synthetic polynucleotide"
  source 1..735: mol_type = other DNA
caagtgcagcttcaggagagcggcccgggactcgtgaagccgtcccagaccctgtccctgacttgcaccgtgtcgggaggaagcatctcgagcggaggctactattggtcgtggattcggcagcaccctggaaagggcctggaatggatcggctacatctactactccggctcgacctactacaacccatcgctgaagtccagagtgacaatctcagtggacacgtccaagaatcagttcagcctgaagctctcttccgtgactgcggccgacaccgccgtgtactactgcgcacgcgctggaattgccgcccggctgaggggtgccttcgacatttggggacagggcaccatggtcaccgtgtcctccggcggcggaggttccgggggtggaggctcaggaggaggggggtccgacatcgtcatgactcagtcgccctcaagcgtcagcgcgtccgtcggggacagagtgatcatcacctgtcgggcgtcccagggaattcgcaactggctggcctggtatcagcagaagcccggaaaggcccccaacctgttgatctacgccgcctcaaacctccaatccggggtgccgagccgcttcagcggctccggttcgggtgccgatttcactctgaccatctcctccctgcaacctgaagatgtggctacctactactgccaaaagtacaactccgcaccttttactttcggaccggggaccaaagtggacattaag

SEQ ID NO: 156 - DNA, 759 bp, synthetic construct
  misc_feature 1..759: note = source = /note="Description of Artificial Sequence: Synthetic polynucleotide"
  source 1..759: mol_type = other DNA
caagtgcagctggtccagtcgggcgccgaggtcaagaagcccgggagctctgtgaaagtgtcctgcaaggcctccgggggcacctttagctcctacgccatctcctgggtccgccaagcaccgggtcaaggcctggagtggatggggggaattatccctatcttcggcactgccaactacgcccagaagttccagggacgcgtgaccattaccgcggacgaatccacctccaccgcttatatggagctgtccagcttgcgctcggaagataccgccgtgtactactgcgcccggaggggtggataccagctgctgagatgggacgtgggcctcctgcggtcggcgttcgacatctggggccagggcactatggtcactgtgtccagcggaggaggcggatcgggaggcggcggatcagggggaggcggttccagctacgtgcttactcaacccccttcggtgtccgtggccccgggacagaccgccagaatcacttgcggaggaaacaacattgggtccaagagcgtgcattggtaccagcagaagccaggacaggcccctgtgctggtgctctacgggaagaacaatcggcccagcggagtgccggacaggttctcgggttcacgctccggtacaaccgcttcactgactatcaccggggcccaggcagaggatgaagcggactactactgttcctcccgggattcatccggcgaccacctccgggtgttcggaaccggaacgaaggtcaccgtgctg

SEQ ID NO: 157 - DNA, 744 bp, synthetic construct
  misc_feature 1..744: note = source = /note="Description of Artificial Sequence: Synthetic polynucleotide"
  source 1..744: mol_type = other DNA
gaagtgcagctccaacagtcaggaccggggctcgtgaagccatcccagaccctgtccctgacttgtgccatctcgggagatagcgtgtcatcgaactccgccgcctggaactggattcggcagagcccgtcccgcggactggagtggcttggaaggacctactaccggtccaagtggtactctttctacgcgatctcgctgaagtcccgcattatcattaaccctgatacctccaagaatcagttctccctccaactgaaatccgtcacccccgaggacacagcagtgtattactgcgcacggagcagccccgaaggactgttcctgtattggtttgacccctggggccaggggactcttgtgaccgtgtcgagcggcggagatgggtccggtggcggtggttcggggggcggcggatcatcatccgaactgacccaggacccggctgtgtccgtggcgctgggacaaaccatccgcattacgtgccagggagactccctgggcaactactacgccacttggtaccagcagaagccgggccaagcccctgtgttggtcatctacgggaccaacaacagaccttccggcatccccgaccggttcagcgcttcgtcctccggcaacactgccagcctgaccatcactggagcgcaggccgaagatgaggccgactactactgcaacagcagagactcctcgggtcatcacctcttgttcggaactggaaccaaggtcaccgtgctg

SEQ ID NO: 158 - DNA, 738 bp, synthetic construct
  misc_feature 1..738: note = source = /note="Description of Artificial Sequence: Synthetic polynucleotide"
  source 1..738: mol_type = other DNA
gaagtgcagctcgtggagtcaggaggcggcctggtccagccgggagggtcccttagactgtcatgcgccgcaagcggattcactttctcctcctatgccatgagctgggtccgccaagcccccggaaagggactggaatgggtgtccgccatctcggggtctggaggctcaacttactacgctgactccgtgaagggacggttcaccattagccgcgacaactccaagaacaccctctacctccaaatgaactccctgcgggccgaggataccgccgtctactactgcgccaaagtggaaggttcaggatcgctggactactggggacagggtactctcgtgaccgtgtcatcgggcggaggaggttccggcggtggcggctccggcggcggagggtcggagatcgtgatgacccagagccctggtactctgagcctttcgccgggagaaagggccaccctgtcctgccgcgcttcccaatccgtgtcctccgcgtacttggcgtggtaccagcagaagccgggacagccccctcggctgctgatcagcggggccagcacccgggcaaccggaatcccagacagattcgggggttccggcagcggcacagatttcaccctgactatttcgaggttggagcccgaggactttgcggtgtattactgtcagcactacgggtcgtcctttaatggctccagcctgttcacgttcggacaggggacccgcctggaaatcaag

SEQ ID NO: 159 - DNA, 723 bp, synthetic construct
  misc_feature 1..723: note = source = /note="Description of Artificial Sequence: Synthetic polynucleotide"
  source 1..723: mol_type = other DNA
gaagtgcaactggtggaaaccggtggcggcctggtgcagcctggaggatcattgaggctgtcatgcgcggccagcggtattaccttctcccggtaccccatgtcctgggtcagacaggccccggggaaagggcttgaatgggtgtccgggatctcggactccggtgtcagcacttactacgccgactccgccaagggacgcttcaccatttcccgggacaactcgaagaacaccctgttcctccaaatgagctccctccgggacgaggatactgcagtgtactactgcgtgacccgcgccgggtccgaggcgtctgacatttggggacagggcactatggtcaccgtgtcgtccggcggagggggctcgggaggcggtggcagcggaggaggagggtccgagatcgtgctgacccaatccccggccaccctctcgctgagccctggagaaagggcaaccttgtcctgtcgcgcgagccagtccgtgagcaactccctggcctggtaccagcagaagcccggacaggctccgagacttctgatctacgacgcttcgagccgggccactggaatccccgaccgcttttcggggtccggctcaggaaccgatttcaccctgacaatctcacggctggagccagaggatttcgccatctattactgccagcagttcggtacttcctccggcctgactttcggaggcggcacgaagctcgaaatcaag

SEQ ID NO: 160 - DNA, 744 bp, synthetic construct
  misc_feature 1..744: note = source = /note="Description of Artificial Sequence: Synthetic polynucleotide"
  source 1..744: mol_type = other DNA
caagtgcagctcgtggaatcgggtggcggactggtgcagccggggggctcacttagactgtcctgcgcggccagcggattcactttctcctcctacgccatgtcctgggtcagacaggcccctggaaagggcctggaatgggtgtccgcaatcagcggcagcggcggctcgacctattacgcggattcagtgaagggcagattcaccatttcccgggacaacgccaagaactccttgtaccttcaaatgaactccctccgcgcggaagataccgcaatctactactgcgctcgggccacttacaagagggaactgcgctactactacgggatggacgtctggggccagggaaccatggtcaccgtgtccagcggaggaggaggatcgggaggaggcggtagcgggggtggagggtcggagatcgtgatgacccagtcccccggcactgtgtcgctgtcccccggcgaacgggccaccctgtcatgtcgggccagccagtcagtgtcgtcaagcttcctcgcctggtaccagcagaaaccgggacaagctccccgcctgctgatctacggagccagcagccgggccaccggtattcctgaccggttctccggttcggggtccgggaccgactttactctgactatctctcgcctcgagccagaggactccgccgtgtattactgccagcagtaccactcctccccgtcctggacgttcggacagggcacaaggctggagattaag

SEQ ID NO: 161 - DNA, 744 bp, synthetic construct
  misc_feature 1..744: note = source = /note="Description of Artificial Sequence: Synthetic polynucleotide"
  source 1..744: mol_type = other DNA
gaggtgcagcttgtggaaaccggtggcggactggtgcagcccggaggaagcctcaggctgtcctgcgccgcgtccggcttcaccttctcctcgtacgccatgtcctgggtccgccaggcccccggaaagggcctggaatgggtgtccgccatctctggaagcggaggttccacgtactacgcggacagcgtcaagggaaggttcacaatctcccgcgataattcgaagaacactctgtaccttcaaatgaacaccctgaaggccgaggacactgctgtgtactactgcgcacgggccacctacaagagagagctccggtactactacggaatggacgtctggggccagggaactactgtgaccgtgtcctcgggagggggtggctccggggggggcggctccggcggaggcggttccgagattgtgctgacccagtcaccttcaactctgtcgctgtccccgggagagagcgctactctgagctgccgggccagccagtccgtgtccaccaccttcctcgcctggtatcagcagaagccggggcaggcaccacggctcttgatctacgggtcaagcaacagagcgaccggaattcctgaccgcttctcggggagcggttcaggcaccgacttcaccctgactatccggcgcctggaacccgaagatttcgccgtgtattactgtcaacagtaccactcctcgccgtcctggacctttggccaaggaaccaaagtggaaatcaag

SEQ ID NO: 162 - DNA, 717 bp, synthetic construct
  misc_feature 1..717: note = source = /note="Description of Artificial Sequence: Synthetic polynucleotide"
  source 1..717: mol_type = other DNA
gaagtgcagctcgtggaaactggaggtggactcgtgcagcctggacggtcgctgcggctgagctgcgctgcatccggcttcaccttcgacgattatgccatgcactgggtcagacaggcgccagggaagggacttgagtgggtgtccggtatcagctggaatagcggctcaatcggatacgcggactccgtgaagggaaggttcaccatttcccgcgacaacgccaagaactccctgtacttgcaaatgaacagcctccgggatgaggacactgccgtgtactactgcgcccgcgtcggaaaagctgtgcccgacgtctggggccagggaaccactgtgaccgtgtccagcggcgggggtggatcgggcggtggagggtccggtggagggggctcagatattgtgatgacccagaccccctcgtccctgtccgcctcggtcggcgaccgcgtgactatcacatgtagagcctcgcagagcatctccagctacctgaactggtatcagcagaagccggggaaggccccgaagctcctgatctacgcggcatcatcactgcaatcgggagtgccgagccggttttccgggtccggctccggcaccgacttcacgctgaccatttcttccctgcaacccgaggacttcgccacttactactgccagcagtcctactccaccccttactccttcggccaaggaaccaggctggaaatcaag

SEQ ID NO: 163 - DNA, 738 bp, synthetic construct
  misc_feature 1..738: note = source = /note="Description of Artificial Sequence: Synthetic polynucleotide"
  source 1..738: mol_type = other DNA
gaagtgcagctcgtggagagcgggggaggattggtgcagcccggaaggtccctgcggctctcctgcactgcgtctggcttcaccttcgacgactacgcgatgcactgggtcagacagcgcccgggaaagggcctggaatgggtcgcctcaatcaactggaagggaaactccctggcctatggcgacagcgtgaagggccgcttcgccatttcgcgcgacaacgccaagaacaccgtgtttctgcaaatgaattccctgcggaccgaggataccgctgtgtactactgcgccagccaccagggcgtggcatactataactacgccatggacgtgtggggaagagggacgctcgtcaccgtgtcctccgggggcggtggatcgggtggaggaggaagcggtggcgggggcagcgaaatcgtgctgactcagagcccgggaactctttcactgtccccgggagaacgggccactctctcgtgccgggccacccagtccatcggctcctccttccttgcctggtaccagcagaggccaggacaggcgccccgcctgctgatctacggtgcttcccaacgcgccactggcattcctgaccggttcagcggcagagggtcgggaaccgatttcacactgaccatttcccgggtggagcccgaagattcggcagtctactactgtcagcattacgagtcctccccttcatggaccttcggtcaagggaccaaagtggagatcaag

SEQ ID NO: 164 - DNA, 723 bp, synthetic construct
  misc_feature 1..723: note = source = /note="Description of Artificial Sequence: Synthetic polynucleotide"
  source 1..723: mol_type = other DNA
gaggtgcagttggtcgaaagcgggggcgggcttgtgcagcctggcggatcactgcggctgtcctgcgcggcatcaggcttcacgttttcttcctacgccatgtcctgggtgcgccaggcccctggaaagggactggaatgggtgtccgcgatttcggggtccggcgggagcacctactacgccgattccgtgaagggccgcttcactatctcgcgggacaactccaagaacaccctctacctccaaatgaatagcctgcgggccgaggataccgccgtctactattgcgctaaggtcgtgcgcgacggaatggacgtgtggggacagggtaccaccgtgacagtgtcctcggggggaggcggtagcggcggaggaggaagcggtggtggaggttccgagattgtgctgactcaatcacccgcgaccctgagcctgtcccccggcgaaagggccactctgtcctgtcgggccagccaatcagtctcctcctcgtacctggcctggtaccagcagaagccaggacaggctccgagactccttatctatggcgcatcctcccgcgccaccggaatcccggataggttctcgggaaacggatcggggaccgacttcactctcaccatctcccggctggaaccggaggacttcgccgtgtactactgccagcagtacggcagcccgcctagattcactttcggccccggcaccaaagtggacatcaag

SEQ ID NO: 165 - DNA, 726 bp, synthetic construct
  misc_feature 1..726: note = source = /note="Description of Artificial Sequence: Synthetic polynucleotide"
  source 1..726: mol_type = other DNA
gaagtgcagctgctggagtccggcggtggattggtgcaaccggggggatcgctcagactgtcctgtgcggcgtcaggcttcaccttctcgagctacgccatgtcatgggtcagacaggcccctggaaagggtctggaatgggtgtccgccatttccgggagcgggggatctacatactacgccgatagcgtgaagggccgcttcaccatttcccgggacaactccaagaacactctctatctgcaaatgaactccctccgcgctgaggacactgccgtgtactactgcgccaaaatccctcagaccggcaccttcgactactggggacaggggactctggtcaccgtcagcagcggtggcggaggttcggggggaggaggaagcggcggcggagggtccgagattgtgctgacccagtcacccggcactttgtccctgtcgcctggagaaagggccaccctttcctgccgggcatcccaatccgtgtcctcctcgtacctggcctggtaccagcagaggcccggacaggccccacggcttctgatctacggagcaagcagccgcgcgaccggtatcccggaccggttttcgggctcgggctcaggaactgacttcaccctcaccatctcccgcctggaacccgaagatttcgctgtgtattactgccagcactacggcagctccccgtcctggacgttcggccagggaactcggctggagatcaag

SEQ ID NO: 166 - DNA, 744 bp, synthetic construct
  misc_feature 1..744: note = source = /note="Description of Artificial Sequence: Synthetic polynucleotide"
  source 1..744: mol_type = other DNA
gaagtgcaactggtggaaaccggtggaggactcgtgcagcctggcggcagcctccggctgagctgcgccgcttcgggattcaccttttcctcctacgcgatgtcttgggtcagacaggcccccggaaaggggctggaatgggtgtcagccatctccggctccggcggatcaacgtactacgccgactccgtgaaaggccggttcaccatgtcgcgcgagaatgacaagaactccgtgttcctgcaaatgaactccctgagggtggaggacaccggagtgtactattgtgcgcgcgccaactacaagagagagctgcggtactactacggaatggacgtctggggacagggaactatggtgaccgtgtcatccggtggagggggaagcggcggtggaggcagcgggggcgggggttcagaaattgtcatgacccagtccccgggaactctttccctctcccccggggaatccgcgactttgtcctgccgggccagccagcgcgtggcctcgaactacctcgcatggtaccagcataagccaggccaagccccttccctgctgatttccggggctagcagccgcgccactggcgtgccggataggttctcgggaagcggctcgggtaccgatttcaccctggcaatctcgcggctggaaccggaggattcggccgtgtactactgccagcactatgactcatccccctcctggacattcggacagggcaccaaggtcgagatcaag

SEQ ID NO: 167 - DNA, 732 bp, synthetic construct
  misc_feature 1..732: note = source = /note="Description of Artificial Sequence: Synthetic polynucleotide"
  source 1..732: mol_type = other DNA
gaagtgcagctgctcgaaaccggtggagggctggtgcagccagggggctccctgaggctttcatgcgccgctagcggattctccttctcctcttacgccatgtcgtgggtccgccaagcccctggaaaaggcctggaatgggtgtccgcgatttccgggagcggaggttcgacctattacgccgactccgtgaagggccgctttaccatctcccgggataactccaagaacactctgtacctccaaatgaactcgctgagagccgaggacaccgccgtgtattactgcgcgaaggcgctggtcggcgcgactggggcattcgacatctggggacagggaactcttgtgaccgtgtcgagcggaggcggcggctccggcggaggagggagcgggggcggtggttccgaaatcgtgttgactcagtccccgggaaccctgagcttgtcacccggggagcgggccactctctcctgtcgcgcctcccaatcgctctcatccaatttcctggcctggtaccagcagaagcccggacaggccccgggcctgctcatctacggcgcttcaaactgggcaacgggaacccctgatcggttcagcggaagcggatcgggtactgactttaccctgaccatcaccagactggaaccggaggacttcgccgtgtactactgccagtactacggcacctcccccatgtacacattcggacagggtaccaaggtcgagattaag

SEQ ID NO: 168 - DNA, 733 bp, synthetic construct
  misc_feature 1..733: note = source = /note="Description of Artificial Sequence: Synthetic polynucleotide"
  source 1..733: mol_type = other DNA
gaagtgcagctgcttgagagcggtggaggtctggtgcagcccgggggatcactgcgcctgtcctgtgccgcgtccggtttcactttctcctcgtacgccatgtcgtgggtcagacaggcaccgggaaagggactggaatgggtgtcagccatttcgggttcggggggcagcacctactacgctgactccgtgaagggccggttcaccatttcccgcgacaactccaagaacaccttgtacctccaaatgaactccctgcgggccgaagataccgccgtgtattactgcgtgctgtggttcggagagggattcgacccgtggggacaaggaacactcgtgactgtgtcatccggcggaggcggcagcggtggcggcggttccggcggcggcggatctgacatcgtgttgacccagtcccctctgagcctgccggtcactcctggcgaaccagccagcatctcctgccggtcgagccagtccctcctgcactccaatgggtacaactacctcgattggtatctgcaaaagccgggccagagcccccagctgctgatctaccttgggtcaaaccgcgcttccggggtgcctgatagattctccgggtccgggagcggaaccgactttaccctgaaaatctcgagggtggaggccgaggacgtcggagtgtactactgcatgcaggcgctccagactcccctgaccttcggaggaggaacgaaggtcgacatcaaga

SEQ ID NO: 169 - DNA, 753 bp, synthetic construct
  misc_feature 1..753: note = source = /note="Description of Artificial Sequence: Synthetic polynucleotide"
  source 1..753: mol_type = other DNA
caagtgcagctcgtggagtcaggcggaggactggtgcagcccgggggctccctgagactttcctgcgcggcatcgggttttaccttctcctcctatgctatgtcctgggtgcgccaggccccgggaaagggactggaatgggtgtccgcaatcagcggtagcgggggctcaacatactacgccgactccgtcaagggtcgcttcactatttcccgggacaactccaagaataccctgtacctccaaatgaacagcctcagggccgaggatactgccgtgtactactgcgccaaagtcggatacgatagctccggttactaccgggactactacggaatggacgtgtggggacagggcaccaccgtgaccgtgtcaagcggcggaggcggttcaggagggggaggctccggcggtggagggtccgaaatcgtcctgactcagtcgcctggcactctgtcgttgtccccgggggagcgcgctaccctgtcgtgtcgggcgtcgcagtccgtgtcgagctcctacctcgcgtggtaccagcagaagcccggacaggcccctagacttctgatctacggcacttcttcacgcgccaccgggatcagcgacaggttcagcggctccggctccgggaccgacttcaccctgaccattagccggctggagcctgaagatttcgccgtgtattactgccaacactacggaaactcgccgccaaagttcacgttcggacccggaaccaagctggaaatcaag

SEQ ID NO: 170 - DNA, 739 bp, synthetic construct
  misc_feature 1..739: note = source = /note="Description of Artificial Sequence: Synthetic polynucleotide"
  source 1..739: mol_type = other DNA
gaagtccaactggtggagtccgggggagggctcgtgcagcccggaggcagccttcggctgtcgtgcgccgcctccgggttcacgttctcatcctacgcgatgtcgtgggtcagacaggcaccaggaaagggactggaatgggtgtccgccattagcggctccggcggtagcacctactatgccgactcagtgaagggaaggttcactatctcccgcgacaacagcaagaacaccctgtacctccaaatgaactctctgcgggccgaggataccgcggtgtactattgcgccaagatgggttggtccagcggatacttgggagccttcgacatttggggacagggcactactgtgaccgtgtcctccgggggtggcggatcgggaggcggcggctcgggtggagggggttccgaaatcgtgttgacccagtcaccgggaaccctctcgctgtccccgggagaacgggctacactgtcatgtagagcgtcccagtccgtggcttcctcgttcctggcctggtaccagcagaagccgggacaggcaccccgcctgctcatctacggagccagcggccgggcgaccggcatccctgaccgcttctccggttccggctcgggcaccgactttactctgaccattagcaggcttgagcccgaggattttgccgtgtactactgccaacactacggggggagccctcgcctgaccttcggaggcggaactaaggtcgatatcaaaa

SEQ ID NO: 171 - protein, 121 aa, synthetic construct
  REGION 1..121: note = source = /note="Description of Artificial Sequence: Synthetic polypeptide"
QVQLQESGPGLVKPSETLSLTCTVSGGSISSSYYYWGWIRQPPGKGLEWIGSIYYSGSAYYNPSLKSRVTISVDTSKNQFSLRLSSVTAADTAVYYCARHWQEWPDAFDIWGQGTMVTVSS

SEQ ID NO: 172 - protein, 123 aa, synthetic construct
  REGION 1..123: note = source = /note="Description of Artificial Sequence: Synthetic polypeptide"
QVNLRESGPALVKPTQTLTLTCTFSGFSLRTSGMCVSWIRQPPGKALEWLARIDWDEDKFYSTSLKTRLTISKDTSDNQVVLRMTNMDPADTATYYCARSGAGGTSATAFDIWGPGTMVTVSS

SEQ ID NO: 173 - protein, 119 aa, synthetic construct
  REGION 1..119: note = source = /note="Description of Artificial Sequence: Synthetic polypeptide"
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAKTIAAVYAFDIWGQGTTVTVSS

SEQ ID NO: 174 - protein, 117 aa, synthetic construct
  REGION 1..117: note = source = /note="Description of Artificial Sequence: Synthetic polypeptide"
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDLRGAFDIWGQGTMVTVSS

SEQ ID NO: 175 - protein, 120 aa, synthetic construct
  REGION 1..120: note = source = /note="Description of Artificial Sequence: Synthetic polypeptide"
QVQLVQSGAEVKKPGASVKVSCKPSGYTVTSHYIHWVRRAPGQGLEWMGMINPSGGVTAYSQTLQGRVTMTSDTSSSTVYMELSSLRSEDTAMYYCAREGSGSGWYFDFWGRGTLVTVSS

SEQ ID NO: 176 - protein, 123 aa, synthetic construct
  REGION 1..123: note = source = /note="Description of Artificial Sequence: Synthetic polypeptide"
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARAGIAARLRGAFDIWGQGTMVTVSS

SEQ ID NO: 177 - protein, 129 aa, synthetic construct
  REGION 1..129: note = source = /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGGYQLLRWDVGLLRSAFDIWGQGTMVTVSS

SEQ ID NO: 178 - protein, 125 aa, synthetic construct
  REGION 1..125: note = source = /note="Description of Artificial Sequence: Synthetic polypeptide"
EVQLQQSGPGLVKPSQTLSLTCAISGDSVSSNSAAWNWIRQSPSRGLEWLGRTYYRSKWYSFYAISLKSRIIINPDTSKNQFSLQLKSVTPEDTAVYYCARSSPEGLFLYWFDPWGQGTLVTVSS

SEQ ID NO: 179 - protein, 118 aa, synthetic construct
  REGION 1..118: note = source = /note="Description of Artificial Sequence: Synthetic polypeptide"
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVEGSGSLDYWGQGTLVTVSS

SEQ ID NO: 180 - protein, 118 aa, synthetic construct
  REGION 1..118: note = source = /note="Description of Artificial Sequence: Synthetic polypeptide"
EVQLVETGGGLVQPGGSLRLSCAASGITFSRYPMSWVRQAPGKGLEWVSGISDSGVSTYYADSAKGRFTISRDNSKNTLFLQMSSLRDEDTAVYYCVTRAGSEASDIWGQGTMVTVSS

SEQ ID NO: 181 - protein, 124 aa, synthetic construct
  REGION 1..124: note = source = /note="Description of Artificial Sequence: Synthetic polypeptide"
QVQLVESGGGLVQPGGSLRLSCAASGFTFSSYAMSWVRQAPGKGLEWVSAISGSGGSTYYADSVKGRFTISRDNAKNSLYLQMNSLRAEDTAIYYCARATYKRELRYYYGMDVWGQGTMVTVSS

SEQ ID NO: 182 - protein, 124 aa, synthetic construct
  REGION 1..124: note = source = /note="Description of Artificial Sequence: Synthetic polypeptide"
EVQLVETGGGLVQPGGSLRLSCAASGFTFSSYAMSWVRQAPGKGLEWVSAISGSGGSTYYADSVKGRFTISRDNSKNTLYLQMNTLKAEDTAVYYCARATYKRELRYYYGMDVWGQGTTVTVSS

SEQ ID NO: 183 - protein, 117 aa, synthetic construct
  REGION 1..117: note = source = /note="Description of Artificial Sequence: Synthetic polypeptide"
EVQLVETGGGLVQPGRSLRLSCAASGFTFDDYAMHWVRQAPGKGLEWVSGISWNSGSIGYADSVKGRFTISRDNAKNSLYLQMNSLRDEDTAVYYCARVGKAVPDVWGQGTTVTVSS

SEQ ID NO: 184 - protein, 122 aa, synthetic construct
  REGION 1..122: note = source = /note="Description of Artificial Sequence: Synthetic polypeptide"
EVQLVESGGGLVQPGRSLRLSCTASGFTFDDYAMHWVRQRPGKGLEWVASINWKGNSLAYGDSVKGRFAISRDNAKNTVFLQMNSLRTEDTAVYYCASHQGVAYYNYAMDVWGRGTLVTVSS

SEQ ID NO: 185 - protein, 117 aa, synthetic construct
  REGION 1..117: note = source = /note="Description of Artificial Sequence: Synthetic polypeptide"
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVVRDGMDVWGQGTTVTVSS

SEQ ID NO: 186 - protein, 118 aa, synthetic construct
  REGION 1..118: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKIPQTGTFDYWGQGTLVTVSS

SEQ ID NO: 187 - protein, 124 aa, synthetic construct
  REGION 1..124: note = source = /note="Description of Artificial Sequence: Synthetic polypeptide"
EVQLVETGGGLVQPGGSLRLSCAASGFTFSSYAMSWVRQAPGKGLEWVSAISGSGGSTYYADSVKGRFTMSRENDKNSVFLQMNSLRVEDTGVYYCARANYKRELRYYYGMDVWGQGTMVTVSS

SEQ ID NO: 188 - protein, 120 aa, synthetic construct
  REGION 1..120: note = source = /note="Description of Artificial Sequence: Synthetic polypeptide"
EVQLLETGGGLVQPGGSLRLSCAASGFSFSSYAMSWVRQAPGKGLEWVSAISGSGGSTYYADSVKGRFTISRDNSKNTLYLQMNSLRAEDTAVYYCAKALVGATGAFDIWGQGTLVTVSS

SEQ ID NO: 189 - protein, 117 aa, synthetic construct
  REGION 1..117: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVLWFGEGFDPWGQGTLVTVSS

SEQ ID NO: 190 - protein, 126 aa, synthetic construct
  REGION 1..126: note = source = /note="Description of Artificial Sequence: Synthetic polypeptide"
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSS

SEQ ID NO: 191 - protein, 122 aa, synthetic construct
  REGION 1..122: note = source = /note="Description of Artificial Sequence: Synthetic polypeptide"
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSS

SEQ ID NO: 192 - protein, 107 aa, synthetic construct
  REGION 1..107: note = source = /note="Description of Artificial Sequence: Synthetic polypeptide"
ETTLTQSPAFMSATPGDKVIISCKASQDIDDAMNWYQQKPGEAPLFIIQSATSPVPGIPPRFSGSGFGTDFSLTINNIESEDAAYYFCLQHDNFPLTFGQGTKLEIK

SEQ ID NO: 193 - protein, 107 aa, synthetic construct
  REGION 1..107: note = source = /note="Description of Artificial Sequence: Synthetic polypeptide"
DIQMTQSPSSLSASVGDRVTITCRASQDIYNNLAWFQLKPGSAPRSLMYAANKSQSGVPSRFSGSASGTDFTLTISSLQPEDFATYYCQHYYRFPYSFGQGTKLEIK

SEQ ID NO: 194 - protein, 112 aa, synthetic construct
  REGION 1..112: note = source = /note="Description of Artificial Sequence: Synthetic polypeptide"
EIVLTQSPLSLPVTPEEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 195 - protein, 108 aa, synthetic construct
  REGION 1..108: note = source = /note="Description of Artificial Sequence: Synthetic polypeptide"
SYVLTQSPSVSAAPGYTATISCGGNNIGTKSVHWYQQKPGQAPLLVIRDDSVRPSKIPGRFSGSNSGNMATLTISGVQAGDEADFYCQVWDSDSEHVVFGGGTKLTVL

SEQ ID NO: 196 - protein, 106 aa, synthetic construct
  REGION 1..106: note = source = /note="Description of Artificial Sequence: Synthetic polypeptide"
SYVLTQPPSVSVSPGQTASITCSGDGLSKKYVSWYQQKAGQSPVVLISRDKERPSGIPDRFSGSNSADTATLTISGTQAMDEADYYCQAWDDTTVVFGGGTKLTVL

SEQ ID NO: 197 - protein, 107 aa, synthetic construct
  REGION 1..107: note = source = /note="Description of Artificial Sequence: Synthetic polypeptide"
DIVMTQSPSSVSASVGDRVIITCRASQGIRNWLAWYQQKPGKAPNLLIYAASNLQSGVPSRFSGSGSGADFTLTISSLQPEDVATYYCQKYNSAPFTFGPGTKVDIK

SEQ ID NO: 198 - protein, 109 aa, synthetic construct
  REGION 1..109: note = source = /note="Description of Artificial Sequence: Synthetic polypeptide"
SYVLTQPPSVSVAPGQTARITCGGNNIGSKSVHWYQQKPGQAPVLVLYGKNNRPSGVPDRFSGSRSGTTASLTITGAQAEDEADYYCSSRDSSGDHLRVFGTGTKVTVL

SEQ ID NO: 199 - protein, 108 aa, synthetic construct
  REGION 1..108: note = source = /note="Description of Artificial Sequence: Synthetic polypeptide"
SSELTQDPAVSVALGQTIRITCQGDSLGNYYATWYQQKPGQAPVLVIYGTNNRPSGIPDRFSASSSGNTASLTITGAQAEDEADYYCNSRDSSGHHLLFGTGTKVTVL

SEQ ID NO: 200 - protein, 113 aa, synthetic construct
  REGION 1..113: note = source = /note="Description of Artificial Sequence: Synthetic polypeptide"
EIVMTQSPGTLSLSPGERATLSCRASQSVSSAYLAWYQQKPGQPPRLLISGASTRATGIPDRFGGSGSGTDFTLTISRLEPEDFAVYYCQHYGSSFNGSSLFTFGQGTRLEIK

SEQ ID NO: 201 - protein, 108 aa, synthetic construct
  REGION 1..108: note = source = /note="Description of Artificial Sequence: Synthetic polypeptide"
EIVLTQSPATLSLSPGERATLSCRASQSVSNSLAWYQQKPGQAPRLLIYDASSRATGIPDRFSGSGSGTDFTLTISRLEPEDFAIYYCQQFGTSSGLTFGGGTKLEIK

SEQ ID NO: 202 - protein, 109 aa, synthetic construct
  REGION 1..109: note = source = /note="Description of Artificial Sequence: Synthetic polypeptide"
EIVMTQSPGTVSLSPGERATLSCRASQSVSSSFLAWYQQKPGQAPRLLIYGASSRATGIPDRFSGSGSGTDFTLTISRLEPEDSAVYYCQQYHSSPSWTFGQGTRLEIK

SEQ ID NO: 203 - protein, 109 aa, synthetic construct
  REGION 1..109: note = source = /note="Description of Artificial Sequence: Synthetic polypeptide"
EIVLTQSPSTLSLSPGESATLSCRASQSVSTTFLAWYQQKPGQAPRLLIYGSSNRATGIPDRFSGSGSGTDFTLTIRRLEPEDFAVYYCQQYHSSPSWTFGQGTKVEIK

SEQ ID NO: 204 - protein, 107 aa, synthetic construct
  REGION 1..107: note = source = /note="Description of Artificial Sequence: Synthetic polypeptide"
DIVMTQTPSSLSASVGDRVTITCRASQSISSYLNWYQQKPGKAPKLLIYAASSLQSGVPSRFSGSGSGTDFTLTISSLQPEDFATYYCQQSYSTPYSFGQGTRLEIK

SEQ ID NO: 205 - protein, 109 aa, synthetic construct
  REGION 1..109: note = source = /note="Description of Artificial Sequence: Synthetic polypeptide"
EIVLTQSPGTLSLSPGERATLSCRATQSIGSSFLAWYQQRPGQAPRLLIYGASQRATGIPDRFSGRGSGTDFTLTISRVEPEDSAVYYCQHYESSPSWTFGQGTKVEIK

SEQ ID NO: 206 - protein, 109 aa, synthetic construct
  REGION 1..109: note = source = /note="Description of Artificial Sequence: Synthetic polypeptide"
EIVLTQSPATLSLSPGERATLSCRASQSVSSSYLAWYQQKPGQAPRLLIYGASSRATGIPDRFSGNGSGTDFTLTISRLEPEDFAVYYCQQYGSPPRFTFGPGTKVDIK

SEQ ID NO: 207 - protein, 109 aa, synthetic construct
  REGION 1..109: note = source = /note="Description of Artificial Sequence: Synthetic polypeptide"
EIVLTQSPGTLSLSPGERATLSCRASQSVSSSYLAWYQQRPGQAPRLLIYGASSRATGIPDRFSGSGSGTDFTLTISRLEPEDFAVYYCQHYGSSPSWTFGQGTRLEIK

SEQ ID NO: 208 - protein, 109 aa, synthetic construct
  REGION 1..109: note = source = /note="Description of Artificial Sequence: Synthetic polypeptide"
EIVMTQSPGTLSLSPGESATLSCRASQRVASNYLAWYQHKPGQAPSLLISGASSRATGVPDRFSGSGSGTDFTLAISRLEPEDSAVYYCQHYDSSPSWTFGQGTKVEIK

SEQ ID NO: 209 - protein, 109 aa, synthetic construct
  REGION 1..109: note = source = /note="Description of Artificial Sequence: Synthetic polypeptide"
EIVLTQSPGTLSLSPGERATLSCRASQSLSSNFLAWYQQKPGQAPGLLIYGASNWATGTPDRFSGSGSGTDFTLTITRLEPEDFAVYYCQYYGTSPMYTFGQGTKVEIK

SEQ ID NO: 210 - protein, 112 aa, synthetic construct
  REGION 1..112: note = source = /note="Description of Artificial Sequence: Synthetic polypeptide"
DIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIK

SEQ ID NO: 211 - protein, 110 aa, synthetic construct
  REGION 1..110: note = source = /note="Description of Artificial Sequence: Synthetic polypeptide"
EIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIK

SEQ ID NO: 212 - protein, 109 aa, synthetic construct
  REGION 1..109: note = source = /note="Description of Artificial Sequence: Synthetic polypeptide"
EIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 213-254 - skipped (42 entries)

SEQ ID NO: 255 - protein, 122 aa, synthetic construct
  REGION 1..122: note = source = /note="Description of Artificial Sequence: Synthetic polypeptide"
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 256 - protein, 117 aa, synthetic construct
  REGION 1..117: note = source = /note="Description of Artificial Sequence: Synthetic polypeptide"
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 257 - protein, 117 aa, synthetic construct
  REGION 1..117: note = source = /note="Description of Artificial Sequence: Synthetic polypeptide"
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 258 - protein, 117 aa, synthetic construct
  REGION 1..117: note = source = /note="Description of Artificial Sequence: Synthetic polypeptide"
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 259 - protein, 107 aa, synthetic construct
  REGION 1..107: note = source = /note="Description of Artificial Sequence: Synthetic polypeptide"
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 260 - protein, 111 aa, synthetic construct
  REGION 1..111: note = source = /note="Description of Artificial Sequence: Synthetic polypeptide"
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 261 - protein, 111 aa, synthetic construct
  REGION 1..111: note = source = /note="Description of Artificial Sequence: Synthetic polypeptide"
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 262 - protein, 111 aa, synthetic construct; identical to SEQ ID NO: 261
  REGION 1..111: note = source = /note="Description of Artificial Sequence: Synthetic polypeptide"

SEQ ID NO: 263 - protein, 244 aa, synthetic construct
  REGION 1..244: note = source = /note="Description of Artificial Sequence: Synthetic polypeptide"
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 264 - protein, 249 aa, synthetic construct
  REGION 1..249: note = source = /note="Description of Artificial Sequence: Synthetic polypeptide"
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 265 - protein, 243 aa, synthetic construct
  REGION 1..243: note = source = /note="Description of Artificial Sequence: Synthetic polypeptide"
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 266 - protein, 243 aa, synthetic construct
  REGION 1..243: note = source = /note="Description of Artificial Sequence: Synthetic polypeptide"
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 267-382 - skipped (116 entries)

SEQ ID NO: 383 - protein, 4 aa, synthetic construct
  REGION 1..4: note = source = /note="Description of Artificial Sequence: Synthetic peptide"
RGDS

SEQ ID NO: 384 - protein, 5 aa, synthetic construct
  REGION 1..5: note = source = /note="Description of Artificial Sequence: Synthetic peptide"
NYAMS

SEQ ID NO: 385 - protein, 5 aa, synthetic construct
  REGION 1..5: note = source = /note="Description of Artificial Sequence: Synthetic peptide"
DYAMH

SEQ ID NO: 386 - protein, 5 aa, synthetic construct
  REGION 1..5: note = source = /note="Description of Artificial Sequence: Synthetic peptide"
NHGMS

SEQ ID NO: 387 - protein, 5 aa, synthetic construct
  REGION 1..5: note = source = /note="Description of Artificial Sequence: Synthetic peptide"
NFGIN

SEQ ID NO: 388 - protein, 5 aa, synthetic construct
  REGION 1..5: note = source = /note="Description of Artificial Sequence: Synthetic peptide"
SDAMT

SEQ ID NO: 389 - protein, 5 aa, synthetic construct
  REGION 1..5: note = source = /note="Description of Artificial Sequence: Synthetic peptide"
NYGIT

SEQ ID NO: 390 - protein, 5 aa, synthetic construct
  REGION 1..5: note = source = /note="Description of Artificial Sequence: Synthetic peptide"
NHGMS

SEQ ID NO: 391 - protein, 5 aa, synthetic construct
  REGION 1..5: note = source = /note="Description of Artificial Sequence: Synthetic peptide"
NHGMS

SEQ ID NO: 392 - protein, 5 aa, synthetic construct
  REGION 1..5: note = source = /note="Description of Artificial Sequence: Synthetic peptide"
NHGMS

SEQ ID NO: 393 - protein, 5 aa, synthetic construct
  REGION 1..5: note = source = /note="Description of Artificial Sequence: Synthetic peptide"
DYYMS

SEQ ID NO: 394 - protein, 5 aa, synthetic construct
  REGION 1..5: note = source = /note="Description of Artificial Sequence: Synthetic peptide"
NHGMS

SEQ ID NO: 395 - protein, 5 aa, synthetic construct
  REGION 1..5: note = source = /note="Description of Artificial Sequence: Synthetic peptide"
DYYMS

SEQ ID NO: 396 - protein, 5 aa, synthetic construct
  REGION 1..5: note = source = /note="Description of Artificial Sequence: Synthetic peptide"
NHGMS

SEQ ID NO: 397 - protein, 5 aa, synthetic construct
  REGION 1..5: note = source = /note="Description of Artificial Sequence: Synthetic peptide"
NHGMS

SEQ ID NO: 398 - protein, 5 aa, synthetic construct
  REGION 1..5: note = source = /note="Description of Artificial Sequence: Synthetic peptide"
NHGMS

SEQ ID NO: 399 - protein, 7 aa, synthetic construct
  REGION 1..7: note = source = /note="Description of Artificial Sequence: Synthetic peptide"
SSYYYWG

SEQ ID NO: 400 - protein, 7 aa, synthetic construct
  REGION 1..7: note = source = /note="Description of Artificial Sequence: Synthetic peptide"
TSGMCVS

SEQ ID NO: 401 - protein, 5 aa, synthetic construct
  REGION 1..5: note = source = /note="Description of Artificial Sequence: Synthetic peptide"
SYSMN

SEQ ID NO: 402 - protein, 5 aa, synthetic construct
  REGION 1..5: note = source = /note="Description of Artificial Sequence: Synthetic peptide"
DYYMS

SEQ ID NO: 403 - protein, 5 aa, synthetic construct
  REGION 1..5: note = source = /note="Description of Artificial Sequence: Synthetic peptide"
SHYIH

SEQ ID NO: 404 - protein, 7 aa, synthetic construct
  REGION 1..7: note = source = /note="Description of Artificial Sequence: Synthetic peptide"
SGGYYWS

SEQ ID NO: 405 - protein, 5 aa, synthetic construct
  REGION 1..5: note = source = /note="Description of Artificial Sequence: Synthetic peptide"
SYAIS

SEQ ID NO: 406 - protein, 7 aa, synthetic construct
  REGION 1..7: note = source = /note="Description of Artificial Sequence: Synthetic peptide"
SNSAAWN

SEQ ID NO: 407 - protein, 5 aa, synthetic construct
  REGION 1..5: note = source = /note="Description of Artificial Sequence: Synthetic peptide"
SYAMS

SEQ ID NO: 408 - protein, 5 aa, synthetic construct
  REGION 1..5: note = source = /note="Description of Artificial Sequence: Synthetic peptide"
RYPMS

SEQ ID NO: 409 - protein, 5 aa, synthetic construct
  REGION 1..5: note = source = /note="Description of Artificial Sequence: Synthetic peptide"
SYAMS

SEQ ID NO: 410 - protein, 5 aa, synthetic construct
  REGION 1..5: note = source = /note="Description of Artificial Sequence: Synthetic peptide"
SYAMS

SEQ ID NO: 411 - protein, 5 aa, synthetic construct
  REGION 1..5: note = source = /note="Description of Artificial Sequence: Synthetic peptide"
DYAMH

SEQ ID NO: 412 - protein, 5 aa, synthetic construct
  REGION 1..5: note = source = /note="Description of Artificial Sequence: Synthetic peptide"
DYAMH

SEQ ID NO: 413 - protein, 5 aa, synthetic construct
  REGION 1..5: note = source = /note="Description of Artificial Sequence: Synthetic peptide"
SYAMS

SEQ ID NO: 414 - protein, 5 aa, synthetic construct
  REGION 1..5: note = source = /note="Description of Artificial Sequence: Synthetic peptide"
SYAMS

SEQ ID NO: 415 - protein, 5 aa, synthetic construct
  REGION 1..5: note = source = /note="Description of Artificial Sequence: Synthetic peptide"
SYAMS

SEQ ID NO: 416 - protein, 5 aa, synthetic construct
  REGION 1..5: note = source = /note="Description of Artificial Sequence: Synthetic peptide"
SYAMS

SEQ ID NO: 417 - protein, 5 aa, synthetic construct
  REGION 1..5: note = source = /note="Description of Artificial Sequence: Synthetic peptide"
SYAMS

SEQ ID NO: 418 - protein, 5 aa, synthetic construct
  REGION 1..5: note = source = /note="Description of Artificial Sequence: Synthetic peptide"
SYAMS

SEQ ID NO: 419 - protein, 5 aa, synthetic construct
  REGION 1..5: note = source = /note="Description of Artificial Sequence: Synthetic peptide"
SYAMS

SEQ ID NO: 420 - protein, 5 aa, synthetic construct
  REGION 1..5: note = source = /note="Description of Artificial Sequence: Synthetic peptide"
NFGMN

SEQ ID NO: 421 - protein, 5 aa, synthetic construct
  REGION 1..5: note = source = /note="Description of Artificial Sequence: Synthetic peptide"
DYSIN

SEQ ID NO: 422 - protein, 5 aa, synthetic construct
  REGION 1..5: note = source = /note="Description of Artificial Sequence: Synthetic peptide"
HYSMN

SEQ ID NO: 423 - protein, 5 aa, synthetic construct
  REGION 1..5: note = source = /note="Description of Artificial Sequence: Synthetic peptide"
HYSMN

SEQ ID NO: 424 - protein, 17 aa, synthetic construct
  REGION 1..17: note = source = /note="Description of Artificial Sequence: Synthetic peptide"
GISRSGENTYYADSVKG

SEQ ID NO: 425 - protein, 17 aa, synthetic construct
  REGION 1..17: note = source = /note="Description of Artificial Sequence: Synthetic peptide"
GISWNSGSIGYADSVKG

SEQ ID NO: 426 - protein, 16 aa, synthetic construct
  REGION 1..16: note = source = /note="Description of Artificial Sequence: Synthetic peptide"
GIVYSGSTYYAASVKG

SEQ ID NO: 427 - protein, 17 aa, synthetic construct
  REGION 1..17: note = source = /note="Description of Artificial Sequence: Synthetic peptide"
WINPKNNNTNYAQKFQG

SEQ ID NO: 428 - protein, 17 aa, synthetic construct
  REGION 1..17: note = source = /note="Description of Artificial Sequence: Synthetic peptide"
VISGSGGTTYYADSVKG

SEQ ID NO: 429 - protein, 17 aa, synthetic construct
  REGION 1..17: note = source = /note="Description of Artificial Sequence: Synthetic peptide"
WISAYNGNTNYAQKFQG

SEQ ID NO: 430 - protein, 16 aa, synthetic construct
  REGION 1..16: note = source = /note="Description of Artificial Sequence: Synthetic peptide"
GIVYSGSTYYAASVKG

SEQ ID NO: 431 - protein, 16 aa, synthetic construct
  REGION 1..16: note = source = /note="Description of Artificial Sequence: Synthetic peptide"
GIVYSGSTYYAASVKG

SEQ ID NO: 432 - protein, 16 aa, synthetic construct
  REGION 1..16: note = source = /note="Description of Artificial Sequence: Synthetic peptide"
GIVYSGSTYYAASVKG

SEQ ID NO: 433 - protein, 17 aa, synthetic construct
  REGION 1..17: note = source = /note="Description of Artificial Sequence: Synthetic peptide"
YISSSGSTIYYADSVKG

SEQ ID NO: 434 - protein, 16 aa, synthetic construct
  REGION 1..16: note = source = /note="Description of Artificial Sequence: Synthetic peptide"
GIVYSGSTYYAASVKG

SEQ ID NO: 435 - protein, 17 aa, synthetic construct
  REGION 1..17: note = source = /note="Description of Artificial Sequence: Synthetic peptide"
YISSSGNTIYYADSVKG

SEQ ID NO: 436 - protein, 16 aa, synthetic construct
  REGION 1..16: note = source = /note="Description of Artificial Sequence: Synthetic peptide"
GIVYSGSTYYAASVKG

SEQ ID NO: 437 - protein, 16 aa, synthetic construct
  REGION 1..16: note = source = /note="Description of Artificial Sequence: Synthetic peptide"
GIVYSGSTYYAASVKG

SEQ ID NO: 438 - protein, 16 aa, synthetic construct
  REGION 1..16: note = source = /note="Description of Artificial Sequence: Synthetic peptide"
GIVYSGSTYYAASVKG

SEQ ID NO: 439 - protein, 16 aa, synthetic construct
  REGION 1..16: note = source = /note="Description of Artificial Sequence: Synthetic peptide"
SIYYSGSAYYNPSLKS

SEQ ID NO: 440 - protein, 16 aa, synthetic construct
  REGION 1..16: note = source = /note="Description of Artificial Sequence: Synthetic peptide"
RIDWDEDKFYSTSLKT

SEQ ID NO: 441 - protein, 17 aa, synthetic construct
  REGION 1..17: note = source = /note="Description of Artificial Sequence: Synthetic peptide"
SISSSSSYIYYADSVKG

SEQ ID NO: 442 - protein, 17 aa, synthetic construct
  REGION 1..17: note = source = /note="Description of Artificial Sequence: Synthetic peptide"
YISSSGSTIYYADSVKG

SEQ ID NO: 443 - protein, 17 aa, synthetic construct
  REGION 1..17: note = source = /note="Description of Artificial Sequence: Synthetic peptide"
MINPSGGVTAYSQTLQG

SEQ ID NO: 444 - protein, 16 aa, synthetic construct
  REGION 1..16: note = source = /note="Description of Artificial Sequence: Synthetic peptide"
YIYYSGSTYYNPSLKS

SEQ ID NO: 445 - protein, 17 aa, synthetic construct
  REGION 1..17: note = source = /note="Description of Artificial Sequence: Synthetic peptide"
GIIPIFGTANYAQKFQG

SEQ ID NO: 446 - protein, 18 aa, synthetic construct
  REGION 1..18: note = source = /note="Description of Artificial Sequence: Synthetic peptide"
RTYYRSKWYSFYAISLKS

SEQ ID NO: 447 - protein, 17 aa, synthetic construct
  REGION 1..17: note = source = /note="Description of Artificial Sequence: Synthetic peptide"
AISGSGGSTYYADSVKG

SEQ ID NO: 448 - protein, 17 aa, synthetic construct
  REGION 1..17: note = source = /note="Description of Artificial Sequence: Synthetic peptide"
GISDSGVSTYYADSAKG

SEQ ID NO: 449 - protein, 17 aa, synthetic construct
  REGION 1..17: note = source = /note="Description of Artificial Sequence: Synthetic peptide"
AISGSGGSTYYADSVKG

SEQ ID NO: 450 - protein, 17 aa, synthetic construct
  REGION 1..17: note = source = /note="Description of Artificial Sequence: Synthetic peptide"
AISGSGGSTYYADSVKG

SEQ ID NO: 451 - protein, 17 aa, synthetic construct
  REGION 1..17: note = source = /note="Description of Artificial Sequence: Synthetic peptide"
GISWNSGSIGYADSVKG

SEQ ID NO: 452 - protein, 17 aa, synthetic construct
  REGION 1..17: note = source = /note="Description of Artificial Sequence: Synthetic peptide"
SINWKGNSLAYGDSVKG

SEQ ID NO: 453 - protein, 17 aa, synthetic construct
  REGION 1..17: note = source = /note="Description of Artificial Sequence: Synthetic peptide"
AISGSGGSTYYADSVKG

SEQ ID NO: 454 - protein, 17 aa, synthetic construct
  REGION 1..17: note = source = /note="Description of Artificial Sequence: Synthetic peptide"
AISGSGGSTYYADSVKG

SEQ ID NO: 455 - protein, 17 aa, synthetic construct
  REGION 1..17: note = source = /note="Description of Artificial Sequence: Synthetic peptide"
AISGSGGSTYYADSVKG

SEQ ID NO: 456 - protein, 17 aa, synthetic construct
  REGION 1..17: note = source = /note="Description of Artificial Sequence: Synthetic peptide"
AISGSGGSTYYADSVKG

SEQ ID NO: 457 - protein, 17 aa, synthetic construct
  REGION 1..17: note = source = /note="Description of Artificial Sequence: Synthetic peptide"
AISGSGGSTYYADSVKG

SEQ ID NO: 458 - protein, 17 aa, synthetic construct
  REGION 1..17: note = source = /note="Description of Artificial Sequence: Synthetic peptide"
AISGSGGSTYYADSVKG

SEQ ID NO: 459 - protein, 17 aa, synthetic construct
  REGION 1..17: note = source = /note="Description of Artificial Sequence: Synthetic peptide"
AISGSGGSTYYADSVKG

SEQ ID NO: 460 - protein, 17 aa, synthetic construct
  REGION 1..17: note = source = /note="Description of Artificial Sequence: Synthetic peptide"
WINTYTGESYFADDFKG

SEQ ID NO: 461 - protein, 17 aa, synthetic construct
  REGION 1..17: note = source = /note="Description of Artificial Sequence: Synthetic peptide"
WINTETREPAYAYDFRG

SEQ ID NO: 462 - protein, 17 aa, synthetic construct
  REGION 1..17: note = source = /note="Description of Artificial Sequence: Synthetic peptide"
RINTESGVPIYADDFKG

SEQ ID NO: 463 - protein, 17 aa, synthetic construct
  REGION 1..17: note = source = /note="Description of Artificial Sequence: Synthetic peptide"
RINTETGEPLYADDFKG

SEQ ID NO: 464 - protein, 11 aa, synthetic construct
  REGION 1..11: note = source = /note="Description of Artificial Sequence: Synthetic peptide"
SPAHYYGGMDV

SEQ ID NO: 465 - protein, 6 aa, synthetic construct
  REGION 1..6: note = source = /note="Description of Artificial Sequence: Synthetic peptide"
HSFLAY

SEQ ID NO: 466 - protein, 7 aa, synthetic construct
  REGION 1..7: note = source = /note="Description of Artificial Sequence: Synthetic peptide"
HGGESDV

SEQ ID NO: 467 - protein, 11 aa, synthetic construct
  REGION 1..11: note = source = /note="Description of Artificial Sequence: Synthetic peptide"
GPYYYQSYMDV

SEQ ID NO: 468 - protein, 14 aa, synthetic construct
  REGION 1..14: note = source = /note="Description of Artificial Sequence: Synthetic peptide"
LDSSGYYYARGPRY

SEQ ID NO: 469 - protein, 9 aa, synthetic construct
  REGION 1..9: note = source = /note="Description of Artificial Sequence: Synthetic peptide"
GPYYYYMDV

SEQ ID NO: 470 - protein, 7 aa, synthetic construct
  REGION 1..7: note = source = /note="Description of Artificial Sequence: Synthetic peptide"
HGGESDV

SEQ ID NO: 471 - protein, 7 aa, synthetic construct
  REGION 1..7: note = source = /note="Description of Artificial Sequence: Synthetic peptide"
HGGESDV

SEQ ID NO: 472 - protein, 7 aa, synthetic construct
  REGION 1..7: note = source = /note="Description of Artificial Sequence: Synthetic peptide"
HGGESDV

SEQ ID NO: 473 - protein, 8 aa, synthetic construct
  REGION 1..8: note = source = /note="Description of Artificial Sequence: Synthetic peptide"
ESGDGMDV

SEQ ID NO: 474 - protein, 7 aa, synthetic construct
  REGION 1..7: note = source = /note="Description of Artificial Sequence: Synthetic peptide"
HGGESDV

SEQ ID NO: 475 - protein, 8 aa, synthetic construct
  REGION 1..8: note = source = /note="Description of Artificial Sequence: Synthetic peptide"
STMVREDY

SEQ ID NO: 476 - protein, 7 aa, synthetic construct
  REGION 1..7: note = source = /note="Description of Artificial Sequence: Synthetic peptide"
HGGESDV

SEQ ID NO: 477 - protein, 7 aa, synthetic construct
  REGION 1..7: note = source = /note="Description of Artificial Sequence: Synthetic peptide"
HGGESDV

SEQ ID NO: 478 - protein, 7 aa, synthetic construct
  REGION 1..7: note = source = /note="Description of Artificial Sequence: Synthetic peptide"
HGGESDV

SEQ ID NO: 479 - protein, 11 aa, synthetic construct
  REGION 1..11: note = source = /note="Description of Artificial Sequence: Synthetic peptide"
HWQEWPDAFDI

SEQ ID NO: 480 - protein, 13 aa, synthetic construct
  REGION 1..13: note = source = /note="Description of Artificial Sequence: Synthetic peptide"
SGAGGTSATAFDI

SEQ ID NO: 481 - protein, 10 aa, synthetic construct
  REGION 1..10: note = source = /note="Description of Artificial Sequence: Synthetic peptide"
TIAAVYAFDI

SEQ ID NO: 482 - protein, 8 aa, synthetic construct
  REGION 1..8: note = source = /note="Description of Artificial Sequence: Synthetic peptide"
DLRGAFDI

SEQ ID NO: 483 - protein, 11 aa, synthetic construct
  REGION 1..11: note = source = /note="Description of Artificial Sequence: Synthetic peptide"
EGSGSGWYFDF

SEQ ID NO: 484 - protein, 13 aa, synthetic construct
  REGION 1..13: note = source = /note="Description of Artificial Sequence: Synthetic peptide"
AGIAARLRGAFDI

SEQ ID NO: 485 - protein, 20 aa, synthetic construct
  REGION 1..20: note = source = /note="Description of Artificial Sequence: Synthetic peptide"
RGGYQLLRWDVGLLRSAFDI

SEQ ID NO: 486 - protein, 13 aa, synthetic construct
  REGION 1..13: note = source = /note="Description of Artificial Sequence: Synthetic peptide"
SSPEGLFLYWFDP

SEQ ID NO: 487 - protein, 9 aa, synthetic construct
  REGION 1..9: note = source = /note="Description of Artificial Sequence: Synthetic peptide"
VEGSGSLDY

SEQ ID NO: 488 - protein, 9 aa, synthetic construct
  REGION 1..9: note = source = /note="Description of Artificial Sequence: Synthetic peptide"
RAGSEASDI

SEQ ID NO: 489 - protein, 15 aa, synthetic construct
  REGION 1..15: note = source = /note="Description of Artificial Sequence: Synthetic peptide"
ATYKRELRYYYGMDV

SEQ ID NO: 490 - protein, 15 aa, synthetic construct
  REGION 1..15: note = source = /note="Description of Artificial Sequence: Synthetic peptide"
ATYKRELRYYYGMDV

SEQ ID NO: 491 - protein, 8 aa, synthetic construct
  REGION 1..8: note = source = /note="Description of Artificial Sequence: Synthetic peptide"
VGKAVPDV

SEQ ID NO: 492 - protein, 13 aa, synthetic construct
  REGION 1..13: note = source = /note="Description of Artificial Sequence: Synthetic peptide"
HQGVAYYNYAMDV

SEQ ID NO: 493 - protein, 8 aa, synthetic construct
  REGION 1..8: note = source = /note="Description of Artificial Sequence: Synthetic peptide"
VVRDGMDV

SEQ ID NO: 494 - protein, 9 aa, synthetic construct
  REGION 1..9: note = source = /note="Description of Artificial Sequence: Synthetic peptide"
IPQTGTFDY

SEQ ID NO: 495 - protein, 15 aa, synthetic construct
  REGION 1..15: note = source = /note="Description of Artificial Sequence: Synthetic peptide"
ANYKRELRYYYGMDV

SEQ ID NO: 496 - protein, 11 aa, synthetic construct
  REGION 1..11: note = source = /note="Description of Artificial Sequence: Synthetic peptide"
ALVGATGAFDI

SEQ ID NO: 497 - protein, 8 aa, synthetic construct
  REGION 1..8: note = source = /note="Description of Artificial Sequence: Synthetic peptide"
WFGEGFDP

SEQ ID NO: 498 - protein, 17 aa, synthetic construct
  REGION 1..17: note = source = /note="Description of Artificial Sequence: Synthetic peptide"
VGYDSSGYYRDYYGMDV

SEQ ID NO: 499 - protein, 13 aa, synthetic construct
  REGION 1..13: note = source = /note="Description of Artificial Sequence: Synthetic peptide"
MGWSSGYLGAFDI

SEQ ID NO: 500 - protein, 13 aa, synthetic construct
  REGION 1..13: note = source = /note="Description of Artificial Sequence: Synthetic peptide"
GEIYYGYDGGFAY

SEQ ID NO: 501 - protein, 8 aa, synthetic construct
  REGION 1..8: note = source = /note="Description of Artificial Sequence: Synthetic peptide"
DYSYAMDY

SEQ ID NO: 502 - protein, 8 aa, synthetic construct
  REGION 1..8: note = source = /note="Description of Artificial Sequence: Synthetic peptide"
DYLYSLDF

SEQ ID NO: 503 - protein, 8 aa, synthetic construct
  REGION 1..8: note = source = /note="Description of Artificial Sequence: Synthetic peptide"
DYLYSCDY

SEQ ID NO: 504 - protein, 12 aa, synthetic construct
  REGION 1..12: note = source = /note="Description of Artificial Sequence: Synthetic peptide"
RASQSISSSFLA

SEQ ID NO: 505 - protein, 16 aa, synthetic construct
  REGION 1..16: note = source = /note="Description of Artificial Sequence: Synthetic peptide"
RSSQSLLHSNGYNYLD

SEQ ID NO: 506 - protein, 16 aa, synthetic construct
  REGION 1..16: note = source = /note="Description of Artificial Sequence: Synthetic peptide"
KSSQSLLRNDGKTPLY

SEQ ID NO: 507 - protein, 16 aa, synthetic construct
  REGION 1..16: note = source = /note="Description of Artificial Sequence: Synthetic peptide"
RSSQSLLHSNGYNYLN

SEQ ID NO: 508 - protein, 11 aa, synthetic construct
  REGION 1..11: note = source = /note="Description of Artificial Sequence: Synthetic peptide"
RASQSISSYLN

SEQ ID NO: 509 - protein, 16 aa, synthetic construct
  REGION 1..16: note = source = /note="Description of Artificial Sequence: Synthetic peptide"
RSSQSLLYSNGYNYVD

SEQ ID NO: 510 - protein, 11 aa, synthetic construct
  REGION 1..11: note = source = /note="Description of Artificial Sequence: Synthetic peptide"
RASQSVSSNLA

SEQ ID NO: 511 - protein, 11 aa, synthetic construct
  REGION 1..11: note = source = /note="Description of Artificial Sequence: Synthetic peptide"
RASQSVSSKLA

SEQ ID NO: 512 - protein, 12 aa, synthetic construct
  REGION 1..12: note = source = /note="Description of Artificial Sequence: Synthetic peptide"
RASQSVGSTNLA

SEQ ID NO: 513 - protein, 11 aa, synthetic construct
  REGION 1..11: note = source = /note="Description of Artificial Sequence: Synthetic peptide"
RASQSISSYLN

SEQ ID NO: 514 - protein, 11 aa, synthetic construct
  REGION 1..11: note = source = /note="Description of Artificial Sequence: Synthetic peptide"
RASQSISSYLN

SEQ ID NO: 515 - protein, 16 aa, synthetic construct
  REGION 1..16: note = source = /note="Description of Artificial Sequence: Synthetic peptide"
KSSESLVHNSGKTYLN

SEQ ID NO: 516 - protein, 11 aa, synthetic construct
  REGION 1..11: note = source = /note="Description of Artificial Sequence: Synthetic peptide"
QASEDINKFLN

SEQ ID NO: 517 - protein, 11 aa, synthetic construct
  REGION 1..11: note = source = /note="Description of Artificial Sequence: Synthetic peptide"
RASQSVGSNLA

SEQ ID NO: 518 - protein, 12 aa, synthetic construct
  REGION 1..12: note = source = /note="Description of Artificial Sequence: Synthetic peptide"
RASQSIGSSSLA

SEQ ID NO: 519 - protein, 11 aa, synthetic construct
  REGION 1..11: note = source = /note="Description of Artificial Sequence: Synthetic peptide"
KASQDIDDAMN

SEQ ID NO: 520 - protein, 11 aa, synthetic construct
  REGION 1..11: note = source = /note="Description of Artificial Sequence: Synthetic peptide"
RASQDIYNNLA

SEQ ID NO: 521 - protein, 16 aa, synthetic construct
  REGION 1..16: note = source = /note="Description of Artificial Sequence: Synthetic peptide"
RSSQSLLHSNGYNYLD

SEQ ID NO: 522 - protein, 11 aa, synthetic construct
  REGION 1..11: note = source = /note="Description of Artificial Sequence: Synthetic peptide"
GGNNIGTKSVH

SEQ ID NO: 523 - protein, 11 aa, synthetic construct
  REGION 1..11: note = source = /note="Description of Artificial Sequence: Synthetic peptide"
SGDGLSKKYVS

SEQ ID NO: 524 - protein, 11 aa, synthetic construct
  REGION 1..11: note = source = /note="Description of Artificial Sequence: Synthetic peptide"
RASQGIRNWLA

SEQ ID NO: 525 - protein, 11 aa, synthetic construct
  REGION 1..11: note = source = /note="Description of Artificial Sequence: Synthetic peptide"
GGNNIGSKSVH

SEQ ID NO: 526 - protein, 11 aa, synthetic construct
  REGION 1..11: note = source = /note="Description of Artificial Sequence: Synthetic peptide"
QGDSLGNYYAT

SEQ ID NO: 527 - protein, 12 aa, synthetic construct
  REGION 1..12: note = source = /note="Description of Artificial Sequence: Synthetic peptide"
RASQSVSSAYLA

SEQ ID NO: 528 - protein, 11 aa, synthetic construct
  REGION 1..11: note = source = /note="Description of Artificial Sequence: Synthetic peptide"
RASQSVSNSLA

SEQ ID NO: 529 - protein, 12 aa, synthetic construct
  REGION 1..12: note = source = /note="Description of Artificial Sequence: Synthetic peptide"
RASQSVSSSFLA

SEQ ID NO: 530 - protein, 12 aa, synthetic construct
  REGION 1..12: note = source = /note="Description of Artificial Sequence: Synthetic peptide"
RASQSVSTTFLA

SEQ ID NO: 531 - protein, 11 aa, synthetic construct
  REGION 1..11: note = source = /note="Description of Artificial Sequence: Synthetic peptide"
RASQSISSYLN

SEQ ID NO: 532 - protein, 12 aa, synthetic construct
  REGION 1..12: note = source = /note="Description of Artificial Sequence: Synthetic peptide"
RATQSIGSSFLA

SEQ ID NO: 533 - protein, 12 aa, synthetic construct
  REGION 1..12: note = source = /note="Description of Artificial Sequence: Synthetic peptide"
RASQSVSSSYLA

SEQ ID NO: 534 - protein, 12 aa, synthetic construct
  REGION 1..12: note = source = /note="Description of Artificial Sequence: Synthetic peptide"
RASQSVSSSYLA

SEQ ID NO: 535 - protein, 12 aa, synthetic construct
  REGION 1..12: note = source = /note="Description of Artificial Sequence: Synthetic peptide"
RASQRVASNYLA

SEQ ID NO: 536 - protein, 12 aa, synthetic construct
  REGION 1..12: note = source = /note="Description of Artificial Sequence: Synthetic peptide"
RASQSLSSNFLA

SEQ ID NO: 537 - protein, 16 aa, synthetic construct
  REGION 1..16: note = source = /note="Description of Artificial Sequence: Synthetic peptide"
RSSQSLLHSNGYNYLD

SEQ ID NO: 538 - protein, 12 aa, synthetic construct
  REGION 1..12: note = source = /note="Description of Artificial Sequence: Synthetic peptide"
RASQSVSSSYLA

SEQ ID NO: 539 - protein, 12 aa, synthetic construct
  REGION 1..12: note = source = /note="Description of Artificial Sequence: Synthetic peptide"
RASQSVASSFLA

SEQ ID NO: 540 - protein, 11 aa, synthetic construct
  REGION 1..11: note = source = /note="Description of Artificial Sequence: Synthetic peptide"
RASQDVNTAVS

SEQ ID NO: 541 - protein, 15 aa, synthetic construct
  REGION 1..15: note = source = /note="Description of Artificial Sequence: Synthetic peptide"
RASESVSVIGAHLIH

SEQ ID NO: 542 - protein, 15 aa, synthetic construct
  REGION 1..15: note = source = /note="Description of Artificial Sequence: Synthetic peptide"
RASESVTILGSHLIY

SEQ ID NO: 543 - protein, 15 aa, synthetic construct
  REGION 1..15: note = source = /note="Description of Artificial Sequence: Synthetic peptide"
RASESVTILGSHLIY

SEQ ID NO: 544 - protein, 7 aa, synthetic construct
  REGION 1..7: note = source = /note="Description of Artificial Sequence: Synthetic peptide"
GASRRAT

SEQ ID NO: 545 - protein, 7 aa, synthetic construct
  REGION 1..7: note = source = /note="Description of Artificial Sequence: Synthetic peptide"
LGSNRAS

SEQ ID NO: 546 - protein, 7 aa, synthetic construct
  REGION 1..7: note = source = /note="Description of Artificial Sequence: Synthetic peptide"
EVSNRFS

SEQ ID NO: 547 - protein, 7 aa, synthetic construct
  REGION 1..7: note = source = /note="Description of Artificial Sequence: Synthetic peptide"
LGSKRAS

SEQ ID NO: 548 - protein, 7 aa, synthetic construct
  REGION 1..7: note = source = /note="Description of Artificial Sequence: Synthetic peptide"
GASTLAS

SEQ ID NO: 549 - protein, 7 aa, synthetic construct
  REGION 1..7: note = source = /note="Description of Artificial Sequence: Synthetic peptide"
LGSNRAS

SEQ ID NO: 550 - protein, 7 aa, synthetic construct
  REGION 1..7: note = source = /note="Description of Artificial Sequence: Synthetic peptide"
GASTRAS

SEQ ID NO: 551 - protein, 7 aa, synthetic construct
  REGION 1..7: note = source = /note="Description of Artificial Sequence: Synthetic peptide"
GASIRAT

SEQ ID NO: 552 - protein, 7 aa, synthetic construct
  REGION 1..7: note = source = /note="Description of Artificial Sequence: Synthetic peptide"
DASNRAT

SEQ ID NO: 553 - protein, 7 aa, synthetic construct
  REGION 1..7: note = source = /note="Description of Artificial Sequence: Synthetic peptide"
AASSLQS

SEQ ID NO: 554 - protein, 7 aa, synthetic construct
  REGION 1..7: note = source = /note="Description of Artificial Sequence: Synthetic peptide"
AASSLQS

SEQ ID NO: 555 - protein, 7 aa, synthetic construct
  REGION 1..7: note = source = /note="Description of Artificial Sequence: Synthetic peptide"
EVSNRDS

SEQ ID NO: 556 - protein, 7 aa, synthetic construct
  REGION 1..7: note = source = /note="Description of Artificial Sequence: Synthetic peptide"
DASTLQT

SEQ ID NO: 557 - protein, 7 aa, synthetic construct
  REGION 1..7: note = source = /note="Description of Artificial Sequence: Synthetic peptide"
GASTRAT

SEQ ID NO: 558 - protein, 7 aa, synthetic construct
  REGION 1..7: note = source = /note="Description of Artificial Sequence: Synthetic peptide"
GASSRAS

SEQ ID NO: 559 - protein, 7 aa, synthetic construct
  REGION 1..7: note = source = /note="Description of Artificial Sequence: Synthetic peptide"
SATSPVP

SEQ ID NO: 560 - protein, 7 aa, synthetic construct
  REGION 1..7: note = source = /note="Description of Artificial Sequence: Synthetic peptide"
AANKSQS

SEQ ID NO: 561 - protein, 7 aa, synthetic construct
  REGION 1..7: note = source = /note="Description of Artificial Sequence: Synthetic peptide"
LGSNRAS

SEQ ID NO: 562 - protein, 7 aa, synthetic construct
  REGION 1..7: note = source = /note="Description of Artificial Sequence: Synthetic peptide"
DDSVRPS

SEQ ID NO: 563 - protein, 7 aa, synthetic construct
  REGION 1..7: note = source = /note="Description of Artificial Sequence: Synthetic peptide"
RDKERPS

SEQ ID NO: 564 - protein, 7 aa, synthetic construct
  REGION 1..7: note = source = /note="Description of Artificial Sequence: Synthetic peptide"
AASNLQS

SEQ ID NO: 565 - protein, 7 aa, synthetic construct
  REGION 1..7: note = source = /note="Description of Artificial Sequence: Synthetic peptide"
GKNNRPS

SEQ ID NO: 566 - protein, 7 aa, synthetic construct
  REGION 1..7: note = source = /note="Description of Artificial Sequence: Synthetic peptide"
GTNNRPS

SEQ ID NO: 567 - protein, 7 aa, synthetic construct
  REGION 1..7: note = source = /note="Description of Artificial Sequence: Synthetic peptide"
GASTRAT

SEQ ID NO: 568 - protein, 7 aa, synthetic construct
  REGION 1..7: note = source = /note="Description of Artificial Sequence: Synthetic peptide"
DASSRAT

SEQ ID NO: 569 - protein, 7 aa, synthetic construct
  REGION 1..7: note = source = /note="Description of Artificial Sequence: Synthetic peptide"
GASSRAT

SEQ ID NO: 570 - protein, 7 aa, synthetic construct
  REGION 1..7: note = source = /note="Description of Artificial Sequence: Synthetic peptide"
GSSNRAT

SEQ ID NO: 571 - protein, 7 aa, synthetic construct
  REGION 1..7: note = source = /note="Description of Artificial Sequence: Synthetic peptide"
AASSLQS

SEQ ID NO: 572 - protein, 7 aa, synthetic construct
  REGION 1..7: note = source = /note="Description of Artificial Sequence: Synthetic peptide"
GASQRAT

SEQ ID NO: 573 - protein, 7 aa, synthetic construct
  REGION 1..7: note = source = /note="Description of Artificial Sequence: Synthetic peptide"
GASSRAT

SEQ ID NO: 574 - protein, 7 aa, synthetic construct
  REGION 1..7: note = source = /note="Description of Artificial Sequence: Synthetic peptide"
GASSRAT

SEQ ID NO: 575 - protein, 7 aa, synthetic construct
  REGION 1..7: note = source = /note="Description of Artificial Sequence: Synthetic peptide"
GASSRAT

SEQ ID NO: 576 - protein, 7 aa, synthetic construct
  REGION 1..7: note = source = /note="Description of Artificial Sequence: Synthetic peptide"
GASNWAT

SEQ ID NO: 577 - protein, 7 aa, synthetic construct
  REGION 1..7: note = source = /note="Description of Artificial Sequence: Synthetic peptide"
LGSNRAS

SEQ ID NO: 578 - protein, 7 aa, synthetic construct
  REGION 1..7: note = source = /note="Description of Artificial Sequence: Synthetic peptide"
GTSSRAT

SEQ ID NO: 579 - protein, 7 aa, synthetic construct
  REGION 1..7: note = source = /note="Description of Artificial Sequence: Synthetic peptide"
GASGRAT

SEQ ID NO: 580 - protein, 7 aa, synthetic construct
  REGION 1..7: note = source = /note="Description of Artificial Sequence: Synthetic peptide"
SASYRYT

SEQ ID NO: 581 - protein, 7 aa, synthetic construct
  REGION 1..7: note = source = /note="Description of Artificial Sequence: Synthetic peptide"
LASNLET

SEQ ID NO: 582 - protein, 7 aa, synthetic construct
  REGION 1..7: note = source = /note="Description of Artificial Sequence: Synthetic peptide"
LASNVQT

SEQ ID NO: 583 - protein, 7 aa, synthetic construct
  REGION 1..7: note = source = /note="Description of Artificial Sequence: Synthetic peptide"
LASNVQT

SEQ ID NO: 584 - protein, 10 aa, synthetic construct
  REGION 1..10: note = source = /note="Description of Artificial Sequence: Synthetic peptide"
QQYHSSPSWT

SEQ ID NO: 585 - protein, 9 aa, synthetic construct
  REGION 1..9: note = source = /note="Description of Artificial Sequence: Synthetic peptide"
MQALQTPYT

SEQ ID NO: 586 - protein, 8 aa, synthetic construct
  REGION 1..8: note = source = /note="Description of Artificial Sequence: Synthetic peptide"
MQNIQFPS

SEQ ID NO: 587 - protein, 9 aa, synthetic construct
  REGION 1..9: note = source = /note="Description of Artificial Sequence: Synthetic peptide"
MQALQTPYT

SEQ ID NO: 588 - protein, 8 aa, synthetic construct
  REGION 1..8: note = source = /note="Description of Artificial Sequence: Synthetic peptide"
QQSYKRAS

SEQ ID NO: 589 - protein, 9 aa, synthetic construct
  REGION 1..9: note = source = /note="Description of Artificial Sequence: Synthetic peptide"
MQGRQFPYS

SEQ ID NO: 590 - protein, 8 aa, synthetic construct
  REGION 1..8: note = source = /note="Description of Artificial Sequence: Synthetic peptide"
QQYGSSLT

SEQ ID NO: 591 - protein, 9 aa, synthetic construct
  REGION 1..9: note = source = /note="Description of Artificial Sequence: Synthetic peptide"
QQYGSSSWT

SEQ ID NO: 592 - protein, 10 aa, synthetic construct
  REGION 1..10: note = source = /note="Description of Artificial Sequence: Synthetic peptide"
QQYGSSPPWT

SEQ ID NO: 593 - protein, 7 aa, synthetic construct
  REGION 1..7: note = source = /note="Description of Artificial Sequence: Synthetic peptide"
QQSYTLA

SEQ ID NO: 594 - protein, 9 aa, synthetic construct
  REGION 1..9: note = source = /note="Description of Artificial Sequence: Synthetic peptide"
QQSYSTPYT

SEQ ID NO: 595 - protein, 9 aa, synthetic construct
  REGION 1..9: note = source = /note="Description of Artificial Sequence: Synthetic peptide"
MQGTHWPGT

SEQ ID NO: 596 - protein, 9 aa, synthetic construct
  REGION 1..9: note = source = /note="Description of Artificial Sequence: Synthetic peptide"
QQYESLPLT

SEQ ID NO: 597 - protein, 10 aa, synthetic construct
  REGION 1..10: note = source = /note="Description of Artificial Sequence: Synthetic peptide"
QQYNDWLPVT

SEQ ID NO: 598 - protein, 10 aa, synthetic construct
  REGION 1..10: note = source = /note="Description of Artificial Sequence: Synthetic peptide"
QQYAGSPPFT

SEQ ID NO: 599 - protein, 9 aa, synthetic construct
  REGION 1..9: note = source = /note="Description of Artificial Sequence: Synthetic peptide"
LQHDNFPLT

SEQ ID NO: 600 - protein, 9 aa, synthetic construct
  REGION 1..9: note = source = /note="Description of Artificial Sequence: Synthetic peptide"
QHYYRFPYS

SEQ ID NO: 601 - protein, 9 aa, synthetic construct
  REGION 1..9: note = source = /note="Description of Artificial Sequence: Synthetic peptide"
MQALQTPYT

SEQ ID NO: 602 - protein, 11 aa, synthetic construct
  REGION 1..11: note = source = /note="Description of Artificial Sequence: Synthetic peptide"
QVWDSDSEHVV

SEQ ID NO: 603 - protein, 9 aa, synthetic construct
  REGION 1..9: note = source = /note="Description of Artificial Sequence: Synthetic peptide"
QAWDDTTVV

SEQ ID NO: 604 - protein, 9 aa, synthetic construct
  REGION 1..9: note = source = /note="Description of Artificial Sequence: Synthetic peptide"
QKYNSAPFT

SEQ ID NO: 605 - protein, 12 aa, synthetic construct
  REGION 1..12: note = source = /note="Description of Artificial Sequence: Synthetic peptide"
SSRDSSGDHLRV

SEQ ID NO: 606 - protein, 11 aa, synthetic construct
  REGION 1..11: note = source = /note="Description of Artificial Sequence: Synthetic peptide"
NSRDSSGHHLL

SEQ ID NO: 607 - protein, 14 aa, synthetic construct
  REGION 1..14: note = source = /note="Description of Artificial Sequence: Synthetic peptide"
QHYGSSFNGSSLFT

SEQ ID NO: 608 - protein, 10 aa, synthetic construct
  REGION 1..10: note = source = /note="Description of Artificial Sequence: Synthetic peptide"
QQFGTSSGLT

SEQ ID NO: 609 - protein, 10 aa, synthetic construct
  REGION 1..10: note = source = /note="Description of Artificial Sequence: Synthetic peptide"
QQYHSSPSWT

SEQ ID NO: 610 - protein, 10 aa, synthetic construct
  REGION 1..10: note = source = /note="Description of Artificial Sequence: Synthetic peptide"
QQYHSSPSWT

SEQ ID NO: 611 - protein, 9 aa, synthetic construct
  REGION 1..9: note = source = /note="Description of Artificial Sequence: Synthetic peptide"
QQSYSTPYS

SEQ ID NO: 612 - protein, 10 aa, synthetic construct
  REGION 1..10: note = source = /note="Description of Artificial Sequence: Synthetic peptide"
QHYESSPSWT

SEQ ID NO: 613 - protein, 10 aa, synthetic construct
  REGION 1..10: note = source = /note="Description of Artificial Sequence: Synthetic peptide"
QQYGSPPRFT

SEQ ID NO: 614 - protein, 10 aa, synthetic construct
  REGION 1..10: note = source = /note="Description of Artificial Sequence: Synthetic peptide"
QHYGSSPSWT

SEQ ID NO: 615 - protein, 10 aa, synthetic construct
  REGION 1..10: note = source = /note="Description of Artificial Sequence: Synthetic peptide"
QHYDSSPSWT

SEQ ID NO: 616 - protein, 10 aa, synthetic construct
  REGION 1..10: note = source = /note="Description of Artificial Sequence: Synthetic peptide"
QYYGTSPMYT

SEQ ID NO: 617 - protein, 9 aa, synthetic construct
  REGION 1..9: note = source = /note="Description of Artificial Sequence: Synthetic peptide"
MQALQTPLT

SEQ ID NO: 618 - protein, 11 aa, synthetic construct
  REGION 1..11: note = source = /note="Description of Artificial Sequence: Synthetic peptide"
QHYGNSPPKFT

SEQ ID NO: 619 - protein, 10 aa, synthetic construct
  REGION 1..10: note = source = /note="Description of Artificial Sequence: Synthetic peptide"
QHYGGSPRLT

SEQ ID NO: 620 - protein, 9 aa, synthetic construct
  REGION 1..9: note = source = /note="Description of Artificial Sequence: Synthetic peptide"
QQHYSTPWT

SEQ ID NO: 621 - protein, 9 aa, synthetic construct
  REGION 1..9: note = source = /note="Description of Artificial Sequence: Synthetic peptide"
LQSRIFPRT

SEQ ID NO: 622 - protein, 9 aa, synthetic construct
  REGION 1..9: note = source = /note="Description of Artificial Sequence: Synthetic peptide"
LQSRTIPRT

SEQ ID NO: 623 - protein, 9 aa, synthetic construct
  REGION 1..9: note = source = /note="Description of Artificial Sequence: Synthetic peptide"
LQSRTIPRT

SEQ ID NO: 624 - protein, 7 aa, synthetic construct
  REGION 1..7: note = source = /note="Description of Artificial Sequence: Synthetic peptide"
GFTFSNY

SEQ ID NO: 625 - protein, 7 aa, synthetic construct
  REGION 1..7: note = source = /note="Description of Artificial Sequence: Synthetic peptide"
GFTFDDY

SEQ ID NO: 626 - protein, 7 aa, synthetic construct
  REGION 1..7: note = source = /note="Description of Artificial Sequence: Synthetic peptide"
GFALSNH

SEQ ID NO: 627 - protein, 7 aa, synthetic construct
  REGION 1..7: note = source = /note="Description of Artificial Sequence: Synthetic peptide"
GYIFDNF

SEQ ID NO: 628 - protein, 7 aa, synthetic construct
  REGION 1..7: note = source = /note="Description of Artificial Sequence: Synthetic peptide"
GFTFSSD

SEQ ID NO: 629 - protein, 7 aa, synthetic construct
  REGION 1..7: note = source = /note="Description of Artificial Sequence: Synthetic peptide"
GYTFSNY

SEQ ID NO: 630 - protein, 7 aa, synthetic construct
  REGION 1..7: note = source = /note="Description of Artificial Sequence: Synthetic peptide"
GFALSNH

SEQ ID NO: 631 - protein, 7 aa, synthetic construct
  REGION 1..7: note = source = /note="Description of Artificial Sequence: Synthetic peptide"
GFALSNH

SEQ ID NO: 632 - protein, 7 aa, synthetic construct
  REGION 1..7: note = source = /note="Description of Artificial Sequence: Synthetic peptide"
GFALSNH

SEQ ID NO: 633 - protein, 7 aa, synthetic construct
  REGION 1..7: note = source = /note="Description of Artificial Sequence: Synthetic peptide"
GFTFSDY

SEQ ID NO: 634 - protein, 7 aa, synthetic construct
  REGION 1..7: note = source = /note="Description of Artificial Sequence: Synthetic peptide"
GFALSNH

SEQ ID NO: 635 - protein, 7 aa, synthetic construct
  REGION 1..7: note = source = /note="Description of Artificial Sequence: Synthetic peptide"
GFTFSDY

SEQ ID NO: 636 - protein, 7 aa, synthetic construct
  REGION 1..7: note = source = /note="Description of Artificial Sequence: Synthetic peptide"
GFALSNH

SEQ ID NO: 637 - protein, 7 aa, synthetic construct
  REGION 1..7: note = source = /note="Description of Artificial Sequence: Synthetic peptide"
GFALSNH

SEQ ID NO: 638 - protein, 7 aa, synthetic construct
  REGION 1..7: note = source = /note="Description of Artificial Sequence: Synthetic peptide"
GFALSNH

SEQ ID NO: 639 - protein, 9 aa, synthetic construct
  REGION 1..9: note = source = /note="Description of Artificial Sequence: Synthetic peptide"
GGSISSSYY

SEQ ID NO: 640 - protein, 9 aa, synthetic construct
  REGION 1..9: note = source = /note="Description of Artificial Sequence: Synthetic peptide"
GFSLRTSGM

SEQ ID NO: 641 - protein, 7 aa, synthetic construct
  REGION 1..7: note = source = /note="Description of Artificial Sequence: Synthetic peptide"
GFTFSSY

SEQ ID NO: 642 - protein, 7 aa, synthetic construct
  REGION 1..7: note = source = /note="Description of Artificial Sequence: Synthetic peptide"
GFTFSDY

SEQ ID NO: 643 - protein, 7 aa, synthetic construct
  REGION 1..7: note = source = /note="Description of Artificial Sequence: Synthetic peptide"
GYTVTSH

SEQ ID NO: 644 - protein, 9 aa, synthetic construct
  REGION 1..9: note = source = /note="Description of Artificial Sequence: Synthetic peptide"
GGSISSGGY

SEQ ID NO: 645 - protein, 7 aa, synthetic construct
  REGION 1..7: note = source = /note="Description of Artificial Sequence: Synthetic peptide"
GGTFSSY

SEQ ID NO: 646 - protein, 9 aa, synthetic construct
  REGION 1..9: note = source = /note="Description of Artificial Sequence: Synthetic peptide"
GDSVSSNSA

SEQ ID NO: 647 - protein, 7 aa, synthetic construct
  REGION 1..7: note = source = /note="Description of Artificial Sequence: Synthetic peptide"
GFTFSSY

SEQ ID NO: 648 - protein, 7 aa, synthetic construct
  REGION 1..7: note = source = /note="Description of Artificial Sequence: Synthetic peptide"
GITFSRY

SEQ ID NO: 649 - protein, 7 aa, synthetic construct
  REGION 1..7: note = source = /note="Description of Artificial Sequence: Synthetic peptide"
GFTFSSY

SEQ ID NO: 650 - protein, 7 aa, synthetic construct
  REGION 1..7: note = source = /note="Description of Artificial Sequence: Synthetic peptide"
GFTFSSY

SEQ ID NO: 651 - protein, 7 aa, synthetic construct
  REGION 1..7: note = source = /note="Description of Artificial Sequence: Synthetic peptide"
GFTFDDY

SEQ ID NO: 652 - protein, 7 aa, synthetic construct
  REGION 1..7: note = source = /note="Description of Artificial Sequence: Synthetic peptide"
GFTFDDY

SEQ ID NO: 653 - protein, 7 aa, synthetic construct
  REGION 1..7: note = source = /note="Description of Artificial Sequence: Synthetic peptide"
GFTFSSY

SEQ ID NO: 654 - protein, 7 aa, synthetic construct
  REGION 1..7: note = source = /note="Description of Artificial Sequence: Synthetic peptide"
GFTFSSY

SEQ ID NO: 655 - protein, 7 aa, synthetic construct
  REGION 1..7: note = source = /note="Description of Artificial Sequence: Synthetic peptide"
GFTFSSY

SEQ ID NO: 656 - protein, 7 aa, synthetic construct
  REGION 1..7: note = source = /note="Description of Artificial Sequence: Synthetic peptide"
GFSFSSY

SEQ ID NO: 657 - protein, 7 aa, synthetic construct
  REGION 1..7: note = source = /note="Description of Artificial Sequence: Synthetic peptide"
GFTFSSY

SEQ ID NO: 658 - protein, 7 aa, synthetic construct
  REGION 1..7: note = source = /note="Description of Artificial Sequence: Synthetic peptide"
GFTFSSY

SEQ ID NO: 659 - protein, 7 aa, synthetic construct
  REGION 1..7: note = source = /note="Description of Artificial Sequence: Synthetic peptide"
GFTFSSY

SEQ ID NO: 660 - protein, 7 aa, synthetic construct
  REGION 1..7: note = source = /note="Description of Artificial Sequence: Synthetic peptide"
GYTFTNF

SEQ ID NO: 661 - protein, 7 aa, synthetic construct
  REGION 1..7: note = source = /note="Description of Artificial Sequence: Synthetic peptide"
GYTFTDY

SEQ ID NO: 662 - protein, 7 aa, synthetic construct
  REGION 1..7: note = source = /note="Description of Artificial Sequence: Synthetic peptide"
GYTFRHY

SEQ ID NO: 663 - protein, 7 aa, synthetic construct
  REGION 1..7: note = source = /note="Description of Artificial Sequence: Synthetic peptide"
GYTFTHY

SEQ ID NO: 664 - protein, 6 aa, synthetic construct
  REGION 1..6: note = source = /note="Description of Artificial Sequence: Synthetic peptide"
SRSGEN

SEQ ID NO: 665 - protein, 6 aa, synthetic construct
  REGION 1..6: note = source = /note="Description of Artificial Sequence: Synthetic peptide"
SWNSGS

SEQ ID NO: 666 - protein, 5 aa, synthetic construct
  REGION 1..5: note = source = /note="Description of Artificial Sequence: Synthetic peptide"
VYSGS

SEQ ID NO: 667 - protein, 6 aa, synthetic construct
  REGION 1..6: note = source = /note="Description of Artificial Sequence: Synthetic peptide"
NPKNNN

SEQ ID NO: 668 - protein, 6 aa, synthetic construct
  REGION 1..6: note = source = /note="Description of Artificial Sequence: Synthetic peptide"
SGSGGT

SEQ ID NO: 669 - protein, 6 aa, synthetic construct
  REGION 1..6: note = source = /note="Description of Artificial Sequence: Synthetic peptide"
SAYNGN

SEQ ID NO: 670 - protein, 5 aa, synthetic construct
  REGION 1..5: note = source = /note="Description of Artificial Sequence: Synthetic peptide"
VYSGS

SEQ ID NO: 671 - protein, 5 aa, synthetic construct
  REGION 1..5: note = source = /note="Description of Artificial Sequence: Synthetic peptide"
VYSGS

SEQ ID NO: 672 - protein, 5 aa, synthetic construct
  REGION 1..5: note = source = /note="Description of Artificial Sequence: Synthetic peptide"
VYSGS

SEQ ID NO: 673 - protein, 6 aa, synthetic construct
  REGION 1..6: note = source = /note="Description of Artificial Sequence: Synthetic peptide"
SSSGST

SEQ ID NO: 674 - protein, 5 aa, synthetic construct
  REGION 1..5: note = source = /note="Description of Artificial Sequence: Synthetic peptide"
VYSGS

SEQ ID NO: 675 - protein, 6 aa, synthetic construct
  REGION 1..6: note = source = /note="Description of Artificial Sequence: Synthetic peptide"
SSSGNT

SEQ ID NO: 676 - protein, 5 aa, synthetic construct
  REGION 1..5: note = source = /note="Description of Artificial Sequence: Synthetic peptide"
VYSGS

SEQ ID NO: 677 - protein, 5 aa, synthetic construct
  REGION 1..5: note = source = /note="Description of Artificial Sequence: Synthetic peptide"
VYSGS

SEQ ID NO: 678 - protein, 5 aa, synthetic construct
  REGION 1..5: note = source = /note="Description of Artificial Sequence: Synthetic peptide"
VYSGS

SEQ ID NO: 679 - protein, 5 aa, synthetic construct
  REGION 1..5: note = source = /note="Description of Artificial Sequence: Synthetic peptide"
YYSGS

SEQ ID NO: 680 - protein, 5 aa, synthetic construct
  REGION 1..5: note = source = /note="Description of Artificial Sequence: Synthetic peptide"
DWDED

SEQ ID NO: 681 - protein, 6 aa, synthetic construct
  REGION 1..6: note = source = /note="Description of Artificial Sequence: Synthetic peptide"
SSSSSY

SEQ ID NO: 682 - protein, 6 aa, synthetic construct
  REGION 1..6: note = source = /note="Description of Artificial Sequence: Synthetic peptide"
SSSGST

SEQ ID NO: 683 - protein, 6 aa, synthetic construct
  REGION 1..6: note = source = /note="Description of Artificial Sequence: Synthetic peptide"
NPSGGV

SEQ ID NO: 684 - protein, 5 aa, synthetic construct
  REGION 1..5: note = source = /note="Description of Artificial Sequence: Synthetic peptide"
YYSGS

SEQ ID NO: 685 - protein, 6 aa, synthetic construct
  REGION 1..6: note = source = /note="Description of Artificial Sequence: Synthetic peptide"
IPIFGT

SEQ ID NO: 686 - protein, 7 aa, synthetic construct
  REGION 1..7: note = source = /note="Description of Artificial Sequence: Synthetic peptide"
YYRSKWY

SEQ ID NO: 687 - protein, 6 aa, synthetic construct
  REGION 1..6: note = source = /note="Description of Artificial Sequence: Synthetic peptide"
SGSGGS

SEQ ID NO: 688 - protein, 6 aa, synthetic construct
  REGION 1..6: note = source = /note="Description of Artificial Sequence: Synthetic peptide"
SDSGVS

SEQ ID NO: 689 - protein, 6 aa, synthetic construct
  REGION 1..6: note = source = /note="Description of Artificial Sequence: Synthetic peptide"
SGSGGS

SEQ ID NO: 690 - protein, 6 aa, synthetic construct
  REGION 1..6: note = source = /note="Description of Artificial Sequence: Synthetic peptide"
SGSGGS

SEQ ID NO: 691 - protein, 6 aa, synthetic construct
  REGION 1..6: note = source = /note="Description of Artificial Sequence: Synthetic peptide"
SWNSGS

SEQ ID NO: 692 - protein, 6 aa, synthetic construct
  REGION 1..6: note = source = /note="Description of Artificial Sequence: Synthetic peptide"
NWKGNS

SEQ ID NO: 693 - protein, 6 aa, synthetic construct
  REGION 1..6: note = source = /note="Description of Artificial Sequence: Synthetic peptide"
SGSGGS

SEQ ID NO: 694 - protein, 6 aa, synthetic construct
  REGION 1..6: note = source = /note="Description of Artificial Sequence: Synthetic peptide"
SGSGGS

SEQ ID NO: 695 - protein, 6 aa, synthetic construct
  REGION 1..6: note = source = /note="Description of Artificial Sequence: Synthetic peptide"
SGSGGS

SEQ ID NO: 696 - protein, 6 aa, synthetic construct
  REGION 1..6: note = source = /note="Description of Artificial Sequence: Synthetic peptide"
SGSGGS

SEQ ID NO: 697 - protein, 6 aa, synthetic construct
  REGION 1..6: note = source = /note="Description of Artificial Sequence: Synthetic peptide"
SGSGGS

SEQ ID NO: 698 - protein, 6 aa, synthetic construct
  REGION 1..6: note = source = /note="Description of Artificial Sequence: Synthetic peptide"
SGSGGS

SEQ ID NO: 699 - protein, 6 aa, synthetic construct
  REGION 1..6: note = source = /note="Description of Artificial Sequence: Synthetic peptide"
SGSGGS

SEQ ID NO: 700 - protein, 6 aa, synthetic construct
  REGION 1..6: note = source = /note="Description of Artificial Sequence: Synthetic peptide"
NTYTGE

SEQ ID NO: 701 - protein, 6 aa, synthetic construct
  REGION 1..6: note = source = /note="Description of Artificial Sequence: Synthetic peptide"
NTETRE

SEQ ID NO: 702 - protein, 6 aa, synthetic construct
  REGION 1..6: note = source = /note="Description of Artificial Sequence: Synthetic peptide"
NTESGV

SEQ ID NO: 703 - protein, 6 aa, synthetic construct
  REGION 1..6: note = source = /note="Description of Artificial Sequence: Synthetic peptide"
NTETGE

SEQ ID NO: 704 - protein, 11 aa, synthetic construct
  REGION 1..11: note = source = /note="Description of Artificial Sequence: Synthetic peptide"
SPAHYYGGMDV

SEQ ID NO: 705 - protein, 6 aa, synthetic construct
  REGION 1..6: note = source = /note="Description of Artificial Sequence: Synthetic peptide"
HSFLAY

SEQ ID NO: 706 - protein, 7 aa, synthetic construct
  REGION 1..7: note = source = /note="Description of Artificial Sequence: Synthetic peptide"
HGGESDV

SEQ ID NO: 707 - protein, 11 aa, synthetic construct
  REGION 1..11: note = source = /note="Description of Artificial Sequence: Synthetic peptide"
GPYYYQSYMDV

SEQ ID NO: 708 - protein, 14 aa, synthetic construct
  REGION 1..14: note = source = /note="Description of Artificial Sequence: Synthetic peptide"
LDSSGYYYARGPRY

SEQ ID NO: 709 - protein, 9 aa, synthetic construct
  REGION 1..9: note = source = /note="Description of Artificial Sequence: Synthetic peptide"
GPYYYYMDV

SEQ ID NO: 710 - protein, 7 aa, synthetic construct
  REGION 1..7: note = source = /note="Description of Artificial Sequence: Synthetic peptide"
HGGESDV

SEQ ID NO: 711 - protein, 7 aa, synthetic construct
  REGION 1..7: note = source = /note="Description of Artificial Sequence: Synthetic peptide"
HGGESDV

SEQ ID NO: 712 - protein, 7 aa, synthetic construct
  REGION 1..7: note = source = /note="Description of Artificial Sequence: Synthetic peptide"
HGGESDV

SEQ ID NO: 713 - protein, 8 aa, synthetic construct
  REGION 1..8: note = source = /note="Description of Artificial Sequence: Synthetic peptide"
ESGDGMDV

SEQ ID NO: 714 - protein, 7 aa, synthetic construct
  REGION 1..7: note = source = /note="Description of Artificial Sequence: Synthetic peptide"
HGGESDV

SEQ ID NO: 715 - protein, 8 aa, synthetic construct
  REGION 1..8: note = source = /note="Description of Artificial Sequence: Synthetic peptide"
STMVREDY

SEQ ID NO: 716 - protein, 7 aa, synthetic construct
  REGION 1..7: note = source = /note="Description of Artificial Sequence: Synthetic peptide"
HGGESDV

SEQ ID NO: 717 - protein, 7 aa, synthetic construct
  REGION 1..7: note = source = /note="Description of Artificial Sequence: Synthetic peptide"
HGGESDV

SEQ ID NO: 718 - protein, 7 aa, synthetic construct
  REGION 1..7: note = source = /note="Description of Artificial Sequence: Synthetic peptide"
HGGESDV

SEQ ID NO: 719 - protein, 11 aa, synthetic construct
  REGION 1..11: note = source = /note="Description of Artificial Sequence: Synthetic peptide"
HWQEWPDAFDI

SEQ ID NO: 720 - protein, 13 aa, synthetic construct
  REGION 1..13: note = source = /note="Description of Artificial Sequence: Synthetic peptide"
SGAGGTSATAFDI

SEQ ID NO: 721 - protein, 10 aa, synthetic construct
  REGION 1..10: note = source = /note="Description of Artificial Sequence: Synthetic peptide"
TIAAVYAFDI

SEQ ID NO: 722 - protein, 8 aa, synthetic construct
  REGION 1..8: note = source = /note="Description of Artificial Sequence: Synthetic peptide"
DLRGAFDI

SEQ ID NO: 723 - protein, 11 aa, synthetic construct
  REGION 1..11: note = source = /note="Description of Artificial Sequence: Synthetic peptide"
EGSGSGWYFDF

SEQ ID NO: 724 - protein, 13 aa, synthetic construct
  REGION 1..13: note = source = /note="Description of Artificial Sequence: Synthetic peptide"
AGIAARLRGAFDI

SEQ ID NO: 725 - protein, 20 aa, synthetic construct
  REGION 1..20: note = source = /note="Description of Artificial Sequence: Synthetic peptide"
RGGYQLLRWDVGLLRSAFDI

SEQ ID NO: 726 - protein, 13 aa, synthetic construct
  REGION 1..13: note = source = /note="Description of Artificial Sequence: Synthetic peptide"
SSPEGLFLYWFDP

SEQ ID NO: 727 - protein, 9 aa, synthetic construct
  REGION 1..9: note = source = /note="Description of Artificial Sequence: Synthetic peptide"
VEGSGSLDY

SEQ ID NO: 728 - protein, 9 aa, synthetic construct
  REGION 1..9: note = source = /note="Description of Artificial Sequence: Synthetic peptide"
RAGSEASDI

SEQ ID NO: 729 - protein, 15 aa, synthetic construct
  REGION 1..15: note = source = /note="Description of Artificial Sequence: Synthetic peptide"
ATYKRELRYYYGMDV

SEQ ID NO: 730 - protein, 15 aa, synthetic construct
  REGION 1..15: note = source = /note="Description of Artificial Sequence: Synthetic peptide"
ATYKRELRYYYGMDV

SEQ ID NO: 731 - protein, 8 aa, synthetic construct
  REGION 1..8: note = source = /note="Description of Artificial Sequence: Synthetic peptide"
VGKAVPDV

SEQ ID NO: 732 - protein, 13 aa, synthetic construct
  REGION 1..13: note = source = /note="Description of Artificial Sequence: Synthetic peptide"
HQGVAYYNYAMDV

SEQ ID NO: 733 - protein, 8 aa, synthetic construct
  REGION 1..8: note = source = /note="Description of Artificial Sequence: Synthetic peptide"
VVRDGMDV

SEQ ID NO: 734 - protein, 9 aa, synthetic construct
  REGION 1..9: note = source = /note="Description of Artificial Sequence: Synthetic peptide"
IPQTGTFDY

SEQ ID NO: 735 - protein, 15 aa, synthetic construct
  REGION 1..15: note = source = /note="Description of Artificial Sequence: Synthetic peptide"
ANYKRELRYYYGMDV

SEQ ID NO: 736 - protein, 11 aa, synthetic construct
  REGION 1..11: note = source = /note="Description of Artificial Sequence: Synthetic peptide"
ALVGATGAFDI

SEQ ID NO: 737 - protein, 8 aa, synthetic construct
  REGION 1..8: note = source = /note="Description of Artificial Sequence: Synthetic peptide"
WFGEGFDP

SEQ ID NO: 738 - protein, 17 aa, synthetic construct
  REGION 1..17: note = source = /note="Description of Artificial Sequence: Synthetic peptide"
VGYDSSGYYRDYYGMDV

SEQ ID NO: 739 - protein, 13 aa, synthetic construct
  REGION 1..13: note = source = /note="Description of Artificial Sequence: Synthetic peptide"
MGWSSGYLGAFDI

SEQ ID NO: 740 - protein, 13 aa, synthetic construct
  REGION 1..13: note = source = /note="Description of Artificial Sequence: Synthetic peptide"
GEIYYGYDGGFAY

SEQ ID NO: 741 - protein, 8 aa, synthetic construct
  REGION 1..8: note = source = /note="Description of Artificial Sequence: Synthetic peptide"
DYSYAMDY

SEQ ID NO: 742 - protein, 8 aa, synthetic construct
  REGION 1..8: note = source = /note="Description of Artificial Sequence: Synthetic peptide"
DYLYSLDF

SEQ ID NO: 743 - protein, 8 aa, synthetic construct
  REGION 1..8: note = source = /note="Description of Artificial Sequence: Synthetic peptide"
DYLYSCDY

SEQ ID NO: 744 - protein, 8 aa, synthetic construct
  REGION 1..8: note = source = /note="Description of Artificial Sequence: Synthetic peptide"
SQSISSSF

SEQ ID NO: 745 - protein, 12 aa, synthetic construct
  REGION 1..12: note = source = /note="Description of Artificial Sequence: Synthetic peptide"
SQSLLHSNGYNY

SEQ ID NO: 746 - protein, 12 aa, synthetic construct
  REGION 1..12: note = source = /note="Description of Artificial Sequence: Synthetic peptide"
SQSLLRNDGKTP

SEQ ID NO: 747 - protein, 12 aa, synthetic construct
  REGION 1..12: note = source = /note="Description of Artificial Sequence: Synthetic peptide"
SQSLLHSNGYNY

SEQ ID NO: 748 - protein, 7 aa, synthetic construct
  REGION 1..7: note = source = /note="Description of Artificial Sequence: Synthetic peptide"
SQSISSY

SEQ ID NO: 749 - protein, 12 aa, synthetic construct
  REGION 1..12: note = source = /note="Description of Artificial Sequence: Synthetic peptide"
SQSLLYSNGYNY

SEQ ID NO: 750 - protein, 7 aa, synthetic construct
  REGION 1..7: note = source = /note="Description of Artificial Sequence: Synthetic peptide"
SQSVSSN

SEQ ID NO: 751 - protein, 7 aa, synthetic construct
  REGION 1..7: note = source = /note="Description of Artificial Sequence: Synthetic peptide"
SQSVSSK

SEQ ID NO: 752 - protein, 8 aa, synthetic construct
  REGION 1..8: note = source = /note="Description of Artificial Sequence: Synthetic peptide"
SQSVGSTN

SEQ ID NO: 753 - protein, 7 aa, synthetic construct
  REGION 1..7: note = source = /note="Description of Artificial Sequence: Synthetic peptide"
SQSISSY

SEQ ID NO: 754 - protein, 7 aa, synthetic construct
  REGION 1..7: note = source = /note="Description of Artificial Sequence: Synthetic peptide"
SQSISSY

SEQ ID NO: 755 - protein, 12 aa, synthetic construct
  REGION 1..12: note = source = /note="Description of Artificial Sequence: Synthetic peptide"
SESLVHNSGKTY

SEQ ID NO: 756 - protein, 7 aa, synthetic construct
  REGION 1..7: note = source = /note="Description of Artificial Sequence: Synthetic peptide"
SEDINKF

SEQ ID NO: 757 - protein, 7 aa, synthetic construct
  REGION 1..7: note = source = /note="Description of Artificial Sequence: Synthetic peptide"
SQSVGSN

SEQ ID NO: 758 - protein, 8 aa, synthetic construct
  REGION 1..8: note = source = /note="Description of Artificial Sequence: Synthetic peptide"
SQSIGSSS

SEQ ID NO: 759 - protein, 7 aa, synthetic construct
  REGION 1..7: note = source = /note="Description of Artificial Sequence: Synthetic peptide"
SQDIDDA

SEQ ID NO: 760 - protein, 7 aa, synthetic construct
  REGION 1..7: note = source = /note="Description of Artificial Sequence: Synthetic peptide"
SQDIYNN

SEQ ID NO: 761 - protein, 12 aa, synthetic construct
  REGION 1..12: note = source = /note="Description of Artificial Sequence: Synthetic peptide"
SQSLLHSNGYNY

SEQ ID NO: 762 - protein, 7 aa, synthetic construct
  REGION 1..7: note = source = /note="Description of Artificial Sequence: Synthetic peptide"
NNIGTKS

SEQ ID NO: 763 - protein, 7 aa, synthetic construct
  REGION 1..7: note = source = /note="Description of Artificial Sequence: Synthetic peptide"
DGLSKKY

SEQ ID NO: 764 - protein, 7 aa, synthetic construct
  REGION 1..7: note = source = /note="Description of Artificial Sequence: Synthetic peptide"
SQGIRNW

SEQ ID NO: 765 - protein, 7 aa, synthetic construct
  REGION 1..7: note = source = /note="Description of Artificial Sequence: Synthetic peptide"
NNIGSKS

SEQ ID NO: 766 - protein, 7 aa, synthetic construct
  REGION 1..7: note = source = /note="Description of Artificial Sequence: Synthetic peptide"
DSLGNYY

SEQ ID NO: 767 - protein, 8 aa, synthetic construct
  REGION 1..8: note = source = /note="Description of Artificial Sequence: Synthetic peptide"
SQSVSSAY

SEQ ID NO: 768 - protein, 7 aa, synthetic construct
  REGION 1..7: note = source = /note="Description of Artificial Sequence: Synthetic peptide"
SQSVSNS

SEQ ID NO: 769 - protein, 8 aa, synthetic construct
  REGION 1..8: note = source = /note="Description of Artificial Sequence: Synthetic peptide"
SQSVSSSF

SEQ ID NO: 770 - protein, 8 aa, synthetic construct
  REGION 1..8: note = source = /note="Description of Artificial Sequence: Synthetic peptide"
SQSVSTTF

SEQ ID NO: 771 - protein, 7 aa, synthetic construct
  REGION 1..7: note = source = /note="Description of Artificial Sequence: Synthetic peptide"
SQSISSY

SEQ ID NO: 772 - protein, 8 aa, synthetic construct
  REGION 1..8: note = source = /note="Description of Artificial Sequence: Synthetic peptide"
TQSIGSSF

SEQ ID NO: 773 - protein, 8 aa, synthetic construct
  REGION 1..8: note = source = /note="Description of Artificial Sequence: Synthetic peptide"
SQSVSSSY

SEQ ID NO: 774 - protein, 8 aa, synthetic construct
  REGION 1..8: note = source = /note="Description of Artificial Sequence: Synthetic peptide"
SQSVSSSY

SEQ ID NO: 775 - protein, 8 aa, synthetic construct
  REGION 1..8: note = source = /note="Description of Artificial Sequence: Synthetic peptide"
SQRVASNY

SEQ ID NO: 776 - protein, 8 aa, synthetic construct
  REGION 1..8: note = source = /note="Description of Artificial Sequence: Synthetic peptide"
SQSLSSNF

SEQ ID NO: 777 - protein, 12 aa, synthetic construct
  REGION 1..12: note = source = /note="Description of Artificial Sequence: Synthetic peptide"
SQSLLHSNGYNY

SEQ ID NO: 778 - protein, 8 aa, synthetic construct
  REGION 1..8: note = source = /note="Description of Artificial Sequence: Synthetic peptide"
SQSVSSSY

SEQ ID NO: 779 - protein, 8 aa, synthetic construct
  REGION 1..8: note = source = /note="Description of Artificial Sequence: Synthetic peptide"
SQSVASSF

SEQ ID NO: 780 - protein, 7 aa, synthetic construct
  REGION 1..7: note = source = /note="Description of Artificial Sequence: Synthetic peptide"
SQDVNTA

SEQ ID NO: 781 - protein, 11 aa, synthetic construct
  REGION 1..11: note = source = /note="Description of Artificial Sequence: Synthetic peptide"
SESVSVIGAHL

SEQ ID NO: 782 - protein, 11 aa, synthetic construct
  REGION 1..11: note = source = /note="Description of Artificial Sequence: Synthetic peptide"
SESVTILGSHL

SEQ ID NO: 783 - protein, 11 aa, synthetic construct
  REGION 1..11: note = source = /note="Description of Artificial Sequence: Synthetic peptide"
SESVTILGSHL

SEQ ID NO: 784-823 - skipped (40 entries)

SEQ ID NO: 824 - protein, 7 aa, synthetic construct
  REGION 1..7: note = source = /note="Description of Artificial Sequence: Synthetic peptide"
YHSSPSW

SEQ ID NO: 825 - protein, 6 aa, synthetic construct
  REGION 1..6: note = source = /note="Description of Artificial Sequence: Synthetic peptide"
ALQTPY

SEQ ID NO: 826 - protein, 5 aa, synthetic construct
  REGION 1..5: note = source = /note="Description of Artificial Sequence: Synthetic peptide"
NIQFP

SEQ ID NO: 827 - protein, 6 aa, synthetic construct
  REGION 1..6: note = source = /note="Description of Artificial Sequence: Synthetic peptide"
ALQTPY

SEQ ID NO: 828 - protein, 5 aa, synthetic construct
  REGION 1..5: note = source = /note="Description of Artificial Sequence: Synthetic peptide"
SYKRA

SEQ ID NO: 829 - protein, 6 aa, synthetic construct
  REGION 1..6: note = source = /note="Description of Artificial Sequence: Synthetic peptide"
GRQFPY

SEQ ID NO: 830 - protein, 5 aa, synthetic construct
  REGION 1..5: note = source = /note="Description of Artificial Sequence: Synthetic peptide"
YGSSL

SEQ ID NO: 831 - protein, 6 aa, synthetic construct
  REGION 1..6: note = source = /note="Description of Artificial Sequence: Synthetic peptide"
YGSSSW

SEQ ID NO: 832 - protein, 7 aa, synthetic construct
  REGION 1..7: note = source = /note="Description of Artificial Sequence: Synthetic peptide"
YGSSPPW

SEQ ID NO: 833 - protein, 4 aa, synthetic construct
  REGION 1..4: note = source = /note="Description of Artificial Sequence: Synthetic peptide"
SYTL

SEQ ID NO: 834 - protein, 6 aa, synthetic construct
  REGION 1..6: note = source = /note="Description of Artificial Sequence: Synthetic peptide"
SYSTPY

SEQ ID NO: 835 - protein, 6 aa, synthetic construct
  REGION 1..6: note = source = /note="Description of Artificial Sequence: Synthetic peptide"
GTHWPG

SEQ ID NO: 836 - protein, 6 aa, synthetic construct
  REGION 1..6: note = source = /note="Description of Artificial Sequence: Synthetic peptide"
YESLPL

SEQ ID NO: 837 - protein, 7 aa, synthetic construct
  REGION 1..7: note = source = /note="Description of Artificial Sequence: Synthetic peptide"
YNDWLPV

SEQ ID NO: 838 - protein, 7 aa, synthetic construct
  REGION 1..7: note = source = /note="Description of Artificial Sequence: Synthetic peptide"
YAGSPPF

SEQ ID NO: 839 - protein, 6 aa, synthetic construct
  REGION 1..6: note = source = /note="Description of Artificial Sequence: Synthetic peptide"
HDNFPL

SEQ ID NO: 840 - protein, 6 aa, synthetic construct
  REGION 1..6: note = source = /note="Description of Artificial Sequence: Synthetic peptide"
YYRFPY

SEQ ID NO: 841 - protein, 6 aa, synthetic construct
  REGION 1..6: note = source = /note="Description of Artificial Sequence: Synthetic peptide"
ALQTPY

SEQ ID NO: 842 - protein, 8 aa, synthetic construct
  REGION 1..8: note = source = /note="Description of Artificial Sequence: Synthetic peptide"
WDSDSEHV

SEQ ID NO: 843 - protein, 6 aa, synthetic construct
  REGION 1..6: note = source = /note="Description of Artificial Sequence: Synthetic peptide"
WDDTTV

SEQ ID NO: 844 - protein, 6 aa, synthetic construct
  REGION 1..6: note = source = /note="Description of Artificial Sequence: Synthetic peptide"
YNSAPF

SEQ ID NO: 845 - protein, 9 aa, synthetic construct
  REGION 1..9: note = source = /note="Description of Artificial Sequence: Synthetic peptide"
RDSSGDHLR

SEQ ID NO: 846 - protein, 8 aa, synthetic construct
  REGION 1..8: note = source = /note="Description of Artificial Sequence: Synthetic peptide"
RDSSGHHL

SEQ ID NO: 847 - protein, 11 aa, synthetic construct
  REGION 1..11: note = source = /note="Description of Artificial Sequence: Synthetic peptide"
YGSSFNGSSLF

SEQ ID NO: 848 - protein, 7 aa, synthetic construct
  REGION 1..7: note = source = /note="Description of Artificial Sequence: Synthetic peptide"
FGTSSGL

SEQ ID NO: 849 - protein, 7 aa, synthetic construct
  REGION 1..7: note = source = /note="Description of Artificial Sequence: Synthetic peptide"
YHSSPSW

SEQ ID NO: 850 - protein, 7 aa, synthetic construct
  REGION 1..7: note = source = /note="Description of Artificial Sequence: Synthetic peptide"
YHSSPSW

SEQ ID NO: 851 - protein, 6 aa, synthetic construct
  REGION 1..6: note = source = /note="Description of Artificial Sequence: Synthetic peptide"
SYSTPY

SEQ ID NO: 852 - protein, 7 aa, synthetic construct
  REGION 1..7: note = source = /note="Description of Artificial Sequence: Synthetic peptide"
YESSPSW

SEQ ID NO: 853 - protein, 7 aa, synthetic construct
  REGION 1..7: note = source = /note="Description of Artificial Sequence: Synthetic peptide"
YGSPPRF

SEQ ID NO: 854 - protein, 7 aa, synthetic construct
  REGION 1..7: note = source = /note="Description of Artificial Sequence: Synthetic peptide"
YGSSPSW

SEQ ID NO: 855 - protein, 7 aa, synthetic construct
  REGION 1..7: note = source = /note="Description of Artificial Sequence: Synthetic peptide"
YDSSPSW

SEQ ID NO: 856 - protein, 7 aa, synthetic construct
  REGION 1..7: note = source = /note="Description of Artificial Sequence: Synthetic peptide"
YGTSPMY

SEQ ID NO: 857 - protein, 6 aa, synthetic construct
  REGION 1..6: note = source = /note="Description of Artificial Sequence: Synthetic peptide"
ALQTPL

SEQ ID NO: 858 - protein, 8 aa, synthetic construct
  REGION 1..8: note = source = /note="Description of Artificial Sequence: Synthetic peptide"
YGNSPPKF

SEQ ID NO: 859 - protein, 7 aa, synthetic construct
  REGION 1..7: note = source = /note="Description of Artificial Sequence: Synthetic peptide"
YGGSPRL

SEQ ID NO: 860 - protein, 6 aa, synthetic construct
  REGION 1..6: note = source = /note="Description of Artificial Sequence: Synthetic peptide"
HYSTPW

SEQ ID NO: 861 - protein, 6 aa, synthetic construct
  REGION 1..6: note = source = /note="Description of Artificial Sequence: Synthetic peptide"
SRIFPR

SEQ ID NO: 862 - protein, 6 aa, synthetic construct
  REGION 1..6: note = source = /note="Description of Artificial Sequence: Synthetic peptide"
SRTIPR

SEQ ID NO: 863 - protein, 6 aa, synthetic construct
  REGION 1..6: note = source = /note="Description of Artificial Sequence: Synthetic peptide"
SRTIPR

SEQ ID NO: 864 - protein, 10 aa, synthetic construct
  REGION 1..10: note = source = /note="Description of Artificial Sequence: Synthetic peptide"
GFTFSNYAMS

SEQ ID NO: 865 - protein, 10 aa, synthetic construct
  REGION 1..10: note = source = /note="Description of Artificial Sequence: Synthetic peptide"
GFTFDDYAMH

SEQ ID NO: 866 - protein, 10 aa, synthetic construct
  REGION 1..10: note = source = /note="Description of Artificial Sequence: Synthetic peptide"
GFALSNHGMS

SEQ ID NO: 867 - protein, 10 aa, synthetic construct
  REGION 1..10: note = source = /note="Description of Artificial Sequence: Synthetic peptide"
GYIFDNFGIN

SEQ ID NO: 868 - protein, 10 aa, synthetic construct
  REGION 1..10: note = source = /note="Description of Artificial Sequence: Synthetic peptide"
GFTFSSDAMT

SEQ ID NO: 869 - protein, 10 aa, synthetic construct
  REGION 1..10: note = source = /note="Description of Artificial Sequence: Synthetic peptide"
GYTFSNYGIT

SEQ ID NO: 870 - protein, 10 aa, synthetic construct
  REGION 1..10: note = source = /note="Description of Artificial Sequence: Synthetic peptide"
GFALSNHGMS

SEQ ID NO: 871 - protein, 10 aa, synthetic construct
  REGION 1..10: note = source = /note="Description of Artificial Sequence: Synthetic peptide"
GFALSNHGMS

SEQ ID NO: 872 - protein, 10 aa, synthetic construct
  REGION 1..10: note = source = /note="Description of Artificial Sequence: Synthetic peptide"
GFALSNHGMS

SEQ ID NO: 873 - protein, 10 aa, synthetic construct
  REGION 1..10: note = source = /note="Description of Artificial Sequence: Synthetic peptide"
GFTFSDYYMS

SEQ ID NO: 874 - protein, 10 aa, synthetic construct
  REGION 1..10: note = source = /note="Description of Artificial Sequence: Synthetic peptide"
GFALSNHGMS

SEQ ID NO: 875 - protein, 10 aa, synthetic construct
  REGION 1..10: note = source = /note="Description of Artificial Sequence: Synthetic peptide"
GFTFSDYYMS

SEQ ID NO: 876 - protein, 10 aa, synthetic construct
  REGION 1..10: note = source = /note="Description of Artificial Sequence: Synthetic peptide"
GFALSNHGMS

SEQ ID NO: 877 - protein, 10 aa, synthetic construct
  REGION 1..10: note = source = /note="Description of Artificial Sequence: Synthetic peptide"
GFALSNHGMS

SEQ ID NO: 878 - protein, 10 aa, synthetic construct
  REGION 1..10: note = source = /note="Description of Artificial Sequence: Synthetic peptide"
GFALSNHGMS

SEQ ID NO: 879 - protein, 12 aa, synthetic construct
  REGION 1..12: note = source = /note="Description of Artificial Sequence: Synthetic peptide"
GGSISSSYYYWG

SEQ ID NO: 880 - protein, 12 aa, synthetic construct
  REGION 1..12: note = source = /note="Description of Artificial Sequence: Synthetic peptide"
GFSLRTSGMCVS

SEQ ID NO: 881 - protein, 10 aa, synthetic construct
  REGION 1..10: note = source = /note="Description of Artificial Sequence: Synthetic peptide"
GFTFSSYSMN

SEQ ID NO: 882 - protein, 10 aa, synthetic construct
  REGION 1..10: note = source = /note="Description of Artificial Sequence: Synthetic peptide"
GFTFSDYYMS

SEQ ID NO: 883 - protein, 10 aa, synthetic construct
  REGION 1..10: note = source = /note="Description of Artificial Sequence: Synthetic peptide"
GYTVTSHYIH

SEQ ID NO: 884 - protein, 12 aa, synthetic construct
  REGION 1..12: note = source = /note="Description of Artificial Sequence: Synthetic peptide"
GGSISSGGYYWS

SEQ ID NO: 885 - protein, 10 aa, synthetic construct
  REGION 1..10: note = source = /note="Description of Artificial Sequence: Synthetic peptide"
GGTFSSYAIS

SEQ ID NO: 886 - protein, 12 aa, synthetic construct
  REGION 1..12: note = source = /note="Description of Artificial Sequence: Synthetic peptide"
GDSVSSNSAAWN

SEQ ID NO: 887 - protein, 10 aa, synthetic construct
  REGION 1..10: note = source = /note="Description of Artificial Sequence: Synthetic peptide"
GFTFSSYAMS

SEQ ID NO: 888 - protein, 10 aa, synthetic construct
  REGION 1..10: note = source = /note="Description of Artificial Sequence: Synthetic peptide"
GITFSRYPMS

SEQ ID NO: 889 - protein, 10 aa, synthetic construct
  REGION 1..10: note = source = /note="Description of Artificial Sequence: Synthetic peptide"
GFTFSSYAMS

SEQ ID NO: 890 - protein, 10 aa, synthetic construct
  REGION 1..10: note = source = /note="Description of Artificial Sequence: Synthetic peptide"
GFTFSSYAMS

SEQ ID NO: 891 - protein, 10 aa, synthetic construct
  REGION 1..10: note = source = /note="Description of Artificial Sequence: Synthetic peptide"
GFTFDDYAMH

SEQ ID NO: 892 - protein, 10 aa, synthetic construct
  REGION 1..10: note = source = /note="Description of Artificial Sequence: Synthetic peptide"
GFTFDDYAMH

SEQ ID NO: 893 - protein, 10 aa, synthetic construct
  REGION 1..10: note = source = /note="Description of Artificial Sequence: Synthetic peptide"
GFTFSSYAMS

SEQ ID NO: 894 - protein, 10 aa, synthetic construct
  REGION 1..10: note = source = /note="Description of Artificial Sequence: Synthetic peptide"
GFTFSSYAMS

SEQ ID NO: 895 - protein, 10 aa, synthetic construct
  REGION 1..10: note = source = /note="Description of Artificial Sequence: Synthetic peptide"
GFTFSSYAMS

SEQ ID NO: 896 - protein, 10 aa, synthetic construct
  REGION 1..10: note = source = /note="Description of Artificial Sequence: Synthetic peptide"
GFSFSSYAMS

SEQ ID NO: 897 - protein, 10 aa, synthetic construct
  REGION 1..10: note = source = /note="Description of Artificial Sequence: Synthetic peptide"
GFTFSSYAMS

SEQ ID NO: 898 - protein, 10 aa, synthetic construct
  REGION 1..10: note = source = /note="Description of Artificial Sequence: Synthetic peptide"
GFTFSSYAMS

SEQ ID NO: 899 - protein, 10 aa, synthetic construct
  REGION 1..10: note = source = /note="Description of Artificial Sequence: Synthetic peptide"
GFTFSSYAMS

SEQ ID NO: 900 - protein, 10 aa, synthetic construct
  REGION 1..10: note = source = /note="Description of Artificial Sequence: Synthetic peptide"
GYTFTNFGMN

SEQ ID NO: 901 - protein, 10 aa, synthetic construct
  REGION 1..10: note = source = /note="Description of Artificial Sequence: Synthetic peptide"
GYTFTDYSIN

SEQ ID NO: 902 - protein, 10 aa, synthetic construct
  REGION 1..10: note = source = /note="Description of Artificial Sequence: Synthetic peptide"
GYTFRHYSMN

SEQ ID NO: 903 - protein, 10 aa, synthetic construct
  REGION 1..10: note = source = /note="Description of Artificial Sequence: Synthetic peptide"
GYTFTHYSMN

SEQ ID NO: 904 - protein, 17 aa, synthetic construct
  REGION 1..17: note = source = /note="Description of Artificial Sequence: Synthetic peptide"
GISRSGENTYYADSVKG

SEQ ID NO: 905 - protein, 17 aa, synthetic construct
  REGION 1..17: note = source = /note="Description of Artificial Sequence: Synthetic peptide"
GISWNSGSIGYADSVKG

SEQ ID NO: 906 - protein, 16 aa, synthetic construct
  REGION 1..16: note = source = /note="Description of Artificial Sequence: Synthetic peptide"
GIVYSGSTYYAASVKG

SEQ ID NO: 907 - protein, 17 aa, synthetic construct
  REGION 1..17: note = source = /note="Description of Artificial Sequence: Synthetic peptide"
WINPKNNNTNYAQKFQG

SEQ ID NO: 908 - protein, 17 aa, synthetic construct
  REGION 1..17: note = source = /note="Description of Artificial Sequence: Synthetic peptide"
VISGSGGTTYYADSVKG

SEQ ID NO: 909 - protein, 17 aa, synthetic construct
  REGION 1..17: note = source = /note="Description of Artificial Sequence: Synthetic peptide"
WISAYNGNTNYAQKFQG

SEQ ID NO: 910 - protein, 16 aa, synthetic construct
  REGION 1..16: note = source = /note="Description of Artificial Sequence: Synthetic peptide"
GIVYSGSTYYAASVKG

SEQ ID NO: 911 - protein, 16 aa, synthetic construct
  REGION 1..16: note = source = /note="Description of Artificial Sequence: Synthetic peptide"
GIVYSGSTYYAASVKG

SEQ ID NO: 912 - protein, 16 aa, synthetic construct
  REGION 1..16: note = source = /note="Description of Artificial Sequence: Synthetic peptide"
GIVYSGSTYYAASVKG

SEQ ID NO: 913 - protein, 17 aa, synthetic construct
  REGION 1..17: note = source = /note="Description of Artificial Sequence: Synthetic peptide"
YISSSGSTIYYADSVKG

SEQ ID NO: 914 - protein, 16 aa, synthetic construct
  REGION 1..16: note = source = /note="Description of Artificial Sequence: Synthetic peptide"
GIVYSGSTYYAASVKG

SEQ ID NO: 915 - protein, 17 aa, synthetic construct
  REGION 1..17: note = source = /note="Description of Artificial Sequence: Synthetic peptide"
YISSSGNTIYYADSVKG

SEQ ID NO: 916 - protein, 16 aa, synthetic construct
  REGION 1..16: note = source = /note="Description of Artificial Sequence: Synthetic peptide"
GIVYSGSTYYAASVKG

SEQ ID NO: 917 - protein, 16 aa, synthetic construct
  REGION 1..16: note = source = /note="Description of Artificial Sequence: Synthetic peptide"
GIVYSGSTYYAASVKG

SEQ ID NO: 918 - protein, 16 aa, synthetic construct
  REGION 1..16: note = source = /note="Description of Artificial Sequence: Synthetic peptide"
GIVYSGSTYYAASVKG

SEQ ID NO: 919 - protein, 16 aa, synthetic construct
  REGION 1..16: note = source = /note="Description of Artificial Sequence: Synthetic peptide"
SIYYSGSAYYNPSLKS

SEQ ID NO: 920 - protein, 16 aa, synthetic construct
  REGION 1..16: note = source = /note="Description of Artificial Sequence: Synthetic peptide"
RIDWDEDKFYSTSLKT

SEQ ID NO: 921 - protein, 17 aa, synthetic construct
  REGION 1..17: note = source = /note="Description of Artificial Sequence: Synthetic peptide"
SISSSSSYIYYADSVKG

SEQ ID NO: 922 - protein, 17 aa, synthetic construct
  REGION 1..17: note = source = /note="Description of Artificial Sequence: Synthetic peptide"
YISSSGSTIYYADSVKG

SEQ ID NO: 923 - protein, 17 aa, synthetic construct
  REGION 1..17: note = source = /note="Description of Artificial Sequence: Synthetic peptide"
MINPSGGVTAYSQTLQG

SEQ ID NO: 924 - protein, 16 aa, synthetic construct
  REGION 1..16: note = source = /note="Description of Artificial Sequence: Synthetic peptide"
YIYYSGSTYYNPSLKS

SEQ ID NO: 925 - protein, 17 aa, synthetic construct
  REGION 1..17: note = source = /note="Description of Artificial Sequence: Synthetic peptide"
GIIPIFGTANYAQKFQG

SEQ ID NO: 926 - protein, 18 aa, synthetic construct
  REGION 1..18: note = source = /note="Description of Artificial Sequence: Synthetic peptide"
RTYYRSKWYSFYAISLKS

SEQ ID NO: 927 - protein, 17 aa, synthetic construct
  REGION 1..17: note = source = /note="Description of Artificial Sequence: Synthetic peptide"
AISGSGGSTYYADSVKG

SEQ ID NO: 928 - protein, 17 aa, synthetic construct
  REGION 1..17: note = source = /note="Description of Artificial Sequence: Synthetic peptide"
GISDSGVSTYYADSAKG

SEQ ID NO: 929 - protein, 17 aa, synthetic construct
  REGION 1..17: note = source = /note="Description of Artificial Sequence: Synthetic peptide"
AISGSGGSTYYADSVKG

SEQ ID NO: 930 - protein, 17 aa, synthetic construct
  REGION 1..17: note = source = /note="Description of Artificial Sequence: Synthetic peptide"
AISGSGGSTYYADSVKG

SEQ ID NO: 931 - protein, 17 aa, synthetic construct
  REGION 1..17: note = source = /note="Description of Artificial Sequence: Synthetic peptide"
GISWNSGSIGYADSVKG

SEQ ID NO: 932 - protein, 17 aa, synthetic construct
  REGION 1..17: note = source = /note="Description of Artificial Sequence: Synthetic peptide"
SINWKGNSLAYGDSVKG

SEQ ID NO: 933 - protein, 17 aa, synthetic construct
  REGION 1..17: note = source = /note="Description of Artificial Sequence: Synthetic peptide"
AISGSGGSTYYADSVKG

SEQ ID NO: 934 - protein, 17 aa, synthetic construct
  REGION 1..17: note = source = /note="Description of Artificial Sequence: Synthetic peptide"
AISGSGGSTYYADSVKG

SEQ ID NO: 935 - protein, 17 aa, synthetic construct
  REGION 1..17: note = source = /note="Description of Artificial Sequence: Synthetic peptide"
AISGSGGSTYYADSVKG

SEQ ID NO: 936 - protein, 17 aa, synthetic construct
  REGION 1..17: note = source = /note="Description of Artificial Sequence: Synthetic peptide"
AISGSGGSTYYADSVKG

SEQ ID NO: 937 - protein, 17 aa, synthetic construct
  REGION 1..17: note = source = /note="Description of Artificial Sequence: Synthetic peptide"
AISGSGGSTYYADSVKG

SEQ ID NO: 938 - protein, 17 aa, synthetic construct
  REGION 1..17: note = source = /note="Description of Artificial Sequence: Synthetic peptide"
AISGSGGSTYYADSVKG

SEQ ID NO: 939 - protein, 17 aa, synthetic construct
  REGION 1..17: note = source = /note="Description of Artificial Sequence: Synthetic peptide"
AISGSGGSTYYADSVKG

SEQ ID NO: 940 - protein, 17 aa, synthetic construct
  REGION 1..17: note = source = /note="Description of Artificial Sequence: Synthetic peptide"
WINTYTGESYFADDFKG

SEQ ID NO: 941 - protein, 17 aa, synthetic construct
  REGION 1..17: note = source = /note="Description of Artificial Sequence: Synthetic peptide"
WINTETREPAYAYDFRG

SEQ ID NO: 942 - protein, 17 aa, synthetic construct
  REGION 1..17: note = source = /note="Description of Artificial Sequence: Synthetic peptide"
RINTESGVPIYADDFKG

SEQ ID NO: 943 - protein, 17 aa, synthetic construct
  REGION 1..17: note = source = /note="Description of Artificial Sequence: Synthetic peptide"
RINTETGEPLYADDFKG

SEQ ID NO: 944 - protein, 11 aa, synthetic construct
  REGION 1..11: note = source = /note="Description of Artificial Sequence: Synthetic peptide"
SPAHYYGGMDV

SEQ ID NO: 945 - protein, 6 aa, synthetic construct
  REGION 1..6: note = source = /note="Description of Artificial Sequence: Synthetic peptide"
HSFLAY

SEQ ID NO: 946 - protein, 7 aa, synthetic construct
  REGION 1..7: note = source = /note="Description of Artificial Sequence: Synthetic peptide"
HGGESDV

SEQ ID NO: 947 - protein, 11 aa, synthetic construct
  REGION 1..11: note = source = /note="Description of Artificial Sequence: Synthetic peptide"
GPYYYQSYMDV

SEQ ID NO: 948 - protein, 14 aa, synthetic construct
  REGION 1..14: note = source = /note="Description of Artificial Sequence: Synthetic peptide"
LDSSGYYYARGPRY

SEQ ID NO: 949 - protein, 9 aa, synthetic construct
  REGION 1..9: note = source = /note="Description of Artificial Sequence: Synthetic peptide"
GPYYYYMDV

SEQ ID NO: 950 - protein, 7 aa, synthetic construct
  REGION 1..7: note = source = /note="Description of Artificial Sequence: Synthetic peptide"
HGGESDV

SEQ ID NO: 951 - protein, 7 aa, synthetic construct
  REGION 1..7: note = source = /note="Description of Artificial Sequence: Synthetic peptide"
HGGESDV

SEQ ID NO: 952 - protein, 7 aa, synthetic construct
  REGION 1..7: note = source = /note="Description of Artificial Sequence: Synthetic peptide"
HGGESDV

SEQ ID NO: 953 - protein, 8 aa, synthetic construct
  REGION 1..8: note = source = /note="Description of Artificial Sequence: Synthetic peptide"
ESGDGMDV

SEQ ID NO: 954 - protein, 7 aa, synthetic construct
  REGION 1..7: note = source = /note="Description of Artificial Sequence: Synthetic peptide"
HGGESDV

SEQ ID NO: 955 - protein, 8 aa, synthetic construct
  REGION 1..8: note = source = /note="Description of Artificial Sequence: Synthetic peptide"
STMVREDY

SEQ ID NO: 956 - protein, 7 aa, synthetic construct
  REGION 1..7: note = source = /note="Description of Artificial Sequence: Synthetic peptide"
HGGESDV

SEQ ID NO: 957 - protein, 7 aa, synthetic construct
  REGION 1..7: note = source = /note="Description of Artificial Sequence: Synthetic peptide"
HGGESDV

SEQ ID NO: 958 - protein, 7 aa, synthetic construct
  REGION 1..7: note = source = /note="Description of Artificial Sequence: Synthetic peptide"
HGGESDV

SEQ ID NO: 959 - protein, 11 aa, synthetic construct
  REGION 1..11: note = source = /note="Description of Artificial Sequence: Synthetic peptide"
HWQEWPDAFDI

SEQ ID NO: 960 - protein, 13 aa, synthetic construct
  REGION 1..13: note = source = /note="Description of Artificial Sequence: Synthetic peptide"
SGAGGTSATAFDI

SEQ ID NO: 961 - protein, 10 aa, synthetic construct
  REGION 1..10: note = source = /note="Description of Artificial Sequence: Synthetic peptide"
TIAAVYAFDI

SEQ ID NO: 962 - protein, 8 aa, synthetic construct
  REGION 1..8: note = source = /note="Description of Artificial Sequence: Synthetic peptide"
DLRGAFDI

SEQ ID NO: 963 - protein, 11 aa, synthetic construct
  REGION 1..11: note = source = /note="Description of Artificial Sequence: Synthetic peptide"
EGSGSGWYFDF

SEQ ID NO: 964 - protein, 13 aa, synthetic construct
  REGION 1..13: note = source = /note="Description of Artificial Sequence: Synthetic peptide"
AGIAARLRGAFDI

SEQ ID NO: 965 - protein, 20 aa, synthetic construct
  REGION 1..20: note = source = /note="Description of Artificial Sequence: Synthetic peptide"
RGGYQLLRWDVGLLRSAFDI

SEQ ID NO: 966 - protein, 13 aa, synthetic construct
  REGION 1..13: note = source = /note="Description of Artificial Sequence: Synthetic peptide"
SSPEGLFLYWFDP

SEQ ID NO: 967 - protein, 9 aa, synthetic construct
  REGION 1..9: note = source = /note="Description of Artificial Sequence: Synthetic peptide"
VEGSGSLDY

SEQ ID NO: 968 - protein, 9 aa, synthetic construct
  REGION 1..9: note = source = /note="Description of Artificial Sequence: Synthetic peptide"
RAGSEASDI

SEQ ID NO: 969 - protein, 15 aa, synthetic construct
  REGION 1..15: note = source = /note="Description of Artificial Sequence: Synthetic peptide"
ATYKRELRYYYGMDV

SEQ ID NO: 970 - protein, 15 aa, synthetic construct
  REGION 1..15: note = source = /note="Description of Artificial Sequence: Synthetic peptide"
ATYKRELRYYYGMDV

SEQ ID NO: 971 - protein, 8 aa, synthetic construct
  REGION 1..8: note = source = /note="Description of Artificial Sequence: Synthetic peptide"
VGKAVPDV

SEQ ID NO: 972 - protein, 13 aa, synthetic construct
  REGION 1..13: note = source = /note="Description of Artificial Sequence: Synthetic peptide"
HQGVAYYNYAMDV

SEQ ID NO: 973 - protein, 8 aa, synthetic construct
  REGION 1..8: note = source = /note="Description of Artificial Sequence: Synthetic peptide"
VVRDGMDV

SEQ ID NO: 974 - protein, 9 aa, synthetic construct
  REGION 1..9: note = source = /note="Description of Artificial Sequence: Synthetic peptide"
IPQTGTFDY

SEQ ID NO: 975 - protein, 15 aa, synthetic construct
  REGION 1..15: note = source = /note="Description of Artificial Sequence: Synthetic peptide"
ANYKRELRYYYGMDV

SEQ ID NO: 976 - protein, 11 aa, synthetic construct
  REGION 1..11: note = source = /note="Description of Artificial Sequence: Synthetic peptide"
ALVGATGAFDI

SEQ ID NO: 977 - protein, 8 aa, synthetic construct
  REGION 1..8: note = source = /note="Description of Artificial Sequence: Synthetic peptide"
WFGEGFDP

SEQ ID NO: 978 - protein, 17 aa, synthetic construct
  REGION 1..17: note = source = /note="Description of Artificial Sequence: Synthetic peptide"
VGYDSSGYYRDYYGMDV

SEQ ID NO: 979 - protein, 13 aa, synthetic construct
  REGION 1..13: note = source = /note="Description of Artificial Sequence: Synthetic peptide"
MGWSSGYLGAFDI

SEQ ID NO: 980 - protein, 13 aa, synthetic construct
  REGION 1..13: note = source = /note="Description of Artificial Sequence: Synthetic peptide"
GEIYYGYDGGFAY

SEQ ID NO: 981 - protein, 8 aa, synthetic construct
  REGION 1..8: note = source = /note="Description of Artificial Sequence: Synthetic peptide"
DYSYAMDY

SEQ ID NO: 982 - protein, 8 aa, synthetic construct
  REGION 1..8: note = source = /note="Description of Artificial Sequence: Synthetic peptide"
DYLYSLDF

SEQ ID NO: 983 - protein, 8 aa, synthetic construct
  REGION 1..8: note = source = /note="Description of Artificial Sequence: Synthetic peptide"
DYLYSCDY

SEQ ID NO: 984 - protein, 12 aa, synthetic construct
  REGION 1..12: note = source = /note="Description of Artificial Sequence: Synthetic peptide"
RASQSISSSFLA

SEQ ID NO: 985 - protein, 16 aa, synthetic construct
  REGION 1..16: note = source = /note="Description of Artificial Sequence: Synthetic peptide"
RSSQSLLHSNGYNYLD

SEQ ID NO: 986 - protein, 16 aa, synthetic construct
  REGION 1..16: note = source = /note="Description of Artificial Sequence: Synthetic peptide"
KSSQSLLRNDGKTPLY

SEQ ID NO: 987 - protein, 16 aa, synthetic construct
  REGION 1..16: note = source = /note="Description of Artificial Sequence: Synthetic peptide"
RSSQSLLHSNGYNYLN

SEQ ID NO: 988 - protein, 11 aa, synthetic construct
  REGION 1..11: note = source = /note="Description of Artificial Sequence: Synthetic peptide"
RASQSISSYLN

SEQ ID NO: 989 - protein, 16 aa, synthetic construct
  REGION 1..16: note = source = /note="Description of Artificial Sequence: Synthetic peptide"
RSSQSLLYSNGYNYVD

SEQ ID NO: 990 - protein, 11 aa, synthetic construct
  REGION 1..11: note = source = /note="Description of Artificial Sequence: Synthetic peptide"
RASQSVSSNLA

SEQ ID NO: 991 - protein, 11 aa, synthetic construct
  REGION 1..11: note = source = /note="Description of Artificial Sequence: Synthetic peptide"
RASQSVSSKLA

SEQ ID NO: 992 - protein, 12 aa, synthetic construct
  REGION 1..12: note = source = /note="Description of Artificial Sequence: Synthetic peptide"
RASQSVGSTNLA

SEQ ID NO: 993 - protein, 11 aa, synthetic construct
  REGION 1..11: note = source = /note="Description of Artificial Sequence: Synthetic peptide"
RASQSISSYLN

SEQ ID NO: 994 - protein, 11 aa, synthetic construct
  REGION 1..11: note = source = /note="Description of Artificial Sequence: Synthetic peptide"
RASQSISSYLN

SEQ ID NO: 995 - protein, 16 aa, synthetic construct
  REGION 1..16: note = source = /note="Description of Artificial Sequence: Synthetic peptide"
KSSESLVHNSGKTYLN

SEQ ID NO: 996 - protein, 11 aa, synthetic construct
  REGION 1..11: note = source = /note="Description of Artificial Sequence: Synthetic peptide"
QASEDINKFLN

SEQ ID NO: 997 - protein, 11 aa, synthetic construct
  REGION 1..11: note = source = /note="Description of Artificial Sequence: Synthetic peptide"
RASQSVGSNLA

SEQ ID NO: 998 - protein, 12 aa, synthetic construct
  REGION 1..12: note = source = /note="Description of Artificial Sequence: Synthetic peptide"
RASQSIGSSSLA

SEQ ID NO: 999 - protein, 11 aa, synthetic construct
  REGION 1..11: note = source = /note="Description of Artificial Sequence: Synthetic peptide"
KASQDIDDAMN

SEQ ID NO: 1000 - protein, 11 aa, synthetic construct
  REGION 1..11: note = source = /note="Description of Artificial Sequence: Synthetic peptide"
RASQDIYNNLA

SEQ ID NO: 1001 - protein, 16 aa, synthetic construct
  REGION 1..16: note = source = /note="Description of Artificial Sequence: Synthetic peptide"
RSSQSLLHSNGYNYLD

SEQ ID NO: 1002 - protein, 11 aa, synthetic construct
  REGION 1..11: note = source = /note="Description of Artificial Sequence: Synthetic peptide"
GGNNIGTKSVH

SEQ ID NO: 1003 - protein, 11 aa, synthetic construct
  REGION 1..11: note = source = /note="Description of Artificial Sequence: Synthetic peptide"
SGDGLSKKYVS

SEQ ID NO: 1004 - protein, 11 aa, synthetic construct
  REGION 1..11: note = source = /note="Description of Artificial Sequence: Synthetic peptide"
RASQGIRNWLA

SEQ ID NO: 1005 - protein, 11 aa, synthetic construct
  REGION 1..11: note = source = /note="Description of Artificial Sequence: Synthetic peptide"
GGNNIGSKSVH

SEQ ID NO: 1006 - protein, 11 aa, synthetic construct
  REGION 1..11: note = source = /note="Description of Artificial Sequence: Synthetic peptide"
QGDSLGNYYAT

SEQ ID NO: 1007 - protein, 12 aa, synthetic construct
  REGION 1..12: note = source = /note="Description of Artificial Sequence: Synthetic peptide"
RASQSVSSAYLA

SEQ ID NO: 1008 - protein, 11 aa, synthetic construct
  REGION 1..11: note = source = /note="Description of Artificial Sequence: Synthetic peptide"
RASQSVSNSLA

SEQ ID NO: 1009 - protein, 12 aa, synthetic construct
  REGION 1..12: note = source = /note="Description of Artificial Sequence: Synthetic peptide"
RASQSVSSSFLA

SEQ ID NO: 1010 - protein, 12 aa, synthetic construct
  REGION 1..12: note = source = /note="Description of Artificial Sequence: Synthetic peptide"
RASQSVSTTFLA

SEQ ID NO: 1011 - protein, 11 aa, synthetic construct
  REGION 1..11: note = source = /note="Description of Artificial Sequence: Synthetic peptide"
RASQSISSYLN

SEQ ID NO: 1012 - protein, 12 aa, synthetic construct
  REGION 1..12: note = source = /note="Description of Artificial Sequence: Synthetic peptide"
RATQSIGSSFLA

SEQ ID NO: 1013 - protein, 12 aa, synthetic construct
  REGION 1..12: note = source = /note="Description of Artificial Sequence: Synthetic peptide"
RASQSVSSSYLA

SEQ ID NO: 1014 - protein, 12 aa, synthetic construct
  REGION 1..12: note = source = /note="Description of Artificial Sequence: Synthetic peptide"
RASQSVSSSYLA

SEQ ID NO: 1015 - protein, 12 aa, synthetic construct
  REGION 1..12: note = source = /note="Description of Artificial Sequence: Synthetic peptide"
RASQRVASNYLA

SEQ ID NO: 1016 - protein, 12 aa, synthetic construct
  REGION 1..12: note = source = /note="Description of Artificial Sequence: Synthetic peptide"
RASQSLSSNFLA

SEQ ID NO: 1017 - protein, 16 aa, synthetic construct
  REGION 1..16: note = source = /note="Description of Artificial Sequence: Synthetic peptide"
RSSQSLLHSNGYNYLD

SEQ ID NO: 1018 - protein, 12 aa, synthetic construct
  REGION 1..12: note = source = /note="Description of Artificial Sequence: Synthetic peptide"
RASQSVSSSYLA

SEQ ID NO: 1019 - protein, 12 aa, synthetic construct
  REGION 1..12: note = source = /note="Description of Artificial Sequence: Synthetic peptide"
RASQSVASSFLA

SEQ ID NO: 1020 - protein, 11 aa, synthetic construct
  REGION 1..11: note = source = /note="Description of Artificial Sequence: Synthetic peptide"
RASQDVNTAVS

SEQ ID NO: 1021 - protein, 15 aa, synthetic construct
  REGION 1..15: note = source = /note="Description of Artificial Sequence: Synthetic peptide"
RASESVSVIGAHLIH

SEQ ID NO: 1022 - protein, 15 aa, synthetic construct
  REGION 1..15: note = source = /note="Description of Artificial Sequence: Synthetic peptide"
RASESVTILGSHLIY

SEQ ID NO: 1023 - protein, 15 aa, synthetic construct
  REGION 1..15: note = source = /note="Description of Artificial Sequence: Synthetic peptide"
RASESVTILGSHLIY

SEQ ID NO: 1024 - protein, 7 aa, synthetic construct
  REGION 1..7: note = source = /note="Description of Artificial Sequence: Synthetic peptide"
GASRRAT

SEQ ID NO: 1025 - protein, 7 aa, synthetic construct
  REGION 1..7: note = source = /note="Description of Artificial Sequence: Synthetic peptide"
LGSNRAS

SEQ ID NO: 1026 - protein, 7 aa, synthetic construct
  REGION 1..7: note = source = /note="Description of Artificial Sequence: Synthetic peptide"
EVSNRFS

SEQ ID NO: 1027 - protein, 7 aa, synthetic construct
  REGION 1..7: note = source = /note="Description of Artificial Sequence: Synthetic peptide"
LGSKRAS

SEQ ID NO: 1028 - protein, 7 aa, synthetic construct
  REGION 1..7: note = source = /note="Description of Artificial Sequence: Synthetic peptide"
GASTLAS

SEQ ID NO: 1029 - protein, 7 aa, synthetic construct
  REGION 1..7: note = source = /note="Description of Artificial Sequence: Synthetic peptide"
LGSNRAS

SEQ ID NO: 1030 - protein, 7 aa, synthetic construct
  REGION 1..7: note = source = /note="Description of Artificial Sequence: Synthetic peptide"
GASTRAS

SEQ ID NO: 1031 - protein, 7 aa, synthetic construct
  REGION 1..7: note = source = /note="Description of Artificial Sequence: Synthetic peptide"
GASIRAT

SEQ ID NO: 1032 - protein, 7 aa, synthetic construct
  REGION 1..7: note = source = /note="Description of Artificial Sequence: Synthetic peptide"
DASNRAT

SEQ ID NO: 1033 - protein, 7 aa, synthetic construct
  REGION 1..7: note = source = /note="Description of Artificial Sequence: Synthetic peptide"
AASSLQS

SEQ ID NO: 1034 - protein, 7 aa, synthetic construct
  REGION 1..7: note = source = /note="Description of Artificial Sequence: Synthetic peptide"
AASSLQS

SEQ ID NO: 1035 - protein, 7 aa, synthetic construct
  REGION 1..7: note = source = /note="Description of Artificial Sequence: Synthetic peptide"
EVSNRDS

SEQ ID NO: 1036 - protein, 7 aa, synthetic construct
  REGION 1..7: note = source = /note="Description of Artificial Sequence: Synthetic peptide"
DASTLQT

SEQ ID NO: 1037 - protein, 7 aa, synthetic construct
  REGION 1..7: note = source = /note="Description of Artificial Sequence: Synthetic peptide"
GASTRAT

SEQ ID NO: 1038 - protein, 7 aa, synthetic construct
  REGION 1..7: note = source = /note="Description of Artificial Sequence: Synthetic peptide"
GASSRAS

SEQ ID NO: 1039 - protein, 7 aa, synthetic construct
  REGION 1..7: note = source = /note="Description of Artificial Sequence: Synthetic peptide"
SATSPVP

SEQ ID NO: 1040 - protein, 7 aa, synthetic construct
  REGION 1..7: note = source = /note="Description of Artificial Sequence: Synthetic peptide"
AANKSQS

SEQ ID NO: 1041 - protein, 7 aa, synthetic construct
  REGION 1..7: note = source = /note="Description of Artificial Sequence: Synthetic peptide"
LGSNRAS

SEQ ID NO: 1042 - protein, 7 aa, synthetic construct
  REGION 1..7: note = source = /note="Description of Artificial Sequence: Synthetic peptide"
DDSVRPS

SEQ ID NO: 1043 - protein, 7 aa, synthetic construct
  REGION 1..7: note = source = /note="Description of Artificial Sequence: Synthetic peptide"
RDKERPS

SEQ ID NO: 1044 - protein, 7 aa, synthetic construct
  REGION 1..7: note = source = /note="Description of Artificial Sequence: Synthetic peptide"
AASNLQS

SEQ ID NO: 1045 - protein, 7 aa, synthetic construct
  REGION 1..7: note = source = /note="Description of Artificial Sequence: Synthetic peptide"
GKNNRPS

SEQ ID NO: 1046 - protein, 7 aa, synthetic construct
  REGION 1..7: note = source = /note="Description of Artificial Sequence: Synthetic peptide"
GTNNRPS

SEQ ID NO: 1047 - protein, 7 aa, synthetic construct
  REGION 1..7: note = source = /note="Description of Artificial Sequence: Synthetic peptide"
GASTRAT

SEQ ID NO: 1048 - protein, 7 aa, synthetic construct
  REGION 1..7: note = source = /note="Description of Artificial Sequence: Synthetic peptide"
DASSRAT

SEQ ID NO: 1049 - protein, 7 aa, synthetic construct
  REGION 1..7: note = source = /note="Description of Artificial Sequence: Synthetic peptide"
GASSRAT

SEQ ID NO: 1050 - protein, 7 aa, synthetic construct
  REGION 1..7: note = source = /note="Description of Artificial Sequence: Synthetic peptide"
GSSNRAT

SEQ ID NO: 1051 - protein, 7 aa, synthetic construct
  REGION 1..7: note = source = /note="Description of Artificial Sequence: Synthetic peptide"
AASSLQS

SEQ ID NO: 1052 - protein, 7 aa, synthetic construct
  REGION 1..7: note = source = /note="Description of Artificial Sequence: Synthetic peptide"
GASQRAT

SEQ ID NO: 1053 - protein, 7 aa, synthetic construct
  REGION 1..7: note = source = /note="Description of Artificial Sequence: Synthetic peptide"
GASSRAT

SEQ ID NO: 1054 - protein, 7 aa, synthetic construct
  REGION 1..7: note = source = /note="Description of Artificial Sequence: Synthetic peptide"
GASSRAT

SEQ ID NO: 1055 - protein, 7 aa, synthetic construct
  REGION 1..7: note = source = /note="Description of Artificial Sequence: Synthetic peptide"
GASSRAT

SEQ ID NO: 1056 - protein, 7 aa, synthetic construct
  REGION 1..7: note = source = /note="Description of Artificial Sequence: Synthetic peptide"
GASNWAT

SEQ ID NO: 1057 - protein, 7 aa, synthetic construct
  REGION 1..7: note = source = /note="Description of Artificial Sequence: Synthetic peptide"
LGSNRAS

SEQ ID NO: 1058 - protein, 7 aa, synthetic construct
  REGION 1..7: note = source = /note="Description of Artificial Sequence: Synthetic peptide"
GTSSRAT

SEQ ID NO: 1059 - protein, 7 aa, synthetic construct
  REGION 1..7: note = source = /note="Description of Artificial Sequence: Synthetic peptide"
GASGRAT

SEQ ID NO: 1060 - protein, 7 aa, synthetic construct
  REGION 1..7: note = source = /note="Description of Artificial Sequence: Synthetic peptide"
SASYRYT

SEQ ID NO: 1061 - protein, 7 aa, synthetic construct
  REGION 1..7: note = source = /note="Description of Artificial Sequence: Synthetic peptide"
LASNLET

SEQ ID NO: 1062 - protein, 7 aa, synthetic construct
  REGION 1..7: note = source = /note="Description of Artificial Sequence: Synthetic peptide"
LASNVQT

SEQ ID NO: 1063 - protein, 7 aa, synthetic construct
  REGION 1..7: note = source = /note="Description of Artificial Sequence: Synthetic peptide"
LASNVQT

SEQ ID NO: 1064 - protein, 10 aa, synthetic construct
  REGION 1..10: note = source = /note="Description of Artificial Sequence: Synthetic peptide"
QQYHSSPSWT

SEQ ID NO: 1065 - protein, 9 aa, synthetic construct
  REGION 1..9: note = source = /note="Description of Artificial Sequence: Synthetic peptide"
MQALQTPYT

SEQ ID NO: 1066 - protein, 8 aa, synthetic construct
  REGION 1..8: note = source = /note="Description of Artificial Sequence: Synthetic peptide"
MQNIQFPS

SEQ ID NO: 1067 - protein, 9 aa, synthetic construct
  REGION 1..9: note = source = /note="Description of Artificial Sequence: Synthetic peptide"
MQALQTPYT

SEQ ID NO: 1068 - protein, 8 aa, synthetic construct
  REGION 1..8: note = source = /note="Description of Artificial Sequence: Synthetic peptide"
QQSYKRAS

SEQ ID NO: 1069 - protein, 9 aa, synthetic construct
  REGION 1..9: note = source = /note="Description of Artificial Sequence: Synthetic peptide"
MQGRQFPYS

SEQ ID NO: 1070 - protein, 8 aa, synthetic construct
  REGION 1..8: note = source = /note="Description of Artificial Sequence: Synthetic peptide"
QQYGSSLT

SEQ ID NO: 1071 - protein, 9 aa, synthetic construct
  REGION 1..9: note = source = /note="Description of Artificial Sequence: Synthetic peptide"
QQYGSSSWT

SEQ ID NO: 1072 - protein, 10 aa, synthetic construct
  REGION 1..10: note = source = /note="Description of Artificial Sequence: Synthetic peptide"
QQYGSSPPWT

SEQ ID NO: 1073 - protein, 7 aa, synthetic construct
  REGION 1..7: note = source = /note="Description of Artificial Sequence: Synthetic peptide"
QQSYTLA

SEQ ID NO: 1074 - protein, 9 aa, synthetic construct
  REGION 1..9: note = source = /note="Description of Artificial Sequence: Synthetic peptide"
QQSYSTPYT

SEQ ID NO: 1075 - protein, 9 aa, synthetic construct
  REGION 1..9: note = source = /note="Description of Artificial Sequence: Synthetic peptide"
MQGTHWPGT

SEQ ID NO: 1076 - protein, 9 aa, synthetic construct
  REGION 1..9: note = source = /note="Description of Artificial Sequence: Synthetic peptide"
QQYESLPLT

SEQ ID NO: 1077 - protein, 10 aa, synthetic construct
  REGION 1..10: note = source = /note="Description of Artificial Sequence: Synthetic peptide"
QQYNDWLPVT

SEQ ID NO: 1078 - protein, 10 aa, synthetic construct
  REGION 1..10: note = source = /note="Description of Artificial Sequence: Synthetic peptide"
QQYAGSPPFT

SEQ ID NO: 1079 - protein, 9 aa, synthetic construct
  REGION 1..9: note = source = /note="Description of Artificial Sequence: Synthetic peptide"
LQHDNFPLT

SEQ ID NO: 1080 - protein, 9 aa, synthetic construct
  REGION 1..9: note = source = /note="Description of Artificial Sequence: Synthetic peptide"
QHYYRFPYS

SEQ ID NO: 1081 - protein, 9 aa, synthetic construct
  REGION 1..9: note = source = /note="Description of Artificial Sequence: Synthetic peptide"
MQALQTPYT

SEQ ID NO: 1082 - protein, 11 aa, synthetic construct
  REGION 1..11: note = source = /note="Description of Artificial Sequence: Synthetic peptide"
QVWDSDSEHVV

SEQ ID NO: 1083 - protein, 9 aa, synthetic construct
  REGION 1..9: note = source = /note="Description of Artificial Sequence: Synthetic peptide"
QAWDDTTVV

SEQ ID NO: 1084 - protein, 9 aa, synthetic construct
  REGION 1..9: note = source = /note="Description of Artificial Sequence: Synthetic peptide"
QKYNSAPFT

SEQ ID NO: 1085 - protein, 12 aa, synthetic construct
  REGION 1..12: note = source = /note="Description of Artificial Sequence: Synthetic peptide"
SSRDSSGDHLRV

SEQ ID NO: 1086 - protein, 11 aa, synthetic construct
  REGION 1..11: note = source = /note="Description of Artificial Sequence: Synthetic peptide"
NSRDSSGHHLL

SEQ ID NO: 1087 - protein, 14 aa, synthetic construct
  REGION 1..14: note = source = /note="Description of Artificial Sequence: Synthetic peptide"
QHYGSSFNGSSLFT

SEQ ID NO: 1088 - protein, 10 aa, synthetic construct
  REGION 1..10: note = source = /note="Description of Artificial Sequence: Synthetic peptide"
QQFGTSSGLT

SEQ ID NO: 1089 - protein, 10 aa, synthetic construct
  REGION 1..10: note = source = /note="Description of Artificial Sequence: Synthetic peptide"
QQYHSSPSWT

SEQ ID NO: 1090 - protein, 10 aa, synthetic construct
  REGION 1..10: note = source = /note="Description of Artificial Sequence: Synthetic peptide"
QQYHSSPSWT

SEQ ID NO: 1091 - protein, 9 aa, synthetic construct
  REGION 1..9: note = source = /note="Description of Artificial Sequence: Synthetic peptide"
QQSYSTPYS

SEQ ID NO: 1092 - protein, 10 aa, synthetic construct
  REGION 1..10: note = source = /note="Description of Artificial Sequence: Synthetic peptide"
QHYESSPSWT

SEQ ID NO: 1093 - protein, 10 aa, synthetic construct
  REGION 1..10: note = source = /note="Description of Artificial Sequence: Synthetic peptide"
QQYGSPPRFT

SEQ ID NO: 1094 - protein, 10 aa, synthetic construct
  REGION 1..10: note = source = /note="Description of Artificial Sequence: Synthetic peptide"
QHYGSSPSWT

SEQ ID NO: 1095 - protein, 10 aa, synthetic construct
  REGION 1..10: note = source = /note="Description of Artificial Sequence: Synthetic peptide"
QHYDSSPSWT

SEQ ID NO: 1096 - protein, 10 aa, synthetic construct
  REGION 1..10: note = source = /note="Description of Artificial Sequence: Synthetic peptide"
QYYGTSPMYT

SEQ ID NO: 1097 - protein, 9 aa, synthetic construct
  REGION 1..9: note = source = /note="Description of Artificial Sequence: Synthetic peptide"
MQALQTPLT

SEQ ID NO: 1098 - protein, 11 aa, synthetic construct
  REGION 1..11: note = source = /note="Description of Artificial Sequence: Synthetic peptide"
QHYGNSPPKFT

SEQ ID NO: 1099 - protein, 10 aa, synthetic construct
  REGION 1..10: note = source = /note="Description of Artificial Sequence: Synthetic peptide"
QHYGGSPRLT

SEQ ID NO: 1100 - protein, 9 aa, synthetic construct
  REGION 1..9: note = source = /note="Description of Artificial Sequence: Synthetic peptide"
QQHYSTPWT

SEQ ID NO: 1101 - protein, 9 aa, synthetic construct
  REGION 1..9: note = source = /note="Description of Artificial Sequence: Synthetic peptide"
LQSRIFPRT

SEQ ID NO: 1102 - protein, 9 aa, synthetic construct
  REGION 1..9: note = source = /note="Description of Artificial Sequence: Synthetic peptide"
LQSRTIPRT

SEQ ID NO: 1103 - protein, 9 aa, synthetic construct
  REGION 1..9: note = source = /note="Description of Artificial Sequence: Synthetic peptide"
LQSRTIPRT

SEQ ID NO: 1104-1117 - skipped (14 entries)

SEQ ID NO: 1118 - protein, 121 aa, synthetic construct
  REGION 1..121: note = source = /note="Description of Artificial Sequence: Synthetic polypeptide"
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 1119 - protein, 108 aa, synthetic construct
  REGION 1..108: note = source = /note="Description of Artificial Sequence: Synthetic polypeptide"
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 1120 - protein, 451 aa, synthetic construct
  VARIANT 451: note = /replace=" "
  REGION 1..451: note = MISC_FEATURE - /note="Variant residues given in the sequence have no preference with respect to those in the annotations for variant positions"
  REGION 1..451: note = source = /note="Description of Artificial Sequence: Synthetic polypeptide"
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1121 - protein, 214 aa, synthetic construct
  REGION 1..214: note = source = /note="Description of Artificial Sequence: Synthetic polypeptide"
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1122 - protein, 486 aa, synthetic construct
  VARIANT 486: note = /replace=" "
  REGION 1..486: note = MISC_FEATURE - /note="Variant residues given in the sequence have no preference with respect to those in the annotations for variant positions"
  REGION 1..486: note = source = /note="Description of Artificial Sequence: Synthetic polypeptide"
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1123 - protein, 119 aa, synthetic construct
  REGION 1..119: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 1124 - protein, 109 aa, synthetic construct
  REGION 1..109: note = source = /note="Description of Artificial Sequence: Synthetic polypeptide"
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIK

SEQ ID NO: 1125 - protein, 449 aa, synthetic construct
  VARIANT 449: note = /replace=" "
  REGION 1..449: note = MISC_FEATURE - /note="Variant residues given in the sequence have no preference with respect to those in the annotations for variant positions"
  REGION 1..449: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1126 - protein, 216 aa, synthetic construct
  REGION 1..216: note = source = /note="Description of Artificial Sequence: Synthetic polypeptide"
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1127 - protein, 117 aa, synthetic construct; identical to SEQ ID NO: 256
  REGION 1..117: note = source = /note="Description of Artificial Sequence: Synthetic polypeptide"

SEQ ID NO: 1128 - protein, 111 aa, synthetic construct
  REGION 1..111: note = source = /note="Description of Artificial Sequence: Synthetic polypeptide"
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 1129 - protein, 447 aa, synthetic construct
  VARIANT 447: note = /replace=" "
  REGION 1..447: note = MISC_FEATURE - /note="Variant residues given in the sequence have no preference with respect to those in the annotations for variant positions"
  REGION 1..447: note = source = /note="Description of Artificial Sequence: Synthetic polypeptide"
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1130 - protein, 218 aa, synthetic construct
  REGION 1..218: note = source = /note="Description of Artificial Sequence: Synthetic polypeptide"
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1131 - protein, 116 aa, synthetic construct
  REGION 1..116: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 1132 - protein, 109 aa, synthetic construct
  REGION 1..109: note = source = /note="Description of Artificial Sequence: Synthetic polypeptide"
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 1133 - protein, 477 aa, synthetic construct
  VARIANT 477: note = /replace=" "
  REGION 1..477: note = MISC_FEATURE - /note="Variant residues given in the sequence have no preference with respect to those in the annotations for variant positions"
  REGION 1..477: note = source = /note="Description of Artificial Sequence: Synthetic polypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIKGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1134 - protein, 486 aa, synthetic construct
  VARIANT 486: note = /replace=" "
  REGION 1..486: note = MISC_FEATURE - /note="Variant residues given in the sequence have no preference with respect to those in the annotations for variant positions"
  REGION 1..486: note = source = /note="Description of Artificial Sequence: Synthetic polypeptide"
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1135 - protein, 494 aa, synthetic construct
  VARIANT 494: note = /replace=" "
  REGION 1..494: note = MISC_FEATURE - /note="Variant residues given in the sequence have no preference with respect to those in the annotations for variant positions"
  REGION 1..494: note = source = /note="Description of Artificial Sequence: Synthetic polypeptide"
EVQLVESGGGLVKPGGSLRLSCAASGFTFSPYSVFWVRQAPGKGLEWVSSINTDSTYKYYADSVKGRFTISRDNAENSIFLQMNSLRAEDTAVYYCARDRSYYAFSSGSLSDYYYGLDVWGQGTLVTVSSGGGGSGGGGSGGGGSGGGGSDIVMTQSPLSLSVTPGEPASISCRSSQSLLHTNLYNYLDWYVQKPGQSPQLLIYLASNRASGVPDRFSGSGSGTDFTLKISRVETEDVGVYYCMQALQIPRTFGQGTKLEIKGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1136 - protein, 486 aa, synthetic construct
  VARIANT 486: note = /replace=" "
  REGION 1..486: note = MISC_FEATURE - /note="Variant residues given in the sequence have no preference with respect to those in the annotations for variant positions"
  REGION 1..486: note = source = /note="Description of Artificial Sequence: Synthetic polypeptide"
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1137 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 1138 - protein, 266 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 1139 - protein, 164 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGFIY

SEQ ID NO: 1140 - protein, 218 aa, Homo sapiens
QVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 1141 - protein, 152 aa, Homo sapiens [sequence omitted]

SEQ ID NO: 1142 - protein, 250 aa, Homo sapiens [sequence omitted]

SEQ ID NO: 1143 - protein, 234 aa, Homo sapiens
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKNDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 1144 - protein, 247 aa, Homo sapiens
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGL

SEQ ID NO: 1145 - protein, 222 aa, Homo sapiens
MPASSPFLLAPKGPPGNMGGPVREPALSVALWLSWGAALGAVACAMALLTQQTELQSLRREVSRLQGTGGPSQNGEGYPWQSLPEQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 1146 - protein, 205 aa, Homo sapiens
MGGPVREPALSVALWLSWGAALGAVACAMALLTQQTELQSLRREVSRLQGTGGPSQNGEGYPWQSLPEQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 1147 - protein, 146 aa, Homo sapiens
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 1148 - skipped

SEQ ID NO: 1149 - protein, 329 aa, synthetic construct
  REGION 1..329: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 1150 - protein, 329 aa, synthetic construct
  REGION 1..329: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 1151 - protein, 231 aa, synthetic construct
  REGION 1..231: note = source = /note="Description of Artificial Sequence: Synthetic polypeptide"
GGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 1152 - protein, 231 aa, synthetic construct
  REGION 1..231: note = source = /note="Description of Artificial Sequence: Synthetic polypeptide"
GGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 1153 - protein, 215 aa, synthetic construct
  REGION 1..215: note = source = /note="Description of Artificial Sequence: Synthetic polypeptide"
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1154 - protein, 449 aa, synthetic construct
  VARIANT 449: note = /replace=" "
  REGION 1..449: note = MISC_FEATURE - /note="Variant residues given in the sequence have no preference with respect to those in the annotations for variant positions"
  REGION 1..449: note = source = /note="Description of Artificial Sequence: Synthetic polypeptide"
EVQLVESGGGLVKPGGSLRLSCAASGFTFSNSGMIWVRQAPGKGLEWVGHIRSKTDGGTTDYAAPVKGRFTISRDDSKNTLYLQMNSLKTEDTAVYYCTTGGSGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 1155 - protein, 688 aa, synthetic construct
  VARIANT 688: note = /replace=" "
  REGION 1..688: note = MISC_FEATURE - /note="Variant residues given in the sequence have no preference with respect to those in the annotations for variant positions"
  REGION 1..688: note = source = /note="Description of Artificial Sequence: Synthetic polypeptide"
EVQLVESGGGLVKPGGSLRLSCAASGFTFSNSGMIWVRQAPGKGLEWVGHIRSKTDGGTTDYAAPVKGRFTISRDDSKNTLYLQMNSLKTEDTAVYYCTTGGSGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1156 - protein, 30 aa, synthetic construct
  REGION 1..30: note = MISC_FEATURE - /note="This sequence may encompass 1-6 'Gly Gly Gly Gly Ser' repeating units"
  REGION 1..30: note = source = /note="Description of Artificial Sequence: Synthetic polypeptide"
  REGION 1..30: note = source = /note="See specification as filed for detailed description of substitutions and preferred embodiments"
GGGGSGGGGSGGGGSGGGGSGGGGSGGGGS

SEQ ID NO: 1157 - protein, 219 aa, synthetic construct
  REGION 1..219: note = source = /note="Description of Artificial Sequence: Synthetic polypeptide"
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1158 - protein, 215 aa, synthetic construct
  REGION 1..215: note = source = /note="Description of Artificial Sequence: Synthetic polypeptide"
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1159 - protein, 441 aa, synthetic construct
  REGION 1..441: note = source = /note="Description of Artificial Sequence: Synthetic polypeptide"
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDISVEWESNGQPENNYKTTPPMLDSDGSFFLYSKLTVDKSRWQQGNVFSCSVMHEALHNHYTQKSLSLSPGK

SEQ ID NO: 1160 - protein, 447 aa, synthetic construct
  REGION 1..447: note = source = /note="Description of Artificial Sequence: Synthetic polypeptide"
EVQLVESGGGLVQPGGSLRLSCAASGFTFSDYYMTWVRQAPGKGLEWVAFIRNRARGYTSDHNASVKGRFTISRDNAKNSLYLQMNSLRAEDTAVYYCARDRPSYYVLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK